SEQUENCE LISTING (45 sequences)

SEQ ID NO: 1 - protein, 89 aa, Homo sapiens
LPAPKNLVVSEVTEDSLRLSWTAPDAAFDSFLIQYQESEKVGEAINLTVPGSERSYDLTGLKPGTEYTVSIYGVKGGHRSNPLSAEFTT

SEQ ID NO: 2 - protein, 594 aa, Artificial Sequence
  piggybac transposase
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 3 - protein, 21 aa, Homo sapiens
MALPVTALLLPLALLLHAARP

SEQ ID NO: 4 - protein, 24 aa, Homo sapiens
IYIWAPLAGTCGVLLLSLVITLYC

SEQ ID NO: 5 - DNA, 72 bp, Homo sapiens
atctacatttgggcaccactggccgggacctgtggagtgctgctgctgagcctggtcatcacactgtactgc

SEQ ID NO: 6 - protein, 112 aa, Homo sapiens
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - DNA, 336 bp, Homo sapiens
cgcgtgaagtttagtcgatcagcagatgccccagcttacaaacagggacagaaccagctgtataacgagctgaatctgggccgccgagaggaatatgacgtgctggataagcggagaggacgcgaccccgaaatgggaggcaagcccaggcgcaaaaaccctcaggaaggcctgtataacgagctgcagaaggacaaaatggcagaagcctattctgagatcggcatgaagggggagcgacggagaggcaaagggcacgatgggctgtaccagggactgagcaccgccacaaaggacacctatgatgctctgcatatgcaggcactgcctccaagg

SEQ ID NO: 8 - protein, 42 aa, Homo sapiens
KRGRKKLLYIFKQPFMRPVQTTQEEDGCSCRFPEEEEGGCEL

SEQ ID NO: 9 - DNA, 126 bp, Homo sapiens
aagagaggcaggaagaaactgctgtatattttcaaacagcccttcatgcgccccgtgcagactacccaggaggaagacgggtgctcctgtcgattccctgaggaagaggaaggcgggtgtgagctg

SEQ ID NO: 10 - protein, 45 aa, Homo sapiens
TTTPAPRPPTPAPTIASQPLSLRPEACRPAAGGAVHTRGLDFACD

SEQ ID NO: 11 - DNA, 135 bp, Homo sapiens
actaccacaccagcacctagaccaccaactccagctccaaccatcgcgagtcagcccctgagtctgagacctgaggcctgcaggccagctgcaggaggagctgtgcacaccaggggcctggacttcgcctgcgac

SEQ ID NO: 12 - protein, 594 aa, Trichoplusia ni
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGYKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 13 - protein, 90 aa, Homo sapiens
MLPAPKNLVVSEVTEDSLRLSWTAPDAAFDSFLIQYQESEKVGEAINLTVPGSERSYDLTGLKPGTEYTVSIYGVKGGHRSNPLSAEFTT

SEQ ID NO: 14 - DNA, 270 bp, Artificial Sequence
  FN3 domain consensus sequence
atgctgcctgcaccaaagaacctggtggtgtctcatgtgacagaggatagtgccagactgtcatggactgctcccgacgcagccttcgatagttttatcatcgtgtaccgggagaacatcgaaaccggcgaggccattgtcctgacagtgccagggtccgaacgctcttatgacctgacagatctgaagcccggaactgagtactatgtgcagatcgccggcgtcaaaggaggcaatatcagcttccctctgtccgcaatcttcaccaca

SEQ ID NO: 15 - protein, 4 aa, Artificial Sequence
  modification of FN3 concensus sequence
TEDS

SEQ ID NO: 16 - protein, 7 aa, Artificial Sequence
  modification of FN3 domain consensus
TAPDAAF

SEQ ID NO: 17 - protein, 6 aa, Artificial Sequence
  modification of FN3 domain concensus
SEKVGE

SEQ ID NO: 18 - protein, 4 aa, Artificial Sequence
  modification of FN3 domain consensus
GSER

SEQ ID NO: 19 - protein, 5 aa, Artificial Sequence
  modification of FN3 domain consensus
GLKPG

SEQ ID NO: 20 - protein, 7 aa, Artificial Sequence
  modification of FN3 domain consensus
KGGHRSN

SEQ ID NO: 21 - protein, 958 aa, Artificial Sequence
  CARTyrin
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGSGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSGSGEGRGSLLTCGDVEENPGPMALPVTALLLPLALLLHAARPMLPAPKNLVVSRITEDSARLSWTAPDAAFDSFPIRYIETLIWGEAIWLDVPGSERSYDLTGLKPGTEYAVVITGVKGGRFSSPLVASFTTTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 22 - DNA, 2874 bp, Artificial Sequence
  CARTyrin
atgggggtccaggtcgagactatttcaccaggggatgggcgaacatttccaaaaaggggccagacttgcgtcgtgcattacaccgggatgctggaggacgggaagaaagtggacagctccagggatcgcaacaagcccttcaagttcatgctgggaaagcaggaagtgatccgaggatgggaggaaggcgtggcacagatgtcagtcggccagcgggccaaactgaccattagccctgactacgcttatggagcaacaggccacccagggatcattccccctcatgccaccctggtcttcgatgtggaactgctgaagctggagggaggaggaggatccggatttggggacgtgggggccctggagtctctgcgaggaaatgccgatctggcttacatcctgagcatggaaccctgcggccactgtctgatcattaacaatgtgaacttctgcagagaaagcggactgcgaacacggactggctccaatattgactgtgagaagctgcggagaaggttctctagtctgcactttatggtcgaagtgaaaggggatctgaccgccaagaaaatggtgctggccctgctggagctggctcagcaggaccatggagctctggattgctgcgtggtcgtgatcctgtcccacgggtgccaggcttctcatctgcagttccccggagcagtgtacggaacagacggctgtcctgtcagcgtggagaagatcgtcaacatcttcaacggcacttcttgccctagtctggggggaaagccaaaactgttctttatccaggcctgtggcggggaacagaaagatcacggcttcgaggtggccagcaccagccctgaggacgaatcaccagggagcaaccctgaaccagatgcaactccattccaggagggactgaggacctttgaccagctggatgctatctcaagcctgcccactcctagtgacattttcgtgtcttacagtaccttcccaggctttgtctcatggcgcgatcccaagtcagggagctggtacgtggagacactggacgacatctttgaacagtgggcccattcagaggacctgcagagcctgctgctgcgagtggcaaacgctgtctctgtgaagggcatctacaaacagatgcccgggtgcttcaattttctgagaaagaaactgttctttaagacttccggatctggagagggaaggggaagcctgctgacctgtggagacgtggaggaaaacccaggaccaatggcactgccagtcaccgccctgctgctgcctctggctctgctgctgcacgcagctagaccaatgctgcctgcaccaaagaacctggtggtgagccggatcacagaggactccgccagactgtcttggaccgcccctgacgccgccttcgattcctttccaatccggtacatcgagacactgatctggggcgaggccatctggctggacgtgcccggctctgagaggagctacgatctgacaggcctgaagcctggcaccgagtatgcagtggtcatcacaggagtgaagggcggcaggttcagctcccctctggtggcctcttttaccacaaccacaacccctgcccccagacctcccacacccgcccctaccatcgcgagtcagcccctgagtctgagacctgaggcctgcaggccagctgcaggaggagctgtgcacaccaggggcctggacttcgcctgcgacatctacatttgggcaccactggccgggacctgtggagtgctgctgctgagcctggtcatcacactgtactgcaagagaggcaggaagaaactgctgtatattttcaaacagcccttcatgcgccccgtgcagactacccaggaggaagacgggtgctcctgtcgattccctgaggaagaggaaggcgggtgtgagctgcgcgtgaagtttagtcgatcagcagatgccccagcttacaaacagggacagaaccagctgtataacgagctgaatctgggccgccgagaggaatatgacgtgctggataagcggagaggacgcgaccccgaaatgggaggcaagcccaggcgcaaaaaccctcaggaaggcctgtataacgagctgcagaaggacaaaatggcagaagcctattctgagatcggcatgaagggggagcgacggagaggcaaagggcacgatgggctgtaccagggactgagcaccgccacaaaggacacctatgatgctctgcatatgcaggcactgcctccaaggggaagtggagaaggacgaggatcactgctgacatgcggcgacgtggaggaaaaccctggcccaatggtcgggtctctgaattgtatcgtcgccgtgagtcagaacatgggcattgggaagaatggcgatttcccatggccacctctgcgcaacgagtcccgatactttcagcggatgacaactacctcctctgtggaagggaaacagaatctggtcatcatgggaaagaaaacttggttcagcattccagagaagaaccggcccctgaaaggcagaatcaatctggtgctgtcccgagaactgaaggagccaccacagggagctcactttctgagccggtccctggacgatgcactgaagctgacagaacagcctgagctggccaacaaagtcgatatggtgtggatcgtcgggggaagttcagtgtataaggaggccatgaatcaccccggccatctgaaactgttcgtcacacggatcatgcaggactttgagagcgatactttctttcctgaaattgacctggagaagtacaaactgctgcccgaatatcctggcgtgctgtccgatgtccaggaagagaaaggcatcaaatacaagttcgaggtctatgagaagaatgac

SEQ ID NO: 23 - protein, 107 aa, Artificial Sequence
  FKBP12 polypeptide
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 24 - DNA, 321 bp, Artificial Sequence
  FKBP12 polypeptide
ggggtccaggtcgagactatttcaccaggggatgggcgaacatttccaaaaaggggccagacttgcgtcgtgcattacaccgggatgctggaggacgggaagaaagtggacagctccagggatcgcaacaagcccttcaagttcatgctgggaaagcaggaagtgatccgaggatgggaggaaggcgtggcacagatgtcagtcggccagcgggccaaactgaccattagccctgactacgcttatggagcaacaggccacccagggatcattccccctcatgccaccctggtcttcgatgtggaactgctgaagctggag

SEQ ID NO: 25 - protein, 5 aa, Artificial Sequence
  linker
GGGGS

SEQ ID NO: 26 - DNA, 15 bp, Artificial Sequence
  linker
ggaggaggaggatcc

SEQ ID NO: 27 - protein, 281 aa, Artificial Sequence
  truncated caspase 9
GFGDVGALESLRGNADLAYISLMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCNFLRKKLFFKTS

SEQ ID NO: 28 - DNA, 843 bp, Artificial Sequence
  truncated caspase 9
tttggggacgtgggggccctggagtctctgcgaggaaatgccgatctggcttacatcctgagcatggaaccctgcggccactgtctgatcattaacaatgtgaacttctgcagagaaagcggactgcgaacacggactggctccaatattgactgtgagaagctgcggagaaggttctctagtctgcactttatggtcgaagtgaaaggggatctgaccgccaagaaaatggtgctggccctgctggagctggctcagcaggaccatggagctctggattgctgcgtggtcgtgatcctgtcccacgggtgccaggcttctcatctgcagttccccggagcagtgtacggaacagacggctgtcctgtcagcgtggagaagatcgtcaacatcttcaacggcacttcttgccctagtctggggggaaagccaaaactgttctttatccaggcctgtggcggggaacagaaagatcacggcttcgaggtggccagcaccagccctgaggacgaatcaccagggagcaaccctgaaccagatgcaactccattccaggagggactgaggacctttgaccagctggatgctatctcaagcctgcccactcctagtgacattttcgtgtcttacagtaccttcccaggctttgtctcatggcgcgatcccaagtcagggagctggtacgtggagacactggacgacatctttgaacagtgggcccattcagaggacctgcagagcctgctgctgcgagtggcaaacgctgtctctgtgaagggcatctacaaacagatgcccgggtgcttcaattttctgagaaagaaactgttctttaagacttcc

SEQ ID NO: 29 - protein, 394 aa, Artificial Sequence
  inducible proapoptotic polypeptide
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGGSGFGDVGALESLRGNADLAYISLMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCNFLRKKLFFKTS

SEQ ID NO: 30 - DNA, 1182 bp, Artificial Sequence
  inducible proapoptotic polypeptide
ggggtccaggtcgagactatttcaccaggggatgggcgaacatttccaaaaaggggccagacttgcgtcgtgcattacaccgggatgctggaggacgggaagaaagtggacagctccagggatcgcaacaagcccttcaagttcatgctgggaaagcaggaagtgatccgaggatgggaggaaggcgtggcacagatgtcagtcggccagcgggccaaactgaccattagccctgactacgcttatggagcaacaggccacccagggatcattccccctcatgccaccctggtcttcgatgtggaactgctgaagctggagggaggaggaggatccgaatttggggacgtgggggccctggagtctctgcgaggaaatgccgatctggcttacatcctgagcatggaaccctgcggccactgtctgatcattaacaatgtgaacttctgcagagaaagcggactgcgaacacggactggctccaatattgactgtgagaagctgcggagaaggttctctagtctgcactttatggtcgaagtgaaaggggatctgaccgccaagaaaatggtgctggccctgctggagctggctcagcaggaccatggagctctggattgctgcgtggtcgtgatcctgtcccacgggtgccaggcttctcatctgcagttccccggagcagtgtacggaacagacggctgtcctgtcagcgtggagaagatcgtcaacatcttcaacggcacttcttgccctagtctggggggaaagccaaaactgttctttatccaggcctgtggcggggaacagaaagatcacggcttcgaggtggccagcaccagccctgaggacgaatcaccagggagcaaccctgaaccagatgcaactccattccaggagggactgaggacctttgaccagctggatgctatctcaagcctgcccactcctagtgacattttcgtgtcttacagtaccttcccaggctttgtctcatggcgcgatcccaagtcagggagctggtacgtggagacactggacgacatctttgaacagtgggcccattcagaggacctgcagagcctgctgctgcgagtggcaaacgctgtctctgtgaagggcatctacaaacagatgcccgggtgcttcaattttctgagaaagaaactgttctttaagacttcc

SEQ ID NO: 31 - protein, 18 aa, Thosea asigna virus
EGRGSLLTCGDVEENPGP

SEQ ID NO: 32 - protein, 21 aa, Artificial Sequence
  GSG-T2A
GSGEGRGSLLTCGDVEENPGP

SEQ ID NO: 33 - DNA, 63 bp, Artificial Sequence
  GSG-T2A
ggatctggagagggaaggggaagcctgctgacctgtggagacgtggaggaaaacccaggacca

SEQ ID NO: 34 - protein, 19 aa, Equine rhinitis A
QCTNYALLKLAGDVESNPG

SEQ ID NO: 35 - protein, 22 aa, Artificial Sequence
  GSG-G2A
SGQCTNYALLKLAGDVESNPGP

SEQ ID NO: 36 - protein, 22 aa, Foot-and-mouth disease virus
VKQTLNFDLLKLAGDVESNPGP

SEQ ID NO: 37 - protein, 25 aa, Artificial Sequence
  GSG-F2A
GSGVKQTLNFDLLKLAGDVESNPGP

SEQ ID NO: 38 - protein, 19 aa, Porcine Teschovirus
ATNFSLLKQAGDVEENPGP

SEQ ID NO: 39 - protein, 22 aa, Artificial Sequence
  GSG-P2A
GSGATNFSLLKQAGDVEENPGP

SEQ ID NO: 40 - DNA, 4897 bp, Artificial Sequence
  PB-EF1a vector
tgtacatagattaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaaccatggacagttagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagaattctaatacgactcactatagggtgtgctgtctcatcattttggcaaagattggccaccaagcttgtcctgcaggagggtcgacgcctctagacgggcggccgctccggatccacgggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgcctattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgagattatcaaaaaggatcttcacctagatccttttaaattaaaaatgaagttttaaatcaatctaaagtatatatgagtaaacttggtctgacagtcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggttcgtctcgtcccggtctcctcccaatgcatgtcaatattggccattagccatattattcattggttatatagcataaatcaatattggctattggccattgcatacgttgtatctatatcataata

SEQ ID NO: 41 - protein, 90 aa, Artificial Sequence
  Centyrin
MLPAPKNLVVSRITEDSARLSWTAPDAAFDSFPIRYIETLIWGEAIWLDVPGSERSYDLTGLKPGTEYAVVITGVKGGRFSSPLVASFTT

SEQ ID NO: 42 - protein, 334 aa, Artificial Sequence
  CARTyrin
MALPVTALLLPLALLLHAARPMLPAPKNLVVSRITEDSARLSWTAPDAAFDSFPIRYIETLIWGEAIWLDVPGSERSYDLTGLKPGTEYAVVITGVKGGRFSSPLVASFTTTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 90 aa, Artificial Sequence; identical to SEQ ID NO: 41
  Centyrin

SEQ ID NO: 44 - DNA, 1002 bp, Artificial Sequence
  CARTyrin
atggcactgccagtcaccgccctgctgctgcctctggctctgctgctgcacgcagctagaccaatgctgcctgcaccaaagaacctggtggtgagccggatcacagaggactccgccagactgtcttggaccgcccctgacgccgccttcgattcctttccaatccggtacatcgagacactgatctggggcgaggccatctggctggacgtgcccggctctgagaggagctacgatctgacaggcctgaagcctggcaccgagtatgcagtggtcatcacaggagtgaagggcggcaggttcagctcccctctggtggcctcttttaccacaaccacaacccctgcccccagacctcccacacccgcccctaccatcgcgagtcagcccctgagtctgagacctgaggcctgcaggccagctgcaggaggagctgtgcacaccaggggcctggacttcgcctgcgacatctacatttgggcaccactggccgggacctgtggagtgctgctgctgagcctggtcatcacactgtactgcaagagaggcaggaagaaactgctgtatattttcaaacagcccttcatgcgccccgtgcagactacccaggaggaagacgggtgctcctgtcgattccctgaggaagaggaaggcgggtgtgagctgcgcgtgaagtttagtcgatcagcagatgccccagcttacaaacagggacagaaccagctgtataacgagctgaatctgggccgccgagaggaatatgacgtgctggataagcggagaggacgcgaccccgaaatgggaggcaagcccaggcgcaaaaaccctcaggaaggcctgtataacgagctgcagaaggacaaaatggcagaagcctattctgagatcggcatgaagggggagcgacggagaggcaaagggcacgatgggctgtaccagggactgagcaccgccacaaaggacacctatgatgctctgcatatgcaggcactgcctccaagg

SEQ ID NO: 45 - DNA, 63 bp, Homo sapiens
atggcactgccagtcaccgccctgctgctgcctctggctctgctgctgcacgcagctagacca

(19) United States
(12) Patent Application Publication
(10) Pub. No.: US 2023/0190811 A1
(43) Pub. Date: Jun. 22, 2023

(54) COMPOSITIONS AND METHODS FOR USE IN THE TREATMENT OF CANCER

(71) Applicant: Poseida Therapeutics, Inc., San Diego, CA (US)

(72) Inventors: Eric M. OSTERTAG, San Diego, CA (US); Matthew SPEAR, San Diego, CA (US)

(21) Appl. No.: 17/996,021

(22) PCT Filed: Apr. 13, 2021
(86) PCT No.: PCT/US2021/027152

§ 371 Date: Oct. 12, 2022

Related U.S. Application Data
(60) Provisional application No. 63/009,569, filed on Apr. 14, 2020.

(51) Int. Cl. A61K 35/17; C07K 16/28; A61P 35/00

(52) U.S. Cl. CPC A61K 35/17; C07K 16/2887; A61P 35/00

(57) ABSTRACT

Disclosed are chimeric antigen receptors (CARs) comprising Centyrins (i.e. CARTyrins), transposons encoding CARs and CARTyrins of the disclosure, cells modified to express CARs and CARTyrins of the disclosure, as well as methods of making and methods of using same for adoptive cell therapy.

### CROSS-REFERENCE TO RELATED APPLICATIONS

This application claims priority to, and the benefit of, U.S. Provisional Application No. 63/009,569, filed Apr. 14, 2020. The contents of this application are incorporated herein by reference in their entirety.

### FIELD OF THE DISCLOSURE

The disclosure is directed to molecular biology, and more, specifically, to chimeric antigen receptors, transposons containing one or more CARs, as well as methods of making and using the same.

### INCORPORATION OF SEQUENCE LISTING

The contents of the text file named “POTH-057_001WO_SequenceListing.txt”, which was created on Apr. 13, 2021 and is 54.6 KB in size, are hereby incorporated by reference in their entirety.

### BACKGROUND

There has been a long-felt but unmet need in the art for a method of directing the specificity of an immune cell without using traditional antibody sequences or fragments thereof. The disclosure provides a superior chimeric antigen receptor.

### SUMMARY

The present disclosure provides a method of treating cancer comprising administering to the subject: a first composition comprising a population of T-cells expressing a chimeric antigen receptor (CAR), wherein the CAR comprises an antigen recognition domain that specifically binds to B-cell maturation antigen (BCMA); and a second composition comprising an anti-CD20 agent. In some embodiments, the method further comprises a third composition comprising at least one lymphodepletion agent. In some embodiments, the anti-CD20 agent is rituximab, ofatumumab, ocrelizumab, iodine i131 tositumomab, obinutuzumab or ibritumomab. In a preferred embodiment, the anti-CD20 agent is rituximab. In some embodiments, the antigen recognition domain comprises a Centyrin, an scFv, a single domain antibody, a VH or a VHH. In some embodiments, the antigen recognition domain comprises a Centyrin. In some embodiments, the antigen recognition domain comprises a VH.

The present disclosure also provides a method of treating cancer comprising administering to the subject: a first composition comprising a population of T-cells expressing a chimeric antigen receptor (CAR), wherein the CAR comprises an antigen recognition domain and a second composition comprising an anti-CD20 agent. In some embodiments, the first composition comprises a first composition comprising a population of T-cells expressing more than one CAR. In some embodiments, each CAR in the more than one CAR binds to a different antigen. In some embodiments, the method further comprises a third composition comprising at least one lymphodepletion agent. In some embodiments, the anti-CD20 agent is rituximab, ofatumumab, ocrelizumab, iodine i131 tositumomab, obinutuzumab or ibritumomab. In a preferred embodiment, the anti-CD20 agent is rituximab. In some embodiments, the antigen recognition domain comprises a Centyrin, an scFv, a single domain antibody, a VH or a VHH. In some embodiments, the Centyrin specifically binds B-cell maturation antigen (BCMA). In some embodiments, the VH specifically binds BCMA. In some embodiments, the Centyrin specifically binds Prostate-specific membrane antigen (PSMA). In some embodiments, the scFv binds Mucin 1 (MUC-1). In some embodiments, the scFv binds MUC1-C.

In some embodiments, the method provides an at least 50% decrease in anti-drug antibody (ADA) response against the first composition in the patient in comparison to a patient that is administered with the first composition but is not administered with the second composition.

In some embodiments, the method provides an at least 75% increase in persistence of the first composition in the patient in comparison to a patient that is administered with the first composition but is not administered with the second composition. In some embodiments, the method provides an at least 90% increase in persistence of the first composition in the patient in comparison to a patient that is administered with the first composition but is not administered with the second composition. In some embodiments, the measure of persistence is the area under the curve (AUC) of a plasma concentration curve.

In some embodiments, the first composition is administered as multiple infusions, wherein the multiple infusion comprises a total dose that is split into a first infusion and a second infusion, and wherein i) the first infusion comprises about one-third of the total dose; and ii) the second infusion comprises about two-thirds of the total dose and is administered at least 10 days after the first infusion.

In some embodiments, the first composition is administered as multiple infusions, wherein the multiple infusion comprises a total dose that is split into a first infusion and a second infusion, and wherein i) the first infusion comprises about two-thirds of the total dose; and ii) the second infusion comprises about one-third of the total dose and is administered at least 10 days after the first infusion.

In some embodiments, the first composition is administered as multiple infusions, wherein the multiple infusion comprises a total dose that is split into a first infusion, a second infusion and a third infusion, and wherein i) the first infusion comprises about one-third of the total dose; ii) the second infusion comprises about one-third of the total dose and is administered at least 10 days after the first infusion; and iii) the third infusion comprises about one-third of the total dose and is administered at least 10 days after the second infusion.

In some embodiments, the time in between the first infusion and the second infusion or the time in between the second infusion and the third infusion is at least 1 week, 2 weeks, 3 weeks, 4 weeks, 5 weeks or 6 weeks.

In some embodiments, the first composition, the second composition and the third composition are administered sequentially. In some embodiments, the first composition, the second composition and/or the third composition are administered concurrently. In some embodiments, the third composition is administered prior to the first composition.

In some embodiments, the third composition is administered in more than one dose. In some embodiments, the third composition is administered once a day and wherein the first dose of the third composition is administered at least 5 days prior to the first infusion of the first composition. In some embodiments, the third composition is administered 3 days, 4 days and 5 days prior to the first infusion of the first composition.

In some embodiments, the second composition is administered prior to the first composition. In some embodiments, the second composition is administered in more than one dose. In some embodiments, a first dose of the second composition is administered at 12 days prior to the first infusion of the first composition, wherein a second dose of the second composition is administered at 5 days prior to the first infusion of the first composition, and wherein subsequent doses are administered once per week after the first infusion of the first composition for at least 8 weeks.

In some embodiments, the subject has not previously been treated with an anti-cancer agent. In some embodiments, the subject does not receive an anti-cancer agent i) within 2 weeks prior to the administration of a first dose of the second composition; or ii) within 5 half-lives of the anti-cancer agent prior to the administration of a first dose of the second composition.

In some embodiments, the subject does not receive an anti-cancer agent following the administration of the first infusion of the first composition. In some embodiments, the subject has not previously been treated with an immunosuppressive agent. In some embodiments, the subject does not receive an immunosuppressive agent i) within 2 weeks prior to the administration of a first dose of the second composition; or ii) within 5 half-lives of the immunosuppressive agent prior to the administration of a first dose of the second composition. In some embodiments, the subject does not receive an immunosuppressive agent following the administration of the first infusion of the first composition.

In some embodiments, the subject has not previously been treated with an anti-inflammatory agent. In some embodiments, the subject does not receive an anti-inflammatory agent i) within 2 weeks prior to the administration of a first dose of the second composition; ii) within 1 week prior to the administration of a first dose of the second composition; or ii) within 5 half-lives of the immunosuppressive agent prior to the administration of a first dose of the second composition. In some embodiments, the subject does not receive an anti-inflammatory agent following the administration of the first infusion of the first composition. In some embodiments, the anti-inflammatory agent is a corticosteroid. In some embodiments, the corticosteroid is prednisone and wherein the prednisone is administered systemically at a dose of at least 5 mg/day.

In some embodiments, the subject has not previously been treated with a granulocyte-colony stimulating factor (G-CSF) or a granulocyte-macrophage colony-stimulating factor (GM-CSF). In some embodiments, the subject does not receive a G-CSF or a GM-CSF i) within 2 weeks prior to the administration of a first dose of the second composition; or ii) within 5 half-lives of the G-CSF or of the GM-CSF prior to the administration of a first dose of the second composition. In some embodiments, the subject does not receive a G-CSF or a GM-CSF following the administration of the last infusion of the first composition and/or within 2 months after the administration of the last infusion of the first composition.

In some embodiments, the subject has not previously been treated with a herbal medication. In some embodiments, the subject does not receive a herbal medication within 2 weeks prior to the administration of a first dose of the second composition. In some embodiments, the subject does not receive a herbal medication following the administration of the last infusion of the first composition and/or within 2 months after the administration of the last infusion of the first composition.

In some embodiments, a first lymphodepletion agent of the third composition and a second lymphodepletion agent of the third composition is administered concurrently. In some embodiments, a first lymphodepletion agent of the third composition and a second lymphodepletion agent of the third composition are administered sequentially. In some embodiments, the first lymphodepletion agent and the second lymphodepletion agent are administered on the same day, wherein the first lymphodepletion agent is administered intravenously over a 30 minute time period and wherein the second lymphodepletion agent is administered intravenously over a 30 minute time period.

In some embodiments, the first lymphodepletion agent or the second lymphodepletion agent is cyclophosphamide or fludarabine. In some embodiments, a dose of the third composition comprises i) 100 mg/m², 200 mg/m², 300 mg/m², 400 mg/m² or 500 mg/m² of cyclophosphamide; ii) 10 mg/m², 20 mg/m², 30 mg/m², 40 mg/m² or 50 mg/m² of fludarabine; or a combination thereof. In some embodiments, the dose of the third composition comprises 300 mg/m² of cyclophosphamide and 30 mg/m² of fludarabine.

In some embodiments, the first composition is administered at a total dose of at least 0.1×10⁶, 0.2×10⁶, 0.25×10⁶, 0.5×10⁶, 0.6×10⁶, 0.7×10⁶, 0.75×10⁶, 0.8×10⁶, 0.9×10⁶, 1×10⁶, 2×10⁶, 3×10⁶, 4×10⁶, 5×10⁶, 6×10⁶, 7×10⁶, 8×10⁶, 9×10⁶, 10×10⁶, 11×10⁶, 12×10⁶, 13×10⁶, 14×10⁶, 15×10⁶, 16×10⁶, 17×10⁶, 18×10⁶, 19×10⁶ or 20×10⁶ cells/kg of the subject's body weight. In some embodiments, the first infusion, the second infusion and/or the third infusion of the first composition is administered using a infusion bag that comprises the first composition at a concentration of about 1×10⁵ cells/mL to about 5×10⁷ cells/mL. In some embodiments, the infusion bag comprises the first composition at a concentration of about 3×10⁵ cells/mL to about 2.4×10⁷ cells/mL.

In some embodiments, the first infusion, the second infusion and/or the third infusion is administered by intravenous infusion at a flow rate of about 0.5 mL/min to about 30 mL/min. In some embodiments, the flow rate is about 1 mL/min to about 20 mL/min. In some embodiments, the total duration of the first infusion, the second infusion and/or the third infusion is about 5 mins to about 30 mins.

In some embodiments, a dose of the second composition comprises 100 mg/m², 125 mg/m², 150 mg/m², 175 mg/m², 200 mg/m², 225 mg/m², 275 mg/m², 300 mg/m², 325 mg/m², 375 mg/m² 400 mg/m², 425 mg/m², 450 mg/m², 475 mg/m² or 500 mg/m² of rituximab. In a preferred embodiment, the dose of the second composition is 375 mg/m² of rituximab.

In some embodiments, the second composition is administered by intravenous infusion and wherein the flow rate of the intravenous infusion is about 25 mg/hr to about 500 mg/hr. In some embodiments, the first dose of the second composition is administered by intravenous infusion at a flow rate of 50 mg/hr and wherein the flow rate is increased every 30 minutes to a maximum of 400 mg/hr. In some embodiments, the second dose and the subsequent dose of the second composition is administered by intravenous infusion at a flow rate of about 100 mg/hr and wherein the flow rate is increased every 30 minutes to a maximum of about 400 mg/hr.

In some embodiments, an acetaminophen, an antihistamine and methylprednisolone are administered 30 minutes prior to each dose of the second composition.

In some embodiments, the cancer is a hematological cancer. In some embodiments, the cancer is multiple myeloma. In some embodiments, the multiple myeloma is relapsed multiple myeloma or refractory multiple myeloma.

In some embodiments, the cancer is a solid cancer. In some embodiments, the cancer is a prostate cancer. In some embodiments, the cancer is a castrate-resistant prostate cancer (CRPC). In some embodiments, the solid cancer is a breast cancer, a colorectal cancer, a lung cancer, an ovarian cancer, a pancreatic cancer or a renal cancer. In some embodiments, the breast cancer is triple negative breast cancer.

The present disclosure provides a unit dose infusion bag comprising 250 mL of a composition comprising a population of T-cells expressing a CAR, wherein the CAR comprises an antigen recognition domain comprising a Centyrin that specifically binds to BCMA, wherein the concentration of the composition is about 3×10⁵ cells/mL to about 2.4×10⁷ cells/mL.

The present disclosure provides a unit dose infusion bag comprising 250 mL of a composition comprising a population of T-cells expressing a CAR, wherein the CAR comprises an antigen recognition domain comprising a VH that specifically binds to BCMA, wherein the concentration of the composition is about 3×10⁵ cells/mL to about 2.4×10⁷ cells/mL.

The present disclosure provides a unit dose infusion bag comprising 250 mL of a composition comprising a population of T-cells expressing a CAR, wherein the CAR comprises an antigen recognition domain comprising a Centyrin that specifically binds to PSMA, wherein the concentration of the composition is about 3×10⁵ cells/mL to about 2.4×10⁷ cells/mL.

The present disclosure provides a unit dose infusion bag comprising 250 mL of a composition comprising a population of T-cells expressing a CAR, wherein the CAR comprises an antigen recognition domain comprising a scFv that specifically binds to MUC1-C, wherein the concentration of the composition is about 3×10⁵ cells/mL to about 2.4×10⁷ cells/mL.

### BRIEF DESCRIPTION OF THE DRAWINGS

FIG. 1 is a schematic diagram depicting a piggyBac CARTyrin construct of the disclosure of 7676 base pairs that includes a transposon comprising a CARTyrin (comprising a CD8a signal peptide, a Centyrin, a CD8a hinge sequence, and a transmembrane sequence, and a CD3z costimulatory domain).

FIG. 2 is a schematic diagram of the amino acid sequence of a P-BCMA-101 construct of the disclosure.

FIGS. 3A-3B is a schematic diagram of the nucleic acid sequence of a P-BCMA-101 construct of the disclosure.

FIG. 4 is a schematic diagram depicting the construction of a CARTyrin of the disclosure and a table contrasting characteristics of Centyrins and antibodies.

FIG. 5A is a series of cell sorting plots depicting CARTyrin expression following electroporation with 5 μg of CARTyrin mRNA.

FIG. 5B, left panel, is a series of cell sorting plots depicting CARTyrin function following challenge with the control K562 cell line and the BCMA expressing H929 cell line and, right panel, a graph showing a quantification of the plots of the left panel (with the addition of data from challenge with the BCMA-expressing line U266).

FIG. 5C is a graph depicting CARTyrin activity as a function of amount of mRNA used during electroporation of T-cells.

FIG. 6 is a schematic diagram depicting an in vivo tumor challenge study timeline using the A08 CARTyrin in mice.

FIG. 7 is a pair of graphs showing a complete (100%) survival of A08 CARTyrin-treated mice. Tumor burden was assessed by presence of M-protein. There was no detectable M-protein in protected animals.

FIG. 8 is a schematic diagram depicting an exemplary inducible truncated caspase 9 polypeptide of the disclosure.

FIG. 9 is a series of flow cytometry plots depicting the abundance of cells moving from an area of live cells (the gated lower right quadrant) to an area populated by apoptotic cells (the upper left quadrant) as a function of increasing dosage of the induction agent (AP1903) in cells modified to express a therapeutic agent (a CARTyrin) alone or in combination with an inducible caspase polypeptide of the disclosure (encoded by an iC9 construct (also known as a “safety switch”) introduced into cells by a piggyBac (PB) transposase) at day 12 post nucleofection.

FIG. 10 is a series of flow cytometry plots depicting the abundance of cells moving from an area of live cells (the gated lower right quadrant) to an area populated by apoptotic cells (the upper left quadrant) as a function of increasing dosage of the induction agent (AP1903) in cells modified to express a therapeutic agent (a CARTyrin) alone or in combination with an inducible caspase polypeptide of the disclosure (encoded by an iC9 construct (also known as a “safety switch”) introduced into cells by a piggyBac (PB) transposase) at day 19 post nucleofection.

FIG. 11 is a pair of graphs depicting a quantification of the aggregated results shown either in FIG. 9 (left graph) or FIG. 10 (right graph). Specifically, these graphs show the impact of the iC9 safety switch on the percent cell viability as a function of the concentration of the induction agent (AP1903) of the iC9 switch for each modified cell type at either day 12 (FIG. 9 and left graph) or day 19 (FIG. 10 and right graph).

FIGS. 12A-D are graphs showing the stable expression and function of BCMA CARTyrin. FIG. 12A is a flow cytometry plot depicting CARTyrin surface expression on P-BCMA-101 following piggyBac (PB) transposition. Mock represents without primary BCMA/Fc/Biotin. FIG. 12B is a flow cytometry plot showing the increase of CARTyrin expression on re-stimulated P-BCMA-101 T cells. FIG. 12C is a graph showing in vitro killing by P-BCMA-101 cells against BCMA+ (H929) cell lines. FIG. 12D are flow cytometry plots showing the proliferation of P-BCMA-101 cells against BCMA-expressing cell line.

FIGS. 13A-D are line graphs showing the in vivo tumor growth and survival of MM.1S-Luc tumor-bearing NSG mice treated with P-BCMA-101 in GLP Safety Study. Female NSG mice were grafted IV with M.1S BCMA+MM cells and dosed IV 17-19 days later with P-BCMA-101 with either vehicle (n=10) (black); 4×10⁶ P-BCMA-101 cells (low dose) (n=19) (red) or 12×10⁶ P-BCMA-101 cells (high dose) (green). No tumor (blue) (n=20). At Day 29, 10 mice from each treatment group were euthanized and submitted for pathology. FIG. 13A is a graph showing the mean±Standard Error of the Mean (SEM) bioluminescent imaging data. FIG. 13B is a graph showing bioluminescent imaging data of each individual mice from each treatment group. FIG. 13C is a survival curve showing the percent survival of mice from the three treatment groups. FIG. 13D is a graph showing the body weight change of mice from each treatment group.

FIG. 14 is a schematic diagram of the overall study design for P-BCMA-101 Single Administration

FIG. 15 is a schematic diagram of the P-BCMA-101 clinical manufacturing and process plan

FIG. 16 is a schematic diagram of the overall study design for P-BCMA-101 Cycle Administration in Cohort A and Cohort C. During Phase 1—Cycle Administration, multiple doses of P-BCMA-101 will be administered intravenously in 2 cycles of 2 weeks.

FIG. 17 is a schematic diagram of the overall study design for P-BCMA-101 Cycle Administration in Cohort B. During Phase 1—Cycle administration, multiple doses of P-BCMA-101 will be administered intravenously in 3 cycles of 2 weeks.

FIG. 18 is a schematic diagram of the overall study design for P-BCMA-101 Phase 1 Combination Administration. In Phase 1—Combination Administration, P-BCMA-101 will be administered in combination with approved therapies, lenalidomide and rituximab.

FIG. 19A is a graph showing the number of copies/ug DNA of P-BCMA-101 over time in multiple myeloma patients treated with P-BCMA-101 alone.

FIG. 19B is a graph showing the number of copies/ug DNA of P-BCMA-101 over time in multiple myeloma patients treated with a combination of P-BCMA-101 and rituximab.

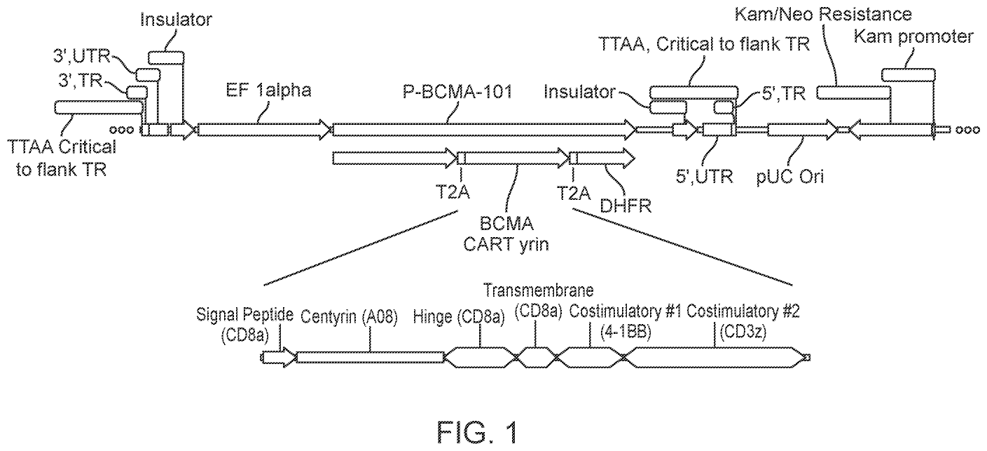

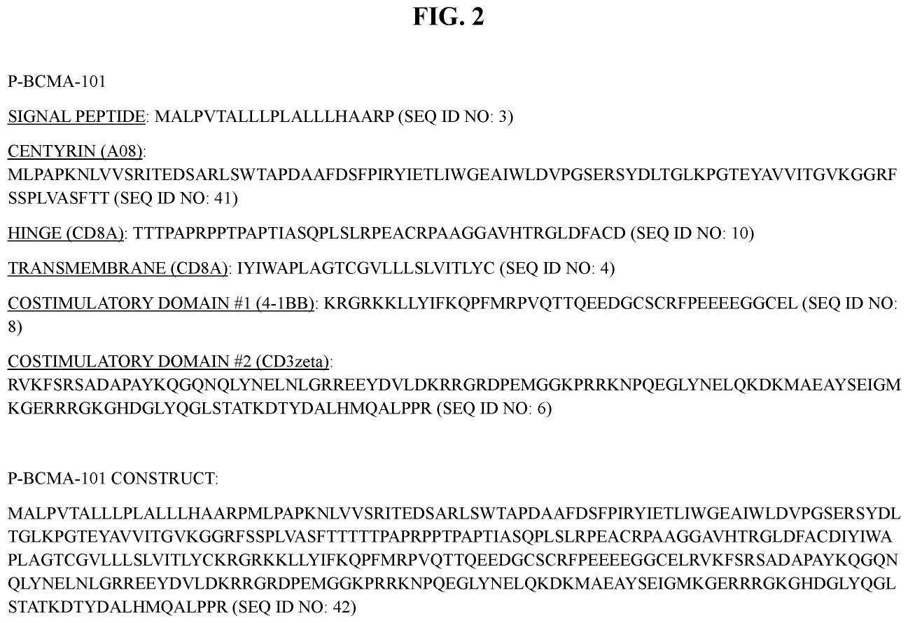

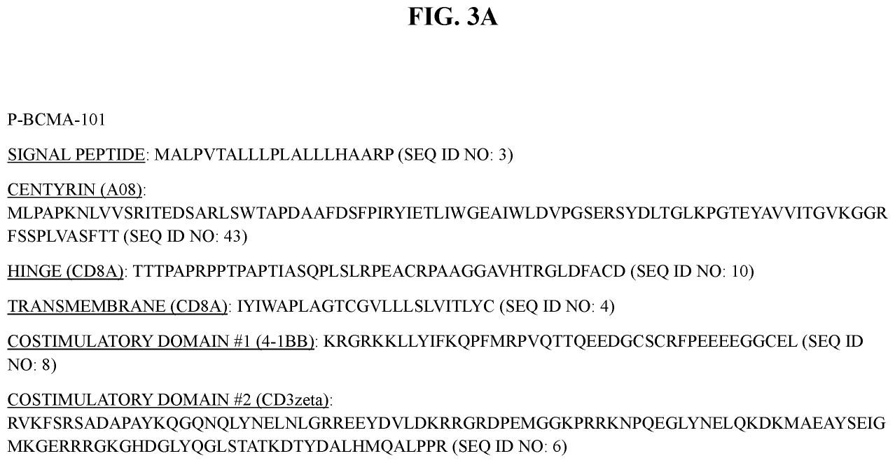

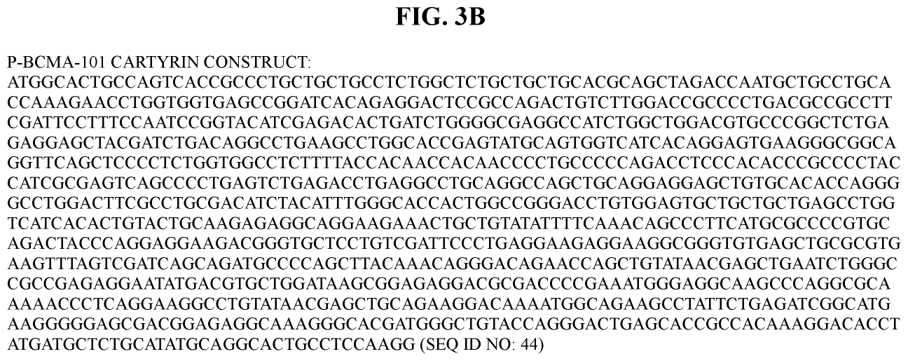

### DETAILED DESCRIPTION

The present disclosure provides a method of treating cancer comprising administering to the subject: a first composition comprising a population of T-cells expressing a chimeric antigen receptor (CAR), wherein the CAR comprises an antigen recognition domain and a second composition comprising an anti-CD20 agent.

In some embodiments, the antigen recognition domain of the CAR comprises a Centyrin, an scFv, a single domain antibody, a VH or a VHH.

In some embodiments, the antigen recognition domain specifically binds to B-cell maturation antigen (BCMA) or Tumor necrosis factor receptor superfamily member 17 (TNFRSF17). In some embodiments, the antigen recognition domain comprises a Centyrin specifically binds BCMA. Non-limiting examples of Centyrins that specifically bind BCMA are disclosed in PCT Publication No. WO2018/014038. In some embodiments, the Centyrin that specifically binds BCMA comprises an amino acid sequence of SEQ ID NO: 41.

In some embodiments, the antigen recognition domain comprises a VH specifically binds BCMA. Non-limiting examples of VHs that specifically bind BCMA are disclosed in PCT Publication No. WO2019/126574.

In some embodiments, the antigen recognition domain specifically binds to Prostate-specific membrane antigen (PSMA). In some embodiments, the antigen recognition domain comprises a Centyrin specifically binds PSMA. Non-limiting examples of Centyrins that specifically bind PSMA are disclosed in PCT Publication No. WO2019/173636.

In some embodiments, the antigen recognition domain specifically binds to Mucin 1 (MUC-1). Human MUC1 is heterodimeric glycoprotein, translated as a single polypeptide and cleaved into N- and C-terminal subunits (MUC1-N and MUC1-C) in the endoplasmic reticulum. In some embodiments, the antigen recognition domain comprises an scFv that specifically binds to MUC1-C. Non-limiting examples of Centyrins that specifically bind MUC1-C are disclosed in PCT Application No. PCT/US2020/066121 and PCT Publication No. WO2018/014039.

Centyrins of the disclosure specifically bind to an antigen. Chimeric antigen receptors of the disclosure comprising one or more Centyrins that specifically bind an antigen may be used to direct the specificity of a cell, (e.g. a cytotoxic immune cell) towards the specific antigen.

Centyrins of the disclosure may comprise a consensus sequence comprising

(SEQ ID NO: 1)
LPAPKNLVVSEVTEDSLRLSWTAPDAAFDSFLIQY
QESEKVGEAINLTVPGSERSYDLTGLKPGTEYTVS
IYGVKGGHRSNPLSAEFTT.

Chimeric antigen receptors of the disclosure may comprise a signal peptide of human CD2, CD3δ, CD3ε, CD3γ, CD3ζ, CD4, CD8α, CD19, CD28, 4-1BB or GM-CSFR. A hinge/spacer domain of the disclosure may comprise a hinge/spacer/stalk of human CD8α, IgG4, and/or CD4. An intracellular domain or endodomain of the disclosure may comprise an intracellular signaling domain of human CD3ζ and may further comprise human 4-1BB, CD28, CD40, ICOS, MyD88, OX-40 intracellular segment, or any combination thereof. Exemplary transmembrane domains include, but are not limited to a human CD2, CD3δ, CD3ε, CD3γ, CD3ζ, CD4, CD8α, CD19, CD28, 4-1BB or GM-CSFR transmembrane domain.

As used herein, the term “P-BCMA-101” refers to a Centyrin that binds to BCMA, CARTyrin that binds to BCMA, a CAR that specifically binds to BCMA, or a T-cell or population of T-cells that express the CARTyrin or CAR that specifically binds to BCMA. In some instances “P-BCMA-101” refers to the construct encoding the CAR or CARTyrin that binds to BCMA (e.g. including surrounding elements such as DHFR and iC9).

In some embodiments, the P-BCMA-101 CARTyrin comprises human CD8a signal peptide comprising an amino acid sequence of SEQ ID NO: 3; an antigen recognition region comprising a Centyrin that specifically binds BCMA comprising an amino acid sequence of SEQ ID NO: 41; a human CD8a hinge region comprising an amino acid sequence of SEQ ID NO: 10; a human CD8a transmembrane region comprising an amino acid sequence of SEQ ID NO: 4; a human 4-1BB costimulatory domain comprising an amino acid sequence of SEQ ID NO: 8; and a CD3zeta costimulatory domain comprising an amino acid sequence of SEQ ID NO: 6. In some embodiments, the P-BCMA-101 CARTyrin comprises an amino acid sequence of SEQ ID NO: 42. In some embodiments, the P-BCMA-101 CARTyrin is encoded by a polynucleotide comprising the nucleic acid sequence of SEQ ID NO: 44.

The disclosure provides genetically modified cells, such as T cells, NK cells, hematopoietic progenitor cells, peripheral blood (PB) derived T cells (including T cells from G-CSF-mobilized peripheral blood), umbilical cord blood (UCB) derived T cells rendered specific for one or more antigens by introducing to these cells a CAR and/or CARTyrin of the disclosure. Cells of the disclosure may be modified by electrotransfer of a transposon encoding a CAR or CARTyrin of the disclosure and a plasmid comprising a sequence encoding a transposase of the disclosure (preferably, the sequence encoding a transposase of the disclosure is an mRNA sequence). Examples of transposons encoding CARTyrins are described in PCT/US2019/021224, incorporated herein by reference in entirety. Examples of transposons encoding CARs are described in PCT/US2018/066936 and PCT/US2017/042457, each incorporated herein in entirety.

Transposons of the disclosure be episomally maintained or integrated into the genome of the recombinant/modified cell. The transposon may be part of a two component piggyBac system that utilizes a transposon and transposase for enhanced non-viral gene transfer. In certain embodiments of this method, the transposon is a plasmid DNA transposon with a sequence encoding the chimeric antigen receptor flanked by two cis-regulatory insulator elements. In certain embodiments, the transposon is a piggyBac transposon. In certain embodiments, and, in particular, those embodiments wherein the transposon is a piggyBac transposon, the transposase is a piggyBac™ or a Super piggyBac™ (SPB) transposase.

In certain embodiments of the methods of the disclosure, the transposon is a plasmid DNA transposon with a sequence encoding the antigen receptor flanked by two cis-regulatory insulator elements. In certain embodiments, the transposon is a piggyBac transposon. In certain embodiments, and, in particular, those embodiments wherein the transposon is a piggyBac transposon, the transposase is a piggyBac™ or a Super piggyBac™ (SPB) transposase. In certain embodiments, and, in particular, those embodiments wherein the transposase is a Super piggyBac™ (SPB) transposase, the sequence encoding the transposase is an mRNA sequence.

In certain embodiments of the methods of the disclosure, the transposase enzyme is a piggyBac™ (PB) transposase enzyme. The piggyBac (PB) transposase enzyme may comprise or consist of an amino acid sequence at least 75%, 80%, 85%, 90%, 95%, 99% or any percentage in between identical to:

(SEQ ID NO: 12)

| | |
|---|---|
| 1 | MGSSLDDEHI LSALLQSDDE LVGEDSDSEI |
| | SDHVSEDDVQ SDTEEAFIDE VHEVQPTSSG |
| 61 | SEILDEQNVI EQPGSSLASN RILTLPQRTI |
| | RGKNKHCWST SKSTRRSRVS ALNIVRSQRG |
| 121 | PTRMCRNIYD PLLCFKLFFT DEIISEIVKW |
| | TNAEISLKRR ESMTGATFRD TNEDEIYAFF |
| 181 | GILVMTAVRK DNHMSTDDLF DRSLSMVYVS |
| | VMSRDRFDFL IRCLRMDDKS IRPTLRENDV |
| 241 | FTPVRKIWDL FIHQCIQNYT PGAHLTIDEQ |
| | LLGFRGRCPF RMYIPNKPSK YGIKILMMCD |
| 301 | SGYKYMINGM PYLGRGTQTN GVPLGEYYVK |
| | ELSKPVHGSC RNITCDNWFT SIPLAKNLLQ |
| 361 | EPYKLTIVGT VRSNKREIPE VLKNSRSRPV |
| | GTSMFCFDGP LTLVSYKPKP AKMVYLLSSC |
| 421 | DEDASINEST GKPQMVMYYN QTKGGVDTLD |
| | QMCSVMTCSR KTNRWPMALL YGMINIACIN |
| 481 | SFIIYSHNVS SKGEKVQSRK KFMRNLYMSL |
| | TSSFMRKRLE APTLKRYLRD NISNILPNEV |
| 541 | PGTSDDSTEE PVMKKRTYCT YCPSKIRRKA |
| | NASCKKCKKV ICREHNIDMC QSCF. |

In certain embodiments of the methods of the disclosure, the transposase enzyme is a piggyBac™ (PB) transposase enzyme that comprises or consists of an amino acid sequence having an amino acid substitution at one or more of positions 30, 165, 282, or 538 of the sequence:

(SEQ ID NO: 12)

| | |
|---|---|
| 1 | MGSSLDDEHI LSALLQSDDE LVGEDSDSEI |
| | SDHVSEDDVQ SDTEEAFIDE VHEVQPTSSG |
| 61 | SEILDEQNVI EQPGSSLASN RILTLPQRTI |
| | RGKNKHCWST SKSTRRSRVS ALNIVRSQRG |
| 121 | PTRMCRNIYD PLLCFKLFFT DEIISEIVKW |
| | TNAEISLKRR ESMTGATFRD TNEDEIYAFF |
| 181 | GILVMTAVRK DNHMSTDDLF DRSLSMVYVS |
| | VMSRDRFDFL IRCLRMDDKS IRPTLRENDV |
| 241 | FTPVRKIWDL FIHQCIQNYT PGAHLTIDEQ |
| | LLGFRGRCPF RMYIPNKPSK YGIKILMMCD |
| 301 | SGYKYMINGM PYLGRGTQTN GVPLGEYYVK |
| | ELSKPVHGSC RNITCDNWFT SIPLAKNLLQ |
| 361 | EPYKLTIVGT VRSNKREIPE VLKNSRSRPV |
| | GTSMFCFDGP LTLVSYKPKP AKMVYLLSSC |
| 421 | DEDASINEST GKPQMVMYYN QTKGGVDTLD |
| | QMCSVMTCSR KTNRWPMALL YGMINIACIN |
| 481 | SFIIYSHNVS SKGEKVQSRK KFMRNLYMSL |
| | TSSFMRKRLE APTLKRYLRD NISNILPNEV |
| 541 | PGTSDDSTEE PVMKKRTYCT YCPSKIRRKA |
| | NASCKKCKKV ICREHNIDMC QSCF. |

In certain embodiments, the transposase enzyme is a piggyBac™ (PB) transposase enzyme that comprises or consists of an amino acid sequence having an amino acid substitution at two or more of positions 30, 165, 282, or 538 of the sequence of SEQ ID NO: 12. In certain embodiments, the transposase enzyme is a piggyBac™ (PB) transposase enzyme that comprises or consists of an amino acid sequence having an amino acid substitution at three or more of positions 30, 165, 282, or 538 of the sequence of SEQ ID NO: 12. In certain embodiments, the transposase enzyme is a piggyBac™ (PB) transposase enzyme that comprises or consists of an amino acid sequence having an amino acid substitution at each of the following positions 30, 165, 282, and 538 of the sequence of SEQ ID NO: 12. In certain embodiments, the amino acid substitution at position 30 of the sequence of SEQ ID NO: 12 is a substitution of a valine (V) for an isoleucine (I). In certain embodiments, the amino acid substitution at position 165 of the sequence of SEQ ID NO: 12 is a substitution of a serine (S) for a glycine (G). In certain embodiments, the amino acid substitution at position 282 of the sequence of SEQ ID NO: 12 is a substitution of a valine (V) for a methionine (M). In certain embodiments, the amino acid substitution at position 538 of the sequence of SEQ ID NO: 12 is a substitution of a lysine (K) for an asparagine (N).

In certain embodiments of the methods of the disclosure, the transposase enzyme is a Super piggyBac™ (sPBo) transposase enzyme. In certain embodiments, the Super piggyBac™ (sPBo) transposase enzymes of the disclosure may comprise or consist of the amino acid sequence of the sequence of SEQ ID NO: 12 wherein the amino acid substitution at position 30 is a substitution of a valine (V) for an isoleucine (I), the amino acid substitution at position 165 is a substitution of a serine (S) for a glycine (G), the amino acid substitution at position 282 is a substitution of a valine (V) for a methionine (M), and the amino acid substitution at position 538 is a substitution of a lysine (K) for an asparagine (N). In certain embodiments, the Super piggyBac™ (sPBo) transposase enzyme may comprise or consist of an amino acid sequence at least 75%, 80%, 85%, 90%, 95%, 99% or any percentage in between identical to:

(SEQ ID NO: 2)

| | |
|---|---|
| 1 | MGSSLDDEHI LSALLQSDDE LVGEDSDSEV |
| | SDHVSEDDVQ SDTEEAFIDE VHEVQPTSSG |
| 61 | SEILDEQNVI EQPGSSLASN RILTLPQRTI |
| | RGKNKHCWST SKSTRRSRVS ALNIVRSQRG |
| 121 | PTRMCRNIYD PLLCFKLFFT DEIISEIVKW |
| | TNAEISLKRR ESMTSATFRD TNEDEIYAFF |
| 181 | GILVMTAVRK DNHMSTDDLF DRSLSMVYVS |
| | VMSRDRFDFL IRCLRMDDKS IRPTLRENDV |
| 241 | FTPVRKIWDL FIHQCIQNYT PGAHLTIDEQ |
| | LLGFRGRCPF RVYIPNKPSK YGIKILMMCD |
| 301 | SGTKYMINGM PYLGRGTQTN GVPLGEYYVK |
| | ELSKPVHGSC RNITCDNWFT SIPLAKNLLQ |
| 361 | EPYKLTIVGT VRSNKREIPE VLKNSRSRPV |
| | GTSMFCFDGP LTLVSYKPKP AKMVYLLSSC |
| 421 | DEDASINEST GKPQMVMYYN QTKGGVDTLD |
| | QMCSVMTCSR KTNRWPMALL YGMINIACIN |
| 481 | SFIIYSHNVS SKGEKVQSRK KFMRNLYMSL |
| | TSSFMRKRLE APTLKRYLRD NISNILPKEV |
| 541 | PGTSDDSTEE PVMKKRTYCT YCPSKIRRKA |
| | NASCKKCKKV ICREHNIDMC QSCF. |

In certain embodiments of the methods of the disclosure, including those embodiments wherein the transposase comprises the above-described mutations at positions 30, 165, 282 and/or 538, the piggyBac™ or Super piggyBac™ transposase enzyme may further comprise an amino acid substitution at one or more of positions 3, 46, 82, 103, 119, 125, 177, 180, 185, 187, 200, 207, 209, 226, 235, 240, 241, 243, 258, 296, 298, 311, 315, 319, 327, 328, 340, 421, 436, 456, 470, 486, 503, 552, 570 and 591 of the sequence of SEQ ID NO: 12 or SEQ ID NO: 2. In certain embodiments, including those embodiments wherein the transposase comprises the above-described mutations at positions 30, 165, 282 and/or 538, the piggyBac™ or Super piggyBac™ transposase enzyme may further comprise an amino acid substitution at one or more of positions 46, 119, 125, 177, 180, 185, 187, 200, 207, 209, 226, 235, 240, 241, 243, 296, 298, 311, 315, 319, 327, 328, 340, 421, 436, 456, 470, 485, 503, 552 and 570. In certain embodiments, the amino acid substitution at position 3 of SEQ ID NO: 12 or SEQ ID NO: 2 is a substitution of an asparagine (N) for a serine (S). In certain embodiments, the amino acid substitution at position 46 of SEQ ID NO: 12 or SEQ ID NO: 2 is a substitution of a serine (S) for an alanine (A). In certain embodiments, the amino acid substitution at position 46 of SEQ ID NO: 12 or SEQ ID NO: 2 is a substitution of a threonine (T) for an alanine (A). In certain embodiments, the amino acid substitution at position 82 of SEQ ID NO: 12 or SEQ ID NO: 2 is a substitution of a tryptophan (W) for an isoleucine (I). In certain embodiments, the amino acid substitution at position 103 of SEQ ID NO: 12 or SEQ ID NO: 2 is a substitution of a proline (P) for a serine (S). In certain embodiments, the amino acid substitution at position 119 of SEQ ID NO: 12 or SEQ ID NO: 2 is a substitution of a proline (P) for an arginine (R). In certain embodiments, the amino acid substitution at position 125 of SEQ ID NO: 12 or SEQ ID NO: 2 is a substitution of an alanine (A) a cysteine (C). In certain embodiments, the amino acid substitution at position 125 of SEQ ID NO: 12 or SEQ ID NO: 2 is a substitution of a leucine (L) for a cysteine (C). In certain embodiments, the amino acid substitution at position 177 of SEQ ID NO: 12 or SEQ ID NO: 2 is a substitution of a lysine (K) for a tyrosine (Y). In certain embodiments, the amino acid substitution at position 177 of SEQ ID NO: 12 or SEQ ID NO: 2 is a substitution of a histidine (H) for a tyrosine (Y). In certain embodiments, the amino acid substitution at position 180 of SEQ ID NO: 12 or SEQ ID NO: 2 is a substitution of a leucine (L) for a phenylalanine (F). In certain embodiments, the amino acid substitution at position 180 of SEQ ID NO: 12 or SEQ ID NO: 2 is a substitution of an isoleucine (I) for a phenylalanine (F). In certain embodiments, the amino acid substitution at position 180 of SEQ ID NO: 12 or SEQ ID NO: 2 is a substitution of a valine (V) for a phenylalanine (F). In certain embodiments, the amino acid substitution at position 185 of SEQ ID NO: 12 or SEQ ID NO: 2 is a substitution of a leucine (L) for a methionine (M). In certain embodiments, the amino acid substitution at position 187 of SEQ ID NO: 12 or SEQ ID NO: 2 is a substitution of a glycine (G) for an alanine (A). In certain embodiments, the amino acid substitution at position 200 of SEQ ID NO: 12 or SEQ ID NO: 2 is a substitution of a tryptophan (W) for a phenylalanine (F). In certain embodiments, the amino acid substitution at position 207 of SEQ ID NO: 12 or SEQ ID NO: 2 is a substitution of a proline (P) for a valine (V). In certain embodiments, the amino acid substitution at position 209 of SEQ ID NO: 12 or SEQ ID NO: 2 is a substitution of a phenylalanine (F) for a valine (V). In certain embodiments, the amino acid substitution at position 226 of SEQ ID NO: 12 or SEQ ID NO: 2 is a substitution of a phenylalanine (F) for a methionine (M). In certain embodiments, the amino acid substitution at position 235 of SEQ ID NO: 12 or SEQ ID NO: 2 is a substitution of an arginine (R) for a leucine (L). In certain embodiments, the amino acid substitution at position 240 of SEQ ID NO: 12 or SEQ ID NO: 12 is a substitution of a lysine (K) for a valine (V). In certain embodiments, the amino acid substitution at position 241 of SEQ ID NO: 12 or SEQ ID NO: 2 is a substitution of a leucine (L) for a phenylalanine (F). In certain embodiments, the amino acid substitution at position 243 of SEQ ID NO: 12 or SEQ ID NO: 2 is a substitution of a lysine (K) for a proline (P). In certain embodiments, the amino acid substitution at position 258 of SEQ ID NO: 12 or SEQ ID NO: 2 is a substitution of a serine (S) for an asparagine (N). In certain embodiments, the amino acid substitution at position 296 of SEQ ID NO: 12 or SEQ ID NO: 2 is a substitution of a tryptophan (W) for a leucine (L). In certain embodiments, the amino acid substitution at position 296 of SEQ ID NO: 12 or SEQ ID NO: 2 is a substitution of a tyrosine (Y) for a leucine (L). In certain embodiments, the amino acid substitution at position 296 of SEQ ID NO: 12 or SEQ ID NO: 2 is a substitution of a phenylalanine (F) for a leucine (L). In certain embodiments, the amino acid substitution at position 298 of SEQ ID NO: 12 or SEQ ID NO: 2 is a substitution of a leucine (L) for a methionine (M). In certain embodiments, the amino acid substitution at position 298 of SEQ ID NO: 12 or SEQ ID NO: 2 is a substitution of an alanine (A) for a methionine (M). In certain embodiments, the amino acid substitution at position 298 of SEQ ID NO: 12 or SEQ ID NO: 2 is a substitution of a valine (V) for a methionine (M). In certain embodiments, the amino acid substitution at position 311 of SEQ ID NO: 12 or SEQ ID NO: 2 is a substitution of an isoleucine (I) for a proline (P). In certain embodiments, the amino acid substitution at position 311 of SEQ ID NO: 12 or SEQ ID NO: 2 is a substitution of a valine for a proline (P). In certain embodiments, the amino acid substitution at position 315 of SEQ ID NO: 12 or SEQ ID NO: 2 is a substitution of a lysine (K) for an arginine (R). In certain embodiments, the amino acid substitution at position 319 of SEQ ID NO: 12 or SEQ ID NO: 2 is a substitution of a glycine (G) for a threonine (T). In certain embodiments, the amino acid substitution at position 327 of SEQ ID NO: 12 or SEQ ID NO: 2 is a substitution of an arginine (R) for a tyrosine (Y). In certain embodiments, the amino acid substitution at position 328 of SEQ ID NO: 12 or SEQ ID NO: 2 is a substitution of a valine (V) for a tyrosine (Y). In certain embodiments, the amino acid substitution at position 340 of SEQ ID NO: 12 or SEQ ID NO: 2 is a substitution of a glycine (G) for a cysteine (C). In certain embodiments, the amino acid substitution at position 340 of SEQ ID NO: 12 or SEQ ID NO: 2 is a substitution of a leucine (L) for a cysteine (C). In certain embodiments, the amino acid substitution at position 421 of SEQ ID NO: 12 or SEQ ID NO: 2 is a substitution of a histidine (H) for the aspartic acid (D). In certain embodiments, the amino acid substitution at position 436 of SEQ ID NO: 12 or SEQ ID NO: 2 is a substitution of an isoleucine (I) for a valine (V). In certain embodiments, the amino acid substitution at position 456 of SEQ ID NO: 12 or SEQ ID NO: 2 is a substitution of a tyrosine (Y) for a methionine (M). In certain embodiments, the amino acid substitution at position 470 of SEQ ID NO: 12 or SEQ ID NO: 2 is a substitution of a phenylalanine (F) for a leucine (L). In certain embodiments, the amino acid substitution at position 485 of SEQ ID NO: 12 or SEQ ID NO: 2 is a substitution of a lysine (K) for a serine (S). In certain embodiments, the amino acid substitution at position 503 of SEQ ID NO: 12 or SEQ ID NO: 2 is a substitution of a leucine (L) for a methionine (M). In certain embodiments, the amino acid substitution at position 503 of SEQ ID NO: 12 or SEQ ID NO: 2 is a substitution of an isoleucine (I) for a methionine (M). In certain embodiments, the amino acid substitution at position 552 of SEQ ID NO: 12 or SEQ ID NO: 2 is a substitution of a lysine (K) for a valine (V). In certain embodiments, the amino acid substitution at position 570 of SEQ ID NO: 12 or SEQ ID NO: 2 is a substitution of a threonine (T) for an alanine (A). In certain embodiments, the amino acid substitution at position 591 of SEQ ID NO: 12 or SEQ ID NO: 2 is a substitution of a proline (P) for a glutamine (Q). In certain embodiments, the amino acid substitution at position 591 of SEQ ID NO: 12 or SEQ ID NO: 2 is a substitution of an arginine (R) for a glutamine (Q).

In certain embodiments of the methods of the disclosure, including those embodiments wherein the transposase comprises the above-described mutations at positions 30, 165, 282 and/or 538, the piggyBac™ transposase enzyme may comprise or the Super piggyBac™ transposase enzyme may further comprise an amino acid substitution at one or more of positions 103, 194, 372, 375, 450, 509 and 570 of the sequence of SEQ ID NO: 12 or SEQ ID NO: 2. In certain embodiments of the methods of the disclosure, including those embodiments wherein the transposase comprises the above-described mutations at positions 30, 165, 282 and/or 538, the piggyBac™ transposase enzyme may comprise or the Super piggyBac™ transposase enzyme may further comprise an amino acid substitution at two, three, four, five, six or more of positions 103, 194, 372, 375, 450, 509 and 570 of the sequence of SEQ ID NO: 12 or SEQ ID NO: 2. In certain embodiments, including those embodiments wherein the transposase comprises the above-described mutations at positions 30, 165, 282 and/or 538, the piggyBac™ transposase enzyme may comprise or the Super piggyBac™ transposase enzyme may further comprise an amino acid substitution at positions 103, 194, 372, 375, 450, 509 and 570 of the sequence of SEQ ID NO: 12 or SEQ ID NO: 2. In certain embodiments, the amino acid substitution at position 103 of SEQ ID NO: 12 or SEQ ID NO: 2 is a substitution of a proline (P) for a serine (S). In certain embodiments, the amino acid substitution at position 194 of SEQ ID NO: 12 or SEQ ID NO: 2 is a substitution of a valine (V) for a methionine (M). In certain embodiments, the amino acid substitution at position 372 of SEQ ID NO: 12 or SEQ ID NO: 2 is a substitution of an alanine (A) for an arginine (R). In certain embodiments, the amino acid substitution at position 375 of SEQ ID NO: 12 or SEQ ID NO: 2 is a substitution of an alanine (A) for a lysine (K). In certain embodiments, the amino acid substitution at position 450 of SEQ ID NO: 12 or SEQ ID NO: 2 is a substitution of an asparagine (N) for an aspartic acid (D). In certain embodiments, the amino acid substitution at position 509 of SEQ ID NO: 12 or SEQ ID NO: 2 is a substitution of a glycine (G) for a serine (S). In certain embodiments, the amino acid substitution at position 570 of SEQ ID NO: 12 or SEQ ID NO: 2 is a substitution of a serine (S) for an asparagine (N). In certain embodiments, the piggyBac™ transposase enzyme may comprise a substitution of a valine (V) for a methionine (M) at position 194 of SEQ ID NO: 12. In certain embodiments, including those embodiments wherein the piggyBac™ transposase enzyme may comprise a substitution of a valine (V) for a methionine (M) at position 194 of SEQ ID NO: 12, the piggyBac™ transposase enzyme may further comprise an amino acid substitution at positions 372, 375 and 450 of the sequence of SEQ ID NO: 12 or SEQ ID NO: 2. In certain embodiments, the piggyBac™ transposase enzyme may comprise a substitution of a valine (V) for a methionine (M) at position 194 of SEQ ID NO: 12, a substitution of an alanine (A) for an arginine (R) at position 372 of SEQ ID NO: 12, and a substitution of an alanine (A) for a lysine (K) at position 375 of SEQ ID NO: 12. In certain embodiments, the piggyBac™ transposase enzyme may comprise a substitution of a valine (V) for a methionine (M) at position 194 of SEQ ID NO: 12, a substitution of an alanine (A) for an arginine (R) at position 372 of SEQ ID NO: 12, a substitution of an alanine (A) for a lysine (K) at position 375 of SEQ ID NO: 12 and a substitution of an asparagine (N) for an aspartic acid (D) at position 450 of SEQ ID NO: 12.

### Scaffold Proteins

Protein scaffolds of the disclosure may be derived from a fibronectin type III (FN3) repeat protein, encoding or complementary nucleic acids, vectors, host cells, compositions, combinations, formulations, devices, and methods of making and using them. In a preferred embodiment, the protein scaffold is comprised of a consensus sequence of multiple FN3 domains from human Tenascin-C (hereinafter “Tenascin”). In a further preferred embodiment, the protein scaffold of the present invention is a consensus sequence of 15 FN3 domains. The protein scaffolds of the disclosure can be designed to bind various molecules, for example, a cellular target protein. In a preferred embodiment, the protein scaffolds of the disclosure can be designed to bind an epitope of a wild type and/or variant form of an antigen.

Protein scaffolds of the disclosure may include additional molecules or moieties, for example, the Fc region of an antibody, albumin binding domain, or other moiety influencing half-life. In further embodiments, the protein scaffolds of the disclosure may be bound to a nucleic acid molecule that may encode the protein scaffold.

The disclosure provides at least one method for expressing at least one protein scaffold based on a consensus sequence of multiple FN3 domains, in a host cell, comprising culturing a host cell as described herein under conditions wherein at least one protein scaffold is expressed in detectable and/or recoverable amounts.

The disclosure provides at least one composition comprising (a) a protein scaffold based on a consensus sequence of multiple FN3 domains and/or encoding nucleic acid as described herein; and (b) a suitable and/or pharmaceutically acceptable carrier or diluent.

The disclosure provides a method of generating libraries of a protein scaffold based on a fibronectin type III (FN3) repeat protein, preferably, a consensus sequence of multiple FN3 domains and, more preferably, a consensus sequence of multiple FN3 domains from human Tenascin. The library is formed by making successive generations of scaffolds by altering (by mutation) the amino acids or the number of amino acids in the molecules in particular positions in portions of the scaffold, e.g., loop regions. Libraries can be generated by altering the amino acid composition of a single loop or the simultaneous alteration of multiple loops or additional positions of the scaffold molecule. The loops that are altered can be lengthened or shortened accordingly. Such libraries can be generated to include all possible amino acids at each position, or a designed subset of amino acids. The library members can be used for screening by display, such as in vitro or CIS display (DNA, RNA, ribosome display, etc.), yeast, bacterial, and phage display.

Protein scaffolds of the disclosure provide enhanced biophysical properties, such as stability under reducing conditions and solubility at high concentrations; they may be expressed and folded in prokaryotic systems, such as E. coli, in eukaryotic systems, such as yeast, and in in vitro transcription/translation systems, such as the rabbit reticulocyte lysate system.

The disclosure provides a method of generating a scaffold molecule that binds to a particular target by panning the scaffold library of the invention with the target and detecting binders. In other related aspects, the disclosure comprises screening methods that may be used to generate or affinity mature protein scaffolds with the desired activity, e.g., capable of binding to target proteins with a certain affinity. Affinity maturation can be accomplished by iterative rounds of mutagenesis and selection using systems, such as phage display or in vitro display. Mutagenesis during this process may be the result of site directed mutagenesis to specific scaffold residues, random mutagenesis due to error-prone PCR, DNA shuffling, and/or a combination of these techniques.

The disclosure provides an isolated, recombinant and/or synthetic protein scaffold based on a consensus sequence of fibronectin type III (FN3) repeat protein, including, without limitation, mammalian-derived scaffold, as well as compositions and encoding nucleic acid molecules comprising at least one polynucleotide encoding protein scaffold based on the consensus FN3 sequence. The disclosure further includes, but is not limited to, methods of making and using such nucleic acids and protein scaffolds, including diagnostic and therapeutic compositions, methods and devices.

The protein scaffolds of the disclosure offer advantages over conventional therapeutics, such as ability to administer locally, orally, or cross the blood-brain barrier, ability to express in E. Coli allowing for increased expression of protein as a function of resources versus mammalian cell expression ability to be engineered into bispecific or tandem molecules that bind to multiple targets or multiple epitopes of the same target, ability to be conjugated to drugs, polymers, and probes, ability to be formulated to high concentrations, and the ability of such molecules to effectively penetrate diseased tissues and tumors.

Moreover, the protein scaffolds possess many of the properties of antibodies in relation to their fold that mimics the variable region of an antibody. This orientation enables the FN3 loops to be exposed similar to antibody complementarity determining regions (CDRs). They should be able to bind to cellular targets and the loops can be altered, e.g., affinity matured, to improve certain binding or related properties.

Three of the six loops of the protein scaffold of the disclosure correspond topologically to the complementarity determining regions (CDRs 1-3), i.e., antigen-binding regions, of an antibody, while the remaining three loops are surface exposed in a manner similar to antibody CDRs. These loops span at or about residues 13-16, 22-28, 38-43, 51-54, 60-64, and 75-81 of SEQ ID NO: 13. Preferably, the loop regions at or about residues 22-28, 51-54, and 75-81 are altered for binding specificity and affinity. One or more of these loop regions are randomized with other loop regions and/or other strands maintaining their sequence as backbone portions to populate a library and potent binders can be selected from the library having high affinity for a particular protein target. One or more of the loop regions can interact with a target protein similar to an antibody CDR interaction with the protein.

Scaffolds of the disclosure may comprise an antibody mimetic.

The term “antibody mimetic” is intended to describe an organic compound that specifically binds a target sequence and has a structure distinct from a naturally-occurring antibody. Antibody mimetics may comprise a protein, a nucleic acid, or a small molecule. The target sequence to which an antibody mimetic of the disclosure specifically binds may be an antigen. Antibody mimetics may provide superior properties over antibodies including, but not limited to, superior solubility, tissue penetration, stability towards heat and enzymes (e.g. resistance to enzymatic degradation), and lower production costs. Exemplary antibody mimetics include, but are not limited to, an affibody, an afflilin, an affimer, an affitin, an alphabody, an anticalin, and avimer (also known as avidity multimer), a DARPin (Designed Ankyrin Repeat Protein), a Fynomer, a Kunitz domain peptide, and a monobody.

Affibody molecules of the disclosure comprise a protein scaffold comprising or consisting of one or more alpha helix without any disulfide bridges. Preferably, affibody molecules of the disclosure comprise or consist of three alpha helices. For example, an affibody molecule of the disclosure may comprise an immunoglobulin binding domain. An affibody molecule of the disclosure may comprise the Z domain of protein A.

Affilin molecules of the disclosure comprise a protein scaffold produced by modification of exposed amino acids of, for example, either gamma-B crystallin or ubiquitin. Affilin molecules functionally mimic an antibody's affinity to antigen, but do not structurally mimic an antibody. In any protein scaffold used to make an affilin, those amino acids that are accessible to solvent or possible binding partners in a properly-folded protein molecule are considered exposed amino acids. Any one or more of these exposed amino acids may be modified to specifically bind to a target sequence or antigen.

Affimer molecules of the disclosure comprise a protein scaffold comprising a highly stable protein engineered to display peptide loops that provide a high affinity binding site for a specific target sequence. Exemplary affimer molecules of the disclosure comprise a protein scaffold based upon a cystatin protein or tertiary structure thereof. Exemplary affimer molecules of the disclosure may share a common tertiary structure of comprising an alpha-helix lying on top of an anti-parallel beta-sheet.

Affitin molecules of the disclosure comprise an artificial protein scaffold, the structure of which may be derived, for example, from a DNA binding protein (e.g. the DNA binding protein Sac7d). Affitins of the disclosure selectively bind a target sequence, which may be the entirety or part of an antigen. Exemplary affitins of the disclosure are manufactured by randomizing one or more amino acid sequences on the binding surface of a DNA binding protein and subjecting the resultant protein to ribosome display and selection. Target sequences of affitins of the disclosure may be found, for example, in the genome or on the surface of a peptide, protein, virus, or bacteria. In certain embodiments of the disclosure, an affitin molecule may be used as a specific inhibitor of an enzyme. Affitin molecules of the disclosure may include heat-resistant proteins or derivatives thereof.

Alphabody molecules of the disclosure may also be referred to as Cell-Penetrating Alphabodies (CPAB). Alphabody molecules of the disclosure comprise small proteins (typically of less than 10 kDa) that bind to a variety of target sequences (including antigens). Alphabody molecules are capable of reaching and binding to intracellular target sequences. Structurally, alphabody molecules of the disclosure comprise an artificial sequence forming single chain alpha helix (similar to naturally occurring coiled-coil structures). Alphabody molecules of the disclosure may comprise a protein scaffold comprising one or more amino acids that are modified to specifically bind target proteins. Regardless of the binding specificity of the molecule, alphabody molecules of the disclosure maintain correct folding and thermostability.

Anticalin molecules of the disclosure comprise artificial proteins that bind to target sequences or sites in either proteins or small molecules. Anticalin molecules of the disclosure may comprise an artificial protein derived from a human lipocalin. Anticalin molecules of the disclosure may be used in place of, for example, monoclonal antibodies or fragments thereof. Anticalin molecules may demonstrate superior tissue penetration and thermostability than monoclonal antibodies or fragments thereof. Exemplary anticalin molecules of the disclosure may comprise about 180 amino acids, having a mass of approximately 20 kDa. Structurally, anticalin molecules of the disclosure comprise a barrel structure comprising antiparallel beta-strands pairwise connected by loops and an attached alpha helix. In preferred embodiments, anticalin molecules of the disclosure comprise a barrel structure comprising eight antiparallel beta-strands pairwise connected by loops and an attached alpha helix.

Avimer molecules of the disclosure comprise an artificial protein that specifically binds to a target sequence (which may also be an antigen). Avimers of the disclosure may recognize multiple binding sites within the same target or within distinct targets. When an avimer of the disclosure recognize more than one target, the avimer mimics function of a bispecific antibody. The artificial protein avimer may comprise two or more peptide sequences of approximately 30-35 amino acids each. These peptides may be connected via one or more linker peptides. Amino acid sequences of one or more of the peptides of the avimer may be derived from an A domain of a membrane receptor. Avimers have a rigid structure that may optionally comprise disulfide bonds and/or calcium. Avimers of the disclosure may demonstrate greater heat stability compared to an antibody.

DARPins (Designed Ankyrin Repeat Proteins) of the disclosure comprise genetically-engineered, recombinant, or chimeric proteins having high specificity and high affinity for a target sequence. In certain embodiments, DARPins of the disclosure are derived from ankyrin proteins and, optionally, comprise at least three repeat motifs (also referred to as repetitive structural units) of the ankyrin protein. Ankyrin proteins mediate high-affinity protein-protein interactions. DARPins of the disclosure comprise a large target interaction surface.

Fynomers of the disclosure comprise small binding proteins (about 7 kDa) derived from the human Fyn SH3 domain and engineered to bind to target sequences and molecules with equal affinity and equal specificity as an antibody.

Kunitz domain peptides of the disclosure comprise a protein scaffold comprising a Kunitz domain. Kunitz domains comprise an active site for inhibiting protease activity. Structurally, Kunitz domains of the disclosure comprise a disulfide-rich alpha+beta fold. This structure is exemplified by the bovine pancreatic trypsin inhibitor. Kunitz domain peptides recognize specific protein structures and serve as competitive protease inhibitors. Kunitz domains of the disclosure may comprise Ecallantide (derived from a human lipoprotein-associated coagulation inhibitor (LACI)).

Monobodies of the disclosure are small proteins (comprising about 94 amino acids and having a mass of about 10 kDa) comparable in size to a single chain antibody. These genetically engineered proteins specifically bind target sequences including antigens. Monobodies of the disclosure may specifically target one or more distinct proteins or target sequences. In preferred embodiments, monobodies of the disclosure comprise a protein scaffold mimicking the structure of human fibronectin, and more preferably, mimicking the structure of the tenth extracellular type III domain of fibronectin. The tenth extracellular type III domain of fibronectin, as well as a monobody mimetic thereof, contains seven beta sheets forming a barrel and three exposed loops on each side corresponding to the three complementarity determining regions (CDRs) of an antibody. In contrast to the structure of the variable domain of an antibody, a monobody lacks any binding site for metal ions as well as a central disulfide bond. Multispecific monobodies may be optimized by modifying the loops BC and FG. Monobodies of the disclosure may comprise an adnectin.

Such a method can comprise administering an effective amount of a composition or a pharmaceutical composition comprising at least one scaffold protein to a cell, tissue, organ, animal or patient in need of such modulation, treatment, alleviation, prevention, or reduction in symptoms, effects or mechanisms. The effective amount can comprise an amount of about 0.001 to 500 mg/kg per single (e.g., bolus), multiple or continuous administration, or to achieve a serum concentration of 0.01-5000 μg/ml serum concentration per single, multiple, or continuous administration, or any effective range or value therein, as done and determined using known methods, as described herein or known in the relevant arts.

### Production and Generation of Scaffold Proteins

At least one scaffold protein of the disclosure can be optionally produced by a cell line, a mixed cell line, an immortalized cell or clonal population of immortalized cells, as well known in the art. See, e.g., Ausubel, et al., ed., Current Protocols in Molecular Biology, John Wiley & Sons, Inc., NY, N.Y. (1987-2001); Sambrook, et al., Molecular Cloning: A Laboratory Manual, 2nd Edition, Cold Spring Harbor, N.Y. (1989); Harlow and Lane, Antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, N.Y., (1997-2001).

Amino acids from a scaffold protein can be altered, added and/or deleted to reduce immunogenicity or reduce, enhance or modify binding, affinity, on-rate, off-rate, avidity, specificity, half-life, stability, solubility or any other suitable characteristic, as known in the art.

Optionally, scaffold proteins can be engineered with retention of high affinity for the antigen and other favorable biological properties. To achieve this goal, the scaffold proteins can be optionally prepared by a process of analysis of the parental sequences and various conceptual engineered products using three-dimensional models of the parental and engineered sequences. Three-dimensional models are commonly available and are familiar to those skilled in the art. Computer programs are available which illustrate and display probable three-dimensional conformational structures of selected candidate sequences and can measure possible immunogenicity (e.g., Immunofilter program of Xencor, Inc. of Monrovia, Calif.). Inspection of these displays permits analysis of the likely role of the residues in the functioning of the candidate sequence, i.e., the analysis of residues that influence the ability of the candidate scaffold protein to bind its antigen. In this way, residues can be selected and combined from the parent and reference sequences so that the desired characteristic, such as affinity for the target antigen(s), is achieved. Alternatively, or in addition to, the above procedures, other suitable methods of engineering can be used.

### Screening of Scaffold Proteins

Screening protein scaffolds for specific binding to similar proteins or fragments can be conveniently achieved using nucleotide (DNA or RNA display) or peptide display libraries, for example, in vitro display. This method involves the screening of large collections of peptides for individual members having the desired function or structure. The displayed nucleotide or peptide sequences can be from 3 to 5000 or more nucleotides or amino acids in length, frequently from 5-100 amino acids long, and often from about 8 to 25 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. Such methods are described in PCT Patent Publication Nos. 91/17271, 91/18980, 91/19818, and 93/08278.

Other systems for generating libraries of peptides have aspects of both in vitro chemical synthesis and recombinant methods. See, PCT Patent Publication Nos. 92/05258, 92/14843, and 96/19256. See also, U.S. Pat. Nos. 5,658,754; and 5,643,768. Peptide display libraries, vector, and screening kits are commercially available from such suppliers as Invitrogen (Carlsbad, Calif.), and Cambridge Antibody Technologies (Cambridgeshire, UK). See, e.g., U.S. Pat. Nos. 4,704,692, 4,939,666, 4,946,778, 5,260,203, 5,455,030, 5,518,889, 5,534,621, 5,656,730, 5,763,733, 5,767,260, 5,856,456, assigned to Enzon; U.S. Pat. Nos. 5,223,409, 5,403,484, 5,571,698, 5,837,500, assigned to Dyax, U.S. Pat. Nos. 5,427,908, 5,580,717, assigned to Affymax; U.S. Pat. No. 5,885,793, assigned to Cambridge Antibody Technologies; U.S. Pat. No. 5,750,373, assigned to Genentech, U.S. Pat. Nos. 5,618,920, 5,595,898, 5,576,195, 5,698,435, 5,693,493, 5,698,417, assigned to Xoma, Colligan, supra; Ausubel, supra; or Sambrook, supra.

The protein scaffolds of the disclosure can bind human or other mammalian proteins with a wide range of affinities (KD). In a preferred embodiment, at least one protein scaffold of the present invention can optionally bind to a target protein with high affinity, for example, with a KD equal to or less than about 10-7 M, such as but not limited to, 0.1-9.9 (or any range or value therein)×10-8, 10-9, 10-10, 10-11, 10-12, 10-13, 10-14, 10-15 or any range or value therein, as determined by surface plasmon resonance or the Kinexa method, as practiced by those of skill in the art.

The affinity or avidity of a protein scaffold for an antigen can be determined experimentally using any suitable method. (See, for example, Berzofsky, et al., “Antibody-Antigen Interactions,” In Fundamental Immunology, Paul, W. E., Ed., Raven Press: New York, N.Y. (1984); Kuby, Janis Immunology, W.H. Freeman and Company: New York, N.Y. (1992); and methods described herein). The measured affinity of a particular protein scaffold-antigen interaction can vary if measured under different conditions (e.g., salt concentration, pH). Thus, measurements of affinity and other antigen-binding parameters (e.g., KD, Kon, Koff) are preferably made with standardized solutions of protein scaffold and antigen, and a standardized buffer, such as the buffer described herein.

Competitive assays can be performed with the protein scaffold of the disclosure in order to determine what proteins, antibodies, and other antagonists compete for binding to a target protein with the protein scaffold of the present invention and/or share the epitope region. These assays as readily known to those of ordinary skill in the art evaluate competition between antagonists or ligands for a limited number of binding sites on a protein. The protein and/or antibody is immobilized or insolubilized before or after the competition and the sample bound to the target protein is separated from the unbound sample, for example, by decanting (where the protein/antibody was preinsolubilized) or by centrifuging (where the protein/antibody was precipitated after the competitive reaction). Also, the competitive binding may be determined by whether function is altered by the binding or lack of binding of the protein scaffold to the target protein, e.g., whether the protein scaffold molecule inhibits or potentiates the enzymatic activity of, for example, a label. ELISA and other functional assays may be used, as well known in the art.

### Nucleic Acid Molecules

Nucleic acid molecules of the disclosure encoding protein scaffolds can be in the form of RNA, such as mRNA, hnRNA, tRNA or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

Isolated nucleic acid molecules of the disclosure can include nucleic acid molecules comprising an open reading frame (ORF), optionally, with one or more introns, e.g., but not limited to, at least one specified portion of at least one protein scaffold; nucleic acid molecules comprising the coding sequence for a protein scaffold or loop region that binds to the target protein; and nucleic acid molecules which comprise a nucleotide sequence substantially different from those described above but which, due to the degeneracy of the genetic code, still encode the protein scaffold as described herein and/or as known in the art. Of course, the genetic code is well known in the art. Thus, it would be routine for one skilled in the art to generate such degenerate nucleic acid variants that code for specific protein scaffolds of the present invention. See, e.g., Ausubel, et al., supra, and such nucleic acid variants are included in the present invention.

As indicated herein, nucleic acid molecules of the disclosure which comprise a nucleic acid encoding a protein scaffold can include, but are not limited to, those encoding the amino acid sequence of a protein scaffold fragment, by itself, the coding sequence for the entire protein scaffold or a portion thereof; the coding sequence for a protein scaffold, fragment or portion, as well as additional sequences, such as the coding sequence of at least one signal leader or fusion peptide, with or without the aforementioned additional coding sequences, such as at least one intron, together with additional, non-coding sequences, including but not limited to, non-coding 5′ and 3′ sequences, such as the transcribed, non-translated sequences that play a role in transcription, mRNA processing, including splicing and polyadenylation signals (for example, ribosome binding and stability of mRNA); an additional coding sequence that codes for additional amino acids, such as those that provide additional functionalities. Thus, the sequence encoding a protein scaffold can be fused to a marker sequence, such as a sequence encoding a peptide that facilitates purification of the fused protein scaffold comprising a protein scaffold fragment or portion.

### Polynucleotides Selectively Hybridizing to a Polynucleotide as Described Herein

The disclosure provides isolated nucleic acids that hybridize under selective hybridization conditions to a polynucleotide disclosed herein. Thus, the polynucleotides of this embodiment can be used for isolating, detecting, and/or quantifying nucleic acids comprising such polynucleotides. For example, polynucleotides of the present invention can be used to identify, isolate, or amplify partial or full-length clones in a deposited library. In some embodiments, the polynucleotides are genomic or cDNA sequences isolated, or otherwise complementary to, a cDNA from a human or mammalian nucleic acid library.

Preferably, the cDNA library comprises at least 80% full-length sequences, preferably, at least 85% or 90% full-length sequences, and, more preferably, at least 95% full-length sequences. The cDNA libraries can be normalized to increase the representation of rare sequences. Low or moderate stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% sequence identity and can be employed to identify orthologous or paralogous sequences.

Optionally, polynucleotides of this invention will encode at least a portion of a protein scaffold encoded by the polynucleotides described herein. The polynucleotides of this invention embrace nucleic acid sequences that can be employed for selective hybridization to a polynucleotide encoding a protein scaffold of the present invention. See, e.g., Ausubel, supra; Colligan, supra, each entirely incorporated herein by reference.

### Construction of Nucleic Acids

The isolated nucleic acids of the disclosure can be made using (a) recombinant methods, (b) synthetic techniques, (c) purification techniques, and/or (d) combinations thereof, as well-known in the art.

The nucleic acids can conveniently comprise sequences in addition to a polynucleotide of the present invention. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the disclosure. For example, a hexa-histidine marker sequence provides a convenient means to purify the proteins of the disclosure. The nucleic acid of the disclosure, excluding the coding sequence, is optionally a vector, adapter, or linker for cloning and/or expression of a polynucleotide of the disclosure.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra; or Sambrook, supra).

### Recombinant Methods for Constructing Nucleic Acids

The isolated nucleic acid compositions of this disclosure, such as RNA, cDNA, genomic DNA, or any combination thereof, can be obtained from biological sources using any number of cloning methodologies known to those of skill in the art. In some embodiments, oligonucleotide probes that selectively hybridize, under stringent conditions, to the polynucleotides of the present invention are used to identify the desired sequence in a cDNA or genomic DNA library. The isolation of RNA, and construction of cDNA and genomic libraries are well known to those of ordinary skill in the art. (See, e.g., Ausubel, supra; or Sambrook, supra).

### Nucleic Acid Screening and Isolation Methods

A cDNA or genomic library can be screened using a probe based upon the sequence of a polynucleotide of the disclosure. Probes can be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by one or more of temperature, ionic strength, pH and the presence of a partially denaturing solvent, such as formamide. For example, the stringency of hybridization is conveniently varied by changing the polarity of the reactant solution through, for example, manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100%, or 70-100%, or any range or value therein. However, it should be understood that minor sequence variations in the probes and primers can be compensated for by reducing the stringency of the hybridization and/or wash medium.

Methods of amplification of RNA or DNA are well known in the art and can be used according to the disclosure without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the entire contents of which references are incorporated herein by reference. (See, e.g., Ausubel, supra; or Sambrook, supra.)

For instance, polymerase chain reaction (PCR) technology can be used to amplify the sequences of polynucleotides of the disclosure and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

### Synthetic Methods for Constructing Nucleic Acids

The isolated nucleic acids of the disclosure can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

### Recombinant Expression Cassettes

The disclosure further provides recombinant expression cassettes comprising a nucleic acid of the disclosure. A nucleic acid sequence of the disclosure, for example, a cDNA or a genomic sequence encoding a protein scaffold of the disclosure, can be used to construct a recombinant expression cassette that can be introduced into at least one desired host cell. A recombinant expression cassette will typically comprise a polynucleotide of the disclosure operably linked to transcriptional initiation regulatory sequences that will direct the transcription of the polynucleotide in the intended host cell. Both heterologous and non-heterologous (i.e., endogenous) promoters can be employed to direct expression of the nucleic acids of the disclosure.

In some embodiments, isolated nucleic acids that serve as promoter, enhancer, or other elements can be introduced in the appropriate position (upstream, downstream or in the intron) of a non-heterologous form of a polynucleotide of the disclosure so as to up or down regulate expression of a polynucleotide of the disclosure. For example, endogenous promoters can be altered in vivo or in vitro by mutation, deletion and/or substitution.

### Vectors and Host Cells

The disclosure also relates to vectors that include isolated nucleic acid molecules of the disclosure, host cells that are genetically engineered with the recombinant vectors, and the production of at least one protein scaffold by recombinant techniques, as is well known in the art. See, e.g., Sambrook, et al., supra; Ausubel, et al., supra, each entirely incorporated herein by reference.

For example, the PB-EF1a vector may be used. The vector comprises the following nucleotide sequence:

(SEQ ID NO: 40)
tgtacatagattaaccctagaaagataatcatatt
gtgacgtacgttaaagataatcatgcgtaaaattg
acgcatgtgttttatcggtctgtatatcgaggttt
atttattaatttgaatagatattaagttttattat
atttacacttacatactaataataaattcaacaaa
caatttatttatgtttatttatttattaaaaaaaa
acaaaaactcaaaatttcttctataaagtaacaaa
acttttatcgaatacctgcagcccgggggatgcag
agggacagcccccccccaaagcccccagggatgta
attacgtccctcccccgctagggggcagcagcgag
ccgcccggggctccgctccggtccggcgctccccc
cgcatccccgagccggcagcgtgcggggacagccc
gggcacggggaaggtggcacgggatcgctttcctc
tgaacgcttctcgctgctctttgagcctgcagaca
cctggggggatacggggaaaagttgactgtgcctt
tcgatcgaaccatggacagttagctttgcaaagat
ggataaagttttaaacagagaggaatctttgcagc
taatggaccttctaggtcttgaaaggagtgggaat
tggctccggtgcccgtcagtgggcagagcgcacat
cgcccacagtccccgagaagttggggggaggggtc
ggcaattgaaccggtgcctagagaaggtggcgcgg
ggtaaactgggaaagtgatgtcgtgtactggctcc
gcctttttcccgagggtgggggagaaccgtatata
agtgcagtagtcgccgtgaacgttctttttcgcaa
cgggtttgccgccagaacacaggtaagtgccgtgt
gtggttcccgcgggcctggcctctttacgggttat
ggcccttgcgtgccttgaattacttccacctggct
gcagtacgtgattcttgatcccgagcttcgggttg
gaagtgggtgggagagttcgaggccttgcgcttaa
ggagccccttcgcctcgtgcttgagttgaggcctg
gcctgggcgctggggccgccgcgtgcgaatctggt
ggcaccttcgcgcctgtctcgctgctttcgataag
tctctagccatttaaaatttttgatgacctgctgc
gacgctttttttctggcaagatagtcttgtaaatg
cgggccaagatctgcacactggtatttcggttttt
ggggccgcgggcggcgacggggcccgtgcgtccca
gcgcacatgttcggcgaggcggggcctgcgagcgc
ggccaccgagaatcggacgggggtagtctcaagct
ggccggcctgctctggtgcctggcctcgcgccgcc
gtgtatcgccccgccctgggcggcaaggctggccc
ggtcggcaccagttgcgtgagcggaaagatggccg
cttcccggccctgctgcagggagctcaaaatggag
gacgcggcgctcgggagagcgggcgggtgagtcac
ccacacaaaggaaaagggcctttccgtcctcagcc
gtcgcttcatgtgactccacggagtaccgggcgcc
gtccaggcacctcgattagttctcgagcttttgga
gtacgtcgtctttaggttggggggaggggttttat
gcgatggagtttccccacactgagtgggtggagac
tgaagttaggccagcttggcacttgatgtaattct
ccttggaatttgccctttttgagtttggatcttgg
ttcattctcaagcctcagacagtggttcaaagttt
ttttcttccatttcaggtgtcgtgagaattctaat
acgactcactatagggtgtgctgtctcatcatttt
ggcaaagattggccaccaagcttgtcctgcaggag
ggtcgacgcctctagacgggcggccgctccggatc
cacgggtaccgatcacatatgcctttaattaaaca
ctagttctatagtgtcacctaaattccctttagtg
agggttaatggccgtaggccgccagaattgggtcc
agacatgataagatacattgatgagtttggacaaa
ccacaactagaatgcagtgaaaaaaatgctttatt
tgtgaaatttgtgatgctattgctttatttgtaac
cattataagctgcaataaacaagttaacaacaaca
attgcattcattttatgtttcaggttcagggggag
gtgtgggaggttttttcggactctaggacctgcgc
atgcgcttggcgtaatcatggtcatagctgtttcc
tgttttccccgtatccccccaggtgtctgcaggct
caaagagcagcgagaagcgttcagaggaaagcgat
cccgtgccaccttccccgtgcccgggctgtccccg
cacgctgccggctcggggatgcggggggagcgccg
gaccggagcggagccccgggcggctcgctgctgcc
ccctagcgggggagggacgtaattacatccctggg
ggctttgggggggggctgtccctctcaccgcggtg
gagctccagcttttgttcgaattggggccccccct
cgagggtatcgatgatatctataacaagaaaatat
atatataataagttatcacgtaagtagaacatgaa
ataacaatataattatcgtatgagttaaatcttaa
aagtcacgtaaaagataatcatgcgtcattttgac
tcacgcggtcgttatagttcaaaatcagtgacact
taccgcattgacaagcacgcctcacgggagctcca
agcggcgactgagatgtcctaaatgcacagcgacg
gattcgcgctatttagaaagagagagcaatatttc
aagaatgcatgcgtcaattttacgcagactatctt
tctagggttaatctagctagccttaagggcgccta
ttgcgttgcgctcactgcccgctttccagtcggga
aacctgtcgtgccagctgcattaatgaatcggcca
acgcgcggggagaggcggtttgcgtattgggcgct
cttccgcttcctcgctcactgactcgctgcgctcg
gtcgttcggctgcggcgagcggtatcagctcactc
aaaggcggtaatacggttatccacagaatcagggg
ataacgcaggaaagaacatgaccaaaatcccttaa
cgtgagttttcgttccactgagcgtcagaccccgt
agaaaagatcaaaggatcttcttgagatccttttt
ttctgcgcgtaatctgctgcttgcaaacaaaaaaa
ccaccgctaccagcggtggtttgtttgccggatca
agagctaccaactctttttccgaaggtaactggct
tcagcagagcgcagataccaaatactgttcttcta
gtgtagccgtagttaggccaccacttcaagaactc
tgtagcaccgcctacatacctcgctctgctaatcc
tgttaccagtggctgctgccagtggcgataagtcg
tgtcttaccgggttggactcaagacgatagttacc
ggataaggcgcagcggtcgggctgaacggggggtt
cgtgcacacagcccagcttggagcgaacgacctac
accgaactgagatacctacagcgtgagctatgaga
aagcgccacgcttcccgaagggagaaaggcggaca
ggtatccggtaagcggcagggtcggaacaggagag
cgcacgagggagcttccagggggaaacgcctggta
tctttatagtcctgtcgggtttcgccacctctgac
ttgagcgtcgatttttgtgatgctcgtcagggggg
cggagcctatggaaaaacgccagcaacgcggcctt
tttacggttcctggccttttgctggccttttgctc
acatgagattatcaaaaaggatcttcacctagatc
cttttaaattaaaaatgaagttttaaatcaatcta
aagtatatatgagtaaacttggtctgacagtcaga
agaactcgtcaagaaggcgatagaaggcgatgcgc
tgcgaatcgggagcggcgataccgtaaagcacgag
gaagcggtcagcccattcgccgccaagctcttcag
caatatcacgggtagccaacgctatgtcctgatag
cggtccgccacacccagccggccacagtcgatgaa
tccagaaaagcggccattttccaccatgatattcg
gcaagcaggcatcgccatgggtcacgacgagatcc
tcgccgtcgggcatgctcgccttgagcctggcgaa
cagttcggctggcgcgagcccctgatgctcttcgt
ccagatcatcctgatcgacaagaccggcttccatc
cgagtacgtgctcgctcgatgcgatgtttcgcttg
gtggtcgaatgggcaggtagccggatcaagcgtat
gcagccgccgcattgcatcagccatgatggatact
ttctcggcaggagcaaggtgagatgacaggagatc
ctgccccggcacttcgcccaatagcagccagtccc
ttcccgcttcagtgacaacgtcgagcacagctgcg
caaggaacgcccgtcgtggccagccacgatagccg
cgctgcctcgtcttgcagttcattcagggcaccgg
acaggtcggtcttgacaaaaagaaccgggcgcccc
tgcgctgacagccggaacacggcggcatcagagca
gccgattgtctgttgtgcccagtcatagccgaata
gcctctccacccaagcggccggagaacctgcgtgc
aatccatcttgttcaatcataatattattgaagca
tttatcagggttcgtctcgtcccggtctcctccca
atgcatgtcaatattggccattagccatattattc
attggttatatagcataaatcaatattggctattg
gccattgcatacgttgtatctatatcataata

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but are not limited to, ampicillin, zeocin (Sh bla gene), puromycin (pac gene), hygromycin B (hygB gene), G418/Geneticin (neo gene), mycophenolic acid, or glutamine synthetase (GS, U.S. Pat. Nos. 5,122,464; 5,770,359; 5,827,739), blasticidin (bsd gene), resistance genes for eukaryotic cell culture as well as ampicillin, zeocin (Sh bla gene), puromycin (pac gene), hygromycin B (hygB gene), G418/Geneticin (neo gene), kanamycin, spectinomycin, streptomycin, carbenicillin, bleomycin, erythromycin, polymyxin B, or tetracycline resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

Expression vectors will preferably but optionally include at least one selectable cell surface marker for isolation of cells modified by the compositions and methods of the disclosure. Selectable cell surface markers of the disclosure comprise surface proteins, glycoproteins, or group of proteins that distinguish a cell or subset of cells from another defined subset of cells. Preferably the selectable cell surface marker distinguishes those cells modified by a composition or method of the disclosure from those cells that are not modified by a composition or method of the disclosure. Such cell surface markers include, e.g., but are not limited to, “cluster of designation” or “classification determinant” proteins (often abbreviated as “CD”) such as a truncated or full length form of CD19, CD271, CD34, CD22, CD20, CD33, CD52, or any combination thereof. Cell surface markers further include the suicide gene marker RQR8 (Philip B et al. Blood. 2014 Aug. 21; 124(8):1277-87).

Expression vectors will preferably but optionally include at least one selectable drug resistance marker for isolation of cells modified by the compositions and methods of the disclosure. Selectable drug resistance markers of the disclosure may comprise wild-type or mutant Neo, DHFR, TYMS, FRANCF, RAD51C, GCS, MDR1, ALDH1, NKX2.2, or any combination thereof.

At least one protein scaffold of the disclosure can be expressed in a modified form, such as a fusion protein, and can include not only secretion signals, but also additional heterologous functional regions. For instance, a region of additional amino acids, particularly charged amino acids, can be added to the N-terminus of a protein scaffold to improve stability and persistence in the host cell, during purification, or during subsequent handling and storage. Also, peptide moieties can be added to a protein scaffold of the disclosure to facilitate purification. Such regions can be removed prior to final preparation of a protein scaffold or at least one fragment thereof. Such methods are described in many standard laboratory manuals, such as Sambrook, supra, Chapters 17.29-17.42 and 18.1-18.74; Ausubel, supra, Chapters 16, 17 and 18.

Those of ordinary skill in the art are knowledgeable in the numerous expression systems available for expression of a nucleic acid encoding a protein of the disclosure. Alternatively, nucleic acids of the disclosure can be expressed in a host cell by turning on (by manipulation) in a host cell that contains endogenous DNA encoding a protein scaffold of the disclosure. Such methods are well known in the art, e.g., as described in U.S. Pat. Nos. 5,580,734, 5,641,670, 5,733,746, and 5,733,761, entirely incorporated herein by reference.

Illustrative of cell cultures useful for the production of the protein scaffolds, specified portions or variants thereof, are bacterial, yeast, and mammalian cells as known in the art. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Ag14, 293 cells, HeLa cells and the like, which are readily available from, for example, American Type Culture Collection, Manassas, Va. (www.atcc.org). Preferred host cells include cells of lymphoid origin, such as myeloma and lymphoma cells. Particularly preferred host cells are P3X63Ag8.653 cells (ATCC Accession Number CRL-1580) and SP2/0-Ag14 cells (ATCC Accession Number CRL-1851). In a particularly preferred embodiment, the recombinant cell is a P3X63Ab8.653 or an SP2/0-Ag14 cell.

Expression vectors for these cells can include one or more of the following expression control sequences, such as, but not limited to, an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present invention are known and/or available, for instance, from the American Type Culture Collection Catalogue of Cell Lines and Hybridomas (www.atcc.org) or other known or commercial sources.

When eukaryotic host cells are employed, polyadenylation or transcription terminator sequences are typically incorporated into the vector. An example of a terminator sequence is the polyadenylation sequence from the bovine growth hormone gene. Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

### Purification of a Protein Scaffold

A protein scaffold can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Protein scaffolds of the disclosure include naturally purified products, products of chemical synthetic procedures, and products produced by recombinant techniques from a prokaryotic or eukaryotic host, including, for example, E. coli, yeast, higher plant, insect and mammalian cells. Depending upon the host employed in a recombinant production procedure, the protein scaffold of the disclosure can be glycosylated or can be non-glycosylated. Such methods are described in many standard laboratory manuals, such as Sambrook, supra, Sections 17.37-17.42; Ausubel, supra, Chapters 10, 12, 13, 16, 18 and 20, Colligan, Protein Science, supra, Chapters 12-14, all entirely incorporated herein by reference.

### Amino Acid Codes

The amino acids that make up protein scaffolds of the disclosure are often abbreviated. The amino acid designations can be indicated by designating the amino acid by its single letter code, its three letter code, name, or three nucleotide codon(s) as is well understood in the art (see Alberts, B., et al., Molecular Biology of The Cell, Third Ed., Garland Publishing, Inc., New York, 1994). A protein scaffold of the disclosure can include one or more amino acid substitutions, deletions or additions, either from natural mutations or human manipulation, as specified herein. Amino acids in a protein scaffold of the disclosure that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to, at least one neutralizing activity. Sites that are critical for protein scaffold binding can also be identified by structural analysis, such as crystallization, nuclear magnetic resonance or photoaffinity labeling (Smith, et al., J. Mol. Biol. 224:899-904 (1992) and de Vos, et al., Science 255:306-312 (1992)).

As those of skill will appreciate, the invention includes at least one biologically active protein scaffold of the disclosure. Biologically active protein scaffolds have a specific activity at least 20%, 30%, or 40%, and, preferably, at least 50%, 60%, or 70%, and, most preferably, at least 80%, 90%, or 95%-99% or more of the specific activity of the native (non-synthetic), endogenous or related and known protein scaffold. Methods of assaying and quantifying measures of enzymatic activity and substrate specificity are well known to those of skill in the art.

In another aspect, the disclosure relates to protein scaffolds and fragments, as described herein, which are modified by the covalent attachment of an organic moiety. Such modification can produce a protein scaffold fragment with improved pharmacokinetic properties (e.g., increased in vivo serum half-life). The organic moiety can be a linear or branched hydrophilic polymeric group, fatty acid group, or fatty acid ester group. In particular embodiments, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms.

The modified protein scaffolds and fragments of the disclosure can comprise one or more organic moieties that are covalently bonded, directly or indirectly, to the antibody. Each organic moiety that is bonded to a protein scaffold or fragment of the disclosure can independently be a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane. For example, polylysine is more soluble in water than in octane. Thus, a protein scaffold modified by the covalent attachment of polylysine is encompassed by the disclosure. Hydrophilic polymers suitable for modifying protein scaffolds of the disclosure can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the protein scaffold of the disclosure has a molecular weight of about 800 to about 150,000 Daltons as a separate molecular entity. For example, PEG5000 and PEG 20,000, wherein the subscript is the average molecular weight of the polymer in Daltons, can be used. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N,N-carbonyl diimidazole) on a fatty acid or fatty acid ester can be coupled to a hydroxyl group on a polymer.

Fatty acids and fatty acid esters suitable for modifying protein scaffolds of the disclosure can be saturated or can contain one or more units of unsaturation. Fatty acids that are suitable for modifying protein scaffolds of the disclosure include, for example, n-dodecanoate (C12, laurate), n-tetradecanoate (C14, myristate), n-octadecanoate (C18, stearate), n-eicosanoate (C20, arachidate), n-docosanoate (C22, behenate), n-triacontanoate (C30), n-tetracontanoate (C40), cis-Δ9-octadecanoate (C18, oleate), all cis-Δ5,8,11,14-eicosatetraenoate (C20, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably, one to about six, carbon atoms.

The modified protein scaffolds and fragments can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups, such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)). An activating group can be bonded directly to the organic group (e.g., hydrophilic polymer, fatty acid, fatty acid ester), or through a linker moiety, for example, a divalent C1-C12 group wherein one or more carbon atoms can be replaced by a heteroatom, such as oxygen, nitrogen or sulfur. Suitable linker moieties include, for example, tetraethylene glycol, —(CH2)3-, —NH—(CH2)6-NH—, —(CH2)2-NH- and —CH2-O—CH2-CH2-O—CH2-CH2O—CH—NH—. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate, as described, or can be reacted with maleic anhydride and the resulting product cyclized to produce an activated maleimido derivative of the fatty acid. (See, for example, Thompson, et al., WO 92/16221, the entire teachings of which are incorporated herein by reference.)

The modified protein scaffolds of the disclosure can be produced by reacting a protein scaffold or fragment with a modifying agent. For example, the organic moieties can be bonded to the protein scaffold in a non-site specific manner by employing an amine-reactive modifying agent, for example, an NHS ester of PEG. Modified protein scaffolds and fragments comprising an organic moiety that is bonded to specific sites of a protein scaffold of the disclosure can be prepared using suitable methods, such as reverse proteolysis (Fisch et al., Bioconjugate Chem., 3:147-153 (1992); Werlen et al., Bioconjugate Chem., 5:411-417 (1994); Kumaran et al., Protein Sci. 6(10):2233-2241 (1997); Itoh et al., Bioorg. Chem., 24(1): 59-68 (1996); Capellas et al., Biotechnol. Bioeng., 56(4):456-463 (1997)), and the methods described in Hermanson, G. T., Bioconjugate Techniques, Academic Press: San Diego, Calif. (1996).

### Protein Scaffold Compositions Comprising Further Therapeutically Active Ingredients

Protein scaffold compounds, compositions or combinations of the present disclosure can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18th Edition, Mack Publishing Co. (Easton, Pa.) 1990. Pharmaceutically acceptable carriers can be routinely selected that are suitable for the mode of administration, solubility and/or stability of the protein scaffold, fragment or variant composition as well known in the art or as described herein.

Pharmaceutical excipients and additives useful in the present composition include, but are not limited to, proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Exemplary protein excipients include serum albumin, such as human serum albumin (HSA), recombinant human albumin (rHA), gelatin, casein, and the like. Representative amino acid/protein components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Carbohydrate excipients suitable for use in the invention include, for example, monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferred carbohydrate excipients for use in the present invention are mannitol, trehalose, and raffinose.

Protein scaffold compositions can also include a buffer or a pH-adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers for use in the present compositions are organic acid salts, such as citrate.

Additionally, protein scaffold compositions of the invention can include polymeric excipients/additives, such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates, such as “TWEEN 20” and “TWEEN 80”), lipids (e.g., phospholipids, fatty acids), steroids (e.g., cholesterol), and chelating agents (e.g., EDTA).

These and additional known pharmaceutical excipients and/or additives suitable for use in the protein scaffold, portion or variant compositions according to the invention are known in the art, e.g., as listed in “Remington: The Science & Practice of Pharmacy”, 19th ed., Williams & Williams, (1995), and in the “Physician's Desk Reference”, 52nd ed., Medical Economics, Montvale, N.J. (1998), the disclosures of which are entirely incorporated herein by reference. Preferred carrier or excipient materials are carbohydrates (e.g., saccharides and alditols) and buffers (e.g., citrate) or polymeric agents. An exemplary carrier molecule is the mucopolysaccharide, hyaluronic acid, which may be useful for intraarticular delivery.

T Cell Isolation from a Leukapheresis Product

A leukapheresis product or blood may be collected from a subject at clinical site using a closed system and standard methods (e.g., a COBE Spectra Apheresis System). Preferably, the product is collected according to standard hospital or institutional Leukapheresis procedures in standard Leukapheresis collection bags. For example, in preferred embodiments of the methods of the disclosure, no additional anticoagulants or blood additives (heparin, etc.) are included beyond those normally used during leukapheresis.

Alternatively, white blood cells (WBC)/Peripheral Blood Mononuclear Cells (PBMC) (using Biosafe Sepax 2 (Closed/Automated)) or T cells (using CliniMACS® Prodigy (Closed/Automated)) may be isolated directly from whole blood. However, in certain subjects (e.g. those diagnosed and/or treated for cancer), the WBC/PBMC yield may be significantly lower when isolated from whole blood than when isolated by leukapheresis.

Either the leukapheresis procedure and/or the direct cell isolation procedure may be used for any subject of the disclosure.

The leukapheresis product, blood, WBC/PBMC composition and/or T-cell composition should be packed in insulated containers and should be kept at controlled room temperature (+19° C. to +25° C.) according to standard hospital of institutional blood collection procedures approved for use with the clinical protocol. The leukapheresis product, blood, WBC/PBMC composition and/or T-cell composition should not be refrigerated.

The cell concentration leukapheresis product, blood, WBC/PBMC composition and/or T-cell composition should not exceed 0.2×10⁹ cells per mL during transportation. Intense mixing of the leukapheresis product, blood, WBC/PBMC composition and/or T-cell composition should be avoided.

If the leukapheresis product, blood, WBC/PBMC composition and/or T-cell composition has to be stored, e.g. overnight, it should be kept at controlled room temperature (same as above). During storage, the concentration of the leukapheresis product, blood, WBC/PBMC composition and/or T-cell composition should never exceed 0.2×10⁹ cell per mL.

Preferably, cells of the leukapheresis product, blood, WBC/PBMC composition and/or T-cell composition should be stored in autologous plasma. In certain embodiments, if the cell concentration of the leukapheresis product, blood, WBC/PBMC composition and/or T-cell composition is higher than 0.2×10⁹ cell per mL, the product should be diluted with autologous plasma.

Preferably, the leukapheresis product, blood, WBC/PBMC composition and/or T-cell composition should not be older than 24 hours when starting the labeling and separation procedure. The leukapheresis product, blood, WBC/PBMC composition and/or T-cell composition may be processed and/or prepared for cell labeling using a closed and/or automated system (e.g., CliniMACS Prodigy).

An automated system may perform additional buffy coat isolation, possibly by ficolation, and/or washing of the cellular product (e.g., the leukapheresis product, blood, WBC/PBMC composition and/or T cell composition).

A closed and/or automated system may be used to prepare and label cells for T-Cell isolation (from, for example, the leukapheresis product, blood, WBC/PBMC composition and/or T cell composition).

Although WBC/PBMCs may be nucleofected directly (which is easier and saves additional steps), the methods of the disclosure may include first isolating T cells prior to nucleofection. The easier strategy of directly nucleofecting PBMC requires selective expansion of CAR+ cells that is mediated via CAR signaling, which by itself is proving to be an inferior expansion method that directly reduces the in vivo efficiency of the product by rendering T cells functionally exhausted. The product may be a heterogeneous composition of CAR+ cells including T cells, NK cells, NKT cells, monocytes, or any combination thereof, which increases the variability in product from patient to patient and makes dosing and CRS management more difficult. Since T cells are thought to be the primary effectors in tumor suppression and killing, T cell isolation for the manufacture of an autologous product may result in significant benefits over the other more heterogeneous composition.

T cells may be isolated directly, by enrichment of labeled cells or depletion of labeled cells in a one-way labeling procedure or, indirectly, in a two-step labeling procedure. According to certain enrichment strategies of the disclosure, T cells may be collected in a Cell Collection Bag and the non-labeled cells (non-target cells) in a Negative Fraction Bag. In contrast to an enrichment strategy of the disclosure, the non-labeled cells (target cells) are collected in a Cell Collection Bag and the labeled cells (non-target cells) are collected in a Negative Fraction Bag or in the Non-Target Cell Bag, respectively. Selection reagents may include, but are not limited to, antibody-coated beads. Antibody-coated beads may either be removed prior to a modification and/or an expansion step, or, retained on the cells prior to a modification and/or an expansion step. One or more of the following non-limiting examples of cellular markers may be used to isolate T-cells: CD3, CD4, CD8, CD25, anti-biotin, CD1c, CD3/CD19, CD3/CD56, CD14, CD19, CD34, CD45RA, CD56, CD62L, CD133, CD137, CD271, CD304, IFN-gamma, TCR alpha/beta, and/or any combination thereof. Methods for the isolation of T-cells may include one or more reagents that specifically bind and/or detectably-label one or more of the following non-limiting examples of cellular markers may be used to isolate T-cells: CD3, CD4, CD8, CD25, anti-biotin, CD1c, CD3/CD19, CD3/CD56, CD14, CD19, CD34, CD45RA, CD56, CD62L, CD133, CD137, CD271, CD304, IFN-gamma, TCR alpha/beta, and/or any combination thereof. These reagents may or may not be “Good Manufacturing Practices” (“GMP”) grade. Reagents may include, but are not limited to, Thermo DynaBeads and Miltenyi CliniMACS products. Methods of isolating T-cells of the disclosure may include multiple iterations of labeling and/or isolation steps. At any point in the methods of isolating T-cells of the disclosure, unwanted cells and/or unwanted cell types may be depleted from a T cell product composition of the disclosure by positively or negatively selecting for the unwanted cells and/or unwanted cell types. A T cell product composition of the disclosure may contain additional cell types that may express CD4, CD8, and/or another T cell marker(s).

Methods of the disclosure for nucleofection of T cells may eliminate the step of T cell isolation by, for example, a process for nucleofection of T cells in a population or composition of WBC/PBMCs that, following nucleofection, includes an isolation step or a selective expansion step via TCR signaling.

Certain cell populations may be depleted by positive or negative selection before or after T cell enrichment and/or sorting. Examples of cell compositions that may be depleted from a cell product composition may include myeloid cells, CD25+ regulatory T cells (T Regs), dendritic cells, macrophages, red blood cells, mast cells, gamma-delta T cells, natural killer (NK) cells, a Natural Killer (NK)-like cell (e.g. a Cytokine Induced Killer (CIK) cell), induced natural killer (iNK) T cells, NK T cells, B cells, or any combination thereof.

T cell product compositions of the disclosure may include CD4+ and CD8+ T-Cells. CD4+ and CD8+ T-Cells may be isolated into separate collection bags during an isolation or selection procedure. CD4+ T cells and CD8+ T cells may be further treated separately, or treated after reconstitution (combination into the same composition) at a particular ratio.

The particular ratio at which CD4+ T cells and CD8+ T cells may be reconstituted may depend upon the type and efficacy of expansion technology used, cell medium, and/or growth conditions utilized for expansion of T-cell product compositions. Examples of possible CD4+: CD8+ ratios include, but are not limited to, 50%:50%, 60%:40%, 40%:60% 75%:25% and 25%:75%.

CD8+ T cells exhibit a potent capacity for tumor cell killing, while CD4+ T cells provide many of the cytokines required to support CD8+ T cell proliferative capacity and function. Because T cells isolated from normal donors are predominantly CD4+, the T-cell product compositions are artificially adjusted in vitro with respect to the CD4+:CD8+ ratio to improve upon the ratio of CD4+ T cells to CD8+ T cells that would otherwise be present in vivo. An optimized ratio may also be used for the ex vivo expansion of the autologous T-cell product composition. In view of the artificially adjusted CD4+:CD8+ ratio of the T-cell product composition, it is important to note that the product compositions of the disclosure may be significantly different and provide significantly greater advantage than any naturally-occurring population of T-cells.

Preferred methods for T cell isolation may include a negative selection strategy for yielding untouched pan T cell, meaning that the resultant T-cell composition includes T-cells that have not been manipulated and that contain a naturally-occurring variety/ratio of T-cells.

Reagents that may be used for positive or negative selection include, but are not limited to, magnetic cell separation beads. Magnetic cell separation beads may or may not be removed or depleted from selected populations of CD4+ T cells, CD8+ T cells, or a mixed population of both CD4+ and CD8+ T cells before performing the next step in a T-cell isolation method of the disclosure.

T cell compositions and T cell product compositions may be prepared for cryopreservation, storage in standard T Cell Culture Medium, and/or genetic modification.

T cell compositions, T cell product compositions, unstimulated T cell compositions, resting T cell compositions or any portion thereof may be cryopreserved using a standard cryopreservation method optimized for storing and recovering human cells with high recovery, viability, phenotype, and/or functional capacity. Commercially-available cryopreservation media and/or protocols may be used. Cryopreservation methods of the disclosure may include a DMSO free cryopreservant (e.g. CryoSOfree™ DMSO-free Cryopreservation Medium) reduce freezing-related toxicity.

T cell compositions, T cell product compositions, unstimulated T cell compositions, resting T cell compositions or any portion thereof may be stored in a culture medium. T cell culture media of the disclosure may be optimized for cell storage, cell genetic modification, cell phenotype and/or cell expansion. T cell culture media of the disclosure may include one or more antibiotics. Because the inclusion of an antibiotic within a cell culture media may decrease transfection efficiency and/or cell yield following genetic modification via nucleofection, the specific antibiotics (or combinations thereof) and their respective concentration(s) may be altered for optimal transfection efficiency and/or cell yield following genetic modification via nucleofection.

T cell culture media of the disclosure may include serum, and, moreover, the serum composition and concentration may be altered for optimal cell outcomes. Human AB serum is preferred over FBS/FCS for culture of T cells because, although contemplated for use in T cell culture media of the disclosure, FBS/FCS may introduce xeno-proteins. Serum may be isolated form the blood of the subject for whom the T-cell composition in culture is intended for administration, thus, a T cell culture medium of the disclosure may comprise autologous serum. Serum-free media or serum-substitute may also be used in T-cell culture media of the disclosure. In certain embodiments of the T-cell culture media and methods of the disclosure, serum-free media or serum-substitute may provide advantages over supplementing the medium with xeno-serum, including, but not limited to, healthier cells that have greater viability, nucleofect with higher efficiency, exhibit greater viability post-nucleofection, display a more desirable cell phenotype, and/or greater/faster expansion upon addition of expansion technologies.

T cell culture media may include a commercially-available cell growth media. Exemplary commercially-available cell growth media include, but are not limited to, PBS, HBSS, OptiMEM, DMEM, RPMI 1640, AIM-V, X-VIVO 15, CellGro DC Medium, CTS OpTimizer T Cell Expansion SFM, TexMACS Medium, PRIME-XV T Cell Expansion Medium, ImmunoCult-XF T Cell Expansion Medium, or any combination thereof.

T cell compositions, T cell product compositions, unstimulated T cell compositions, resting T cell compositions or any portion thereof may be prepared for genetic modification. Preparation of T cell compositions, T cell product compositions, unstimulated T cell compositions, resting T cell compositions or any portion thereof for genetic modification may include cell washing and/or resuspension in a desired nucleofection buffer. Cryopreserved T-cell compositions may be thawed and prepared for genetic modification by nucleofection. Cryopreserved cells may be thawed according to standard or known protocols. Thawing and preparation of cryopreserved cells may be optimized to yield cells that have greater viability, nucleofect with higher efficiency, exhibit greater viability post-nucleofection, display a more desirable cell phenotype, and/or greater/faster expansion upon addition of expansion technologies. For example, Grifols Albutein (25% human albumin) may be used in the thawing and/or preparation process.

### Genetic Modification of an Autologous T Cell Product Composition

T cell compositions, T cell product compositions, unstimulated T cell compositions, resting T cell compositions or any portion thereof may be genetically modified using, for example, a nucleofection strategy such as electroporation. The total number of cells to be nucleofected, the total volume of the nucleofection reaction, and the precise timing of the preparation of the sample may be optimized to yield cells that have greater viability, nucleofect with higher efficiency, exhibit greater viability post-nucleofection, display a more desirable cell phenotype, and/or greater/faster expansion upon addition of expansion technologies.

Nucleofection and/or electroporation may be accomplished using, for example, Lonza Amaxa, MaxCyte PulseAgile, Harvard Apparatus BTX, and/or Invitrogen Neon. Non-metal electrode systems, including, but not limited to, plastic polymer electrodes, may be preferred for nucleofection.

Prior to genetic modification by nucleofection, T cell compositions, T cell product compositions, unstimulated T cell compositions, resting T cell compositions or any portion thereof may be resuspended in a nucleofection buffer. Nucleofection buffers of the disclosure include commercially-available nucleofection buffers. Nucleofection buffers of the disclosure may be optimized to yield cells that have greater viability, nucleofect with higher efficiency, exhibit greater viability post-nucleofection, display a more desirable cell phenotype, and/or greater/faster expansion upon addition of expansion technologies. Nucleofection buffers of the disclosure may include, but are not limited to, PBS, HBSS, OptiMEM, BTXpress, Amaxa Nucleofector, Human T cell nucleofection buffer and any combination thereof. Nucleofection buffers of the disclosure may comprise one or more supplemental factors to yield cells that have greater viability, nucleofect with higher efficiency, exhibit greater viability post-nucleofection, display a more desirable cell phenotype, and/or greater/faster expansion upon addition of expansion technologies. Exemplary supplemental factors include, but are not limited to, recombinant human cytokines, chemokines, interleukins and any combination thereof. Exemplary cytokines, chemokines, and interleukins include, but are not limited to, IL2, IL7, IL12, IL15, IL21, IL1, IL3, IL4, IL5, IL6, IL8, CXCL8, IL9, IL10, IL11, IL13, IL14, IL16, IL17, IL18, IL19, IL20, IL22, IL23, IL25, IL26, IL27, IL28, IL29, IL30, IL31, IL32, IL33, IL35, IL36, GM-CSF, IFN-gamma, IL-1 alpha/IL-1F1, IL-1 beta/IL-1F2, IL-12 p70, IL-12/IL-35 p35, IL-13, IL-17/IL-17A, IL-17A/F Heterodimer, IL-17F, IL-18/IL-1F4, IL-23, IL-24, IL-32, IL-32 beta, IL-32 gamma, IL-33, LAP (TGF-beta 1), Lymphotoxin-alpha/TNF-beta, TGF-beta, TNF-alpha, TRANCE/TNFSF11/RANK L and any combination thereof. Exemplary supplemental factors include, but are not limited to, salts, minerals, metabolites or any combination thereof. Exemplary salts, minerals, and metabolites include, but are not limited to, HEPES, Nicotinamide, Heparin, Sodium Pyruvate, L-Glutamine, MEM Non-Essential Amino Acid Solution, Ascorbic Acid, Nucleosides, FBS/FCS, Human serum, serum-substitute, anti-biotics, pH adjusters, Earle's Salts, 2-Mercaptoethanol, Human transferrin, Recombinant human insulin, Human serum albumin, Nucleofector PLUS Supplement, KCL, MgCl2, Na2HPO4, NAH2PO4, Sodium lactobionate, Manitol, Sodium succinate, Sodium Chloride, CINa, Glucose, Ca(NO3)2, Tris/HCl, K2HPO4, KH2PO4, Polyethylenimine, Poly-ethylene-glycol, Poloxamer 188, Poloxamer 181, Poloxamer 407, Poly-vinylpyrrolidone, Pop313, Crown-5, and any combination thereof. Exemplary supplemental factors include, but are not limited to, media such as PBS, HBSS, OptiMEM, DMEM, RPMI 1640, AIM-V, X-VIVO 15, CellGro DC Medium, CTS OpTimizer T Cell Expansion SFM, TexMACS Medium, PRIME-XV T Cell Expansion Medium, ImmunoCult-XF T Cell Expansion Medium and any combination thereof. Exemplary supplemental factors include, but are not limited to, inhibitors of cellular DNA sensing, metabolism, differentiation, signal transduction, the apoptotic pathway and combinations thereof. Exemplary inhibitors include, but are not limited to, inhibitors of TLR9, MyD88, IRAK, TRAF6, TRAF3, IRF-7, NF-KB, Type 1 Interferons, pro-inflammatory cytokines, cGAS, STING, Sec5, TBK1, IRF-3, RNA pol III, RIG-1, IPS-1, FADD, RIP1, TRAF3, AIM2, ASC, Caspase1, Pro-IL1B, PI3K, Akt, Wnt3A, inhibitors of glycogen synthase kinase-3β (GSK-3 β) (e.g. TWS119), Bafilomycin, Chloroquine, Quinacrine, AC-YVAD-CMK, Z-VAD-FMK, Z-IETD-FMK and any combination thereof. Exemplary supplemental factors include, but are not limited to, reagents that modify or stabilize one or more nucleic acids in a way to enhance cellular delivery, enhance nuclear delivery or transport, enhance the facilitated transport of nucleic acid into the nucleus, enhance degradation of epi-chromosomal nucleic acid, and/or decrease DNA-mediated toxicity. Exemplary reagents that modify or stabilize one or more nucleic acids include, but are not limited to, pH modifiers, DNA-binding proteins, lipids, phospholipids, CaPO4, net neutral charge DNA binding peptides with or without NLS sequences, TREX1 enzyme, and any combination thereof.

Transposition reagents, including a transposon and a transposase, may be added to a nucleofection reaction of the disclosure prior to, simultaneously with, or after an addition of cells to a nucleofection buffer (optionally, contained within a nucleofection reaction vial or cuvette). Transposons of the disclosure may comprise plasmid DNA, linearized plasmid DNA, a PCR product, DOGGYBONE™ DNA, an mRNA template, a single or double-stranded DNA, a protein-nucleic acid combination or any combination thereof. Transposons of the disclosure may comprised one or more sequences that encode one or more TTAA site(s), one or more inverted terminal repeat(s) (ITRs), one or more long terminal repeat(s) (LTRs), one or more insulator(s), one or more promotor(s), one or more full-length or truncated gene(s), one or more polyA signal(s), one or more self-cleaving 2A peptide cleavage site(s), one or more internal ribosome entry site(s) (IRES), one or more enhancer(s), one or more regulator(s), one or more replication origin(s), and any combination thereof.

Transposons of the disclosure may comprise one or more sequences that encode one or more full-length or truncated gene(s). Full-length and/or truncated gene(s) introduced by transposons of the disclosure may encode one or more of a signal peptide, a Centyrin, a single chain variable fragment (scFv), a hinge, a transmembrane domain, a costimulatory domain, a chimeric antigen receptor (CAR), a chimeric T-cell receptor (CAR-T), a CARTyrin (a CAR-T comprising a Centyrin), a receptor, a ligand, a cytokine, a drug resistance gene, a tumor antigen, an allo or auto antigen, an enzyme, a protein, a peptide, a poly-peptide, a fluorescent protein, a mutein or any combination thereof.

Transposons of the disclosure may be prepared in water, TAE, TBE, PBS, HBSS, media, a supplemental factor of the disclosure or any combination thereof.

Transposons of the disclosure may be designed to optimize clinical safety and/or improve manufacturability. As a non-limiting example, transposons of the disclosure may be designed to optimize clinical safety and/or improve manufacturability by eliminating unnecessary sequences or regions and/or including a non-antibiotic selection marker. Transposons of the disclosure may or may not be GMP grade.

Transposase enzymes of the disclosure may be encoded by one or more sequences of plasmid DNA, mRNA, protein, protein-nucleic acid combination or any combination thereof.

Transposase enzymes of the disclosure may be prepared in water, TAE, TBE, PBS, HBSS, media, a supplemental factor of the disclosure or any combination thereof. Transposase enzymes of the disclosure or the sequences/constructs encoding or delivering them may or may not be GMP grade.

Transposons and transposase enzymes of the disclosure may be delivered to a cell by any means.

Although compositions and methods of the disclosure include delivery of a transposon and/or transposase of the disclosure to a cell by plasmid DNA (pDNA), the use of a plasmid for delivery may allow the transposon and/or transposase to be integrated into the chromosomal DNA of the cell, which may lead to continued transposase expression. Accordingly, transposon and/or transposase enzymes of the disclosure may be delivered to a cell as either mRNA or protein to remove any possibility for chromosomal integration.

Transposons and transposases of the disclosure may be pre-incubated alone or in combination with one another prior to the introduction of the transposon and/or transposase into a nucleofection reaction. The absolute amounts of each of the transposon and the transposase, as well as the relative amounts, e.g., a ratio of transposon to transposase may be optimized.

Following preparation of nucleofection reaction, optionally, in a vial or cuvette, the reaction may be loaded into a nucleofector apparatus and activated for delivery of an electric pulse according to the manufacturer's protocol. Electric pulse conditions used for delivery of a transposon and/or a transposase of the disclosure (or a sequence encoding a transposon and/or a transposase of the disclosure) to a cell may be optimized for yielding cells with enhanced viability, higher nucleofection efficiency, greater viability post-nucleofection, desirable cell phenotype, and/or greater/faster expansion upon addition of expansion technologies. When using Amaxa nucleofector technology, each of the various nucleofection programs for the Amaxa 2B or 4D nucleofector are contemplated.

Following a nucleofection reaction of the disclosure, cells may be gently added to a cell medium. For example, when T cells undergo the nucleofection reaction, the T cells may be added to a T cell medium. Post-nucleofection cell media of the disclosure may comprise any one or more commercially-available media. Post-nucleofection cell media of the disclosure (including post-nucleofection T cell media of the disclosure) may be optimized to yield cells with greater viability, higher nucleofection efficiency, exhibit greater viability post-nucleofection, display a more desirable cell phenotype, and/or greater/faster expansion upon addition of expansion technologies. Post-nucleofection cell media of the disclosure (including post-nucleofection T cell media of the disclosure) may comprise PBS, HBSS, OptiMEM, DMEM, RPMI 1640, AIM-V, X-VIVO 15, CellGro DC Medium, CTS OpTimizer T Cell Expansion SFM, TexMACS Medium, PRIME-XV T Cell Expansion Medium, ImmunoCult-XF T Cell Expansion Medium and any combination thereof. Post-nucleofection cell media of the disclosure (including post-nucleofection T cell media of the disclosure) may comprise one or more supplemental factors of the disclosure to enhance viability, nucleofection efficiency, viability post-nucleofection, cell phenotype, and/or greater/faster expansion upon addition of expansion technologies. Exemplary supplemental factors include, but are not limited to, recombinant human cytokines, chemokines, interleukins and any combination thereof. Exemplary cytokines, chemokines, and interleukins include, but are not limited to, IL2, IL7, IL12, IL15, IL21, IL1, IL3, IL4, IL5, IL6, IL8, CXCL8, IL9, IL10, IL11, IL13, IL14, IL16, IL17, IL18, IL19, IL20, IL22, IL23, IL25, IL26, IL27, IL28, IL29, IL30, IL31, IL32, IL33, IL35, IL36, GM-CSF, IFN-gamma, IL-1 alpha/IL-1F1, IL-1 beta/IL-1F2, IL-12 p70, IL-12/IL-35 p35, IL-13, IL-17/IL-17A, IL-17A/F Heterodimer, IL-17F, IL-18/IL-1F4, IL-23, IL-24, IL-32, IL-32 beta, IL-32 gamma, IL-33, LAP (TGF-beta 1), Lymphotoxin-alpha/TNF-beta, TGF-beta, TNF-alpha, TRANCE/TNFSF11/RANK L and any combination thereof. Exemplary supplemental factors include, but are not limited to, salts, minerals, metabolites or any combination thereof. Exemplary salts, minerals, and metabolites include, but are not limited to, HEPES, Nicotinamide, Heparin, Sodium Pyruvate, L-Glutamine, MEM Non-Essential Amino Acid Solution, Ascorbic Acid, Nucleosides, FBS/FCS, Human serum, serum-substitute, anti-biotics, pH adjusters, Earle's Salts, 2-Mercaptoethanol, Human transferrin, Recombinant human insulin, Human serum albumin, Nucleofector PLUS Supplement, KCL, MgCl2, Na2HPO4, NAH2PO4, Sodium lactobionate, Manitol, Sodium succinate, Sodium Chloride, CINa, Glucose, Ca(NO3)2, Tris/HCl, K2HPO4, KH2PO4, Polyethylenimine, Poly-ethylene-glycol, Poloxamer 188, Poloxamer 181, Poloxamer 407, Poly-vinylpyrrolidone, Pop313, Crown-5, and any combination thereof. Exemplary supplemental factors include, but are not limited to, media such as PBS, HBSS, OptiMEM, DMEM, RPMI 1640, AIM-V, X-VIVO 15, CellGro DC Medium, CTS OpTimizer T Cell Expansion SFM, TexMACS Medium, PRIME-XV T Cell Expansion Medium, ImmunoCult-XF T Cell Expansion Medium and any combination thereof. Exemplary supplemental factors include, but are not limited to, inhibitors of cellular DNA sensing, metabolism, differentiation, signal transduction, the apoptotic pathway and combinations thereof. Exemplary inhibitors include, but are not limited to, inhibitors of TLR9, MyD88, IRAK, TRAF6, TRAF3, IRF-7, NF-KB, Type 1 Interferons, pro-inflammatory cytokines, cGAS, STING, Sec5, TBK1, IRF-3, RNA pol III, RIG-1, IPS-1, FADD, RIP1, TRAF3, AIM2, ASC, Caspase1, Pro-IL1B, PI3K, Akt, Wnt3A, inhibitors of glycogen synthase kinase-3β (GSK-3 β) (e.g. TWS119), Bafilomycin, Chloroquine, Quinacrine, AC-YVAD-CMK, Z-VAD-FMK, Z-IETD-FMK and any combination thereof. Exemplary supplemental factors include, but are not limited to, reagents that modify or stabilize one or more nucleic acids in a way to enhance cellular delivery, enhance nuclear delivery or transport, enhance the facilitated transport of nucleic acid into the nucleus, enhance degradation of epi-chromosomal nucleic acid, and/or decrease DNA-mediated toxicity. Exemplary reagents that modify or stabilize one or more nucleic acids include, but are not limited to, pH modifiers, DNA-binding proteins, lipids, phospholipids, CaPO4, net neutral charge DNA binding peptides with or without NLS sequences, TREX1 enzyme, and any combination thereof.

Post-nucleofection cell media of the disclosure (including post-nucleofection T cell media of the disclosure) may be used at room temperature or pre-warmed to, for example to between 32° C. to 37° C., inclusive of the endpoints. Post-nucleofection cell media of the disclosure (including post-nucleofection T cell media of the disclosure) may be pre-warmed to any temperature that maintains or enhances cell viability and/or expression of a transposon or portion thereof of the disclosure.

Post-nucleofection cell media of the disclosure (including post-nucleofection T cell media of the disclosure) may be contained in tissue culture flasks or dishes, G-Rex flasks, Bioreactor or cell culture bags, or any other standard receptacle. Post-nucleofection cell cultures of the disclosure (including post-nucleofection T cell cultures of the disclosure) may be kept still, or, alternatively, they may be perturbed (e.g. rocked, swirled, or shaken).

Post-nucleofection cell cultures may comprise genetically-modified cells. Post-nucleofection T cell cultures may comprise genetically-modified T cells. Genetically modified cells of the disclosure may be either rested for a defined period of time or stimulated for expansion by, for example, the addition of a T Cell Expander technology. In certain embodiments, genetically modified cells of the disclosure may be either rested for a defined period of time or immediately stimulated for expansion by, for example, the addition of a T Cell Expander technology. Genetically modified cells of the disclosure may be rested to allow them sufficient time to acclimate, time for transposition to occur, and/or time for positive or negative selection, resulting in cells with enhanced viability, higher nucleofection efficiency, greater viability post-nucleofection, desirable cell phenotype, and/or greater/faster expansion upon addition of expansion technologies. Genetically modified cells of the disclosure may be rested, for example, for 1, 2, 3, 4, 5, 6, 7, 8, 9, 10, 11, 12, 13, 14, 15, 16, 17, 18, 19, 20, 21, 22, 23, 24, or more hours. In certain embodiments, genetically modified cells of the disclosure may be rested, for example, for an overnight. In certain aspects, an overnight is about 12 hours. Genetically modified cells of the disclosure may be rested, for example, for 1, 2, 3, 4, 5, 6, 7, 8, 9, 10, 11, 12, 13, 14 or more days.

Genetically modified cells of the disclosure may be selected following a nucleofection reaction and prior to addition of an expander technology. For optimal selection of genetically-modified cells, the cells may be allowed to rest in a post-nucleofection cell medium for at least 2-14 days to facilitate identification of modified cells (e.g., differentiation of modified from non-modified cells).

As early as 24-hours post-nucleofection, expression of a CAR/CARTyrin and selection marker of the disclosure may be detectable in modified T cells upon successful nucleofection of a transposon of the disclosure. Due to epi-chromosomal expression of the transposon, expression of a selection marker alone may not differentiate modified T cells (those cells in which the transposon has been successfully integrated) from unmodified T cells (those cells in which the transposon was not successfully integrated). When epi-chromosomal expression of the transposon obscures the detection of modified cells by the selection marker, the nucleofected cells (both modified and unmodified cells) may be rested for a period of time (e.g. 2-14 days) to allow the cells to cease expression or lose all epi-chromosomal transposon expression. Following this extended resting period, only modified T cells should remain positive for expression of selection marker. The length of this extended resting period may be optimized for each nucleofection reaction and selection process. When epi-chromosomal expression of the transposon obscures the detection of modified cells by the selection marker, selection may be performed without this extended resting period, however, an additional selection step may be included at a later time point (e.g. either during or after the expansion stage).

Selection of genetically modified cells of the disclosure may be performed by any means. In certain embodiments of the methods of the disclosure, selection of genetically modified cells of the disclosure may be performed by isolating cells expressing a specific selection marker. Selection markers of the disclosure may be encoded by one or more sequences in the transposon. Selection markers of the disclosure may be expressed by the modified cell as a result of successful transposition (i.e., not encoded by one or more sequences in the transposon). In certain embodiments, genetically modified cells of the disclosure contain a selection marker that confers resistance to a deleterious compound of the post-nucleofection cell medium. The deleterious compound may comprise, for example, an antibiotic or a drug that, absent the resistance conferred by the selection marker to the modified cells, would result in cell death. Exemplary selection markers include, but are not limited to, wild type (WT) or mutant forms of one or more of the following genes: neo, DHFR, TYMS, ALDH, MDR1, MGMT, FANCF, RAD51C, GCS, and NKX2.2. Exemplary selection markers include, but are not limited to, a surface-expressed selection marker or surface-expressed tag may be targeted by Ab-coated magnetic bead technology or column selection, respectively. A cleavable tag such as those used in protein purification may be added to a selection marker of the disclosure for efficient column selection, washing, and elution. In certain embodiments, selection markers of the disclosure are not expressed by the modified cells (including modified T cells) naturally and, therefore, may be useful in the physical isolation of modified cells (by, for example, cell sorting techniques). Exemplary selection markers of the disclosure are not expressed by the modified cells (including modified T cells) naturally include, but are not limited to, full-length, mutated, or truncated forms of CD271, CD19 CD52, CD34, RQR8, CD22, CD20, CD33 and any combination thereof.

Genetically modified cells of the disclosure may be selective expanded following a nucleofection reaction. In certain embodiments, modified T cells comprising a CAR/CARTyrin may be selectively expanded by CAR/CARTyrin stimulation. Modified T cells comprising a CAR/CARTyrin may be stimulated by contact with a target-covered reagent (e.g. a tumor line or a normal cell line expressing a target or expander beads covered in a target). Alternatively, modified T cells comprising a CAR/CARTyrin may be stimulated by contact with an irradiated tumor cell, an irradiated allogeneic normal cell, an irradiated autologous PBMC. To minimize contamination of cell product compositions of the disclosure with a target-expressing cell used for stimulation, for example, when the cell product composition may be administered directly to a subject, the stimulation may be performed using expander beads coated with CAR/CARTyrin target protein. Selective expansion of modified T cells comprising a CAR/CARTyrin by CAR/CARTyrin stimulation may be optimized to avoid functionally-exhausting the modified T-cells.

Selected genetically-modified cells of the disclosure may be cryopreserved, rested for a defined period of time, or stimulated for expansion by the addition of a Cell Expander technology. Selected genetically-modified cells of the disclosure may be cryopreserved, rested for a defined period of time, or immediately stimulated for expansion by the addition of a Cell Expander technology. When the selected genetically-modified cells are T cells, the T cells may be stimulated for expansion by the addition of a T-Cell Expander technology. Selected genetically modified cells of the disclosure may be rested, for example, for 1, 2, 3, 4, 5, 6, 7, 8, 9, 10, 11, 12, 13, 14, 15, 16, 17, 18, 19, 20, 21, 22, 23, 24, or more hours. In certain embodiments, selected genetically modified cells of the disclosure may be rested, for example, for an overnight. In certain aspects, an overnight is about 12 hours. Selected genetically modified cells of the disclosure may be rested, for example, for 1, 2, 3, 4, 5, 6, 7, 8, 9, 10, 11, 12, 13, 14 or more days. Selected genetically modified cells of the disclosure may be rested for any period of time resulting in cells with enhanced viability, higher nucleofection efficiency, greater viability post-nucleofection, desirable cell phenotype, and/or greater/faster expansion upon addition of expansion technologies.

Selected genetically-modified cells (including selected genetically-modified T cells of the disclosure) may be cryopreserved using any standard cryopreservation method, which may be optimized for storing and/or recovering human cells with high recovery, viability, phenotype, and/or functional capacity. Cryopreservation methods of the disclosure may include commercially-available cryopreservation media and/or protocols.

A transposition efficiency of selected genetically-modified cells (including selected genetically-modified T cells of the disclosure) may be assessed by any means. For example, prior to the application of an expander technology, expression of the transposon by selected genetically-modified cells (including selected genetically-modified T cells of the disclosure) may be measured by fluorescence-activated cell sorting (FACS). Determination of a transposition efficiency of selected genetically-modified cells (including selected genetically-modified T cells of the disclosure) may include determining a percentage of selected cells expressing the transposon (e.g. a CAR). Alternatively, or in addition, a purity of T cells, a Mean Fluorescence Intensity (MFI) of the transposon expression (e.g. CAR expression), an ability of a CAR (delivered in the transposon) to mediate degranulation and/or killing of a target cell expressing the CAR ligand, and/or a phenotype of selected genetically-modified cells (including selected genetically-modified T cells of the disclosure) may be assessed by any means.

Cell product compositions of the disclosure may be released for administration to a subject upon meeting certain release criteria. Exemplary release criteria may include, but are not limited to, a particular percentage of modified, selected and/or expanded T cells expressing detectable levels of a CAR on the cell surface.

### Genetic Modification of an Autologous T Cell Product Composition

Genetically-modified cells (including genetically-modified T cells) of the disclosure may be expanded using an expander technology. Expander technologies of the disclosure may comprise a commercially-available expander technology. Exemplary expander technologies of the disclosure include stimulation a genetically-modified T cell of the disclosure via the TCR. While all means for stimulation of a genetically-modified T cell of the disclosure are contemplated, stimulation a genetically-modified T cell of the disclosure via the TCR is a preferred method, yielding a product with a superior level of killing capacity.

To stimulate a genetically-modified T cell of the disclosure via the TCR, Thermo Expander DynaBeads may be used at a 3:1 bead to T cell ratio. If the expander beads are not biodegradable, the beads may be removed from the expander composition. For example, the beads may be removed from the expander composition after about 5 days. To stimulate a genetically-modified T cell of the disclosure via the TCR, a Miltenyi T Cell Activation/Expansion Reagent may be used. To stimulate a genetically-modified T cell of the disclosure via the TCR, StemCell Technologies' ImmunoCult Human CD3/CD28 or CD3/CD28/CD2 T Cell Activator Reagent may be used. This technology may be preferred since the soluble tetrameric antibody complexes would degrade after a period and would not require removal from the process.

Artificial antigen presenting cells (APCs) may be engineered to co-express the target antigen and may be used to stimulate a cell or T-cell of the disclosure through a TCR and/or CAR of the disclosure. Artificial APCs may comprise or may be derived from a tumor cell line (including, for example, the immortalized myelogenous leukemia line K562) and may be engineered to co-express multiple costimulatory molecules or technologies (such as CD28, 4-1BBL, CD64, mbIL-21, mbIL-15, CAR target molecule, etc.). When artificial APCs of the disclosure are combined with costimulatory molecules, conditions may be optimized to prevent the development or emergence of an undesirable phenotype and functional capacity, namely terminally-differentiated effector T cells.

Irradiated PBMCs (auto or allo) may express some target antigens, such as CD19, and may be used to stimulate a cell or T-cell of the disclosure through a TCR and/or CAR of the disclosure. Alternatively, or in addition, irradiated tumor cells may express some target antigens and may be used to stimulate a cell or T-cell of the disclosure through a TCR and/or CAR of the disclosure.

Plate-bound and/or soluble anti-CD3, anti-CD2 and/or anti-CD28 stimulate may be used to stimulate a cell or T-cell of the disclosure through a TCR and/or CAR of the disclosure.

Antigen-coated beads may display target protein and may be used to stimulate a cell or T-cell of the disclosure through a TCR and/or CAR of the disclosure. Alternatively, or in addition, expander beads coated with a CAR/CARTyrin target protein may be used to stimulate a cell or T-cell of the disclosure through a TCR and/or CAR of the disclosure.

Expansion methods drawn to stimulation of a cell or T-cell of the disclosure through the TCR or CAR/CARTyrin and via surface-expressed CD2, CD3, CD28, 4-1BB, and/or other markers on genetically-modified T cells.

An expansion technology may be applied to a cell of the disclosure immediately post-nucleofection until approximately 24 hours post-nucleofection. While various cell media may be used during an expansion procedure, a desirable T Cell Expansion Media of the disclosure may yield cells with, for example, greater viability, cell phenotype, total expansion, or greater capacity for in vivo persistence, engraftment, and/or CAR-mediated killing. Cell media of the disclosure may be optimized to improve/enhance expansion, phenotype, and function of genetically-modified cells of the disclosure. A preferred phenotype of expanded T cells may include a mixture of T stem cell memory, T central, and T effector memory cells. Expander Dynabeads may yield mainly central memory T cells which may lead to superior performance in the clinic.

Exemplary T cell expansion media of the disclosure may include, in part or in total, PBS, HBSS, OptiMEM, DMEM, RPMI 1640, AIM-V, X-VIVO 15, CellGro DC Medium, CTS OpTimizer T Cell Expansion SFM, TexMACS Medium, PRIME-XV T Cell Expansion Medium, ImmunoCult-XF T Cell Expansion Medium, or any combination thereof. T cell expansion media of the disclosure may further include one or more supplemental factors. Supplemental factors that may be included in a T cell expansion media of the disclosure enhance viability, cell phenotype, total expansion, or increase capacity for in vivo persistence, engraftment, and/or CAR-mediated killing. Supplemental factors that may be included in a T cell expansion media of the disclosure include, but are not limited to, recombinant human cytokines, chemokines, and/or interleukins such as IL2, IL7, IL12, IL15, IL21, IL1, IL3, IL4, IL5, IL6, IL8, CXCL8, IL9, IL10, IL11, IL13, IL14, IL16, IL17, IL18, IL19, IL20, IL22, IL23, IL25, IL26, IL27, IL28, IL29, IL30, IL31, IL32, IL33, IL35, IL36, GM-CSF, IFN-gamma, IL-1 alpha/IL-1F1, IL-1 beta/IL-1F2, IL-12 p70, IL-12/IL-35 p35, IL-13, IL-17/IL-17A, IL-17A/F Heterodimer, IL-17F, IL-18/IL-1F4, IL-23, IL-24, IL-32, IL-32 beta, IL-32 gamma, IL-33, LAP (TGF-beta 1), Lymphotoxin-alpha/TNF-beta, TGF-beta, TNF-alpha, TRANCE/TNFSF11/RANK L, or any combination thereof. Supplemental factors that may be included in a T cell expansion media of the disclosure include, but are not limited to, salts, minerals, and/or metabolites such as HEPES, Nicotinamide, Heparin, Sodium Pyruvate, L-Glutamine, MEM Non-Essential Amino Acid Solution, Ascorbic Acid, Nucleosides, FBS/FCS, Human serum, serum-substitute, anti-biotics, pH adjusters, Earle's Salts, 2-Mercaptoethanol, Human transferrin, Recombinant human insulin, Human serum albumin, Nucleofector PLUS Supplement, KCL, MgCl2, Na2HPO4, NAH2PO4, Sodium lactobionate, Manitol, Sodium succinate, Sodium Chloride, CINa, Glucose, Ca(NO3)2, Tris/HCl, K2HPO4, KH2PO4, Polyethylenimine, Poly-ethylene-glycol, Poloxamer 188, Poloxamer 181, Poloxamer 407, Poly-vinylpyrrolidone, Pop313, Crown-5 or any combination thereof. Supplemental factors that may be included in a T cell expansion media of the disclosure include, but are not limited to, inhibitors of cellular DNA sensing, metabolism, differentiation, signal transduction, and/or the apoptotic pathway such as inhibitors of TLR9, MyD88, IRAK, TRAF6, TRAF3, IRF-7, NF-KB, Type 1 Interferons, pro-inflammatory cytokines, cGAS, STING, Sec5, TBK1, IRF-3, RNA pol III, RIG-1, IPS-1, FADD, RIP1, TRAF3, AIM2, ASC, Caspase1, Pro-IL1B, PI3K, Akt, Wnt3A, inhibitors of glycogen synthase kinase-3β (GSK-3 β) (e.g. TWS119), Bafilomycin, Chloroquine, Quinacrine, AC-YVAD-CMK, Z-VAD-FMK, Z-IETD-FMK, or any combination thereof.

Supplemental factors that may be included in a T cell expansion media of the disclosure include, but are not limited to, reagents that modify or stabilize nucleic acids in a way to enhance cellular delivery, enhance nuclear delivery or transport, enhance the facilitated transport of nucleic acid into the nucleus, enhance degradation of epi-chromosomal nucleic acid, and/or decrease DNA-mediated toxicity, such as pH modifiers, DNA-binding proteins, lipids, phospholipids, CaPO4, net neutral charge DNA binding peptides with or without NLS sequences, TREX1 enzyme, or any combination thereof.

Genetically-modified cells of the disclosure may be selected during the expansion process by the use of selectable drugs or compounds. For example, in certain embodiments, when a transposon of the disclosure may encode a selection marker that confers to genetically-modified cells resistance to a drug added to the culture medium, selection may occur during the expansion process and may require approximately 1-14 days of culture for selection to occur. Examples of drug resistance genes that may be used as selection markers encoded by a transposon of the disclosure, include, but are not limited to, wild type (WT) or mutant forms of the genes neo, DHFR, TYMS, ALDH, MDR1, MGMT, FANCF, RAD51C, GCS, NKX2.2, or any combination thereof. Examples of corresponding drugs or compounds that may be added to the culture medium to which a selection marker may confer resistance include, but are not limited to, G418, Puromycin, Ampicillin, Kanamycin, Methotrexate, Mephalan, Temozolomide, Vincristine, Etoposide, Doxorubicin, Bendamustine, Fludarabine, Aredia (Pamidronate Disodium), Becenum (Carmustine), BiCNU (Carmustine), Bortezomib, Carfilzomib, Carmubris (Carmustine), Carmustine, Clafen (Cyclophosphamide), Cyclophosphamide, Cytoxan (Cyclophosphamide), Daratumumab, Darzalex (Daratumumab), Doxil (Doxorubicin Hydrochloride Liposome), Doxorubicin Hydrochloride Liposome, Dox-SL (Doxorubicin Hydrochloride Liposome), Elotuzumab, Empliciti (Elotuzumab), Evacet (Doxorubicin Hydrochloride Liposome), Farydak (Panobinostat), Ixazomib Citrate, Kyprolis (Carfilzomib), Lenalidomide, LipoDox (Doxorubicin Hydrochloride Liposome), Mozobil (Plerixafor), Neosar (Cyclophosphamide), Ninlaro (Ixazomib Citrate), Pamidronate Disodium, Panobinostat, Plerixafor, Pomalidomide, Pomalyst (Pomalidomide), Revlimid (Lenalidomide), Synovir (Thalidomide), Thalidomide, Thalomid (Thalidomide), Velcade (Bortezomib), Zoledronic Acid, Zometa (Zoledronic Acid), or any combination thereof.

A T-Cell Expansion process of the disclosure may occur in a cell culture bag in a WAVE Bioreactor, a G-Rex flask, or in any other suitable container and/or reactor.

A cell or T-cell culture of the disclosure may be kept steady, rocked, swirled, or shaken.

A cell or T-cell expansion process of the disclosure may optimize certain conditions, including, but not limited to culture duration, cell concentration, schedule for T cell medium addition/removal, cell size, total cell number, cell phenotype, purity of cell population, percentage of genetically-modified cells in growing cell population, use and composition of supplements, the addition/removal of expander technologies, or any combination thereof.

A cell or T-cell expansion process of the disclosure may continue until a predefined endpoint prior to formulation of the resultant expanded cell population. For example, a cell or T-cell expansion process of the disclosure may continue for a predetermined amount of time: at least, 2, 4, 6, 8, 10, 12, 14, 16, 18, 20, 22, 24 hours; at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days; at least 1, 2, 3, 4, 5, 6, 7, 8, 9, 10, 11, 12 weeks; at least 1, 2, 3, 4, 5, 6, months, or at least 1 year. A cell or T-cell expansion process of the disclosure may continue until the resultant culture reaches a predetermined overall cell density: 1, 10, 100, 1000, 104, 105, 106, 107, 108, 109, 1010 cells per volume (μl, ml, L) or any density in between. A cell or T-cell expansion process of the disclosure may continue until the genetically-modified cells of a resultant culture demonstrate a predetermined level of expression of a transposon of the disclosure: 1%, 10%, 20%, 30%, 40%, 50%, 60%, 70%, 80%, 90%, or 100% or any percentage in between of a threshold level of expression (a minimum, maximum or mean level of expression indicating the resultant genetically-modified cells are clinically-efficacious). A cell or T-cell expansion process of the disclosure may continue until the proportion of genetically-modified cells of a resultant culture to the proportion of unmodified cells reaches a predetermined threshold: at least 1:10, 1:9, 1:8, 1:7, 1:6, 1:5, 1:4, 1:3, 1:2, 1:1, 2:1, 2:1, 4:1, 5:1, 6:1, 7:1, 8:1, 9:1 10:1 or any ratio in between.

### Analysis of Genetically-Modified Autologous T Cells for Release

A percentage of genetically-modified cells may be assessed during or after an expansion process of the disclosure. Cellular expression of a transposon by a genetically-modified cell of the disclosure may be measured by fluorescence-activated cell sorting (FACS). For example, FACS may be used to determine a percentage of cells or T cells expressing a CAR of the disclosure. Alternatively, or in addition, a purity of genetically-modified cells or T cells, the Mean Fluorescence Intensity (MFI) of a CAR expressed by a genetically-modified cell or T cell of the disclosure, an ability of the CAR to mediate degranulation and/or killing of a target cell expressing the CAR ligand, and/or a phenotype of CAR+ T cells may be assessed.

Compositions of the disclosure intended for administration to a subject may be required to meet one or more “release criteria” that indicate that the composition is safe and efficacious for formulation as a pharmaceutical product and/or administration to a subject. Release criteria may include a requirement that a composition of the disclosure (e.g. a T-cell product of the disclosure) comprises a particular percentage of T cells expressing detectable levels of a CAR of the disclosure on their cell surface.

The expansion process should be continued until a specific criterion has been met (e.g. achieving a certain total number of cells, achieving a particular population of memory cells, achieving a population of a specific size).

Certain criterion signal a point at which the expansion process should end. For example, cells should be formulated, reactivated, or cryopreserved once they reach a cell size of 300 fL (otherwise, cells reaching a size above this threshold may start to die). Cryopreservation immediately once a population of cells reaches an average cell size of less than 300 fL may yield better cell recovery upon thawing and culture because the cells haven't yet reached a fully quiescent state prior to cryopreservation (a fully quiescent size is approximately 180 fL). Prior to expansion, T cells of the disclosure may have a cell size of about 180 fL, but may more than quadruple their cell size to approximately 900 fL at 3 days post-expansion. Over the next 6-12 days, the population of T-cells will slowly decrease cell size to full quiescence at 180 fL.

A process for preparing a cell population for formulation may include, but is not limited to the steps of, concentrating the cells of the cell population, washing the cells, and/or further selection of the cells via drug resistance or magnetic bead sorting against a particular surface-expressed marker. A process for preparing a cell population for formulation may further include a sorting step to ensure the safety and purity of the final product. For example, if a tumor cell from a patient has been used to stimulate a genetically-modified T-cell of the disclosure or that have been genetically-modified in order to stimulate a genetically-modified T-cell of the disclosure that is being prepared for formulation, it is critical that no tumor cells from the patient are included in the final product.

Cell Product Infusion and/or Cryopreservation for Infusion

A pharmaceutical formulation of the disclosure may be distributed into bags for infusion, cryopreservation, and/or storage.

A pharmaceutical formulation of the disclosure may be cryopreserved using a standard protocol and, optionally, an infusible cryopreservation medium. For example, a DMSO free cryopreservant (e.g. CryoSOfree™ DMSO-free Cryopreservation Medium) may be used to reduce freezing-related toxicity. A cryopreserved pharmaceutical formulation of the disclosure may be stored for infusion to a patient at a later date. An effective treatment may require multiple administrations of a pharmaceutical formulation of the disclosure and, therefore, pharmaceutical formulations may be packaged in pre-aliquoted “doses” that may be stored frozen but separated for thawing of individual doses.

A pharmaceutical formulation of the disclosure may be stored at room temperature. An effective treatment may require multiple administrations of a pharmaceutical formulation of the disclosure and, therefore, pharmaceutical formulations may be packaged in pre-aliquoted “doses” that may be stored together but separated for administration of individual doses.

A pharmaceutical formulation of the disclosure may be archived for subsequent re-expansion and/or selection for generation of additional doses to the same patient in the case of an allogenic therapy who may need an administration at a future date following, for example, a remission and relapse of a condition.

### Formulations

As noted above, the disclosure provides for stable formulations, which preferably comprise a phosphate buffer with saline or a chosen salt, as well as preserved solutions and formulations containing a preservative as well as multi-use preserved formulations suitable for pharmaceutical or veterinary use, comprising at least one protein scaffold in a pharmaceutically acceptable formulation. Preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, polymers, or mixtures thereof in an aqueous diluent. Any suitable concentration or mixture can be used as known in the art, such as about 0.0015%, or any range, value, or fraction therein. Non-limiting examples include, no preservative, about 0.1-2% m-cresol (e.g., 0.2, 0.3. 0.4, 0.5, 0.9, 1.0%), about 0.1-3% benzyl alcohol (e.g., 0.5, 0.9, 1.1, 1.5, 1.9, 2.0, 2.5%), about 0.001-0.5% thimerosal (e.g., 0.005, 0.01), about 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like.

As noted above, the invention provides an article of manufacture, comprising packaging material and at least one vial comprising a solution of at least one protein scaffold with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The invention further comprises an article of manufacture, comprising packaging material, a first vial comprising lyophilized at least one protein scaffold, and a second vial comprising an aqueous diluent of prescribed buffer or preservative, wherein said packaging material comprises a label that instructs a patient to reconstitute the at least one protein scaffold in the aqueous diluent to form a solution that can be held over a period of twenty-four hours or greater.

The at least one protein scaffold used in accordance with the present invention can be produced by recombinant means, including from mammalian cell or transgenic preparations, or can be purified from other biological sources, as described herein or as known in the art.

The range of at least one protein scaffold in the product of the present invention includes amounts yielding upon reconstitution, if in a wet/dry system, concentrations from about 1.0 μg/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

Preferably, the aqueous diluent optionally further comprises a pharmaceutically acceptable preservative. Preferred preservatives include those selected from the group consisting of phenol, m-cresol, p-cresol, o-cresol, chlorocresol, benzyl alcohol, alkylparaben (methyl, ethyl, propyl, butyl and the like), benzalkonium chloride, benzethonium chloride, sodium dehydroacetate and thimerosal, or mixtures thereof. The concentration of preservative used in the formulation is a concentration sufficient to yield an anti-microbial effect. Such concentrations are dependent on the preservative selected and are readily determined by the skilled artisan.

Other excipients, e.g., isotonicity agents, buffers, antioxidants, and preservative enhancers, can be optionally and preferably added to the diluent. An isotonicity agent, such as glycerin, is commonly used at known concentrations. A physiologically tolerated buffer is preferably added to provide improved pH control. The formulations can cover a wide range of pHs, such as from about pH 4 to about pH 10, and preferred ranges from about pH 5 to about pH 9, and a most preferred range of about 6.0 to about 8.0. Preferably, the formulations of the present invention have a pH between about 6.8 and about 7.8. Preferred buffers include phosphate buffers, most preferably, sodium phosphate, particularly, phosphate buffered saline (PBS).

Other additives, such as a pharmaceutically acceptable solubilizers like Tween 20 (polyoxyethylene (20) sorbitan monolaurate), Tween 40 (polyoxyethylene (20) sorbitan monopalmitate), Tween 80 (polyoxyethylene (20) sorbitan monooleate), Pluronic F68 (polyoxyethylene polyoxypropylene block copolymers), and PEG (polyethylene glycol) or non-ionic surfactants, such as polysorbate 20 or 80 or poloxamer 184 or 188, Pluronic® polyls, other block co-polymers, and chelators, such as EDTA and EGTA, can optionally be added to the formulations or compositions to reduce aggregation. These additives are particularly useful if a pump or plastic container is used to administer the formulation. The presence of pharmaceutically acceptable surfactant mitigates the propensity for the protein to aggregate.

The formulations of the present invention can be prepared by a process which comprises mixing at least one protein scaffold and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing the at least one protein scaffold and preservative in an aqueous diluent is carried out using conventional dissolution and mixing procedures. To prepare a suitable formulation, for example, a measured amount of at least one protein scaffold in buffered solution is combined with the desired preservative in a buffered solution in quantities sufficient to provide the protein and preservative at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed formulations can be provided to patients as clear solutions or as dual vials comprising a vial of lyophilized at least one protein scaffold that is reconstituted with a second vial containing water, a preservative and/or excipients, preferably, a phosphate buffer and/or saline and a chosen salt, in an aqueous diluent. Either a single solution vial or dual vial requiring reconstitution can be reused multiple times and can suffice for a single or multiple cycles of patient treatment and thus can provide a more convenient treatment regimen than currently available.

The present claimed articles of manufacture are useful for administration over a period ranging from immediate to twenty-four hours or greater. Accordingly, the presently claimed articles of manufacture offer significant advantages to the patient. Formulations of the invention can optionally be safely stored at temperatures of from about 2° C. to about 40° C. and retain the biological activity of the protein for extended periods of time, thus allowing a package label indicating that the solution can be held and/or used over a period of 6, 12, 18, 24, 36, 48, 72, or 96 hours or greater. If preserved diluent is used, such label can include use up to 1-12 months, one-half, one and a half, and/or two years.

The solutions of at least one protein scaffold of the invention can be prepared by a process that comprises mixing at least one protein scaffold in an aqueous diluent. Mixing is carried out using conventional dissolution and mixing procedures. To prepare a suitable diluent, for example, a measured amount of at least one protein scaffold in water or buffer is combined in quantities sufficient to provide the protein and, optionally, a preservative or buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed products can be provided to patients as clear solutions or as dual vials comprising a vial of lyophilized at least one protein scaffold that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available.

The claimed products can be provided indirectly to patients by providing to pharmacies, clinics, or other such institutions and facilities, clear solutions or dual vials comprising a vial of lyophilized at least one protein scaffold that is reconstituted with a second vial containing the aqueous diluent. The clear solution in this case can be up to one liter or even larger in size, providing a large reservoir from which smaller portions of the at least one protein scaffold solution can be retrieved one or multiple times for transfer into smaller vials and provided by the pharmacy or clinic to their customers and/or patients.

Recognized devices comprising single vial systems include pen-injector devices for delivery of a solution, such as BD Pens, BD Autojector®, Humaject®, NovoPen®, B-D® Pen, AutoPen®, and OptiPen®, GenotropinPen®, Genotronorm Pen®, Humatro Pen®, Reco-Pen®, Roferon Pen®, Biojector®, Iject®, J-tip Needle-Free Injector®, Intraject®, Medi-Ject®, e.g., as made or developed by Becton Dickinson (Franklin Lakes, N.J., www.bectondickenson.com), Disetronic (Burgdorf, Switzerland, www.disetronic.com; Bioject, Portland, Oreg. (www.bioject.com); National Medical Products, Weston Medical (Peterborough, UK, www.weston-medical.com), Medi-Ject Corp (Minneapolis, Minn., www.mediject.com), and similarly suitable devices. Recognized devices comprising a dual vial system include those pen-injector systems for reconstituting a lyophilized drug in a cartridge for delivery of the reconstituted solution, such as the HumatroPen®. Examples of other devices suitable include pre-filled syringes, auto-injectors, needle free injectors and needle free IV infusion sets.

The products presently claimed include packaging material. The packaging material provides, in addition to the information required by the regulatory agencies, the conditions under which the product can be used. The packaging material of the present invention provides instructions to the patient to reconstitute at least one protein scaffold in the aqueous diluent to form a solution and to use the solution over a period of 2-24 hours or greater for the two vial, wet/dry, product. For the single vial, solution product, the label indicates that such solution can be used over a period of 2-24 hours or greater. The presently claimed products are useful for human pharmaceutical product use.

The formulations of the present invention can be prepared by a process that comprises mixing at least one protein scaffold and a selected buffer, preferably, a phosphate buffer containing saline or a chosen salt. Mixing at least one protein scaffold and buffer in an aqueous diluent is carried out using conventional dissolution and mixing procedures. To prepare a suitable formulation, for example, a measured amount of at least one protein scaffold in water or buffer is combined with the desired buffering agent in water in quantities sufficient to provide the protein and buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed stable or preserved formulations can be provided to patients as clear solutions or as dual vials comprising a vial of lyophilized protein scaffold that is reconstituted with a second vial containing a preservative or buffer and excipients in an aqueous diluent. Either a single solution vial or dual vial requiring reconstitution can be reused multiple times and can suffice for a single or multiple cycles of patient treatment and thus provides a more convenient treatment regimen than currently available.

Other formulations or methods of stabilizing the protein scaffold may result in other than a clear solution of lyophilized powder comprising the protein scaffold. Among non-clear solutions are formulations comprising particulate suspensions, said particulates being a composition containing the protein scaffold in a structure of variable dimension and known variously as a microsphere, microparticle, nanoparticle, nanosphere, or liposome. Such relatively homogenous, essentially spherical, particulate formulations containing an active agent can be formed by contacting an aqueous phase containing the active agent and a polymer and a nonaqueous phase followed by evaporation of the nonaqueous phase to cause the coalescence of particles from the aqueous phase as taught in U.S. Pat. No. 4,589,330. Porous microparticles can be prepared using a first phase containing active agent and a polymer dispersed in a continuous solvent and removing said solvent from the suspension by freeze-drying or dilution-extraction-precipitation as taught in U.S. Pat. No. 4,818,542. Preferred polymers for such preparations are natural or synthetic copolymers or polymers selected from the group consisting of gelatin agar, starch, arabinogalactan, albumin, collagen, polyglycolic acid, polylactic aced, glycolide-L(−) lactide poly(epsilon-caprolactone, poly(epsilon-caprolactone-CO-lactic acid), poly(epsilon-caprolactone-CO-glycolic acid), poly(s-hydroxy butyric acid), polyethylene oxide, polyethylene, poly(alkyl-2-cyanoacrylate), poly(hydroxyethyl methacrylate), polyamides, poly(amino acids), poly(2-hydroxyethyl DL-aspartamide), poly(ester urea), poly(L-phenylalanine/ethylene glycol/1,6-diisocyanatohexane) and poly(methyl methacrylate). Particularly preferred polymers are polyesters, such as polyglycolic acid, polylactic aced, glycolide-L(−) lactide poly(epsilon-caprolactone, poly(epsilon-caprolactone-CO-lactic acid), and poly(epsilon-caprolactone-CO-glycolic acid. Solvents useful for dissolving the polymer and/or the active include: water, hexafluoroisopropanol, methylenechloride, tetrahydrofuran, hexane, benzene, or hexafluoroacetone sesquihydrate. The process of dispersing the active containing phase with a second phase may include pressure forcing said first phase through an orifice in a nozzle to affect droplet formation.

Dry powder formulations may result from processes other than lyophilization, such as by spray drying or solvent extraction by evaporation or by precipitation of a crystalline composition followed by one or more steps to remove aqueous or nonaqueous solvent. Preparation of a spray-dried protein scaffold preparation is taught in U.S. Pat. No. 6,019,968. The protein scaffold-based dry powder compositions may be produced by spray drying solutions or slurries of the protein scaffold and, optionally, excipients, in a solvent under conditions to provide a respirable dry powder. Solvents may include polar compounds, such as water and ethanol, which may be readily dried. Protein scaffold stability may be enhanced by performing the spray drying procedures in the absence of oxygen, such as under a nitrogen blanket or by using nitrogen as the drying gas. Another relatively dry formulation is a dispersion of a plurality of perforated microstructures dispersed in a suspension medium that typically comprises a hydrofluoroalkane propellant as taught in WO 9916419. The stabilized dispersions may be administered to the lung of a patient using a metered dose inhaler. Equipment useful in the commercial manufacture of spray dried medicaments are manufactured by Buchi Ltd. or Niro Corp.

At least one protein scaffold in either the stable or preserved formulations or solutions described herein, can be administered to a patient in accordance with the present invention via a variety of delivery methods including SC or IM injection; transdermal, pulmonary, transmucosal, implant, osmotic pump, cartridge, micro pump, or other means appreciated by the skilled artisan, as well-known in the art.

### Therapeutic Applications

The present invention also provides a method for modulating or treating a disease, in a cell, tissue, organ, animal, or patient, as known in the art or as described herein, using at least one protein scaffold of the present invention, e.g., administering or contacting the cell, tissue, organ, animal, or patient with a therapeutic effective amount of protein scaffold. The present invention also provides a method for modulating or treating a disease, in a cell, tissue, organ, animal, or patient including, but not limited to, a malignant disease.

The present invention also provides a method for modulating or treating at least one cancer or malignant disease in a cell, tissue, organ, animal or patient, including, but not limited to, at least one of: leukemia, acute leukemia, acute lymphoblastic leukemia (ALL), acute lymphocytic leukemia, B-cell, T-cell or FAB ALL, acute myeloid leukemia (AML), acute myelogenous leukemia, chronic myelocytic leukemia (CML), chronic lymphocytic leukemia (CLL), hairy cell leukemia, myelodyplastic syndrome (MDS), a lymphoma, Hodgkin's disease, a malignant lymphoma, non-Hodgkin's lymphoma, Burkitt's lymphoma, multiple myeloma, relapsed multiple myeloma, refractory multiple myeloma, Kaposi's sarcoma, colorectal carcinoma, pancreatic carcinoma, nasopharyngeal carcinoma, malignant histiocytosis, paraneoplastic syndrome/hypercalcemia of malignancy, solid tumors, bladder cancer, breast cancer, triple negative breast cancer, colorectal cancer, endometrial cancer, head cancer, neck cancer, hereditary nonpolyposis cancer, Hodgkin's lymphoma, liver cancer, lung cancer, non-small cell lung cancer, ovarian cancer, pancreatic cancer, prostate cancer, castrate-resistant prostate cancer, renal cell carcinoma, testicular cancer, adenocarcinomas, sarcomas, malignant melanoma, hemangioma, metastatic disease, cancer related bone resorption, cancer related bone pain, and the like. In some embodiments, the cancer is multiple myeloma. In some embodiments, the cancer is relapsed multiple myeloma. In some embodiments, the cancer is refractory multiple myeloma. In some embodiments, the cancer is a solid cancer. In some embodiments, the cancer is a prostate cancer. In some embodiments, the cancer is a castrate-resistant prostate cancer (CRPC). In some embodiments, the solid cancer is a breast cancer, a colorectal cancer, a lung cancer, an ovarian cancer, a pancreatic cancer or a renal cancer. In some embodiments, the breast cancer is triple negative breast cancer.

The present disclosure provides a method of treating cancer comprising administering to the subject: a first composition comprising a population of T-cells expressing a chimeric antigen receptor (CAR), wherein the CAR comprises an antigen recognition domain and a second composition comprising an anti-CD20 agent. In some embodiments, the anti-CD20 agent is rituximab, ofatumumab, ocrelizumab, iodine i131 tositumomab, obinutuzumab or ibritumomab. In some embodiments, the anti-CD20 agent is rituximab (RITUXAN®).

In some embodiments, administration of a CAR-T and an anti-CD20 agent to a subject results in increased in vivo survival and persistence of the CAR-T in the subject in comparison to a subject administered with a CAR-T alone. In some embodiments, the CAR-T copies/mL or CAR-T copies/ug DNA in a blood sample is determined over a period of time, as a proxy for persistence. In some embodiments, the area under the curve (AUC) of a plasma concentration curve (i.e. the area defined by the plasma concentration curve at the top and the x-axis (time) at the bottom) is used as a measure of persistence. In some embodiments, the AUC of a plasma concentration curve post-100 days (time) is used as a measure of persistence. An increased persistence of CAR-T in a subject provides the superior effect of improved response to treatment. An increased AUC in a subject provides the superior effect of improved response to treatment. An “increased” or “enhanced” amount is typically a “statistically significant” amount, and may include an increase that is at least 1%, 2%, 3%, 4%, 5%, 6%, 7%, 8%, 9%, 10%, 20%, 30%, 40%, 50%, 60%, 70%, 75%, 80%, 85%, 90%, 95%, 100%, 150%, 200%, 250%, 300%, 350%, 400%, 450%, 500%, 600%, 700%, 800%, 900%, 1000%, 1500%, 2000% or 3000% (including incremental all percentages in between e.g. 11%, 12%, 13%, 14%, 15%) more than a response in the subject administered with CAR alone. An “increased” or “enhanced” amount may include an increase that is 1%-10%, 10%-20%, 20%-30%, 30%-40%, 40%-50%, 50%-60%, 60%-70%, 70%-80%, 80%-90%, 90%-100%, 100%-150% or 150%-200% more than a response in the subject administered with the CAR-T alone. In some embodiments, the persistence of a CAR-T in a subject administered with a CAR-T and an anti-CD20 agent is at least 75% higher in comparison to a subject administered with CAR-T alone. In some embodiments, the persistence of a CAR-T in a subject administered with a CAR-T and an anti-CD20 agent is at least 90% higher in comparison to a subject administered with CAR-T alone.

In some embodiments, administration of a CAR and an anti-CD20 agent to a subject results increased in vivo persistence and expansion of the CAR in the subject in comparison to a subject administered with a CAR alone. In some embodiments, administration of a CAR and an anti-CD20 agent to a subject results in increased in vivo persistence but does not impact the expansion of the CAR in the subject in comparison to a subject administered with a CAR alone.

As used herein, the term “anti-drug antibody”, or “ADA”, refers to antibodies generated in a subject against a therapeutic protein present in the subject. A classical anti-drug antibody (ADA) response is understood in the art to result from systemic administration of a recombinant therapeutic protein to the subject. Moreover, as used herein with respect to CAR-T therapeutics, an ADA response is intended to encompass the antibody responses observed in the herein-described studies wherein antibodies were generated that bind to the CAR-T (i.e., antibodies generated against the P-BCMA-101 CAR-T).

In some embodiments, administration of a CAR-T and an anti-CD20 agent to a subject results decreased anti-drug antibody (ADA) response to the CAR-T in the subject in comparison to a subject administered with a CAR-T alone. In some embodiments, the ADA response is measured in peripheral blood over time using a meso scale discovery (MSD) assay. An decrease in ADA response in a subject provides a superior effect of improved response to treatment. An “decreased” or “lowered” amount is typically a “statistically significant” amount, and may include an decrease that is 1%, 2%, 3%, 4%, 5%, 6%, 7%, 8%, 9%, 10%, 20%, 30%, 40%, 50%, 60%, 70%, 80%, 90%, 100% (including incremental all percentages in between e.g. 11%, 12%, 13%, 14%, 15%) lower than a response in the subject administered with CAR-T alone. An “decreased” or “lowered” amount may include a decrease that is 1%-10%, 10%-20%, 20%-30%, 30%-40%, 40%-50%, 50%-60%, 60%-70%, 70%-80%, 80%-90% or 90%-100% less than a response in the subject administered with the CAR-T alone. In some embodiments, the anti-drug antibody response against the CAR-T in a subject administered with a CAR-T and an anti-CD20 agent is at least 50% lower in comparison to a subject administered with CAR-T alone.

Any method of the present invention can comprise administering an effective amount of a composition or pharmaceutical composition comprising at least one protein scaffold to a cell, tissue, organ, animal or patient in need of such modulation, treatment or therapy. Such a method can optionally further comprise co-administration or combination therapy for treating such diseases or disorders, wherein the administering of said at least one protein scaffold, specified portion or variant thereof, further comprises administering, before concurrently, and/or after, at least one selected from at least one of an alkylating agent, a mitotic inhibitor, and a radiopharmaceutical. Suitable dosages are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000); Nursing 2001 Handbook of Drugs, 21st edition, Springhouse Corp., Springhouse, Pa., 2001; Health Professional's Drug Guide 2001, ed., Shannon, Wilson, Stang, Prentice-Hall, Inc, Upper Saddle River, N.J. each of which references are entirely incorporated herein by reference.

Preferred doses can optionally include about 0.1-99 and/or 100-500 mg/kg/administration, or any range, value or fraction thereof, or to achieve a serum concentration of about 0.1-5000 μg/ml serum concentration per single or multiple administration, or any range, value or fraction thereof. A preferred dosage range for the protein scaffold of the present invention is from about 1 mg/kg, up to about 3, about 6 or about 12 mg/kg of body weight of the patient.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 to 100 milligrams per kilogram of body weight. Ordinarily 0.1 to 50, and preferably, 0.1 to 10 milligrams per kilogram per administration or in sustained release form is effective to obtain desired results.

As a non-limiting example, treatment of humans or animals can be provided as a one-time or periodic dosage of at least one protein scaffold of the present invention about 0.1 to 100 mg/kg or any range, value or fraction thereof per day, on at least one of day 1-40, or, alternatively or additionally, at least one of week 1-52, or, alternatively or additionally, at least one of 1-20 years, or any combination thereof, using single, infusion or repeated doses.

Dosage forms (composition) suitable for internal administration generally contain from about 0.001 milligram to about 500 milligrams of active ingredient per unit or container. In these pharmaceutical compositions the active ingredient will ordinarily be present in an amount of about 0.5-99.999% by weight based on the total weight of the composition.

In some embodiments, the first composition comprising a population of T cells expressing a P-BCMA-101 CAR is administered at a total dose of at least 0.1×10⁶, 0.2×10⁶, 0.25×10⁶, 0.5×10⁶, 0.6×10⁶, 0.7×10⁶, 0.75×10⁶, 0.8×10⁶, 0.9×10⁶, 1×10⁶, 2×10⁶, 3×10⁶, 4×10⁶, 5×10⁶, 6×10⁶, 7×10⁶, 8×10⁶, 9×10⁶, 10×10⁶, 11×10⁶, 12×10⁶, 13×10⁶, 14×10⁶, 15×10⁶, 16×10⁶, 17×10⁶, 18×10⁶, 19×10⁶ or 20×10⁶ cells/kg of the subject's body weight. In some embodiments, the CAR-T cells are administered at a dose of 0.25×10⁶ cells/kg/dose. In some embodiments, the CAR-T cells are administered at a dose of 0.75×10⁶ cells/kg/dose. In some embodiments, the CAR-T cells are administered at a dose of 2×10⁶ cells/kg/dose. In some embodiments, the CAR-T cells are administered at a dose of 6×10⁶ cells/kg/dose. In some embodiments, the CAR-T cells are administered at a dose of 10×10⁶ cells/kg/dose. In some embodiments, the CAR-T cells are administered at a dose of 15×10⁶ cells/kg/dose.

For parenteral administration, the protein scaffold can be formulated as a solution, suspension, emulsion, particle, powder, or lyophilized powder in association, or separately provided, with a pharmaceutically acceptable parenteral vehicle. Examples of such vehicles are water, saline, Ringer's solution, dextrose solution, and about 1-10% human serum albumin. Liposomes and nonaqueous vehicles, such as fixed oils, can also be used. The vehicle or lyophilized powder can contain additives that maintain isotonicity (e.g., sodium chloride, mannitol) and chemical stability (e.g., buffers and preservatives). The formulation is sterilized by known or suitable techniques.

Suitable pharmaceutical carriers are described in the most recent edition of Remington's Pharmaceutical Sciences, A. Osol, a standard reference text in this field.

### Alternative Administration

Many known and developed modes can be used according to the present invention for administering pharmaceutically effective amounts of at least one protein scaffold according to the present invention. While pulmonary administration is used in the following description, other modes of administration can be used according to the present invention with suitable results. Protein scaffolds of the present invention can be delivered in a carrier, as a solution, emulsion, colloid, or suspension, or as a dry powder, using any of a variety of devices and methods suitable for administration by inhalation or other modes described here within or known in the art.

### Parenteral Formulations and Administration

Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446 entirely incorporated herein by reference.

### Alternative Delivery

The invention further relates to the administration of at least one protein scaffold by parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal means. At least one protein scaffold composition can be prepared for use for parenteral (subcutaneous, intramuscular or intravenous) or any other administration particularly in the form of liquid solutions or suspensions; for use in vaginal or rectal administration particularly in semisolid forms, such as, but not limited to, creams and suppositories; for buccal, or sublingual administration, such as, but not limited to, in the form of tablets or capsules; or intranasally, such as, but not limited to, the form of powders, nasal drops or aerosols or certain agents; or transdermally, such as not limited to a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement;” Hsieh, D. S., Eds., pp. 59-90 (Marcel Dekker, Inc. New York 1994, entirely incorporated herein by reference), or with oxidizing agents that enable the application of formulations containing proteins and peptides onto the skin (WO 98/53847), or applications of electric fields to create transient transport pathways, such as electroporation, or to increase the mobility of charged drugs through the skin, such as iontophoresis, or application of ultrasound, such as sonophoresis (U.S. Pat. Nos. 4,309,989 and 4,767,402) (the above publications and patents being entirely incorporated herein by reference).

### Pulmonary/Nasal Administration

For pulmonary administration, preferably, at least one protein scaffold composition is delivered in a particle size effective for reaching the lower airways of the lung or sinuses. According to the invention, at least one protein scaffold can be delivered by any of a variety of inhalation or nasal devices known in the art for administration of a therapeutic agent by inhalation. These devices capable of depositing aerosolized formulations in the sinus cavity or alveoli of a patient include metered dose inhalers, nebulizers, dry powder generators, sprayers, and the like. Other devices suitable for directing the pulmonary or nasal administration of protein scaffolds are also known in the art. All such devices can use formulations suitable for the administration for the dispensing of protein scaffold in an aerosol. Such aerosols can be comprised of either solutions (both aqueous and nonaqueous) or solid particles.

Metered dose inhalers like the Ventolin metered dose inhaler, typically use a propellant gas and require actuation during inspiration (See, e.g., WO 94/16970, WO 98/35888). Dry powder inhalers like Turbuhaler™ (Astra), Rotahaler® (Glaxo), Diskus® (Glaxo), Spiros™ inhaler (Dura), devices marketed by Inhale Therapeutics, and the Spinhaler® powder inhaler (Fisons), use breath-actuation of a mixed powder (U.S. Pat. No. 4,668,218 Astra, EP 237507 Astra, WO 97/25086 Glaxo, WO 94/08552 Dura, U.S. Pat. No. 5,458,135 Inhale, WO 94/06498 Fisons, entirely incorporated herein by reference). Nebulizers like AERx™ Aradigm, the Ultravent® nebulizer (Mallinckrodt), and the Acorn II® nebulizer (Marquest Medical Products) (U.S. Pat. No. 5,404,871 Aradigm, WO 97/22376), the above references entirely incorporated herein by reference, produce aerosols from solutions, while metered dose inhalers, dry powder inhalers, etc. generate small particle aerosols. These specific examples of commercially available inhalation devices are intended to be a representative of specific devices suitable for the practice of this invention, and are not intended as limiting the scope of the invention.

Preferably, a composition comprising at least one protein scaffold is delivered by a dry powder inhaler or a sprayer. There are several desirable features of an inhalation device for administering at least one protein scaffold of the present invention. For example, delivery by the inhalation device is advantageously reliable, reproducible, and accurate. The inhalation device can optionally deliver small dry particles, e.g., less than about 10 μm, preferably about 1-5 μm, for good respirability.

### Administration of Protein Scaffold Compositions as a Spray

A spray including protein scaffold composition can be produced by forcing a suspension or solution of at least one protein scaffold through a nozzle under pressure. The nozzle size and configuration, the applied pressure, and the liquid feed rate can be chosen to achieve the desired output and particle size. An electrospray can be produced, for example, by an electric field in connection with a capillary or nozzle feed. Advantageously, particles of at least one protein scaffold composition delivered by a sprayer have a particle size less than about 10 μm, preferably, in the range of about 1 μm to about 5 μm, and, most preferably, about 2 μm to about 3 μm.

Formulations of at least one protein scaffold composition suitable for use with a sprayer typically include protein scaffold composition in an aqueous solution at a concentration of about 0.1 mg to about 100 mg of at least one protein scaffold composition per ml of solution or mg/gm, or any range, value, or fraction therein. The formulation can include agents, such as an excipient, a buffer, an isotonicity agent, a preservative, a surfactant, and, preferably, zinc. The formulation can also include an excipient or agent for stabilization of the protein scaffold composition, such as a buffer, a reducing agent, a bulk protein, or a carbohydrate. Bulk proteins useful in formulating protein scaffold compositions include albumin, protamine, or the like. Typical carbohydrates useful in formulating protein scaffold compositions include sucrose, mannitol, lactose, trehalose, glucose, or the like. The protein scaffold composition formulation can also include a surfactant, which can reduce or prevent surface-induced aggregation of the protein scaffold composition caused by atomization of the solution in forming an aerosol. Various conventional surfactants can be employed, such as polyoxyethylene fatty acid esters and alcohols, and polyoxyethylene sorbitol fatty acid esters. Amounts will generally range between 0.001 and 14% by weight of the formulation. Especially preferred surfactants for purposes of this invention are polyoxyethylene sorbitan monooleate, polysorbate 80, polysorbate 20, or the like. Additional agents known in the art for formulation of a protein, such as protein scaffolds, or specified portions or variants, can also be included in the formulation.

### Administration of Protein Scaffold Compositions by a Nebulizer

Protein scaffold compositions of the invention can be administered by a nebulizer, such as jet nebulizer or an ultrasonic nebulizer. Typically, in a jet nebulizer, a compressed air source is used to create a high-velocity air jet through an orifice. As the gas expands beyond the nozzle, a low-pressure region is created, which draws a solution of protein scaffold composition through a capillary tube connected to a liquid reservoir. The liquid stream from the capillary tube is sheared into unstable filaments and droplets as it exits the tube, creating the aerosol. A range of configurations, flow rates, and baffle types can be employed to achieve the desired performance characteristics from a given jet nebulizer. In an ultrasonic nebulizer, high-frequency electrical energy is used to create vibrational, mechanical energy, typically employing a piezoelectric transducer. This energy is transmitted to the formulation of protein scaffold composition either directly or through a coupling fluid, creating an aerosol including the protein scaffold composition. Advantageously, particles of protein scaffold composition delivered by a nebulizer have a particle size less than about 10 μm, preferably, in the range of about 1 μm to about 5 μm, and, most preferably, about 2 μm to about 3 μm.

Formulations of at least one protein scaffold suitable for use with a nebulizer, either jet or ultrasonic, typically include a concentration of about 0.1 mg to about 100 mg of at least one protein scaffold per ml of solution. The formulation can include agents, such as an excipient, a buffer, an isotonicity agent, a preservative, a surfactant, and, preferably, zinc. The formulation can also include an excipient or agent for stabilization of the at least one protein scaffold composition, such as a buffer, a reducing agent, a bulk protein, or a carbohydrate. Bulk proteins useful in formulating at least one protein scaffold compositions include albumin, protamine, or the like. Typical carbohydrates useful in formulating at least one protein scaffold include sucrose, mannitol, lactose, trehalose, glucose, or the like. The at least one protein scaffold formulation can also include a surfactant, which can reduce or prevent surface-induced aggregation of the at least one protein scaffold caused by atomization of the solution in forming an aerosol. Various conventional surfactants can be employed, such as polyoxyethylene fatty acid esters and alcohols, and polyoxyethylene sorbital fatty acid esters. Amounts will generally range between about 0.001 and 4% by weight of the formulation. Especially preferred surfactants for purposes of this invention are polyoxyethylene sorbitan mono-oleate, polysorbate 80, polysorbate 20, or the like. Additional agents known in the art for formulation of a protein, such as protein scaffold, can also be included in the formulation.

### Administration of Protein Scaffold Compositions by a Metered Dose Inhaler

In a metered dose inhaler (MDI), a propellant, at least one protein scaffold, and any excipients or other additives are contained in a canister as a mixture including a liquefied compressed gas. Actuation of the metering valve releases the mixture as an aerosol, preferably containing particles in the size range of less than about 10 μm, preferably, about 1 μm to about 5 μm, and, most preferably, about 2 μm to about 3 μm. The desired aerosol particle size can be obtained by employing a formulation of protein scaffold composition produced by various methods known to those of skill in the art, including jet-milling, spray drying, critical point condensation, or the like. Preferred metered dose inhalers include those manufactured by 3M or Glaxo and employing a hydrofluorocarbon propellant. Formulations of at least one protein scaffold for use with a metered-dose inhaler device will generally include a finely divided powder containing at least one protein scaffold as a suspension in a non-aqueous medium, for example, suspended in a propellant with the aid of a surfactant. The propellant can be any conventional material employed for this purpose, such as chlorofluorocarbon, a hydrochlorofluorocarbon, a hydrofluorocarbon, or a hydrocarbon, including trichlorofluoromethane, dichlorodifluoromethane, dichlorotetrafluoroethanol and 1,1,1,2-tetrafluoroethane, HFA-134a (hydrofluoroalkane-134a), HFA-227 (hydrofluoroalkane-227), or the like. Preferably, the propellant is a hydrofluorocarbon. The surfactant can be chosen to stabilize the at least one protein scaffold as a suspension in the propellant, to protect the active agent against chemical degradation, and the like. Suitable surfactants include sorbitan trioleate, soya lecithin, oleic acid, or the like. In some cases, solution aerosols are preferred using solvents, such as ethanol. Additional agents known in the art for formulation of a protein can also be included in the formulation. One of ordinary skill in the art will recognize that the methods of the current invention can be achieved by pulmonary administration of at least one protein scaffold composition via devices not described herein.

### Oral Formulations and Administration

Formulations for oral administration rely on the co-administration of adjuvants (e.g., resorcinols and nonionic surfactants, such as polyoxyethylene oleyl ether and n-hexadecylpolyethylene ether) to increase artificially the permeability of the intestinal walls, as well as the co-administration of enzymatic inhibitors (e.g., pancreatic trypsin inhibitors, diisopropylfluorophosphate (DFF) and trasylol) to inhibit enzymatic degradation. Formulations for delivery of hydrophilic agents including proteins and protein scaffolds and a combination of at least two surfactants intended for oral, buccal, mucosal, nasal, pulmonary, vaginal transmembrane, or rectal administration are taught in U.S. Pat. No. 6,309,663. The active constituent compound of the solid-type dosage form for oral administration can be mixed with at least one additive, including sucrose, lactose, cellulose, mannitol, trehalose, raffinose, maltitol, dextran, starches, agar, arginates, chitins, chitosans, pectins, gum tragacanth, gum arabic, gelatin, collagen, casein, albumin, synthetic or semisynthetic polymer, and glyceride. These dosage forms can also contain other type(s) of additives, e.g., inactive diluting agent, lubricant, such as magnesium stearate, paraben, preserving agent, such as sorbic acid, ascorbic acid, .alpha.-tocopherol, antioxidant such as cysteine, disintegrator, binder, thickener, buffering agent, sweetening agent, flavoring agent, perfuming agent, etc.

Tablets and pills can be further processed into enteric-coated preparations. The liquid preparations for oral administration include emulsion, syrup, elixir, suspension and solution preparations allowable for medical use. These preparations can contain inactive diluting agents ordinarily used in said field, e.g., water. Liposomes have also been described as drug delivery systems for insulin and heparin (U.S. Pat. No. 4,239,754). More recently, microspheres of artificial polymers of mixed amino acids (proteinoids) have been used to deliver pharmaceuticals (U.S. Pat. No. 4,925,673). Furthermore, carrier compounds described in U.S. Pat. Nos. 5,879,681 and 5,871,753 and used to deliver biologically active agents orally are known in the art.

### Mucosal Formulations and Administration

A formulation for orally administering a bioactive agent encapsulated in one or more biocompatible polymer or copolymer excipients, preferably, a biodegradable polymer or copolymer, affording microcapsules which due to the proper size of the resultant microcapsules results in the agent reaching and being taken up by the folliculi lymphatic aggregati, otherwise known as the “Peyer's patch,” or “GALT” of the animal without loss of effectiveness due to the agent having passed through the gastrointestinal tract. Similar folliculi lymphatic aggregati can be found in the bronchei tubes (BALT) and the large intestine. The above-described tissues are referred to in general as mucosally associated lymphoreticular tissues (MALT). For absorption through mucosal surfaces, compositions and methods of administering at least one protein scaffold include an emulsion comprising a plurality of submicron particles, a mucoadhesive macromolecule, a bioactive peptide, and an aqueous continuous phase, which promotes absorption through mucosal surfaces by achieving mucoadhesion of the emulsion particles (U.S. Pat. No. 5,514,670). Mucous surfaces suitable for application of the emulsions of the present invention can include corneal, conjunctival, buccal, sublingual, nasal, vaginal, pulmonary, stomachic, intestinal, and rectal routes of administration. Formulations for vaginal or rectal administration, e.g., suppositories, can contain as excipients, for example, polyalkyleneglycols, vaseline, cocoa butter, and the like. Formulations for intranasal administration can be solid and contain as excipients, for example, lactose or can be aqueous or oily solutions of nasal drops. For buccal administration, excipients include sugars, calcium stearate, magnesium stearate, pregelinatined starch, and the like (U.S. Pat. No. 5,849,695).

### Transdermal Formulations and Administration

For transdermal administration, the at least one protein scaffold is encapsulated in a delivery device, such as a liposome or polymeric nanoparticles, microparticle, microcapsule, or microspheres (referred to collectively as microparticles unless otherwise stated). A number of suitable devices are known, including microparticles made of synthetic polymers, such as polyhydroxy acids, such as polylactic acid, polyglycolic acid and copolymers thereof, polyorthoesters, polyanhydrides, and polyphosphazenes, and natural polymers, such as collagen, polyamino acids, albumin and other proteins, alginate and other polysaccharides, and combinations thereof (U.S. Pat. No. 5,814,599).

### Prolonged Administration and Formulations

It can be desirable to deliver the compounds of the present invention to the subject over prolonged periods of time, for example, for periods of one week to one year from a single administration. Various slow release, depot or implant dosage forms can be utilized. For example, a dosage form can contain a pharmaceutically acceptable non-toxic salt of the compounds that has a low degree of solubility in body fluids, for example, (a) an acid addition salt with a polybasic acid, such as phosphoric acid, sulfuric acid, citric acid, tartaric acid, tannic acid, pamoic acid, alginic acid, polyglutamic acid, naphthalene mono- or di-sulfonic acids, polygalacturonic acid, and the like; (b) a salt with a polyvalent metal cation, such as zinc, calcium, bismuth, barium, magnesium, aluminum, copper, cobalt, nickel, cadmium and the like, or with an organic cation formed from e.g., N,N′-dibenzyl-ethylenediamine or ethylenediamine; or (c) combinations of (a) and (b), e.g., a zinc tannate salt. Additionally, the compounds of the present invention or, preferably, a relatively insoluble salt, such as those just described, can be formulated in a gel, for example, an aluminum monostearate gel with, e.g., sesame oil, suitable for injection. Particularly preferred salts are zinc salts, zinc tannate salts, pamoate salts, and the like. Another type of slow release depot formulation for injection would contain the compound or salt dispersed for encapsulation in a slow degrading, non-toxic, non-antigenic polymer, such as a polylactic acid/polyglycolic acid polymer for example as described in U.S. Pat. No. 3,773,919. The compounds or, preferably, relatively insoluble salts, such as those described above, can also be formulated in cholesterol matrix silastic pellets, particularly for use in animals. Additional slow release, depot or implant formulations, e.g., gas or liquid liposomes, are known in the literature (U.S. Pat. No. 5,770,222 and “Sustained and Controlled Release Drug Delivery Systems”, J. R. Robinson ed., Marcel Dekker, Inc., N.Y., 1978).

### Infusion of Modified Cells as Adoptive Cell Therapy

The disclosure provides modified cells that express one or more CARs and/or CARTyrins of the disclosure that have been selected and/or expanded for administration to a subject in need thereof. Modified cells of the disclosure may be formulated for storage at any temperature including room temperature and body temperature. Modified cells of the disclosure may be formulated for cryopreservation and subsequent thawing. Modified cells of the disclosure may be formulated in a pharmaceutically acceptable carrier for direct administration to a subject from sterile packaging. Modified cells of the disclosure may be formulated in a pharmaceutically acceptable carrier with an indicator of cell viability and/or CAR/CARTyrin expression level to ensure a minimal level of cell function and CAR/CARTyrin expression. Modified cells of the disclosure may be formulated in a pharmaceutically acceptable carrier at a prescribed density with one or more reagents to inhibit further expansion and/or prevent cell death.

### Inducible Proapoptotic Polypeptides

Inducible proapoptotic polypeptides of the disclosure are superior to existing inducible polypeptides because the inducible proapoptotic polypeptides of the disclosure are far less immunogenic. While inducible proapoptotic polypeptides of the disclosure are recombinant polypeptides, and, therefore, non-naturally occurring, the sequences that are recombined to produce the inducible proapoptotic polypeptides of the disclosure do not comprise non-human sequences that the host human immune system could recognize as “non-self” and, consequently, induce an immune response in the subject receiving an inducible proapoptotic polypeptide of the disclosure, a cell comprising the inducible proapoptotic polypeptide or a composition comprising the inducible proapoptotic polypeptide or the cell comprising the inducible proapoptotic polypeptide.

The disclosure provides inducible proapoptotic polypeptides comprising a ligand binding region, a linker, and a proapoptotic peptide, wherein the inducible proapoptotic polypeptide does not comprise a non-human sequence. In certain embodiments, the non-human sequence comprises a restriction site. In certain embodiments, the proapoptotic peptide is a caspase polypeptide. In certain embodiments, the caspase polypeptide is a caspase 9 polypeptide. In certain embodiments, the caspase 9 polypeptide is a truncated caspase 9 polypeptide. Inducible proapoptotic polypeptides of the disclosure may be non-naturally occurring.

Caspase polypeptides of the disclosure include, but are not limited to, caspase 1, caspase 2, caspase 3, caspase 4, caspase 5, caspase 6, caspase 7, caspase 8, caspase 9, caspase 10, caspase 11, caspase 12, and caspase 14. Caspase polypeptides of the disclosure include, but are not limited to, those caspase polypeptides associated with apoptosis including caspase 2, caspase 3, caspase 6, caspase 7, caspase 8, caspase 9, and caspase 10. Caspase polypeptides of the disclosure include, but are not limited to, those caspase polypeptides that initiate apoptosis, including caspase 2, caspase 8, caspase 9, and caspase 10. Caspase polypeptides of the disclosure include, but are not limited to, those caspase polypeptides that execute apoptosis, including caspase 3, caspase 6, and caspase 7.

Caspase polypeptides of the disclosure may be encoded by an amino acid or a nucleic acid sequence having one or more modifications compared to a wild type amino acid or a nucleic acid sequence. The nucleic acid sequence encoding a caspase polypeptide of the disclosure may be codon optimized. The one or more modifications to an amino acid and/or nucleic acid sequence of a caspase polypeptide of the disclosure may increase an interaction, a cross-linking, a cross-activation, or an activation of the caspase polypeptide of the disclosure compared to a wild type amino acid or a nucleic acid sequence. Alternatively, or in addition, the one or more modifications to an amino acid and/or nucleic acid sequence of a caspase polypeptide of the disclosure may decrease the immunogenicity of the caspase polypeptide of the disclosure compared to a wild type amino acid or a nucleic acid sequence.

Caspase polypeptides of the disclosure may be truncated compared to a wild type caspase polypeptide. For example, a caspase polypeptide may be truncated to eliminate a sequence encoding a Caspase Activation and Recruitment Domain (CARD) to eliminate or minimize the possibility of activating a local inflammatory response in addition to initiating apoptosis in the cell comprising an inducible caspase polypeptide of the disclosure. The nucleic acid sequence encoding a caspase polypeptide of the disclosure may be spliced to form a variant amino acid sequence of the caspase polypeptide of the disclosure compared to a wild type caspase polypeptide. Caspase polypeptides of the disclosure may be encoded by recombinant and/or chimeric sequences. Recombinant and/or chimeric caspase polypeptides of the disclosure may include sequences from one or more different caspase polypeptides. Alternatively, or in addition, recombinant and/or chimeric caspase polypeptides of the disclosure may include sequences from one or more species (e.g. a human sequence and a non-human sequence). Caspase polypeptides of the disclosure may be non-naturally occurring.

The ligand binding region of an inducible proapoptotic polypeptide of the disclosure may include any polypeptide sequence that facilitates or promotes the dimerization of a first inducible proapoptotic polypeptide of the disclosure with a second inducible proapoptotic polypeptide of the disclosure, the dimerization of which activates or induces cross-linking of the proapoptotic polypeptides and initiation of apoptosis in the cell.

The ligand-binding (“dimerization”) region may comprise any polypeptide or functional domain thereof that will allow for induction using a natural or unnatural ligand (i.e. and induction agent), for example, an unnatural synthetic ligand. The ligand-binding region may be internal or external to the cellular membrane, depending upon the nature of the inducible proapoptotic polypeptide and the choice of ligand (i.e. induction agent). A wide variety of ligand-binding polypeptides and functional domains thereof, including receptors, are known. Ligand-binding regions of the disclosure may include one or more sequences from a receptor. Of particular interest are ligand-binding regions for which ligands (for example, small organic ligands) are known or may be readily produced. These ligand-binding regions or receptors may include, but are not limited to, the FKBPs and cyclophilin receptors, the steroid receptors, the tetracycline receptor, and the like, as well as “unnatural” receptors, which can be obtained from antibodies, particularly the heavy or light chain subunit, mutated sequences thereof, random amino acid sequences obtained by stochastic procedures, combinatorial syntheses, and the like. In certain embodiments, the ligand-binding region is selected from the group consisting of a FKBP ligand-binding region, a cyclophilin receptor ligand-binding region, a steroid receptor ligand-binding region, a cyclophilin receptors ligand-binding region, and a tetracycline receptor ligand-binding region.

The ligand-binding regions comprising one or more receptor domain(s) may be at least about 50 amino acids, and fewer than about 350 amino acids, usually fewer than 200 amino acids, either as the natural domain or truncated active portion thereof. The binding region may, for example, be small (<25 kDa, to allow efficient transfection in viral vectors), monomeric, nonimmunogenic, have synthetically accessible, cell permeable, nontoxic ligands that can be configured for dimerization.

The ligand-binding regions comprising one or more receptor domain(s) may be intracellular or extracellular depending upon the design of the inducible proapoptotic polypeptide and the availability of an appropriate ligand (i.e. induction agent). For hydrophobic ligands, the binding region can be on either side of the membrane, but for hydrophilic ligands, particularly protein ligands, the binding region will usually be external to the cell membrane, unless there is a transport system for internalizing the ligand in a form in which it is available for binding. For an intracellular receptor, the inducible proapoptotic polypeptide or a transposon or vector comprising the inducible proapoptotic polypeptide may encode a signal peptide and transmembrane domain 5′ or 3′ of the receptor domain sequence or may have a lipid attachment signal sequence 5′ of the receptor domain sequence. Where the receptor domain is between the signal peptide and the transmembrane domain, the receptor domain will be extracellular.

Antibodies and antibody subunits, e.g., heavy or light chain, particularly fragments, more particularly all or part of the variable region, or fusions of heavy and light chain to create high-affinity binding, can be used as a ligand binding region of the disclosure. Antibodies that are contemplated include ones that are an ectopically expressed human product, such as an extracellular domain that would not trigger an immune response and generally not expressed in the periphery (i.e., outside the CNS/brain area). Such examples, include, but are not limited to low affinity nerve growth factor receptor (LNGFR), and embryonic surface proteins (i.e., carcinoembryonic antigen). Yet further, antibodies can be prepared against haptenic molecules, which are physiologically acceptable, and the individual antibody subunits screened for binding affinity. The cDNA encoding the subunits can be isolated and modified by deletion of the constant region, portions of the variable region, mutagenesis of the variable region, or the like, to obtain a binding protein domain that has the appropriate affinity for the ligand. In this way, almost any physiologically acceptable haptenic compound can be employed as the ligand or to provide an epitope for the ligand. Instead of antibody units, natural receptors can be employed, where the binding region or domain is known and there is a useful or known ligand for binding.

For multimerizing the receptor, the ligand for the ligand-binding region/receptor domains of the inducible proapoptotic polypeptides may be multimeric in the sense that the ligand can have at least two binding sites, with each of the binding sites capable of binding to a ligand receptor region (i.e. a ligand having a first binding site capable of binding the ligand-binding region of a first inducible proapoptotic polypeptide and a second binding site capable of binding the ligand-binding region of a second inducible proapoptotic polypeptide, wherein the ligand-binding regions of the first and the second inducible proapoptotic polypeptides are either identical or distinct). Thus, as used herein, the term “multimeric ligand binding region” refers to a ligand-binding region of an inducible proapoptotic polypeptide of the disclosure that binds to a multimeric ligand. Multimeric ligands of the disclosure include dimeric ligands. A dimeric ligand of the disclosure may have two binding sites capable of binding to the ligand receptor domain. In certain embodiments, multimeric ligands of the disclosure are a dimer or higher order oligomer, usually not greater than about tetrameric, of small synthetic organic molecules, the individual molecules typically being at least about 150 Da and less than about 5 kDa, usually less than about 3 kDa. A variety of pairs of synthetic ligands and receptors can be employed. For example, in embodiments involving natural receptors, dimeric FK506 can be used with an FKBP12 receptor, dimerized cyclosporin A can be used with the cyclophilin receptor, dimerized estrogen with an estrogen receptor, dimerized glucocorticoids with a glucocorticoid receptor, dimerized tetracycline with the tetracycline receptor, dimerized vitamin D with the vitamin D receptor, and the like. Alternatively higher orders of the ligands, e.g., trimeric can be used. For embodiments involving unnatural receptors, e.g., antibody subunits, modified antibody subunits, single chain antibodies comprised of heavy and light chain variable regions in tandem, separated by a flexible linker, or modified receptors, and mutated sequences thereof, and the like, any of a large variety of compounds can be used. A significant characteristic of the units comprising a multimeric ligand of the disclosure is that each binding site is able to bind the receptor with high affinity, and preferably, that they are able to be dimerized chemically. Also, methods are available to balance the hydrophobicity/hydrophilicity of the ligands so that they are able to dissolve in serum at functional levels, yet diffuse across plasma membranes for most applications.

Activation of inducible proapoptotic polypeptides of the disclosure may be accomplished through, for example, chemically induced dimerization (CID) mediated by an induction agent to produce a conditionally controlled protein or polypeptide. Proapoptotic polypeptides of the disclosure not only inducible, but the induction of these polypeptides is also reversible, due to the degradation of the labile dimerizing agent or administration of a monomeric competitive inhibitor.

In certain embodiments, the ligand binding region comprises a FK506 binding protein 12 (FKBP12) polypeptide. In certain embodiments, the ligand binding region comprises a FKBP12 polypeptide having a substitution of valine (V) for phenylalanine (F) at position 36 (F36V). In certain embodiments, in which the ligand binding region comprises a FKBP12 polypeptide having a substitution of valine (V) for phenylalanine (F) at position 36 (F36V), the induction agent may comprise AP1903, a synthetic drug (CAS Index Name: 2-Piperidinecarboxylic acid, 1-[(2S)-1-oxo-2-(3,4,5-trimethoxyphenyl)butyl]-, 1,2-ethanediylbis[imino(2-oxo-2,1-ethanediyl)oxy-3,1-phenylene[(1R)-3-(3,4-dimethoxyphenyl)propylidene]]ester, [2S-[1(R*),2R*[S*[S*[1(R*),2R*]]]]]-(9C1) CAS Registry Number: 195514-63-7; Molecular Formula: C78H98N4O20; Molecular Weight: 1411.65)). In certain embodiments, in which the ligand binding region comprises a FKBP12 polypeptide having a substitution of valine (V) for phenylalanine (F) at position 36 (F36V), the induction agent may comprise AP20187 (CAS Registry Number: 195514-80-8 and Molecular Formula: C82H107N5O20). In certain embodiments, the induction agent is an AP20187 analog, such as, for example, AP1510. As used herein, the induction agents AP20187, AP1903 and AP1510 may be used interchangeably.

AP1903 API is manufactured by Alphora Research Inc. and AP1903 Drug Product for Injection is made by Formatech Inc. It is formulated as a 5 mg/mL solution of AP1903 in a 25% solution of the non-ionic solubilizer Solutol HS 15 (250 mg/mL, BASF). At room temperature, this formulation is a clear, slightly yellow solution. Upon refrigeration, this formulation undergoes a reversible phase transition, resulting in a milky solution. This phase transition is reversed upon re-warming to room temperature. The fill is 2.33 mL in a 3 mL glass vial (approximately 10 mg AP1903 for Injection total per vial). Upon determining a need to administer AP1903, patients may be, for example, administered a single fixed dose of AP1903 for Injection (0.4 mg/kg) via IV infusion over 2 hours, using a non-DEHP, non-ethylene oxide sterilized infusion set. The dose of AP1903 is calculated individually for all patients, and is not be recalculated unless body weight fluctuates by ≥10%. The calculated dose is diluted in 100 mL in 0.9% normal saline before infusion. In a previous Phase I study of AP1903, 24 healthy volunteers were treated with single doses of AP1903 for Injection at dose levels of 0.01, 0.05, 0.1, 0.5 and 1.0 mg/kg infused IV over 2 hours. AP1903 plasma levels were directly proportional to dose, with mean Cmax values ranging from approximately 10-1275 ng/mL over the 0.01-1.0 mg/kg dose range. Following the initial infusion period, blood concentrations demonstrated a rapid distribution phase, with plasma levels reduced to approximately 18, 7, and 1% of maximal concentration at 0.5 mg/kg, 2 and 10 hours post-dose, respectively. AP1903 for Injection was shown to be safe and well tolerated at all dose levels and demonstrated a favorable pharmacokinetic profile. Iuliucci J D, et al., J Clin Pharmacol. 41: 870-9, 2001.

The fixed dose of AP1903 for injection used, for example, may be 0.4 mg/kg intravenously infused over 2 hours. The amount of AP1903 needed in vitro for effective signaling of cells is 10-100 nM (1600 Da MW). This equates to 16-160 μg/L or ˜0.016-1.6 μg/kg (1.6-160 μg/kg). Doses up to 1 mg/kg were well-tolerated in the Phase I study of AP1903 described above. Therefore, 0.4 mg/kg may be a safe and effective dose of AP1903 for this Phase I study in combination with the therapeutic cells.

The amino acid and/or nucleic acid sequence encoding ligand binding of the disclosure may contain sequence one or more modifications compared to a wild type amino acid or nucleic acid sequence. For example, the amino acid and/or nucleic acid sequence encoding ligand binding region of the disclosure may be a codon-optimized sequence. The one or more modifications may increase the binding affinity of a ligand (e.g. an induction agent) for the ligand binding region of the disclosure compared to a wild type polypeptide. Alternatively, or in addition, the one or more modifications may decrease the immunogenicity of the ligand binding region of the disclosure compared to a wild type polypeptide. Ligand binding regions of the disclosure and/or induction agents of the disclosure may be non-naturally occurring.

Inducible proapoptotic polypeptides of the disclosure comprise a ligand binding region, a linker and a proapoptotic peptide, wherein the inducible proapoptotic polypeptide does not comprise a non-human sequence. In certain embodiments, the non-human sequence comprises a restriction site. The linker may comprise any organic or inorganic material that permits, upon dimerization of the ligand binding region, interaction, cross-linking, cross-activation, or activation of the proapoptotic polypeptides such that the interaction or activation of the proapoptotic polypeptides initiates apoptosis in the cell. In certain embodiments, the linker is a polypeptide. In certain embodiments, the linker is a polypeptide comprising a G/S rich amino acid sequence (a “GS” linker). In certain embodiments, the linker is a polypeptide comprising the amino acid sequence GGGGS (SEQ ID NO: 25). In preferred embodiments, the linker is a polypeptide and the nucleic acid encoding the polypeptide does not contain a restriction site for a restriction endonuclease. Linkers of the disclosure may be non-naturally occurring.

Inducible proapoptotic polypeptides of the disclosure may be expressed in a cell under the transcriptional regulation of any promoter capable of initiating and/or regulating the expression of an inducible proapoptotic polypeptide of the disclosure in that cell. The term “promoter” as used herein refers to a promoter that acts as the initial binding site for RNA polymerase to transcribe a gene. For example, inducible proapoptotic polypeptides of the disclosure may be expressed in a mammalian cell under the transcriptional regulation of any promoter capable of initiating and/or regulating the expression of an inducible proapoptotic polypeptide of the disclosure in a mammalian cell, including, but not limited to native, endogenous, exogenous, and heterologous promoters. Preferred mammalian cells include human cells. Thus, inducible proapoptotic polypeptides of the disclosure may be expressed in a human cell under the transcriptional regulation of any promoter capable of initiating and/or regulating the expression of an inducible proapoptotic polypeptide of the disclosure in a human cell, including, but not limited to, a human promoter or a viral promoter. Exemplary promoters for expression in human cells include, but are not limited to, a human cytomegalovirus (CMV) immediate early gene promoter, a SV40 early promoter, a Rous sarcoma virus long terminal repeat, β-actin promoter, a rat insulin promoter and a glyceraldehyde-3-phosphate dehydrogenase promoter, each of which may be used to obtain high-level expression of an inducible proapoptotic polypeptide of the disclosure. The use of other viral or mammalian cellular or bacterial phage promoters which are well known in the art to achieve expression of an inducible proapoptotic polypeptide of the disclosure is contemplated as well, provided that the levels of expression are sufficient for initiating apoptosis in a cell. By employing a promoter with well-known properties, the level and pattern of expression of the protein of interest following transfection or transformation can be optimized.

Selection of a promoter that is regulated in response to specific physiologic or synthetic signals can permit inducible expression of the inducible proapoptotic polypeptide of the disclosure. The ecdysone system (Invitrogen, Carlsbad, Calif.) is one such system. This system is designed to allow regulated expression of a gene of interest in mammalian cells. It consists of a tightly regulated expression mechanism that allows virtually no basal level expression of a transgene, but over 200-fold inducibility. The system is based on the heterodimeric ecdysone receptor of Drosophila, and when ecdysone or an analog such as muristerone A binds to the receptor, the receptor activates a promoter to turn on expression of the downstream transgene high levels of mRNA transcripts are attained. In this system, both monomers of the heterodimeric receptor are constitutively expressed from one vector, whereas the ecdysone-responsive promoter, which drives expression of the gene of interest, is on another plasmid. Engineering of this type of system into a vector of interest may therefore be useful. Another inducible system that may be useful is the Tet-Off™ or Tet-On™ system (Clontech, Palo Alto, Calif.) originally developed by Gossen and Bujard (Gossen and Bujard, Proc. Natl. Acad. Sci. USA, 89:5547-5551, 1992; Gossen et al., Science, 268:1766-1769, 1995). This system also allows high levels of gene expression to be regulated in response to tetracycline or tetracycline derivatives such as doxycycline. In the Tet-On™ system, gene expression is turned on in the presence of doxycycline, whereas in the Tet-Off™ system, gene expression is turned on in the absence of doxycycline. These systems are based on two regulatory elements derived from the tetracycline resistance operon of E. coli: the tetracycline operator sequence (to which the tetracycline repressor binds) and the tetracycline repressor protein. The gene of interest is cloned into a plasmid behind a promoter that has tetracycline-responsive elements present in it. A second plasmid contains a regulatory element called the tetracycline-controlled transactivator, which is composed, in the Tet-Off™ system, of the VP16 domain from the herpes simplex virus and the wild-type tetracycline repressor. Thus in the absence of doxycycline, transcription is constitutively on. In the Tet-On™ system, the tetracycline repressor is not wild type and in the presence of doxycycline activates transcription. For gene therapy vector production, the Tet-Off™ system may be used so that the producer cells could be grown in the presence of tetracycline or doxycycline and prevent expression of a potentially toxic transgene, but when the vector is introduced to the patient, the gene expression would be constitutively on.

In some circumstances, it is desirable to regulate expression of a transgene in a gene therapy vector. For example, different viral promoters with varying strengths of activity are utilized depending on the level of expression desired. In mammalian cells, the CMV immediate early promoter is often used to provide strong transcriptional activation. The CMV promoter is reviewed in Donnelly, J. J., et al., 1997. Annu. Rev. Immunol. 15:617-48. Modified versions of the CMV promoter that are less potent have also been used when reduced levels of expression of the transgene are desired. When expression of a transgene in hematopoietic cells is desired, retroviral promoters such as the LTRs from MLV or MMTV are often used. Other viral promoters that are used depending on the desired effect include SV40, RSV LTR, HIV-1 and HIV-2 LTR, adenovirus promoters such as from the ElA, E2A, or MLP region, AAV LTR, HSV-TK, and avian sarcoma virus.

In other examples, promoters may be selected that are developmentally regulated and are active in particular differentiated cells. Thus, for example, a promoter may not be active in a pluripotent stem cell, but, for example, where the pluripotent stem cell differentiates into a more mature cell, the promoter may then be activated.

Similarly tissue specific promoters are used to effect transcription in specific tissues or cells so as to reduce potential toxicity or undesirable effects to non-targeted tissues. These promoters may result in reduced expression compared to a stronger promoter such as the CMV promoter, but may also result in more limited expression, and immunogenicity (Bojak, A., et al., 2002. Vaccine. 20:1975-79; Cazeaux, N., et al., 2002. Vaccine 20:3322-31). For example, tissue specific promoters such as the PSA associated promoter or prostate-specific glandular kallikrein, or the muscle creatine kinase gene may be used where appropriate.

Examples of tissue specific or differentiation specific promoters include, but are not limited to, the following: B29 (B cells); CD14 (monocytic cells); CD43 (leukocytes and platelets); CD45 (hematopoietic cells); CD68 (macrophages); desmin (muscle); elastase-1 (pancreatic acinar cells); endoglin (endothelial cells); fibronectin (differentiating cells, healing tissues); and Flt-1 (endothelial cells); GFAP (astrocytes).

In certain indications, it is desirable to activate transcription at specific times after administration of the gene therapy vector. This is done with such promoters as those that are hormone or cytokine regulatable. Cytokine and inflammatory protein responsive promoters that can be used include K and T kininogen (Kageyama et al., (1987) J. Biol. Chem., 262, 2345-2351), c-fos, TNF-alpha, C-reactive protein (Arcone, et al., (1988) Nucl. Acids Res., 16(8), 3195-3207), haptoglobin (Oliviero et al., (1987) EMBO J., 6, 1905-1912), serum amyloid A2, C/EBP alpha, IL-1, IL-6 (Poli and Cortese, (1989) Proc. Nat'l Acad. Sci. USA, 86, 8202-8206), Complement C3 (Wilson et al., (1990) Mol. Cell. Biol., 6181-6191), IL-8, alpha-1 acid glycoprotein (Prowse and Baumann, (1988) Mol Cell Biol, 8, 42-51), alpha-1 antitrypsin, lipoprotein lipase (Zechner et al., Mol. Cell. Biol., 2394-2401, 1988), angiotensinogen (Ron, et al., (1991) Mol. Cell. Biol., 2887-2895), fibrinogen, c-jun (inducible by phorbol esters, TNF-alpha, UV radiation, retinoic acid, and hydrogen peroxide), collagenase (induced by phorbol esters and retinoic acid), metallothionein (heavy metal and glucocorticoid inducible), Stromelysin (inducible by phorbol ester, interleukin-1 and EGF), alpha-2 macroglobulin and alpha-1 anti-chymotrypsin. Other promoters include, for example, SV40, MMTV, Human Immunodeficiency Virus (MV), Moloney virus, ALV, Epstein Barr virus, Rous Sarcoma virus, human actin, myosin, hemoglobin, and creatine.

It is envisioned that any of the above promoters alone or in combination with another can be useful depending on the action desired. Promoters, and other regulatory elements, are selected such that they are functional in the desired cells or tissue. In addition, this list of promoters should not be construed to be exhaustive or limiting; other promoters that are used in conjunction with the promoters and methods disclosed herein.

### EXAMPLES

### Example 1—Characterization of P-BCMA-101 (a/k/a Anti-BCMA CARTyrin (A08))

Expression of CARTyrins of the disclosure was evaluated following mRNA electroporation of a sequence encoding a CARTyrin into T cells. Functionality of CARTyrin-expressing T cells was measured by degranulation against tumor lines. Characterization further assays correlations with functionality.

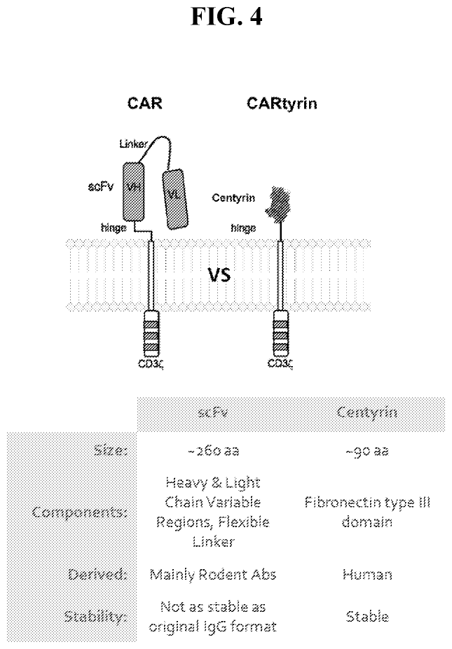

FIG. 4 depicts the structure of the A08 anti-BCMA CARTyrin.

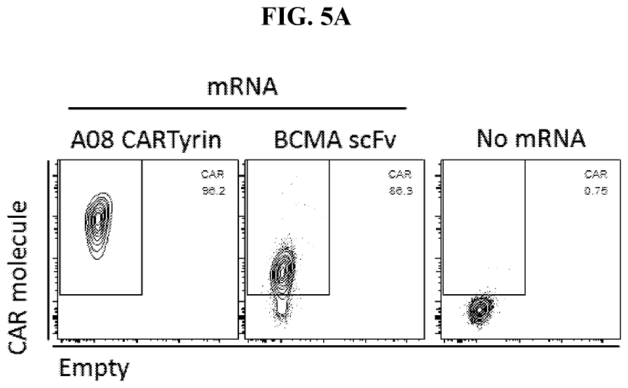

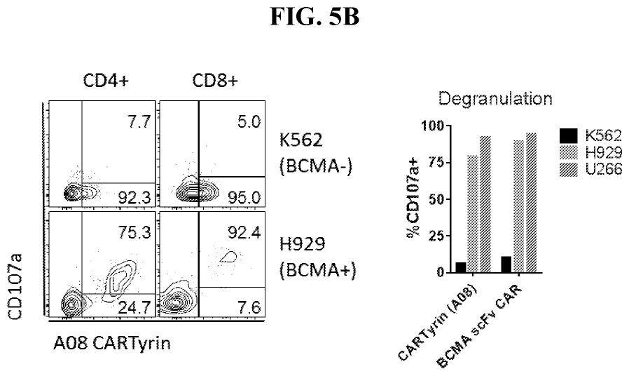

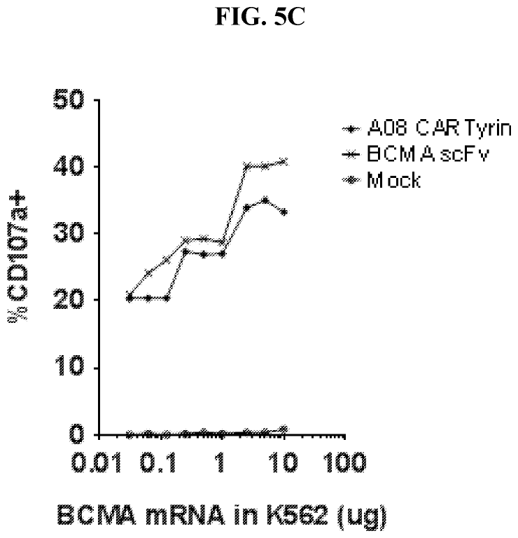

FIGS. 5-8 demonstrate the in vitro and in vivo characterization of P-BCMA-101 (encoding the A08 anti-BCMA CARTyrin).

In vitro evaluation of the A08 CARTyrin demonstrated high levels of surface expression following lentiviral transduction of human primary T cells and strong cytotoxic function (e.g. proliferation) against BCMA+ tumor cells (see FIGS. 5A-C). Following this strong performance in vitro, the ability of the A08 CARTyrin to function in vivo was evaluated.

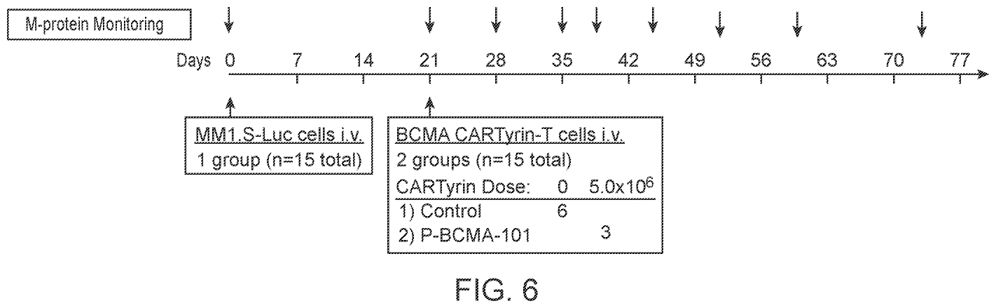

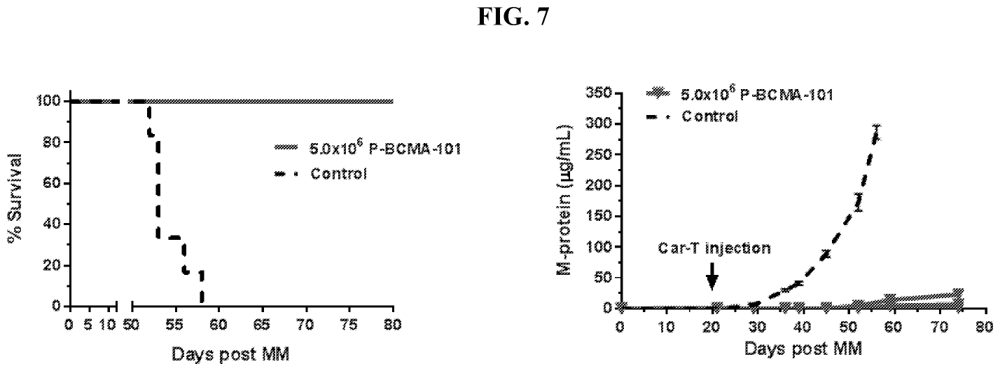

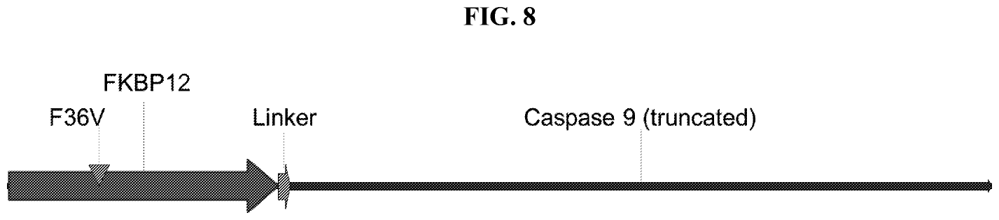

FIG. 6 depicts the treatment schedule for an in vivo study in mice using the A08 CARTyrin. Results of this study show that 100% of mice treated with P-BCMA-101 (encoding the A08 CARTyrin) survived to day 21 (see FIG. 7). This complete survival of treated animals at day 21 was accompanied by a showing of zero tumor burden (as assessed by M-protein abundance, which was not detectable in these animals at day 21) (see FIG. 7). FIG. 8 provides a series of photographs further illustrating tumor burden in control animal as well as those treated with P-BCMA-101. Animals expressing the A08 CARTyrin demonstrate a reduce tumor burden compared to controls.

### Example 2—Expression and Function of PiggyBac Integrated IC9 Safety Switch into Human Pan T-Cells

Human pan T-cells were nucleofected using an Amaxa 4D nucleofector with one of four piggyBac transposons. Modified T cells receiving the “mock” condition were nucleofected with an empty piggyBac transposon. Modified T cells received either a piggyBac transposon containing a therapeutic agent alone (a sequence encoding a CARTyrin) or a piggyBac transposon containing an integrated iC9 sequence and a therapeutic agent (a sequence encoding a CARTyrin).

FIG. 8 provides a schematic diagram of the iC9 safety switch, which contains a ligand binding region, a linker, and a truncated caspase 9 polypeptide. Specifically, the iC9 polypeptide contains a ligand binding region comprising a FK506 binding protein 12 (FKBP12) polypeptide including a substitution of valine (V) for phenylalanine (F) at position 36 (F36V). The FKBP12 polypeptide of the iC9 polypeptide is encoded by an amino acid sequence comprising GVQVETISPGDGRTFPKRGQTCVVHYTGMLEDGKKVDSSRDRNKPFKFMLGKQEVI RGWEEGVAQMSVGQRAKLTISPDYAYGATGHPGIIPPHATLVFDVELLKLE (SEQ ID NO: 23). The FKBP12 polypeptide of the iC9 polypeptide is encoded by a nucleic acid sequence comprising GGGGTCCAGGTCGAGACTATTTCACCAGGGGATGGGCGAACATTTCCAAAAAGG GGCCAGACTTGCGTCGTGCATTACACCGGGATGCTGGAGGACGGGAAGAAAGTG GACAGCTCCAGGGATCGCAACAAGCCCTTCAAGTTCATGCTGGGAAAGCAGGAA GTGATCCGAGGATGGGAGGAAGGCGTGGCACAGATGTCAGTCGGCCAGCGGGCC AAACTGACCATTAGCCCTGACTACGCTTATGGAGCAACAGGCCACCCAGGGATC ATTCCCCCTCATGCCACCCTGGTCTTCGAT GTGGAACTGCTGAAGCTGGAG (SEQ ID NO: 24). The linker region of the iC9 polypeptide is encoded by an amino acid comprising GGGGS (SEQ ID NO: 25) and a nucleic acid sequence comprising GGAGGAGGAGGATCC (SEQ ID NO: 26). The nucleic acid sequence encoding the linker region of the iC9 polypeptide is encoded by an amino acid comprising GFGDVGALESLRGNADLAYISLMEPCGHCLIINNVNFCRESGLRTRTGSNIDCEKLRR RFSSLHFMVEVKGDLTAKKMVLALLELAQQDHGALDCCVVVILSHGCQASHLQFPG AVYGTDGCPVSVEKIVNIFNGTSCPSLGGKPKLFFIQACGGEQKDHGFEVASTSPEDE SPGSNPEPDATPFQEGLRTFDQLDAISSLPTPSDIFVSYSTFPGFVSWRDPKSGSWYVE TLDDIFEQWAHSEDLQSLLLRVANAVSVKGIYKQMPGCNFLRKKLFFKTS (SEQ ID NO: 27). The nucleic acid sequence encoding the linker region of the iC9 polypeptide is encoded by a nucleic acid sequence comprising

(SEQ ID NO: 28)
TTTGGGGACGTGGGGGCCCTGGAGTCTCTGCGAGG
AAATGCCGATCTGGCTTACATCCTGAGCATGGAAC
CCTGCGGCCACTGTCTGATCATTAACAATGTGAAC
TTCTGCAGAGAAAGCGGACTGCGAACACGGACTGG
CTCCAATATTGACTGTGAGAAGCTGCGGAGAAGGT
TCTCTAGTCTGCACTTTATGGTCGAAGTGAAAGGG
GATCTGACCGCCAAGAAAATGGTGCTGGCCCTGCT
GGAGCTGGCTCAGCAGGACCATGGAGCTCTGGATT
GCTGCGTGGTCGTGATCCTGTCCCACGGGTGCCAG
GCTTCTCATCTGCAGTTCCCCGGAGCAGTGTACGG
AACAGACGGCTGTCCTGTCAGCGTGGAGAAGATCG
TCAACATCTTCAACGGCACTTCTTGCCCTAGTCTG
GGGGGAAAGCCAAAACTGTTCTTTATCCAGGCCTG
TGGCGGGGAACAGAAAGATCACGGCTTCGAGGTGG
CCAGCACCAGCCCTGAGGACGAATCACCAGGGAGC
AACCCTGAACCAGATGCAACTCCATTCCAGGAGGG
ACTGAGGACCTTTGACCAGCTGGATGCTATCTCAA
GCCTGCCCACTCCTAGTGACATTTTCGTGTCTTAC
AGTACCTTCCCAGGCTTTGTCTCATGGCGCGATCC
CAAGTCAGGGAGCTGGTACGTGGAGACACTGGACG
ACATCTTTGAACAGTGGGCCCATTCAGAGGACCTG
CAGAGCCTGCTGCTGCGAGTGGCAAACGCTGTCTC
TGTGAAGGGCATCTACAAACAGATGCCCGGGTGCT
TCAATTTTCTGAGAAAGAAACTGTTCTTTAAGACT
TCC.

To test the iC9 safety switch, each of the four modified T cells were incubated for 24 hours with 0, 0.1 nM, 1 nM, 10 nM, 100 nM or 1000 nM AP1903 (an induction agent for AP1903). Viability was assessed by flow cytometry using 7-aminoactinomycin D (7-AAD), a fluorescent intercalator, as a marker for cells undergoing apoptosis.

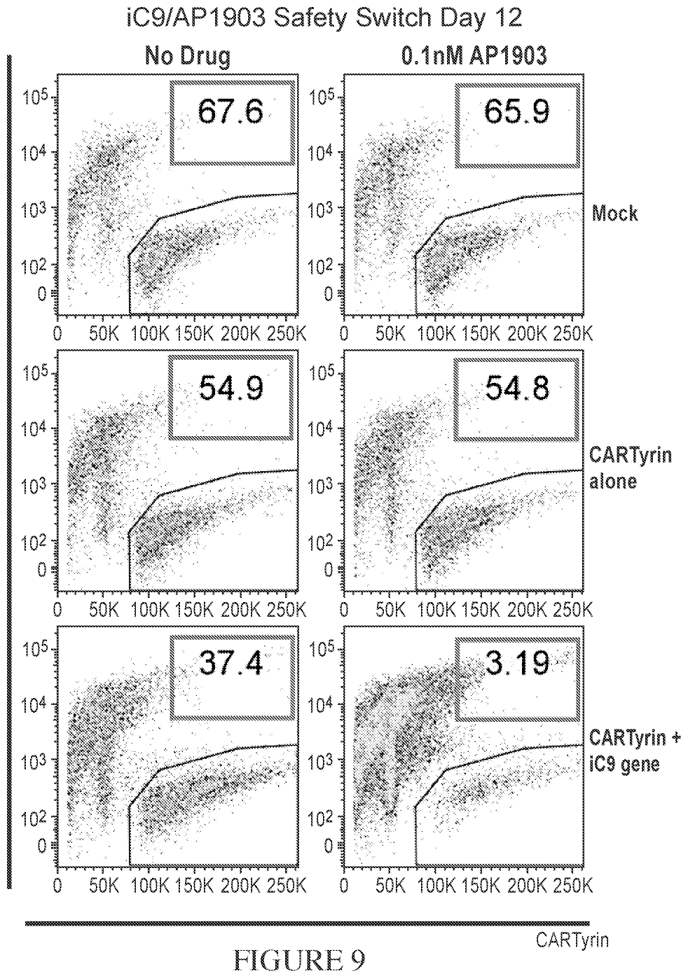

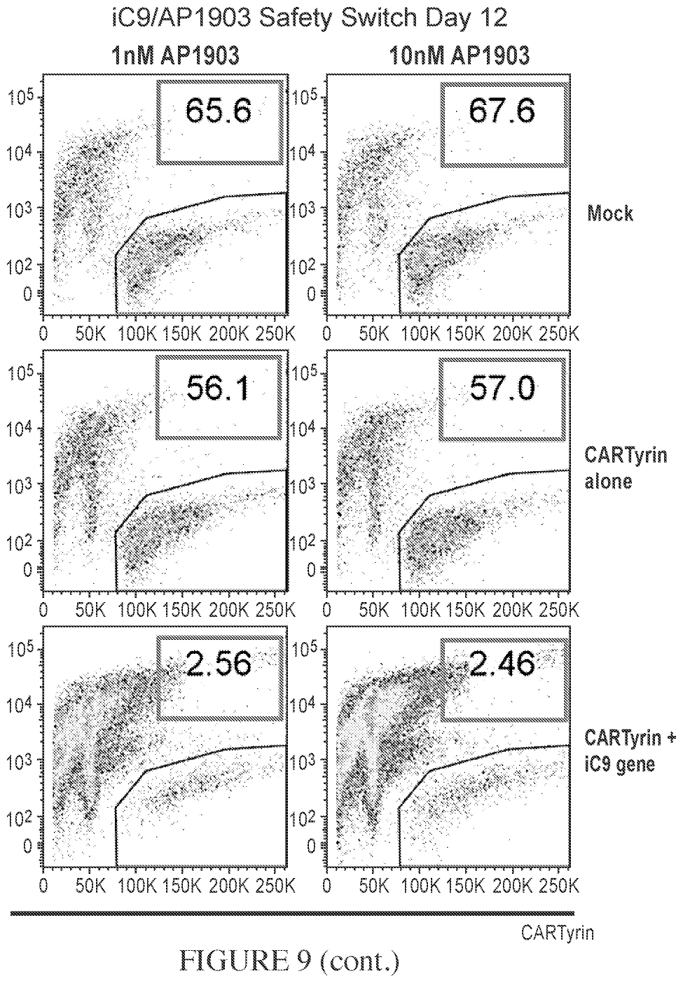

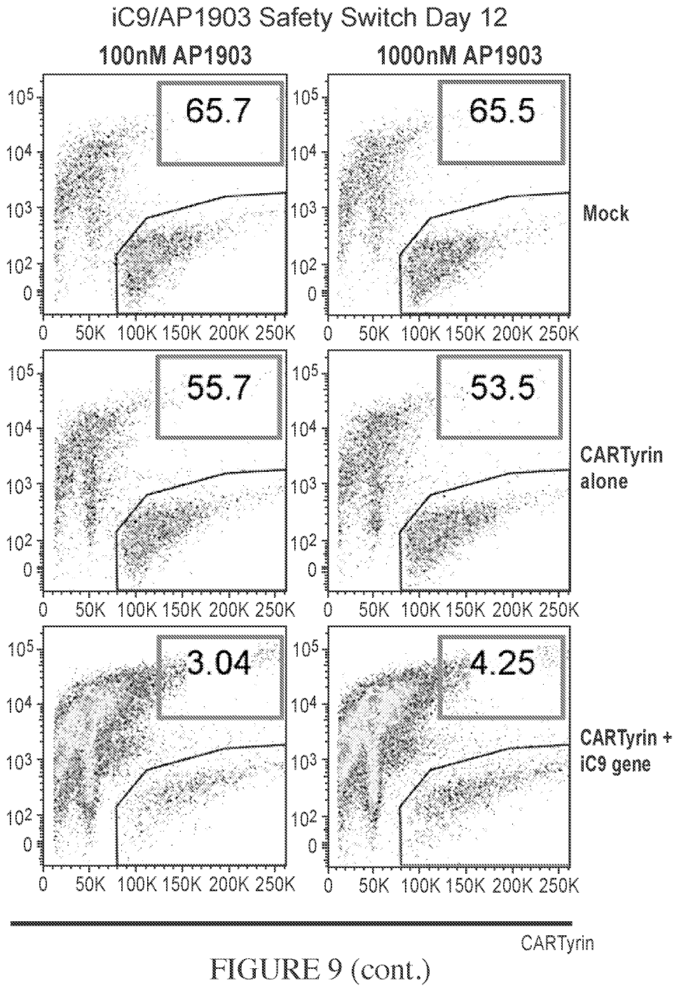

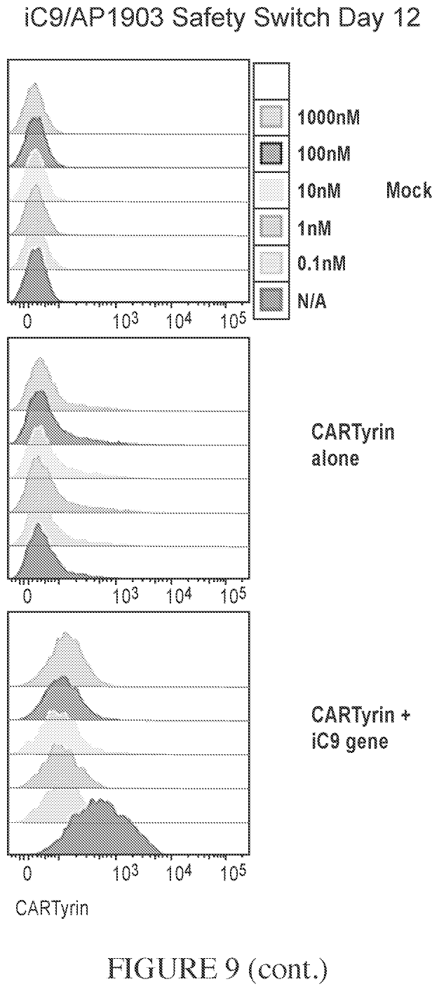

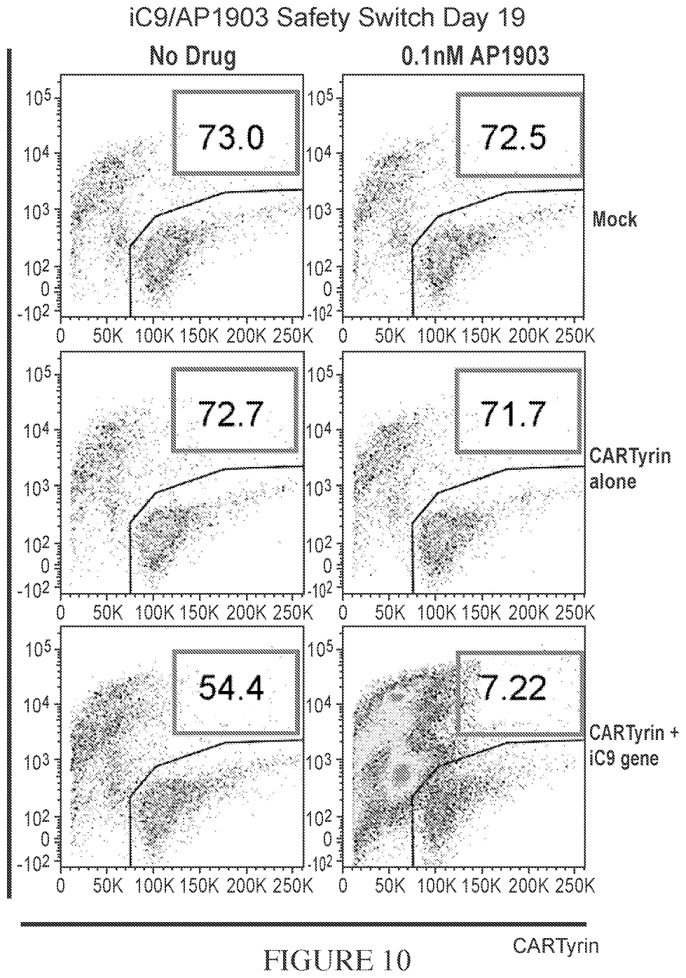

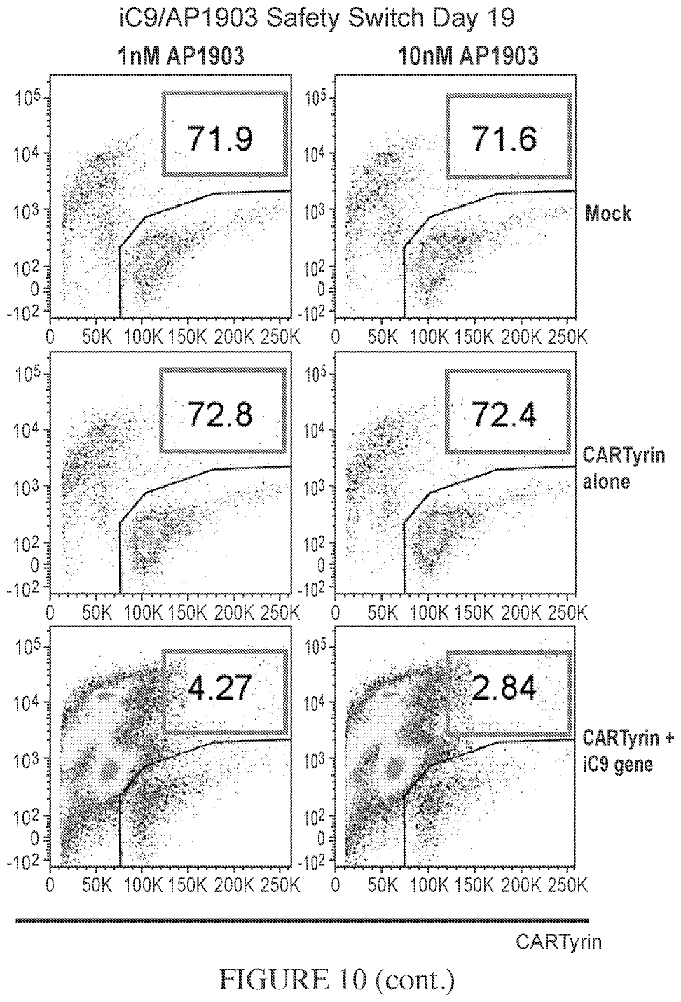

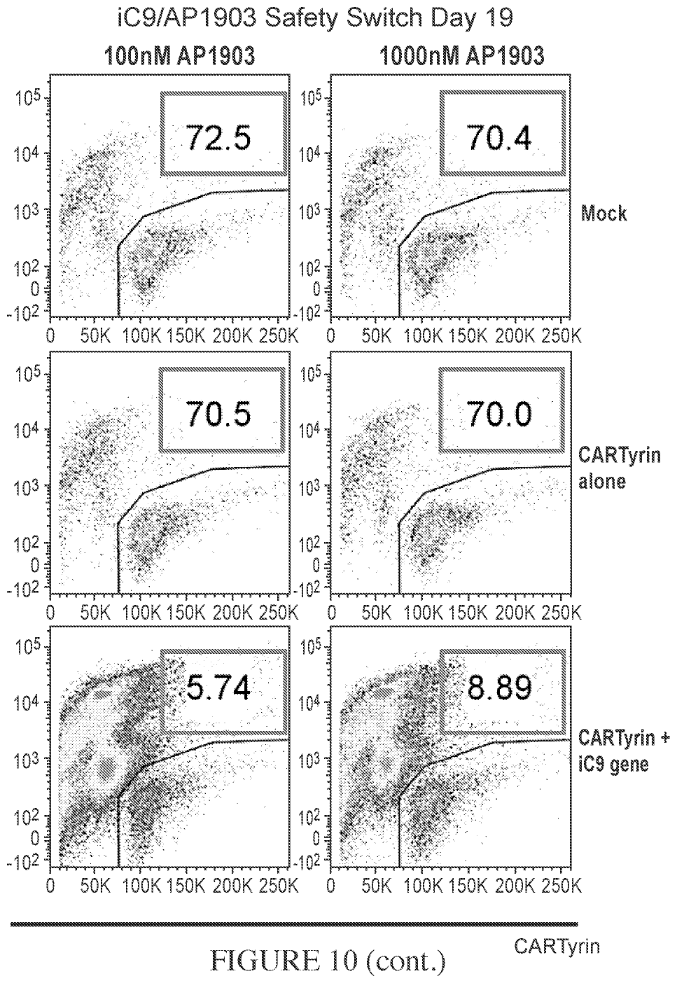

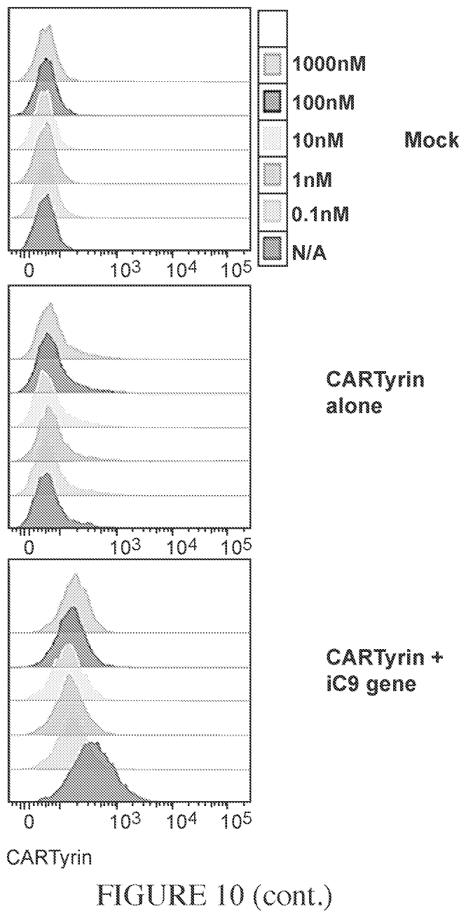

Cell viability was assessed at day 12 (see FIG. 9). The data demonstrate a shift of cell populations from the lower right to the upper left quadrants with increasing concentration of the induction agent in cells containing the iC9 construct; however, this effect is not observed in cells lacking the iC9 construct (those receiving only the CARTyrin), in which cells are evenly distributed among these two areas regardless of the concentration of the induction agent. Moreover, cell viability was assessed at day 19 (see FIG. 10). The data reveal the same trend as shown in FIG. 9 (day 12 post-nucleofection); however, the population shift to the upper left quadrant is more pronounced at this later time point (day 19 post-nucleofection).

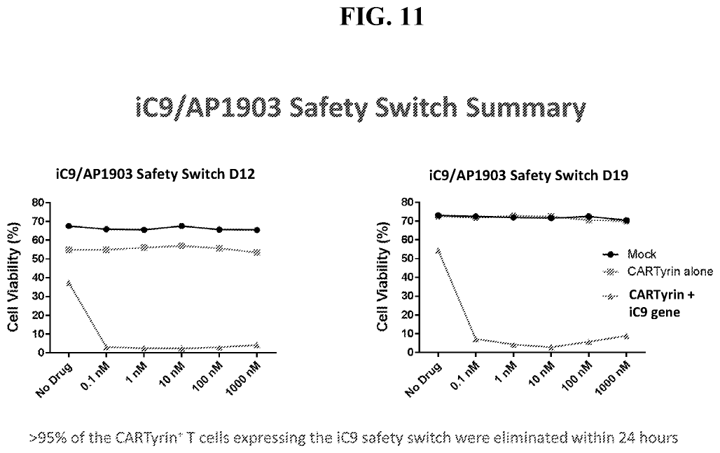

A quantification of the aggregated results was performed and is provided in FIG. 11, showing the significant impact of the iC9 safety switch on the percent cell viability as a function of the concentration of the induction agent (AP1903) of the iC9 switch for each modified cell type at either day 12 (FIG. 9 and left graph) or day 19 (FIG. 10 and right graph). The presence of the iC9 safety switch induces apoptosis in a significant majority of cells by day 12 and the effect is even more dramatic by day 19.

The results of this study show that the iC9 safety switch is extremely effective at eliminating active cells upon contact with an induction agent (e.g. AP1903) because AP1903 induces apoptosis at even the lowest concentrations of the study (0.1 nM). Furthermore, the iC9 safety switch may be functionally expressed as part of a tricistronic vector.

### Example 3: Open-Label, Multicenter, Phase 1 Study to Assess the Safety of P-BCMA-101 in Subjects with Relapsed/Refractory Multiple Myeloma (MM) Followed by a Phase 2 Assessment of Response and Safety (Prime)

Name of Investigational Product: P-BCMA-101 and Rimiducid Autologous CAR-T Cells Engineered to Contain an Anti-B-Cell Maturation Antigen (BCMA) Centyrin Coupled to TCR (and 4-1BB Signaling Domains (CARTyrin).

Brief Description of Investigational Product: P-BCMA-101 is comprised of T cells genetically modified using an electroporation-based, non-viral (DNA transposon) gene delivery system called the piggyBac® (PB) DNA modification system, which efficiently moves DNA from a plasmid to a chromosome via a “cut and paste” mechanism. Compared to lentivirus or y-retrovirus transduction, PB offers advantages, including a safer insertion profile, higher levels of transgene expression, stable and longer duration transgene expression, and a highly enriched, favorable T stem cell memory (Tscm) phenotype.

P-BCMA-101 cells are autologous T cells harvested from the subject and modified to contain 3 major components: an anti-BCMA Centyrin chimeric antigen receptor (CARTyrin) gene, a dihydrofolate reductase (DHFR) resistance gene, and an inducible caspase 9 (iC9)-based safety switch gene.

The CARTyrin expression cassette encodes for an extracellular BCMA-binding Centyrin protein fused to a CD8a signal/leader peptide, CD8a hinge/spacer, a CD8a transmembrane domain, an intracellular 4-1BB signaling domain and a T-cell receptor (TCR) (chain signaling domain (e.g. See FIG. 4). As compared with binding domains consisting of an antibody-based single chain variable region (scFv), the CARTyrin binding domain is a fully human protein that is smaller, more stable and potentially less immunogenic, but with similar antigen binding characteristics that enable specific recognition and killing of BCMA-expressing MM cells. CARTyrins are also designed to avoid T cell exhaustion.

The DHFR selection gene is used during manufacturing for ex vivo selection of transposed P-BCMA-101 T cells to produce a more homogeneous product.

The “safety switch” is an extra safety feature not found in most CAR-T cells designed to allow for rapid ablation of P-BCMA-101 cells, if indicated, by intravenous administration of the activation agent, rimiducid, a synthetic small molecule dimerizer drug (see Section 15.3).

Test Product, Dose, Route of Administration: Phase 1: Single Administration—dose levels of P-BCMA-101 administered intravenously as a single dose. Dose levels will be tested by cohort in the 3+3 escalation design described in the Study Design.

Phase 1: Cycle Administration—multiple doses of P-BCMA-101 administered intravenously in 2 cycles (Cohort A and Cohort C) or 3 cycles (Cohort B) of 2 weeks each. The total dose administered will follow the 3+3 design starting at ≤the maximum tolerated dose (MTD) as determined during single dose escalation. In the first cycle for both Cohorts A and B, ⅓ the total dose will be administered. In Cohort A up to ⅔ the total dose will be administered in the 2^{nd} cycle. In Cohort B up to ⅓ the total dose will be administered in each of the 2^{nd} and 3^{rd} cycles. In Cohort C up to ⅔ the total dose will be administered in the 1^{st} cycle and up to ⅓ the total dose will be administered in the 2^{nd} cycle.

Phase 1: Combination Administration—P-BCMA-101 will be administered in combination with approved therapies: lenalidomide (Cohort R: 10-25 mg orally daily for 21 of every 28 days beginning 1 week before P-BCMA-101 infusion; and Cohort RP: 10-25 mg orally daily for one week before apheresis then 21 of every 28 days beginning 1 week before P-BCMA-101 infusion) and rituximab (Cohort RIT: 375 mg/m² 12 and 5 days before P-BCMA-101 infusion, then every 8 weeks). The dose of P-BCMA-101 administered will follow the 3+3 design starting at ≤the MTD as determined during dose escalation.

Phase 2: P-BCMA-101 administered intravenously as a total dose of 6-15×10⁶ cells/kg.

Rimiducid may be administered intravenously at 0.4 mg/kg if clinically indicated.

Subjects who meet the criteria in Section 15.4 may be eligible to receive another infusion of P-BCMA-101.

Reference Therapy: None

Primary Objectives: The primary objective of this study is:

Phase 1—To determine the safety and maximum tolerated dose (MTD) of P-BCMA-101 based on dose limiting toxicities (DLT)

Phase 2—To assess the safety and efficacy of P-BCMA-101

Primary Endpoints:

Phase 1—Number of subjects with DLT at each dose level to define an MTD

Phase 2—Safety and tolerability based on adverse events (AEs), examinations, and standard laboratory studies

Overall response rate (ORR) and duration of response (DOR) by International Myeloma Working Group Criteria (Kumar, 2016) as assessed by an independent review committee (IRC)

Secondary Objectives: The secondary objectives of this study are to evaluate:

Phase 1—the safety and feasibility of P-BCMA-101; the anti-myeloma effect of P-BCMA-101; the effect of cell dose to guide selection of doses for further assessment in Phase 2/3 studies

Phase 2—Incidence and severity of cytokine release syndrome (CRS); additional efficacy endpoints

Secondary Endpoints: The following secondary endpoints will be evaluated:

Phase 1—Ability to generate protocol-proscribed doses of P-BCMA-101; Safety and tolerability based on AEs, examinations, and standard laboratory studies; CRS graded using Lee criteria (Lee, 2014); Efficacy based on International Myeloma Working Group (IMWG) Uniform Response Criteria (Rajkumar, 2011; Kumar 2016; Cavo, 2017); Overall response rate (ORR); Time to response (TTR); Duration of response (DOR); Progression free survival (PFS); and Overall survival (OS).

Phase 2—CRS graded using Lee criteria (Lee, 2014); Rate of IL-6 antagonist, corticosteroid, and rimiducid use; OS, PFS, TTR, minimal residual disease (MRD) negative rate

Exploratory Objectives: Exploratory objective of this study are to:

Phase 1—Evaluate the relationship between MM plasma cell BCMA expression, circulating soluble BCMA and clinical response

Phase 1 and 2—Characterize the expansion and functional persistence of the P-BCMA-101 cells; Evaluate the relationship between putative CRS markers and efficacy or safety; and Evaluate the effect of rimiducid on P-BCMA-101-related adverse events, if indicated

Exploratory Endpoints: The following exploratory endpoints will be assessed during the course of the study:

Phase 1—BCMA and/or other biomarkers in bone marrow; Soluble BCMA and/or other biomarker levels in blood

Phase 1 and 2—P-BCMA-101 cells (e.g. number of vector copies/mL in blood and bone marrow of P-BCMA-101 cells); P-BCMA-101 cell subset composition and clonality; CRS markers: C-reactive protein (CRP), ferritin, TL-6, IL-2, TNF-α, and interferon gamma (IFN-γ)

Subject Population and Number: Adults with confirmed relapsed/refractory MM. Up to approximately 120 subjects are planned for Phase 1. Approximately 100 evaluable subjects will be treated in Phase 2.

Study Design

The study will be conducted in multiple parts, a Phase 1, open-label, single ascending dose (SAD) phase; a Phase 1, multiple dose, cycle administration phase; a Phase 1, combination administration with lenalidomide or rituximab phase; and a Phase 2, open-label, efficacy and safety phase, in adult subjects with relapsed/refractory MM.

Only sites that are experienced in managing oncology subjects and stem-cell/bone marrow transplant with the resources to manage the types of acute emergent events expected with chimeric antigen receptor (CAR)-T cell administration will be selected to participate in this study. A Safety Committee will meet regularly to review data throughout the study.

Subjects meeting the protocol entry criteria will be eligible to enroll in the study. After a subject enrolls, leukapheresis will be performed to obtain peripheral blood mononuclear cells (PBMCs) which will be sent to a manufacturing site to produce P-BCMA-101 CARTyrin-T cells. The cells will then be returned to the investigational site and, after a standard chemotherapy-based conditioning regimen, will be administered to the subject as described below.

Phase 1—Phase 1 of the study is comprised of an open-label, multi-center, single ascending dose (SAD), multiple cohort study; a multiple dose cycle administration cohort study; and a combination administration study, in up to approximately 120 adult subjects. Phase 1 of the study will follow a 3+3 design of dose-escalating cohorts, wherein 3 subjects are initially planned to be dosed with P-BCMA-101 T cells for each cohort (Table 1). The Safety Committee may recommend enrollment of additional subjects in a cohort to further evaluate the outcomes observed at that dose level. For each of the first 2 cohorts, dosing of the first 3 subjects will be staggered. If Grade 3-related toxicity, CRS, or DLT is reported, the Safety Committee will review the data and determine whether to proceed to the next subject. The Safety Committee will review the data at the end of each cohort to determine progression to the next cohort. At the discretion of the Safety Committee, beginning with the 3rd cohort, dosing of the first and second subject in each cohort will be staggered.

DLT is defined as any National Cancer Institute—Common Terminology Criteria for Adverse Events (NCI CTCAE) Grade≥3 event at least possibly related to P-BCMA-101, including uncontrollable expansion of P-BCMA-101 cells, and not attributable to the underlying disease or lymphodepleting chemotherapy regimen with onset within the first 28 days following the last P-BCMA-101 infusion with the following exceptions:

Grade 3 or 4 neutropenia with or without neutropenic fever resolving within 28 days following the last P-BCMA-101 cell infusion
Grade 3 fever
Grade 3 or 4 thrombocytopenia, with or without bleeding due to thrombocytopenia, resolving within 28 days following the last P-BCMA-101 cell infusion
Grade 3 or 4 anemia and lymphopenia
Grade 3 or 4 hypogammaglobulinemia

### Alopecia

Grade 3 or 4 nausea, vomiting or diarrhea which responds to medical treatment within 24 hours
Immediate hypersensitivity reactions (fever, rash, bronchospasm) occurring within 2 hours of cell infusion (related to cell infusion) that are reversible to a Grade 2 or less within 6 hours of cell administration with standard antihistamine-based therapy
Grade 3 encephalopathy that recovers to less than Grade 2 within 28 days
Grade 3 CRS per Lee criteria (Lee, 2014) that resolves within 14 days
Grade 3 non-hematological laboratory abnormalities that recover to ≤Grade 2 in 14 days
Grade 4 non-hematological laboratory abnormalities that recover to ≤Grade 2 in 7 days

TABLE 1
Dose Escalation Guidelines

| Outcome: | |
|---|---|
| (# subjects with | |
| DLT) | Action: |
| 0 out of 3 subjects | Escalate dose for next cohort of 3 subjects |
| 1 out of 3 subjects | Treat next 3 subjects at the same dose |
| ≥2 out of 3 subjects | Halt dose escalation; treat at least 6 subjects |
| | at a lower dose to determine the MTD¹ |
| 1 out of 6 subjects | Escalate dose for next cohort of 3 subjects |
| ≥2 out of 6 subjects | Halt dose escalation; treat at least 6 subjects |
| | at a lower dose to determine the MTD¹ |

¹MTD—the highest dose for which no more than 1 of 6 treated subjects exhibits DLT

1. MTD—the Highest Dose for which No More than 1 of 6 Treated Subjects Exhibits DLT

The 3+3 dose escalation will be conducted as follows, with the Safety Committee reviewing the data at the end of each cohort to determine the outcome. Beginning with Cohort 1, at least 3 subjects will be dosed in the cohort. If no DLT through Day 28 after last dose is observed in the first 3 subjects, then escalation may proceed to the next cohort. If DLT is observed in 1 of the first 3 subjects, then at least 3 additional subjects will be treated at this dose level. If no further DLT is observed, escalation may proceed. If DLTs are observed in 2 or more of 3 or 6 subjects, the MTD will be considered to be at the next lower dose level and further enrollment may take place at a lower dose level, or an intermediate dose level may be tested at the discretion of the Safety Committee. In the event that 2 or more subjects experience DLTs in Cohort 1, the Safety Committee, after reviewing available data, may elect to dose 3 subjects in Cohort −1 with the same 3+3 expansion rules. In the event that 2 or more subjects experience DLTs in Cohort −1, the Safety Committee, based on consideration of safety and efficacy data to assess risk vs. benefit, may elect to dose 3 subjects at a lower dose with the same 3+3 expansion rules or discontinue the study.

Proposed Doses (P-BCMA-101 cells/kg/dose) include:

Cohort minus 1: 0.25×10⁶

### Cohort 1: 0.75×10⁶

### Cohort 2: 2×10⁶

### Cohort 3: 6×10⁶

### Cohort 4: 10×10⁶

### Cohort 5: 15×10⁶

Additional subjects may be dosed in a cohort at the direction of the Safety Committee based on the safety and efficacy data from that cohort to further evaluate the effects of P-BCMA-101, provided the dose does not exceed the MTD. If Cohort 5 is completed without concluding an overall MTD, the Safety Committee may elect to assess further escalation cohorts in 5-10×10⁶ P-BCMA-101 cells/kg increments.

Phase 1—Cycle Administration: In the Phase 1 cycle dose administration portion of the study, multiple doses of P-BCMA-101 will be administered intravenously in 2 cycles (Cohort A and Cohort C) or 3 cycles (Cohort B) of 2 weeks each. The total dose administered will follow the 3+3 design starting at ≤the MTD as determined during single dose escalation. In the first cycle for both Cohorts A and B, ⅓ the total dose will be administered. In Cohort A up to ⅔ the total dose will be administered in the 2nd cycle. In Cohort B up to ⅓ the total dose will be administered in each of the 2nd and 3rd cycles. In Cohort C up to ⅔ the total dose will be administered in the 1st cycle and up to ⅓ the total dose will be administered in the 2nd cycle. The same 3+3 dose escalation and/or de-escalation rules described for single administration will be utilized. These procedures are detailed in Section 15.5.

Phase 1—Combination Administration: P-BCMA-101 will be administered in combination with approved therapies: lenalidomide (Cohort R and Cohort RP) and rituximab (Cohort RIT). The dose of P-BCMA-101 administered will follow the 3+3 design starting at :5 the MTD as determined during dose escalation. The same 3+3 dose escalation and/or de-escalation rules described for single administration will be utilized. These procedures are detailed in Section 15.6.

Phase 2—Phase 2 of the study is an open-label, multi-center study in approximately 100 adult subjects with relapsed and/or refractory MM. Subjects will receive a total dose of 6-15×10⁶ cells/kg (per the schedule determined in Phase 1).

Study visits—Treated subjects in Phase 1 and Phase 2 will undergo serial measurements of safety, tolerability and response (myeloma staging). These measures will be obtained at Screening, Enrollment or Baseline Visit and the Conditioning Chemotherapy Period. The P-BCMA-101 Administration Period and follow-up visits in both Phase 1 and Phase 2 will be at Day 10, Week 2, 3, 4, 6, 8, Month 3, 4, 5, 6, 7, 8, 9, and then every 3 months thereafter for up to 24 months after P-BCMA-101 administration. After completing or withdrawing from this protocol, consenting subjects who have received P-BCMA-101 should enroll in a separate protocol that allows for continued follow-up for a total of 15 years after last dosing to evaluate long-term safety.

Screening Visit—Consented subjects will undergo a Screening Visit to determine eligibility. Subjects who meet all the inclusion criteria and none of the exclusion criteria will return for the Enrollment and Leukapheresis Visits.

Enrollment Visit—Eligible subjects will return for an Enrollment visit to provide samples and measurements that must be collected in advance of Leukapheresis. Enrollment assessments are to be conducted 14 days (±3 days) prior to Leukapheresis or have medical monitor approval.

Leukapheresis Visit—Eligible subjects who enroll will return and undergo leukapheresis to obtain PBMCs for P-BCMA-101 manufacturing. This visit should occur within ˜28 days of the Screening Visit. Once the product is manufactured, subjects will return for the combination therapy (if applicable), Conditioning Chemotherapy and P-BCMA-101 cell Administration Periods approximately 4 weeks after the Leukapheresis Visit. If P-BCMA-101 cells that meet release criteria cannot be manufactured from the leukapheresis sample, a second leukapheresis and manufacturing may be attempted. If the second attempt also fails, the subject will be withdrawn from the study and considered not to have undergone study treatment.

Subjects who experience rapid disease progression following the Leukapheresis Visit and prior to the Conditioning Chemotherapy and P-BCMA-101 cell Administration Period may be administered salvage therapy at the discretion of the Investigator. Salvage therapy should not be used unless necessary and will be determined by the subject's clinical history (previously used agents are preferred, and medical monitor approval needed) at the discretion of the Investigator. If a subject receives salvage therapy, the Conditioning Chemotherapy and P-BCMA-101 cell Administration Period should be scheduled at least 2 weeks or 5 half-lives after the date of the last treatment of salvage therapy and the subject should meet the criteria described in Sections 4 and 6 regarding entry criteria (including those for measurable MM) and concomitant medications. The subject's response to the salvage therapy will be evaluated by the Investigator and medical monitor to determine whether the subject will remain eligible to receive the Investigational Product.

Subjects will be permitted to receive radiation therapy or plasmapheresis and exchange for palliative purposes throughout the study period.

Baseline Visit (Day −12 to Day −6)—Once the product is manufactured, during the week prior to starting Conditioning Chemotherapy, subjects will return for Baseline assessments and to confirm continued eligibility. The following assessments should be repeated within 72 hours prior to Day −5: Mini Mental Status Exam (MMSE), physical exam, vital signs, chemistry panel including electrolytes and magnesium, hematology including B and T cell counts, coagulation, assessment of circulating myeloma/plasma cells, and pregnancy test (if applicable). A baseline myeloma response assessment must be conducted within 7 days of initiating conditioning chemotherapy and combination therapy. The baseline Fresh Sample of Bone Marrow and Tumor is not used to confirm eligibility and the 7-day window is intended to provide flexibility for subjects and investigators; if a new bone marrow biopsy/aspirate was performed and provided during Screening, this does not need to be repeated during the Baseline visit.

Conditioning Chemotherapy and P-BCMA-101 Cell Administration Period—Before dosing with the P-BCMA-101 cell infusion, subjects will receive a conditioning lymphodepletion chemotherapy regimen of 300 mg/m² of cyclophosphamide and 30 mg/m² of fludarabine, with each chemotherapy agent given intravenously daily for 3 consecutive days (Day −5 through Day −3). Subjects should continue to meet entry criteria at the time of initiation of conditioning chemotherapy or have medical monitor approval. For subjects in Cohort R, Cohort RP and Cohort RIT, the combination therapy should be administered prior to conditioning chemotherapy on applicable days.

After 2 rest days following the lymphodepletion chemotherapy regimen, subjects will be dosed with P-BCMA-101 administered intravenously over approximately 5 to 20 mins (Day 0) (subjects should be pre-medicated with acetaminophen and diphenhydramine). Prior studies conducted with CAR-T therapies have observed peak toxicity to occur within 3-7 days of investigational product administration. Study subjects will be closely monitored during and after the infusion and for approximately 7 days afterwards. This observation period will include serial assessments of AEs, including the emergence of P-BCMA-101 cell-related toxicities, such as CRS, for all subjects. CRS will be graded using the Lee criteria (Lee, 2014). Guidance regarding grading and management of AEs can be found in Section 8 of this protocol and in the Study Reference Manual. Guidance for the use of rimiducid for significant P-BCMA-101-related toxicity can be found in Section 15.3 and the Study Reference Manual.

Subjects may be admitted to the hospital for the P-BCMA-101 administration if the Investigator deems appropriate based on an individual patient's risks. Admission is not required, but subjects should remain within 50 miles of the hospital through approximately 14 days after last dose of P-BCMA-101 and assessed for admission in case of symptoms of CRS or neurotoxicity such as fever. If admitted, subjects will not be discharged until they are assessed as stable by the Investigator. Subjects may be maintained as an inpatient before P-BCMA-101 administration during lymphodepleting chemotherapy or after the above criteria are met as the Investigator deems appropriate.

Follow up visits: Day 10, Week 2, 3, 4, 6, 8, Month 3, 4, 5, 6, 7, 8, 9, 12, 15, 18, 21 and 24. Subjects will return for regular follow up after last dose of P-BCMA-101 and will undergo serial assessment of safety, tolerability and anti-myeloma response as specified in the Schedule of Events.

Repeat Administration: If sufficient P-BCMA-101 cells remain from manufacturing when a subject's disease progresses, with Safety Committee approval additional cells may be administered up to the highest dose level that has successfully completed dose-limiting toxicity assessment. In order to receive an additional P-BCMA-101 T-cell infusion, subjects will be assigned a new subject identification number, they will have to meet all eligibility criteria as described for the initial dosing, and will undergo the same screening, enrollment, conditioning chemotherapy, and follow-up procedures except for leukapheresis. Retreatment procedures are outlined in Section 15.4.

Safety Monitoring: A Safety Committee comprised of the Investigators and a clinical representative of the sponsor will be established and will review data regularly for all subjects and for each cohort to determine dose escalation and enrollment.

Inclusion Criteria:

1. Must have signed written, informed consent;
2. Males or females, ≥18 years of age;
3. Must have a confirmed diagnosis of active MM as defined by the IMWG criteria at initial diagnosis (Rajkumar, 2014);
4. Must have measurable MM as defined by at least 1 of the following criteria: Phase 1:
Serum M-protein greater or equal to 0.5 g/dL (5 g/L);
Urine M-protein greater or equal to 200 mg/24 h;
Serum free light chain (FLC) assay: involved FLC level greater or equal to 10 mg/dL (100 mg/L) provided serum FLC ratio is abnormal;
Bone marrow plasma cells>30% of total bone marrow cells, or other measurable bone disease (e.g., plasmacytomas measurable by PET or CT) (with medical monitor approval)

### Phase 2:

Serum M-protein greater or equal to 1.0 g/dL (10 g/L);
Urine M-protein greater or equal to 200 mg/24 h;
Serum FLC assay: involved FLC level greater or equal to 10 mg/dL (100 mg/L) provided serum FLC ratio is abnormal
5. Must have relapsed/refractory MM as defined by the following:

### Phase 1:

Received at least 3 prior lines of therapy, which must have contained a proteasome inhibitor and immunomodulatory agent (IMiD); OR
Received at least 2 prior lines of therapy if “double-refractory” to a proteasome inhibitor and IMiD, defined as progression on or within 60 days of treatment with these agents.

### Phase 2:

Received at least 3 prior lines of therapy which must have contained a proteasome inhibitor, an IMiD, and CD38 targeted therapy with at least 2 of the prior lines in the form of triplet combinations, and undergone ≥2 cycles of each line unless PD was the best response; AND Refractory to the most recent line of therapy; AND
Undergone ASCT or not be a candidate for ASCT.
Note: induction therapy, autologous stem cell transplant (ASCT), and maintenance therapy, if given sequentially without intervening progression, should be considered as single line. 6. Must be willing to practice birth control from the time of Screening and throughout the study (both males and females of childbearing potential).
Females on cohorts R, RP or RIT must commit either to abstain continuously from sexual intercourse or to use two methods of reliable birth control, beginning 4 weeks prior to initiating treatment, during therapy, during dose interruptions and continuing for 4 weeks following discontinuation of lenalidomide and 12 months after last dose of rituximab.
Males in cohort R or RP must always use a latex or synthetic condom during any sexual contact with females of reproductive potential while taking lenalidomide and for up to 4 weeks after discontinuing lenalidomide, even if they have undergone a successful vasectomy.
Male patients taking lenalidomide must not donate sperm.
7. Must have a negative serum pregnancy test at Screening and a negative urine test within 3 days prior to initiating the lymphodepletion chemotherapy regimen (females of childbearing potential).
Female subjects in cohort R and RP must have two negative pregnancy tests prior to initiating lenalidomide. The first test should be performed within 10-14 days and the second test within 24 hours prior to subject starting lenalidomide therapy and then weekly during the first month, then monthly thereafter in females with regular menstrual cycles or every 2 weeks in females with irregular menstrual cycles.
8. Must be at least 90 days since autologous stem cell transplant, if performed.
9. Must have adequate vital organ function, defined as follows (or medical monitor approval): Serum creatinine<2.0 mg/dL and estimated creatinine clearance>30 mL/min as calculated using the Cockcroft-Gault formula and not dialysis-dependent.
Absolute neutrophil count>1000/μL and platelet count>50,000/μL (>30,000/μL if bone marrow plasma cells are >50% of cellularity).
Adequate absolute CD3 count estimated for obtaining target cell dose based on dosage cohort (Phase 2: absolute lymphocyte count>300/μL)
Hemoglobin>8 g/dL (transfusion and/or growth factor support is allowable).
Serum glutamic oxaloacetic transaminase (SGOT)<3×the upper limit of normal and total bilirubin<2.0 mg/dL (unless there is a molecularly documented history of Gilbert's syndrome).
Left ventricular ejection fraction (LVEF)>45%. LVEF assessment must have been performed within 4 weeks of enrollment.
10. Must have recovered from toxicities due to prior therapies, with the exception of peripheral neuropathy, to Grade<2 according to the NCI CTCAE Version 4.03 criteria or to the subject's prior baseline.
11. Must have an Eastern Cooperative Oncology Group (ECOG) performance status of 0 to 1.

Exclusion Criteria: 1. Is pregnant or lactating; 2. Has inadequate venous access and/or contraindications to leukapheresis; 3. Has active hemolytic anemia, plasma cell leukemia, Waldenstrom's macroglobulinemia, POEMS syndrome (polyneuropathy, organomegaly, endocrinopathy, monoclonal protein, and skin changes), disseminated intravascular coagulation, leukostasis, or amyloidosis; 4. Has an active second malignancy (not disease-free for at least 5 years) in addition to MM, excluding low-risk neoplasms such as non-metastatic basal cell or squamous cell skin carcinoma; 5. Has active autoimmune disease, such as psoriasis, multiple sclerosis, lupus, rheumatoid arthritis, etc. (the medical monitor will determine if a disease is active and autoimmune); 6. Has a history of significant central nervous system (CNS) disease, such as stroke, epilepsy, etc. (the medical monitor will determine if significant); 7. Has an active systemic infection (e.g. causing fevers or requiring antimicrobial treatment); 8. Has hepatitis B or C virus, human immunodeficiency virus (HIV), or human T-lymphotropic virus (HTLV) infection, or any immunodeficiency syndrome; 9. Has New York Heart Association (NYHA) Class III or IV heart failure, unstable angina, or a history of myocardial infarction or significant arrhythmia (e.g. atrial fibrillation, sustained [>30 seconds] ventricular tachyarrhythmias, etc.); 10. Has any psychiatric or medical disorder (e.g. cardiovascular, endocrine, renal, gastrointestinal, genitourinary, immunodeficiency or pulmonary disorder not otherwise specified) that would, in the opinion of the Investigator or medical monitor, preclude safe participation in and/or adherence to the protocol (including medical conditions or laboratory findings that indicate a significant probability of not qualifying for or being unable to undergo adequate leukapheresis, conditioning chemotherapy and/or CAR-T cell administration); 11. Has received prior gene therapy or gene-modified cellular immunotherapy (or have approval of the medical monitor). Subject may have received non-gene-modified autologous T-cells or stem cells in association with an anti-myeloma treatment; 12. Has received anti-cancer medications within 2 weeks or 5 half-lives (whichever is longer or have medical monitor approval) of the time of initiating conditioning chemotherapy; 13. Has received immunosuppressive medications within 2 weeks of the time of initiating leukapheresis, and/or expected to require them while on study (the medical monitor will determine if a medication is considered immunosuppressive). Generally, all non-essential medications (including supplements, herbal medications, etc.) should be discontinued from 2 weeks before leukapheresis until 2 months after P-BCMA-101 administration due to the potential for unappreciated immunosuppressive effects; 14. Has received systemic corticosteroid therapy≥5 mg/day of prednisone or equivalent dose of another corticosteroid within 2 weeks of either the required leukapheresis or 1 week or 5 half-lives (whichever is shorter) of the administration of P-BCMA-101 or is expected to require it during the course of the study. (Topical and inhaled steroids are permitted. Systemic corticosteroids are contraindicated after receiving P-BCMA-101 cells outside of study-specific guidance); 15. Has CNS metastases or symptomatic CNS involvement (including leptomeningeal carcinomatosis, cranial neuropathies or mass lesions and spinal cord compression) of their myeloma; 16. Has a history of severe immediate hypersensitivity reaction to any of the agents used in this study; 17. Has a history of having undergone allogeneic stem cell transplantation, or any other allogeneic or xenogeneic transplant, or has undergone autologous transplantation within 90 days; 18. Unable to take acetylsalicylic acid (ASA) (325 mg) daily as prophylactic anticoagulation. Patients intolerant to ASA may use warfarin or low molecular weight heparin) (Cohorts R and RP only); 19. History of thromboembolic disease within the past 6 months, regardless of anticoagulation (Cohorts R and RP only).

Duration of Study: Subjects will be followed for up to 2 years after the last dose in this study, following which consenting subjects will roll over into a long-term safety follow-up protocol for a total of 15 years follow-up post-last dose.

Schedule of Events: See Table 2, for single administration Schedule of Events—Screening through Conditioning Chemotherapy, and Table 3, Schedule of Events—P-BCMA-101 Administration and Follow-Up. The Schedules of Events for retreatment of subjects with P-BCMA-101 is described in Table 8 and Table 9 (see Section 15.4). For rimiducid treatment see the Schedule of Events in Table 7. The Schedule of Events for subjects in Cycle Administration cohorts is described in Table 10, Table 11, Table 12, and Table 13 (see Section 15.5). The Schedule of Events for subjects in Combination Administration cohorts is described in Table 14 and Table 15 (see Section 15.6).

Criteria for pausing dosing or stopping the study: If a study-defined DLT or any treatment related death occurs, dosing of new subjects will be paused until the Safety Committee meets, reviews the event(s), and determines forward plans, which might include stopping the study, reducing subsequent dose levels, instituting additional safety procedures or a study amendment, continuing the study as planned or other measures as appropriate to the event. As described above, if 2 or more subjects have DLTs in a cohort during Phase 1, and a ≥10% incidence of ≥Grade 4 or ≥30% incidence of ≥Grade 3 CRS or neurotoxicity at or below a corresponding dose level in Phase 2 with >10 patients treated, that dose level will have exceeded the MTD and any further dosing would take place at a lower dose level.

Statistical Methodology: The demographic and baseline characteristics, safety, and efficacy data will be summarized using appropriate descriptive statistics. Data analyses will be provided by dose cohort, as well as for all subjects combined where appropriate. Descriptive statistics including means, medians, standard deviations, and ranges will be calculated for continuous variables, and categorical data will be summarized using counts and percentages. For response rate endpoints, point estimates and two-sided exact binomial 95% confidence intervals will be computed. Time-to-event variables will be summarized using the Kaplan-Meier method.

Treatment-emergent AEs (TEAEs) will be summarized using counts and percentages of subjects by cohort, and for all subjects combined. TEAEs will also be summarized by severity and relationship. Concomitant medications will be summarized using counts and percentages of subjects by dose cohort.

Vital signs, electrocardiogram (ECG) measurements, and laboratory results will be summarized using descriptive statistics for observed values and change from baseline values by cohort. Laboratory results will also be summarized relative to the normal range (below, within, or above) by cohort.

The Phase 1 part of the study is a standard 3+3 design of dose cohorts intended to determine a dose below which a 33% incidence of DLTs occurs. Thus, up to 120 subjects may be enrolled to include the possibility of 18 cohorts of 6 subjects during dose escalation, cycle administration and combination administration, as well as subjects who might be enrolled to replace those who discontinue prior to completion of the DLT evaluation period or further assess findings in a cohort.

For the Phase 2 part of the study response rate endpoints will be tested to exclude a response rate of ≤30% as obtained with the recently approved standard of care agent daratumumab at p<0.05. Time-to-event variables will be summarized using the Kaplan-Meier method. With a 100-subject sample, the Phase 2 part of the trial will have 90% power to detect a 15-percentage point improvement over a 30% response rate. This power calculation is based on an exact test for a binomial proportion with a 1-sided 0.05 significance level. A futility analysis will be conducted once 35 subjects are enrolled, received P-BCMA-101, and followed up for 4 months or progressed prior to 4-month follow-up. This analysis set is called Futility Analysis Set (FAS). The futility analysis will use Futility Index (FI) which is equal to 1 minus the Conditional Power (CP) based on the observed proportion of BOR in FAS. The study may be stopped if FI is above 0.80 (that is, if CP falls below 0.20).

Subjects who receive additional infusions of P-BCMA-101 will also be analyzed as separate subgroups for all outcomes afterwards.

TABLE 2
1. Schedule of Events-Screening through Conditioning Chemotherapy (Single Administration)

| | | | | Baseline | Baseline | | | | | |
|---|---|---|---|---|---|---|---|---|---|---|
| | Screening | Enrollment¹² | | (within 7 days | (within 72 | | | | | |
| | (within 28 | (2 weeks ± 3 | | prior to | hours prior to | Conditioning Chemotherapy¹¹ | | | | |
| | days prior to | days prior to | Leuka- | Conditioning | Conditioning | Days | | | | |
| Procedures | leukapheresis) | leukapheresis) | pheresis¹³ | Chemotherapy) | Chemotherapy) | −5 | −4 | −3 | −2 | −1 |
| Informed Consent | X | | | | | | | | | |
| Inclusion/Exclusion Criteria | X | | | | | X | | | | |
| Medical History and Disease | X | | | | | | | | | |
| Assessment¹ | | | | | | | | | | |
| ECOG Performance Status | X | | | | | X | | | | |
| Mini Mental Status Exam (MMSE) | X | | | | X | X | | | | |
| Height and Weight² | X | X¹² | X | | | | | | | |
| 12-Lead ECG³ | X | | | | | | | | | |
| Echocardiogram | X | | | | | | | | | |
| Archival Tumor to Central Lab | X | | | | | | | | | |
| Samples for Myeloma Response^{4,14} | X⁴ | | | X⁴ | | | | | | |
| Fresh Sample of Bone Marrow and | X⁴ | | | X⁴ | | | | | | |
| Tumor^{4,14} | | | | | | | | | | |
| Blood Sample for BCMA and | | X¹² | | | | | | | | |
| Other Biomarkers¹⁴ | | | | | | | | | | |
| Physical Exam | X | | | | X | X | | | | |
| (Including Neurological Exam) | | | | | | | | | | |
| Vital Signs (BP, HR, RR, | X | | | | X | X | X | X | | |
| O² sat, temp) | | | | | | | | | | |
| Pregnancy Test (serum or urine)⁵ | X | | | | X | | | | | |
| Blood Sample for HIV, | X | | | | | | | | | |
| Hepatitis B and C, | | | | | | | | | | |
| and HTLV Screening¹⁴ | | | | | | | | | | |
| Chemistry Panel, Hematology | X | X | | | X | | X | X | | |
| including B and T cell counts, | | | | | | | | | | |
| Coagulation⁶ | | | | | | | | | | |
| Assessment of circulating | X | X | | | X | | | | | |
| myeloma/plasma cells⁶ | | | | | | | | | | |
| Leukapheresis | | | X¹³ | | | | | | | |
| Fludarabine/Cyclophosphamide | | | | | | X | X | X | | |
| Blood Sample for P-BCMA-101 | | X¹² | | | | | | | | |
| T cells^{7,14} | | | | | | | | | | |
| Blood Sample for Serum IgG¹⁴ | | X¹² | | | | | | | | |
| Blood Sample for Immunogenicity | | X¹² | | | | | | | | |
| Assays¹⁴ | | | | | | | | | | |
| Blood Sample for Cytokine Release | | X¹² | | | | | | | | |
| Syndrome markers (CRS)⁸: | | | | | | | | | | |
| e.g. CRP, Ferritin, Cytokines IL-6, | | | | | | | | | | |
| IL-2, IL-15, TNF-α, and IFN-γ^{8,14} | | | | | | | | | | |
| PET/CT^{4,9} | | | | X^{4,9} | | | | | | |
| Adverse Events | | X | X | X | X | X | X | X | | |
| Concomitant Medication¹⁰ | X¹⁰ | X¹² | X | X | X | X | X | X | | |

¹Including MM measurement results for the 6 months prior to Screening required (including serum protein electrophoresis [SPEP], urine protein electrophoresis [UPEP], serum immunofixation [SIFE], urine immunofixation [UIFE], serum free light chains [FLC], minimal residual disease [MRD] and as clinically indicated, PET/CT and/or bone marrow/tumor biopsy/aspirate [Bone marrow biopsy], if available).
²Height obtained at Screening Visit only
³12-Lead ECG and echocardiogram obtained at Screening.
⁴Myeloma Response will be assessed for Screening (may be obtained from any result within the last month), baseline within 7 days of initiating conditioning chemotherapy (note: 2 or more assessments must be performed between completion of the last myeloma therapy, including salvage therapy, and initiation of lymphodepleting chemotherapy), Days 0 (before P-BCMA-101 administration-thus the 24-hr urine collection is to be started on Day −1 so as to be completed before P-BCMA-101 administration), and as clinically indicated per the International Myeloma Working Group criteria and based on the standard assessments, including SPEP, UPEP, SIFE, UIFE, and serum FLC. Bone marrow biopsy, aspirate and MRD (analyzed by central lab) must be done within 7 days of initiating conditioning chemotherapy, then as clinically indicated, or have a medical monitor exemption. A sample of bone marrow aspirate and a sample of bone marrow biopsy are to be submitted (as described in the Study Reference Manual) for assessment of MRD, plasma cell frequency, P-BCMA-101 cells, BCMA, and other biomarkers. A portion of any extramedullary biopsy should also be submitted for assessment of P-BCMA-101 cells, BCMA, and other biomarkers. If a new bone marrow biopsy/aspirate was performed and provided during screening, this does not need to be repeated during the Baseline visit. PET/CT is to be conducted as clinically indicated.
⁵Females of childbearing potential will have a serum pregnancy test at Screening, and a urine pregnancy test within 72 hours (3 days) prior to initiating conditioning chemotherapy.
⁶Blood chemistries will include sodium, potassium, chloride, magnesium, bicarbonate, blood urea nitrogen, creatinine, calcium, albumin, glucose, LDH, total protein, ALT, AST, bilirubin (total and direct), alkaline phosphatase, PTT and PT or INR.
Hematological studies include:
Complete blood count (CBC)
Platelets
B (CD19) and T cell counts (CD3) and CD4 and CD8 at all timepoints except Days −3, −4
Assessment of circulating myeloma/plasma cells (e.g. by flow cytometry or CBC with manual differential) is required at timepoints prior to P-BCMA-101 administration. Contact the sponsor and refer to exclusion criteria #3 and #10 if circulating myeloma/plasma cells are identified in the Enrollment & Baseline sample.
⁷P-BCMA-101 cells will be assessed; e.g. number of vector copies/mL of blood of P-BCMA-101 cells, surface marker phenotype, clonality, etc. at Enrollment. The cellular characterization and phenotyping assessments conducted on the sample at each time point will vary depending on the temporal relevance of each assay.
⁸CRS will be graded using Lee criteria (Lee, 2014).
⁹PET/CT to be obtained at Baseline within 7 days of initiating conditioning chemotherapy.
¹⁰Allergies and all prescription and nonprescription medication, vitamins, herbal and nutritional supplements, taken by the subject during the 30 days prior to Screening will be recorded at the Screening Visit. All medications, supplements and allergies (including severity of any current symptoms) should be recorded at each visit. Unnecessary medications/supplements should be discontinued prior to Screening, if possible, at the investigator’s judgement.
¹¹Subjects should continue to meet entry criteria at the time of initiation of conditioning chemotherapy or have medical monitor approval. The following assessments should be repeated within 72 hours of Day −5 to re-assess entry criteria: MMSE (should include sample of handwriting by the subject); physical exam; vital signs; pregnancy; and chemistry panel, hematology including B and T cell counts, coagulation.
¹²Enrollment assessments are to be conducted 14 days (±3 days) prior to leukapheresis or have medical monitor approval.
¹³Leukapheresis should take place within~28 days following screening or have medical monitor approval. Guidance for conducting leukapheresis may be found in Section 5.1 and the Study Reference Manual (in particular characterization of the product and process including midpoint and end counts with CBC with manual diff, platelets, B and T cells (CD4 and 8), myeloma cells, flow cytometry, machine performance are performed at the investigative site apheresis center during apheresis and reported prior to shipment of the apheresis product from the apheresis center to other locations for analysis, characterization and manufacturing of P-BCMA-101 cells).
¹⁴Laboratory studies labeled as “sample for/of” or “blood sample for” are sent to a central laboratory, whereas those not so labeled are generally conducted at the investigational site. A subject enrolled prior to the approval of P-BCMA-101-001 A3 (amendment 3) will continue to have assessments for myeloma response; HIV, Hepatitis B and C, and HTLV Screening; Serum IgG; and Cytokine Release Syndrome markers (CRS) conducted at the investigational site.

TABLE 3
2. Schedule of Events-P-BCMA-101 Administration and Follow-Up (Single Administration)

| | | | | | | Post-Treatment Follow-up Visits (visit windows) | | | | |
|---|---|---|---|---|---|---|---|---|---|---|
| | P-BCMA-101 | | | | | relative to Day 0⁸ | | | | |
| | Administration⁸ | | | | | Week | | Week | | q3 months |
| | Days | | | | | 2 & 3 | Week 4 | 6 & 8 | Month 3 | to Month 24 |
| Procedures | 0⁷ | 1 | 4 | 7 | 10⁹ | (±2 days) | (±7 days) | (±7 days) | (±2 weeks) | (±2 weeks) |
| Mini Mental Status Exam (MMSE) | X | X | X | X | X | X | X | X | | X |
| 12-Lead ECG¹ | X | X | X | X | | | X | | X | X |
| Samples for Myeloma Response^{2,11} | X2 | | | | | X | X | X | Mo 3, 4, 5 | +Mo 6, 7, 8, 9 |
| Fresh Sample of Bone Marrow and Tumor^{2,11} | | | | | | | X | | X | Mo 6, 12 |
| Blood Sample for BCMA and Other Biomarkers¹¹ | X | | | | | X | X | X | X | X |
| Physical Exam (Including Neurological Exam) | X | X | X | X | X | X | X | X | X | X |
| Vital Signs (BP, HR, RR, O2 sat, temp) | X | X | X | X | X | X | X | X | X | X |
| Pregnancy Test (urine)³ | | | | | | X | X | X | X | X |
| Chemistry Panel, Hematology including | X | X | X | X | X | X | X | X | X | X |
| B and T cell counts, Coagulation⁴ | | | | | | | | | | |
| P-BCMA-101 Cell Infusion (IV) | X | | | | | | | | | |
| Blood Sample for P-BCMA-101 T cells^{5,11} | X5 | | X | X | X | X | X | X | Mo 3, 4, 5 | +Mo 6, 7, 8, 9 |
| Blood Sample for Serum IgG¹¹ | X | | | | | | X | X | X | X |
| Blood Sample for Immunogenicity Assays¹¹ | | | | | | X | X | | X | X |
| Blood Sample for Cytokine Release Syndrome | X | X | X | X | X | X | X | X | Mo 3, 4, 5 | X |
| markers (CRS)6 e.g. CRP, Ferritin, | | | | | | | | | | |
| Cytokines IL-6, IL-2, IL-15, TNF-α, | | | | | | | | | | |
| and IFN-γ^{6,11} | | | | | | | | | | |
| PET/CT¹² | | | | | | | | (X¹²) | (X¹²) | (X¹²) |
| Adverse Events | X | X | X | X | X | X | X | X | X | X |
| Concomitant Medication¹⁰ | X | X | X | X | X | X | X | X | X | X |

¹12-Lead ECG conducted at Days 0, 1, 4, 7, Week 4, Month 3 and every 3 months thereafter.
²Myeloma Response will be assessed at Days 0 (before P-BCMA-101 administration-thus the 24-hr urine collection is to be started on Day −1 so as to be completed before P-BCMA-101 administration), Weeks 2, 3, 4, 6, 8 and Months 3, 4, 5, 6, 7, 8, 9, 12, 15, 18, 21 and 24 and as clinically indicated (e.g. <1 week after an assessment demonstrating a response for confirmation) per the International Myeloma Working Group criteria and based on the standard assessments, including serum protein electrophoresis (SPEP), urine protein electrophoresis (UPEP), serum immunofixation (SIFE), urine immunofixation (UIFE), and serum free light chains (FLC). Bone marrow biopsy, aspirate and minimal residual disease (MRD) (analyzed by central labs) must be done at Week 4, Months 3, 6 and 12 then as clinically indicated, or have a medical monitor exemption. A sample of marrow aspirate and a sample of bone marrow biopsy are to be submitted (as described in the Study Reference Manual) for assessment of MRD, plasma cell frequency, P-BCMA-101 cells, BCMA, and other biomarkers. A portion of any extramedullary biopsy should also be submitted for assessment of P-BCMA-101 cells, BCMA, and other biomarkers. PET/CT is to be conducted as clinically indicated.
³Females of childbearing potential will have a urine pregnancy test at all indicated visits after Screening.
⁴Blood chemistries will include sodium, potassium, chloride, magnesium, bicarbonate, blood urea nitrogen, creatinine, calcium, albumin, glucose, LDH, total protein, ALT, AST, bilirubin (total and direct), alkaline phosphatase, PTT and PT or INR. Assessments for tumor lysis (uric acid and phosphate) will be performed at baseline and Days 1, 4, 7, then as clinically indicated.
Hematological studies include:
Complete blood count
Platelets
B (CD 19) and T cell counts (CD3) and CD4 and CD8
⁵P-BCMA-101 cells will be assessed; e.g. number of vector copies/mL of blood of P-BCMA-101 cells, surface marker phenotype, clonality, etc. At Baseline, Days 0 (1 hour after P-BCMA-101 administration), 4, 7, 10, Weeks, 2, 3, 4, 6, 8, Month 3, 4, 5, 6, 7, 8, 9 and every 3 months thereafter. The cellular characterization and phenotyping assessments conducted on the sample at each time point will vary depending on the temporal relevance of each assay.
⁶CRS will be graded using Lee criteria (Lee, 2014).
⁷On Day 0 MMSE (should include sample of handwriting by the subject), 12-Lead ECG, physical exam, chemistry panel, hematology and coagulation should be obtained before and approximately 1 hour after P-BCMA-101 administration (+/−15 minutes). Vital signs (temperature, respiration rate, pulse, O2 saturation and blood pressure) will be taken before and after P-BCMA-101 administration, then every 15 minutes (+/−5 minutes) for at least one hour and until these signs are satisfactory and stable. Other Day 0 assessments and samples should be obtained before P-BCMA-101 administration unless otherwise indicated. After administration of P-BCMA-101 these assessments (or any other assessments deemed clinically indicated by the Investigator) will be conducted as described in Table 3, and as often as clinically indicated by the adverse events observed or by the institution’s standards.
⁸If a subject discontinues from this study after P-BCMA-101 administration, the events for the next visit scheduled should be performed prior to initiating alternative medical, radiation or surgical intervention and recorded as an end-of-study visit for this study. If the subject is enrolling into Protocol P-BCMA-101-002, the subject should be consented and enrolled into that protocol following the subject discontinuing from Protocol P-BCMA-101-001 and the events for the first visit of Protocol P-BCMA-101-002 performed and recorded per the schedule prescribed by that protocol relative to the administration of P-BCMA-101 on this protocol.
⁹Day 10 assessments may be performed up to 2 days after Day 10 if needed, but not on the same day as week 2 assessments.
¹⁰All medications, supplements and allergies (including severity of any current symptoms) should be recorded at each visit.
¹¹Laboratory studies labeled as “sample for/of” or “blood sample for” are sent to a central laboratory, whereas those not so labeled are generally conducted at the investigational site. A subject enrolled prior to the approval of P-BCMA-101-001 A3 (amendment 3) will continue to have assessments for myeloma response; HIV, Hepatitis B and C, and HTLV Screening; Serum IgG; and Cytokine Release Syndrome markers (CRS) conducted at the investigational site.
¹²PET/CT to be obtained at Baseline within 7 days of initiating conditioning chemotherapy, then as clinically indicated (e.g. every 8 weeks if soft tissue plasmacytomas are found at baseline that require imaging as part of response assessments or to confirm response).

### Example 4—P-BCMA-101 T Cell Therapy in Multiple Myeloma

1. Introduction

Multiple myeloma (MM) is generally an incurable and fatal disease which typically runs a course of multiple relapses and recurrences. Currently available therapies are inadequate and there remains an unmet need for an effective and durable MM therapy. Chimeric antigen receptor T cell (CAR-T) immunotherapy is emerging as an important potential therapeutic approach for cancer, including MM. B-cell maturation antigen (BCMA) is an attractive target given that BCMA is expressed in MM cells, but among non-malignant cells, BCMA expression is largely restricted to plasma cells and a subset of B cells. Clinical data in MM patients has recently been published using two similar BCMA-targeted CAR-T cell products (NCI/NIH, University of Pennsylvania and BlueBird Bio studies) demonstrating the safety and efficacy of the approach (Ali, 2016; Cohen, 2016; Berdeja, 2016). In vitro and in vivo studies have shown that the P-BCMA-101 T cells bind to BCMA+ tumor lines with high affinity and specificity, resulting in robust degranulation and cytotoxicity. The unmet medical need, when taken together with the available preclinical and clinical data, and the potential safety and efficacy advantages of this construct, provide the rationale for evaluating P-BCMA-101 in patients with recurrent or relapsing MM. The information below is also described in detail in the Investigator's Brochure.

1.1 Multiple Myeloma

Multiple myeloma (MM) is a treatable but typically incurable plasma cell malignancy that often runs an aggressive and lethal clinical course. It is estimated that about 26,850 new cases of MM occurred in the U.S. in 2015 with approximately 11,240 deaths. The diagnosis is most common in the 6th and 7th decades of life (Howlader, 2015).

The hallmark feature of MM is monoclonal expansion of plasma cells in the bone marrow with accompanying excessive production of monoclonal antibodies (mAbs) that produce an “M spike” on serum protein electrophoresis (Raab, 2009). The clinical features of the disease result from bone marrow infiltration by the malignant clone, high levels of circulating monoclonal antibody (mAb) and/or free light chains, depressed immunity and end-organ damage. The classic signs and symptoms of MM include anemia, bleeding due to thrombocytopenia, frequent infections due to leukopenia and low antibody production, bone pain due to bone lesions and fractures and renal impairment due to high levels of M protein accumulation and hypercalcemia.

Recent advances in the understanding of MM's pathophysiology and the introduction of novel therapeutic agents have contributed toward the management of this disease with dramatic improvements in survival observed in the last 2 decades. Median survival increased from about 2 years in the 1980s up to 5-6 years or more today (Engelhardt, 2010). Treatment regimens tend to be comprised of two to three agents; most all patients receive a proteasome inhibitor (bortezomib or carfilzomib) and an immunomodulatory agent (IMiD) (lenalidomide, pomalidomide, thalidomide) both early and late in the course of treatment of their disease. In addition, eligible patients may undergo autologous hematopoietic stem cell transplantation and/or, less frequently, allogeneic hematopoietic stem cell transplantation. In 2015, several new therapies were approved in the United States including 2 mAbs (daratumumab and elotuzumab), a pan histone deacetylase (HDAC) inhibitor (panabinostat), and an oral proteasome inhibitor (ixazomib). The long-term impact of these recent approvals remains to be determined, but they do not appear to produce cures in the relapsed and/or refractory setting, and most patients will ultimately relapse and die (Kumar, 2008). Tumors that relapse tend to recur more aggressively with each relapse.

Responses to treatment are less durable with subsequent disease progression resulting in treatment-refractory disease associated with shortened survival times (Kumar, 2012).

1.2 Rationale for P-BCMA-101 T Cell Therapy in Multiple Myeloma

1.2.1. P-BCMA-101 T Cell Therapy

P-BCMA-101 is an autologous Centyrin-based CAR-T cell therapy, referred to as CARTyrin T cells. It is a biologic product designed to target MM cells expressing the cell surface antigen BCMA and to direct cytotoxic effects to the targeted cell (Tai, 2015). The mechanism of action of P-BCMA-101 is the same as that of other CAR-T approaches (e.g. Ali, 2016; Berdeja, 2016). Each patient's peripheral blood mononuclear cells (PBMCs) will be harvested by leukapheresis, then will be used to generate the PBCMA-101 investigational product for that individual patient via electroporation of transposase ribonucleic acid (RNA) along with a DNA plasmid encoding a PB transposon carrying the CARTyrin (i.e. the piggyBac™ (PB) DNA modification system), followed by culture/expansion.

P-BCMA-101 cells are designed to express 3 major components: an anti-BCMA Centyrin chimeric antigen receptor (CARTyrin) gene, a dihydrofolate reductase (DHFR) resistance gene, and an inducible caspase 9 (iC9)-based safety switch gene (Hermanson, 2016).

The CARTyrin expression cassette encodes for an extracellular BCMA-binding Centyrin protein fused to a CD8a signal/leader peptide, CD8a hinge/spacer, a CD8a transmembrane domain, an intracellular 4-1BB signaling domain and a T-cell receptor (TCR) (chain signaling domain. As compared with binding domains consisting of an antibody-based single chain variable region (scFv), the CARTyrin binding domain is a fully human protein that is smaller, more stable and potentially less immunogenic, but with similar antigen binding characteristics that enable recognition and killing of BCMA-expressing MM cells. This construct is also designed to avoid T cell exhaustion.

The DHFR resistance gene is used during manufacturing of the T cells for ex vivo selection of CARTyrin expressing T cells to produce a more homogenous product with the intent to enhance potency and safety.

The safety switch is an extra safety feature not found in most CAR-T cells designed to allow for rapid ablation of P-BCMA-101 cells by intravenous administration of rimiducid, a synthetic dimerizer drug (see Section 15.3).

As of 14 Jul. 2019, 36 subjects had been treated with P-BCMA-101 cells (34 in Phase 1 and 2 in Phase 2), 3 of whom have received a second administration of P-BCMA-101. All Phase 1 dose escalation cohorts (1-5: 0.75-15×10⁶ P-BCMA-101 cells/kg/dose) had successfully been completed with good safety and efficacy reported through the highest dose level. No DLTs had been reported and enrollment of new subjects is continuing in expansions of cohorts, suggesting additional cohorts should be assessed. Patients were heavily pre-treated (3-18 prior therapies), most had failed IMiDs, proteasome inhibitors, daratumumab and ASCT. Patients were treated with 48-1545×10⁶ total P-BCMA-101 cells/kg. Circulating P-BCMA-101 cells have been detected in the blood of patients by PCR, with expansion peaking at 2-3 weeks. Above a threshold level reached in Cohorts 2-3, greater response appears to occur in patients with broader peaks, suggesting there may be additional benefit in repeat or fractionated dosing. Although of unknown significance, indications of anti-CAR-T antibodies have been seen in some patients. The most common TEAEs (>30%) were neutropenia, WBC decreased, thrombocytopenia, anemia, nausea, constipation and febrile neutropenia. Only 4 subjects had cases of CRS (1 subject had Grade 2 at 2×10⁶ cells/kg and 3 subjects had Grade 2 at 15×10⁶ cells/kg) and 1 case of CRES (Grade 2 at 6×10⁶ cells/kg, transient confusion) had been reported. Repeat administrations of P-BCMA-101 were also well tolerated. Twenty-nine of the Phase 1 subjects were evaluable for response by IMWG criteria and had completed at least one myeloma response assessment, with 17 thus far demonstrating a response (1/2 at ˜0.75×10⁶ cells/kg, 5/7 at ˜2×10⁶ cells/kg, 4/9 (+2 MR) at ˜6×10⁶ cells/kg, 3/4 (+1 MR) at ˜10×10⁶ cells/kg, and 4/7 at ˜15×10⁶ cells/kg). Based on these results the study was further expanded with a Phase 2 portion to further characterize the safety and efficacy at dose levels 3-5.

1.2.2. BCMA as a Therapeutic Target for CAR-T Cells

Myeloma has several characteristics that make it suitable to treat with adoptive T cell therapy. First, myeloma is primarily a disease of the bone marrow and adoptive T cell therapy targeting CD19 has been particularly successful in bone marrow-predominant diseases, such as acute lymphoblastic leukemia (ALL) (Brentjens, 2013). Second, autologous stem cell transplantation is the standard of care in myeloma, and lymphodepletion may enhance the efficacy of adoptive T cell therapy (Brentjens, 2011; Pegram, 2012). Third, unlike all other treatments for myeloma, an allo-SCT is potentially curative, however, its application is limited by transplant-associated toxicity, mortality, patient eligibility and availability of suitable donors. CAR-T cell therapy has the potential to be a safer way to achieve such anti-tumor efficacy (Milone, 2015).

The utility of CD19 as a target is limited by the fact that it is infrequently expressed on the malignant plasma cells of MM; therefore, other antigens have been explored, with particular attention to antigens expressed by tumor cells but not normal tissues. One attractive target is BCMA. The rationale for selecting this target is that BCMA is detected in MM cells, but among non-malignant cells, BCMA expression is largely restricted to plasma cells and a subset of B cells, and therefore may have less potential for on-target, off-tumor effects.

MM tumor cell recognition occurs when the BCMA-specific CAR expressed on the surface of a P-BCMA-101 T cell binds to BCMA antigen expressed on the surface of a MM tumor cell. Signaling and activation is mediated by the intracytoplasmic signaling domains 4-1BB and CD3˜ encoded within the CAR. Activation can lead to direct cytotoxicity of MM tumor targeted by CAR-T cell-mediated release of granzyme and perforin. Tumor killing can also be mediated by activation of other components of the immune system through release of cytokines by CD4+ T cells. Long-term eradication and prevention against tumor relapse may be provided either by immediate tumor ablation or by long-term memory CAR-T cells that remain after the initial tumor response. Thus, there may be particular advantage to having CAR-T cells that are of the T stem cell memory phenotype (Tscm) and T central memory phenotype (Tcm). In addition, release of non-BMCA tumor-associated antigens during CAR-T-mediated tumor cell lysis may lead to the priming and/or reactivation of non-engineered tumor-specific cells of the adaptive immune system, a phenomenon termed ‘epitope-spreading’, that may also assist in the long-term eradication and prevention against tumor relapse.

In a nonclinical study conducted by Carpenter, et al. (Carpenter, 2013) anti-BCMA-CAR T cells exhibited specific anti-BCMA functions, including cytokine production, proliferation, cytotoxicity, and in vivo tumor eradication. Importantly, anti-BCMA-CAR T cells recognized and killed primary MM cells. The authors concluded that BCMA is a suitable target for CAR-expressing T cells, and adoptive transfer of anti-BCMA-CAR T cells is a promising new strategy for treating MM (Carpenter, 2013).

Data was published that demonstrated the first clinical proof of concept for T cells expressing anti-BCMA CAR in subjects with relapsed/refractory MM from a study performed at the National Cancer Institute (NCI). Patients (N=16) were enrolled at 4 dose levels, including: 0.3×10⁶; 1×10⁶; 3×10⁶ and 9×10⁶ cells/kg-body wt. Responses were dose-dependent and included partial response (N=3), very good partial response (N=4) and stringent complete response (N=1). At the highest dose level, the response rate was 100%. Data on duration of response is not yet available. Tolerability was consistent with expectations from other CAR-T studies. Toxicities attributable to anti-BCMA CAR-T cells were minimal in patients treated at the low dose level. Subsequently, patients exhibited signs of CRS correlating with dose level. No unexpected damage to non-hematopoietic organs was observed (Ali, 2016; Kochenderfer, 2016). Initial results of another study utilizing anti-BCMA CAR-T cells by Cohen et al. (Cohen, 2016) have recently been presented. In this study, 6 patients were treated with 1-5×10⁸ CAR-T cells. Four patients responded: minimal response (N=2), very good partial response (N=1) and minimal residual disease negative (MRD negative) stringent complete response (N=1). Five patients developed CRS, of which 1 was Grade 3 with neurotoxicity that responded to treatment without sequelae. CAR-T cell expansion appeared to correlate with efficacy. In an ongoing study of a third anti-BCMA CAR-T construct presented by Berdeja et al., 11 patients were enrolled at 3 fixed dose levels; 5×10⁷; 15×10⁷ and 45×10⁷ CAR-T+ cells per patient (80×10⁷ and 120×10⁷ CAR-T+ cells per patient are planned for upcoming cohorts). The response rate was impressive and dose-dependent, including partial response (N=4), very good partial response (N=1) and stringent complete response (N=2), with 2 patients becoming MRD negative. Tolerability was better than what has been reported in other CAR-T studies. CRS was seen in 70-80% of the patients, but it was limited to Grade 1-2. No neurotoxicity or other significant or unexpected toxicities were reported (Berdeja, 2016). The relative paucity of toxicity in these studies has been variously attributed to use of the 4-1BB costimulatory domain, lessened disease burden and/or more gradual exposure of the T-cells to myeloma cells, compared to the anti-CD19 products used in leukemias which have generated the most publications.

Lenalidomide (Lenalidomide, 2019) is a member of the class of immunomodulatory imide drugs (IMiDs) known for significant activity against myeloma as well as pleiotropic effects on the immune system, and is approved for the treatment of myeloma, myelodysplastic syndrome and lymphomas in the United States (Moreau, 2019; Fink, 2015). In addition to its direct anti-myeloma properties, it is hypothesized lenalidomide could enhance the efficacy of CAR-T cells such as P-BCMA-101. Lenalidomide treatment increases the frequency of naïve and stem cell memory T cells, and not only are these two T subsets the preferred starting material for the P-BCMA-101 manufacturing process, but they have also been associated with improved clinical outcome in CAR-T cell products (Fostier, 2018; Barnett, 2016a; Cohen, 2019). Inclusion of lenalidomide directly in the manufacturing process is not being proposed as this approach may increase the number and effector function of CAR-T cells produced (Wang, 2018). Thus, including lenalidomide treatment prior to apheresis is hypothesized to improve the quality of the T cells in the input material and, subsequently, the manufactured CAR-T cell product. Co-administration of lenalidomide with CAR-T cells is being proposed as it has been shown to augment CAR-T cell effector functions and overall anti-myeloma activity both in vitro and in mouse models (Otihal, 2016; Wang, 2018; Works, 2019). Moreover, a similar combination with lenalidomide and an anti-BCMA CAR-T cell product is currently being explored in at least one other clinical trial (NCT03070327).

Rituximab (Rituximab, 2019) is an anti-CD20 antibody therapeutic that depletes CD20+ cells, thus B cells, has an excellent safety and efficacy profile and is approved for the treatment of a number of lymphomas, leukemias and autoimmune diseases in the United States (Salles, 2017). Therapeutic synergy is expected between P-BCMA-101 and rituximab for several reasons. CD20 expression is well-documented in a subset of multiple myeloma, possibly including multiple myeloma stem cells (Flores-Montero, 2016; Matsui, 2018; Kapoor, 2008). Although there is some controversy as to whether multiple myeloma stem cells uniformly express CD20, there is consensus that myeloma differentiation parallels normal B cell development, in which BCMA-CD20+ cells ultimately mature into BCMA+CD20− cells (Johnsen, 2016; Bodker, 2018). A combination strategy to target BCMA+ cells with CAR-T cells and CD20+ cells with rituximab could eradicate the mature neoplasm as well as any pre-malignant cells that may give rise to relapse. Moreover, lymphopenia is known to contribute to the efficacy of CAR-T cell therapies and rituximab has been found to prolong this condition (Cohen, 2019; Yutaka, 2015). Finally, anti-CAR antibodies have been seen in patients with multiple myeloma treated with CAR-T cells, potentially limiting product durability and suggesting that the clinical responses might be improved with depletion of the endogenous B cell compartment (Xu, 2019).

1.2.3. P-BCMA-101 Design and Rationale

As described above, P-BCMA-101 cells are designed to express 3 major components: an anti-BCMA Centyrin chimeric antigen receptor (CARTyrin) gene, a dihydrofolate reductase (DHFR) resistance gene, and an inducible caspase 9 (iC9)-based safety switch gene (Hermanson, 2016). Binding of BCMA antigen expressed on the surface of a MM tumor cell by the BCMA-specific CARTyrin triggers signaling and activation within the P-BCMA-101 cells that is mediated by the CARTyrin-encoded intracytoplasmic signaling domains. Beyond the unique anti-BCMA moiety encoded by the CARTyrin, the main differences between the P-BCMA-101 approach and most other CAR-T cells is the manufacturing process.

Whereas genetic modification of autologous T cells for expression of CAR molecules is generally accomplished via lentivirus or γ-retrovirus transduction, P-BCMA-101 is manufactured using an electroporation-based non-viral (DNA transposon) gene delivery system called the piggyBac (PB) DNA modification system (Nakazawa, 2013) which efficiently moves DNA from a plasmid to a chromosome via a “cut and paste” mechanism and have been used extensively as a human gene transfer method, including CAR-T production (Woodard, 2015; Fraser, 1996; Singh, 2013; Huls, 2013). Compared to viral-based delivery, PB offers advantages, including a safer insertion profile (Cunningham, 2015), larger transgene capacity (enabling the delivery of genetic components to enhance safety and efficacy), higher level and more stable transgene expression (Cunningham, 2015), longer duration transgene expression (Mossine, 2013) as well as a preponderance of the highly favorable TSCM phenotype. In the case of P-BCMA-101, a Super piggyBac transposase (SPB) is used, which is an engineered, hyperactive enzyme that catalyzes the integration of PB transposons into TTAA sites in the target genome, with a more defined and safer integration profile. While the genetic cargo capacity of lentivirus and γ-retrovirus is limited to ˜10-20 Kb, the piggyBac DNA modification system has a demonstrated cargo limit>300 Kb, allowing transfer of multiple beneficial genes. Moreover, as compared with lentivirus, PB-mediated introduction allows for high-level durable expression of the transgenes (Cunningham, 2015). Finally, manufacture of CAR-T cells using this method increases the percentage of cells with a stem-cell memory phenotype (Tscm), which may increase safety and efficacy. Efficacy has been demonstrated in unprecedented response durability and re-control of relapsing tumors in murine MM models (including a MM.1S p53 knockout (KO) model designed recapitulate patients with aggressive myeloma and adverse prognostic indicators, such as del17p and other TP53 abnormalities). It is hypothesized that by expanding and differentiating gradually, Tscm cells may decrease the probability of acute adverse effects. It was observed that >60% of P-BCMA-101 T cells on average exhibited a Tscm phenotype as characterized by positive expression of CD45RA, CD62L, and CD197 (CCR7). In addition, P-BCMA-101 is largely (<10% on average) negative for expression of inhibitory receptors CD279 (PD-1), Tim-3, and Lag-3 (Barnett, 2016a). Thus, in addition to advantages with respect to safety, CAR-T cell manufacturing using PB (i.e. P-BCMA-101) may offer efficacy advantages.

1.2.4. Summary of Nonclinical Studies with P-BCMA-101

1.2.4.1. Product Candidate Screening and Selection

The anti-BCMA-binding CARTyrin in P-BCMA-101 was selected from a panel of different CARTyrins. Selection was based upon their comparative performance in both in vitro and in vivo preclinical studies (Barnett, 2016b). The panel of different CARTyrins was constructed using different Centyrins that were previously identified to specifically recognize BCMA protein, to bind with variable affinities, and to demonstrate a monomeric nature. These different CARTyrin candidates were evaluated for functional activity against BCMA+ tumor lines in in vitro studies that measured their ability to degranulate, which is a surrogate marker for cytotoxic killing capacity, as well as their ability to directly lyse target tumor cells. These studies were performed to identify the lead BCMA CARTyrin molecule used in this protocol.

1.2.4.1.1. In Vitro Screening

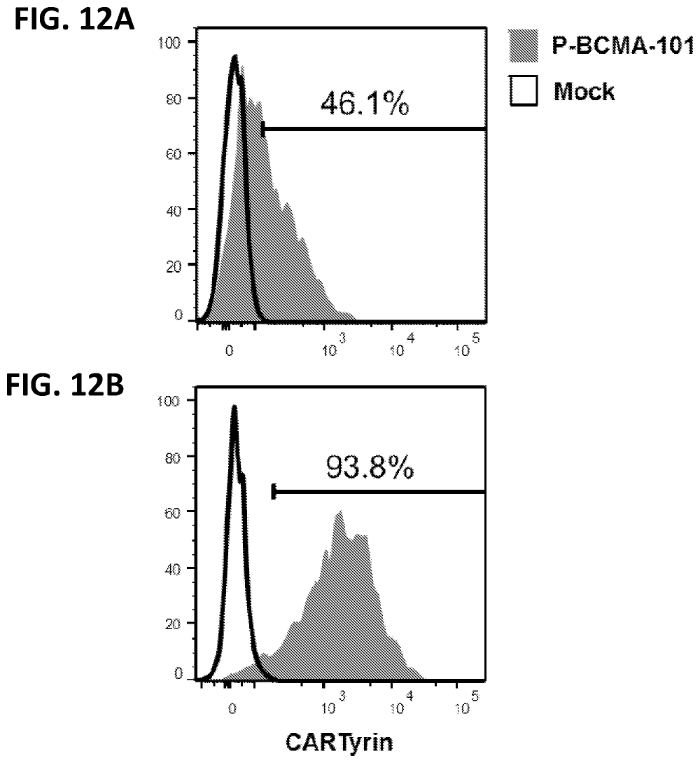

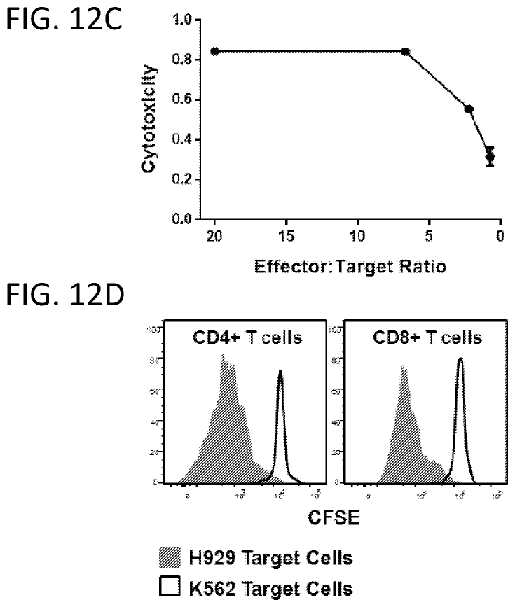

Upon selection of the candidate CARTyrin and to support in vivo evaluation, P-BCMA-101 cells were produced using PB transposition and phenotypic and functional characterization were performed. The CARTyrin was detected on the surface of P-BCMA-101 T cells (FIG. 12A) and was dramatically increased upon re-stimulation (FIG. 12B). To test the killing function of these T cells in vitro, P-BMCA-101 cells were co-cultured with BCMA-expressing cells and then target cell killing was measured (FIG. 12C). P-BCMA-101 cells exhibited strong cytotoxic function against the BCMA+ myeloma cell line H929. Next, the capacity for P-BCMA-101 cell proliferation upon co-culture with a BCMA+ cell line was assessed after 4 days. Both CD4+ and CD8+ T cells exhibited a robust capacity for proliferation; the proliferation index for CD4+ T cells was 3.0±0.09 and 3.4±0.03 for CD8+ T cells (FIG. 12C). These data show that PB-transposed P-BCMA-101 cells expressed detectable levels of the CARTyrin on the cell surface, specifically killed BCMA+ target cells, and proliferated when exposed to BCMA+ cell targets (Hermanson, 2016).

1.2.4.1.2. In Vivo Screening in MM Xenograft Models

In vivo assays were performed in MM xenografts using a luciferase-expressing MM.1S p53 WT and knockout (TP53KO) MM cell lines injected intravenously (IV) into NSG mice to determine the best CARTyrin+ T cell product candidates in terms of in vivo anti-tumor efficacy (MD Anderson Cancer Center).

In the MM.1S p53 WT experiment, two doses of the P-BCMA-101 cells were assessed: 0.5×10⁶ and 5.0×10⁶. Control animals received no T cells. All mice exhibited significant tumor burden by day 21, and P-BCMA-101 cells were injected on day 22. Control mice not receiving any T cells demonstrated progressive tumor growth and eventually succumbed at approximately 55 to 60 days after MM.1S tumor injection. In contrast, dose-dependent antitumor activity was observed in mice injected with the P-BCMA-101 cells. A dose of 5×10⁶ P-BCMA-101 cells/mouse showed the maximal antitumor effect, with mice showing rapid clearance of tumors as early as 3-7 days after T cell injection. Consistent with the above results observed by bioluminescent imaging, maximal improvement in survival was also observed in the mice treated with P-BCMA-101 cells with two mice treated with 5×10⁶ P-BCMA-101 cells surviving beyond 100 days. M-protein levels in the serum of the mice were also consistent with the imaging and survival data. Indeed, the M-protein level was undetectable in two of these mice even beyond 100 days. Although tumor recurred in the mice at or after day 59, tumor re-subsequently responded without additional P-BCMA-101 administration in two of the three mice treated at the highest dose level as determined by both imaging, as well as serum M-protein levels. Together, these results demonstrate that P-BCMA-101 cells exhibit strong dose-dependent efficacy in a human MM.1S myeloma cell line xenograft model. The data further indicate that P-BCMA-101 cells appear to persist in the mouse model and result in complete regression of tumor recurrences without administration of additional P-BCMA-101. This unique observation and the overall durability of responses to study termination (92 days) has been attributed to the favorable Tscm phenotype of P-BCMA-101 T cells (Hermanson, 2016).

Similar results were observed in an MM.1S TP53KO model (MD Anderson Cancer Center), which is designed to be p53 null to recapitulate patients with aggressive myeloma and adverse prognostic indicators, such as del17p and other TP53 mutations. All mice in the untreated group died within 50 days, whereas 100% of the mice survived in the P-BCMA-101-treated group until the end of the experiment at 90 days. This is a unique finding as numerous existing multiple myeloma therapeutics have been tested in this model and all failed to control this aggressive tumor line (personal communication R. Orlowski).

1.2.4.2. P-BCMA-101 Preclinical Toxicology

1.2.4.2.1 In Vitro Binding of the P-BCMA-101 Centyrin to a Human Protein Panel

In vitro human protein screening technology was used to identify potential secondary target binding of the Centyrin from P-BCMA-101, in the form of a Centyrin-Fc fusion protein to facilitate the assay. The Centyrin-Fc fusion protein was screened for binding against 4300+ human proteins. The vast majority of these human proteins represent cell surface membrane proteins, each individually expressed in human HEK293 cells. This study demonstrated the high specificity of Centyrin binding to its intended target, BCMA.

1.2.4.2.2. P-BCMA-101 Reactivity Against Normal Human Tissues

The potential reactivity of P-BCMA-101 cells against a panel of normal human tissue cell lines was examined in order to assess whether P-BCMA-101 CAR-T cells might exhibit any direct toxicity against normal, healthy human tissues. P-BCMA-101 T-cells were co-cultured with normal human cell types or BCMA+MM1.S positive control cells and potential T-cell reactivity against the normal human cell types was evaluated using luminescent or flow cytometric-based readout. Nine cell lines were tested and no reactivity was observed other than to the BCMA+MM1.S positive control cells.

1.2.4.2.3. Single-Dose GLP Safety and Efficacy Study of P-BCMA-101 CAR-T Cells in MM1.S Myeloma Tumor-Bearing NSG Mice

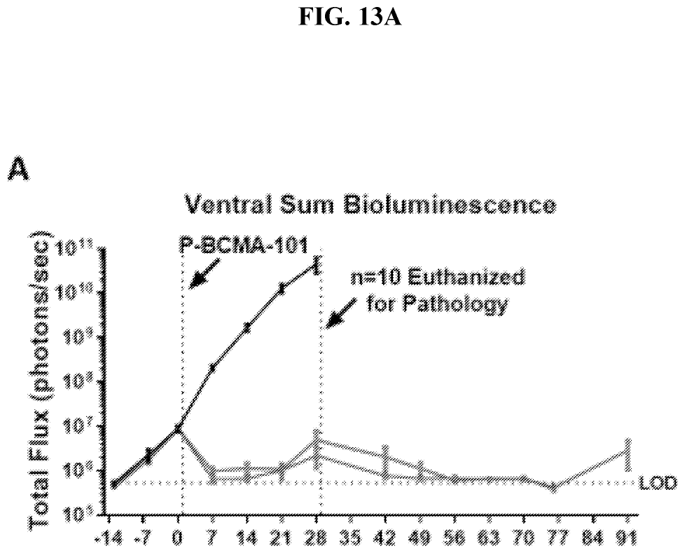

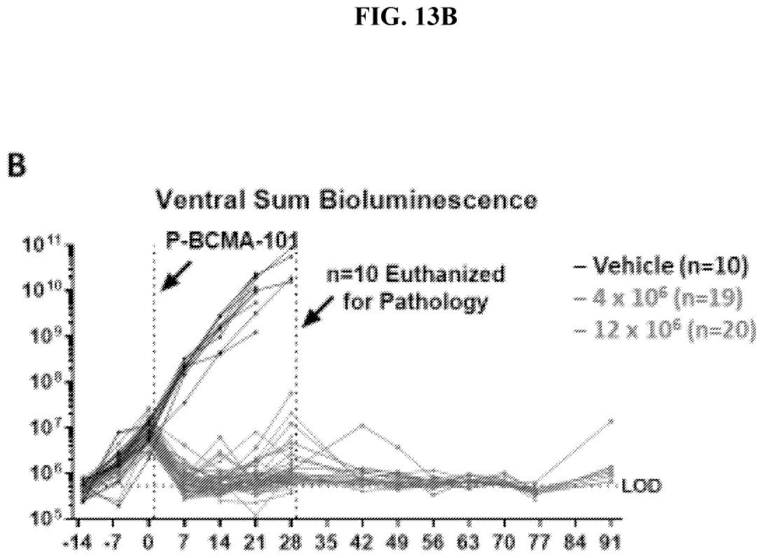

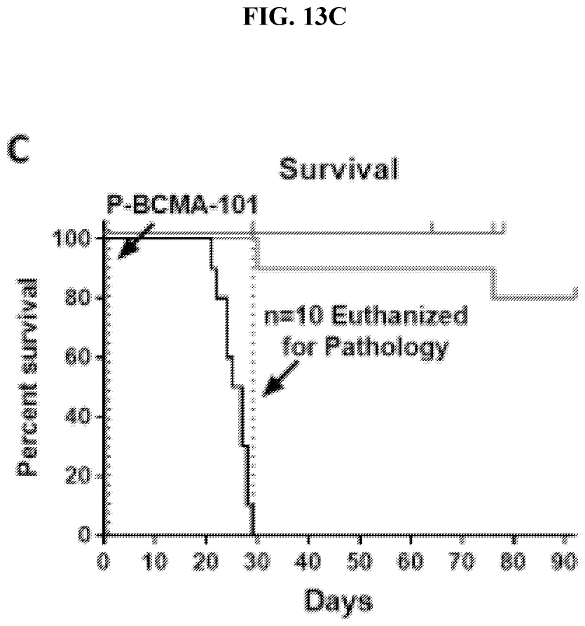

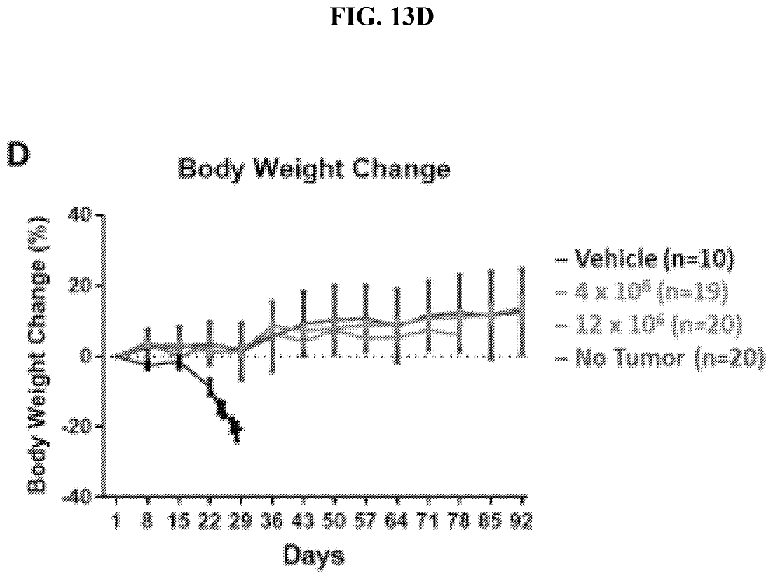

P-BCMA-101 has been evaluated for safety and efficacy in the context of a GLP compliant hybrid pharmacology-toxicology study in MM tumor-bearing female NSG mice performed at SRI International (FIG. 13A-D). The objectives of this study were to assess the anti-tumor efficacy and safety of P-BCMA-101 cells in mice bearing human MM.1S-Luc myeloma cells after a single intravenous (IV) dose and monitored for up to 3 months post-P-BCMA-101 administration. Female NSG mice were grafted IV with MM.1S BCMA+MM cells and dosed IV 17-19 days later with P-BCMA-101 cells at either a low dose (4×10⁶ cells) or high dose (1.2×10⁷ cells), or were left untreated and were monitored for tumor burden and toxicity for up to 90 days. Mice not receiving any P-BCMA-101 cells developed significant tumor burden and were all sacrificed on or prior to Day 29 as anticipated, whereas mice treated with P-BCMA-101 saw notable regression or elimination of their tumor burden as determined by BLI and M-protein analyses. While tumor almost completely disappeared in P-BCMA-101 treated groups, mice in Group 4 (high-dose P-BCMA-101) exhibited clinical findings consistent with xenogeneic GVHD, including hunched posture and ruffled fur, squinting eyes, alopecia, and discolored, thickened skin/fur. These remaining Group 4 mice survived until they were sacrificed on or prior to day 78. Unlike control (tumor-only) mice, P-BCMA-101-treated mice did not lose body weight over the course of the experiment. NSG mice are known to lack T, B and NK cells and exhibit reduced function of dendritic cells and macrophages. Commonly, transplant of human lymphocytes into this strain results in a GVHD-like syndrome, resulting in death. Overall, there were no target-organ related toxicities observed in this study that could be attributed to an effect of the P-BCMA-101 cells on any organs of these mice. There was no tumor formation, nor were there any notable gross or histopathological findings at terminal necropsy. Findings were consistent with either the severely compromised immune state of these animals or were related to xenogeneic GVHD due to administration of human T cells to the mice. As expected, the P-BCMA-101 cells persisted in the mice at days 29 and 92 post-dose, and appeared to exhibit a highly undifferentiated memory cell phenotype, which might also explain the exceptional and unprecedented anti-tumor efficacy observed in this study. Overall, there were no other overt toxicities or clear target organs identified in this study that could be attributable to IV dosing with PBMCA-101. Since xenogeneic GVHD is a common occurrence in NSG mice treated with human cells, the findings do not reflect true target organ toxicity, but rather an artificial graft vs. host response to treatment of mice with human cells. Thus, as no intolerable dose was found, the highest dose tested (1.2×10⁷ cells per mouse) was considered to be the maximum tolerated dose. A NOAEL could not be defined for P-BCMA-101 cells due to the appearance of GVHD, but there was no clear target organ toxicity. The proposed starting dose for the FIH Phase 1 study with P-BCMA-101 is set at 0.75×10⁶ cells/kg. For a small 60-kg MM patient, this is more than 800-fold lower than the tolerable and efficacious high-dose level of 1.2×10⁷ cells (6×10⁸ cells/kg) in this study.

1.3. Potential Risks and Benefits

1.3.1. Benefit Assessment

Based on data with P-BCMA-101 and other CAR-T cells (including anti-BCMA CAR-T cells) it is reasonable to expect that P-BCMA-101 may exert an anti-tumor effect. This is strongly supported by the data in Section 1.2. As described above, P-BCMA-101 bears a number of features intended to improve efficacy, such as the high Tscm phenotype proportion and the Centyrin binding domain, compared to previous anti-BCMA CAR-T products.

1.3.2. Risk Assessment

Participation in this study will expose the subject to genetically engineered autologous T cells. The risk is acceptable based on clinical experience with similar products. The potential safety concerns for P-BCMA-101 are effectively the same as for other CAR-T cell products. The primary adverse effect seen in CAR-T cell studies is CRS and associated symptoms related to the activation of the CAR-T cells through their intended mechanism of action (significant increases in cytokines have also been reported to correlate with efficacy). Descriptions and guidelines for management of these toxicities are provided in Section 6.3. Although not yet reported with BCMA-targeted CAR-T cells, other theoretical concerns with CAR-T cells include: 1) “on-target/on-tumor” toxicity, manifest as tumor lysis syndrome (TLS) through rapid destruction of myeloma tumor cells; 2) “on-target/off-tumor” toxicity related to destruction of BCMA+ non-tumor cells in healthy tissues (other than hypogammaglobulinemia); 3) “off-target” toxicity due to potential cross-reactivity of the BCMA-binding domain with BCMA-negative targets; and; 4) immune-related reactions secondary to the development of anti-CAR-T antibodies. Many of these questions are addressed in the data in Section 1.2. The potential for tumorigenicity is another hypothetical risk that is considered negligible (Tey, 2014; Hackett, 2013), based upon the terminal differentiation of these cells, insertion profile and character of the gene addition materials and process, as well as the extensive clinical experience to date with long-term follow up of patients receiving other CAR-T cell products (Jena, 2010). At least one previous CAR-T study used a similar DNA transposon approach, and was found to be well-tolerated in a Phase 1 clinical trial at doses up 1.2×10⁹ total CAR-T cells (Singh, 2013; Huls, 2013). Potential risks of the moderate conditioning chemotherapy regimen utilized in this protocol are expected to be typical for these agents, in particular cytopenias, gastrointestinal effects, and infertility. Hemorrhagic cystitis, pulmonary, cardiac and neurologic effects have also been reported.

The proposed starting dose (0.75×10⁶ CAR-T cells/kg) in this Phase 1 study is ˜3-fold lower than that reported in one clinical trial of CAR-T cells targeting BCMA (Cohen 2016) and approximately the same as others (Ali, 2016; Berdeja, 2016) who described toxicity as mild through doses up to 10-fold higher. It is conservatively 800-fold lower than the tolerable and efficacious high dose level in the mouse GLP toxicology study, further supporting a large margin of safety for the proposed FIH trial. Moreover, the only notable toxicity observed in the high-dose P-BCMA-101 treated group of the mouse GLP toxicology study was GVHD, a toxicity not expected to be relevant to an autologous human product in patients.

As described above, P-BCMA-101 was designed with a number of features to increase safety, such as such as the high Tscm proportion, the small, poorly immunogenic Centyrin-binding domain and the DIFR selection gene.

This study incorporates several further measures to address potential risks, including the following: step-wise escalation of the T cell dose, staggered enrollment of subjects, treatment in specialized academic centers experienced with management of toxicities associated with autologous T cell therapies, guidelines for management of toxicities, and a Safety Review Committee to evaluate safety throughout the study. As of 14 Jul. 2019, the most common TEAEs (>30%) in Phase 1 were neutropenia, WBC decreased, thrombocytopenia, anemia, nausea, constipation, and febrile neutropenia. Only 4 subjects had cases of CRS (1 subject had Grade 2 at 2×10⁶ cells/kg and 3 subjects had Grade 2 at 15×10⁶ cells/kg) and 1 case of CRES (Grade 2 at 6×10⁶ cells/kg) had been reported.

1.3.3. Overall Benefit: Risk Conclusion

The known and potential risks anticipated with P-BCMA-101 T cell therapy appear justified by the potential benefits that may be afforded to subjects with relapsed/refractory MM, a fatal malignancy.

2. Study Objective and Endpoints

Primary Objectives: The primary objective of this study is:

Phase 1—To determine the safety and maximum tolerated dose (MTD) of P-BCMA-101 based on dose limiting toxicities (DLT)
Phase 2—To assess the safety and efficacy of P-BCMA-101

Primary Endpoints:

Phase 1—Number of subjects with DLT at each dose level to define an MTD
Phase 2—Safety and tolerability based on adverse events (AEs), examinations, and standard laboratory studies; Overall response rate (ORR) and duration of response (DOR) by International Myeloma Working Group Criteria (Kumar, 2016) as assessed by an independent review committee (IRC)

Secondary Objectives: The secondary objectives of this study are to evaluate:

Phase 1—the safety and feasibility of P-BCMA-101; the anti-myeloma effect of P-BCMA-101; the effect of cell dose to guide selection of doses for further assessment in Phase 2/3 studies
Phase 2—Incidence and severity of cytokine release syndrome (CRS); additional efficacy endpoints

Secondary Endpoints: The following secondary endpoints will be evaluated:

Phase 1—Ability to generate protocol-proscribed doses of P-BCMA-101; Safety and tolerability based on AEs, examinations, and standard laboratory studies; CRS graded using Lee criteria (Lee, 2014); Efficacy based on International Myeloma Working Group (IMWG) Uniform Response Criteria (Rajkumar, 2011; Kumar 2016; Cavo, 2017); Overall response rate (ORR); Time to response (TTR); Duration of response (DOR); Progression free survival (PFS); and Overall survival (OS).

Phase 2—CRS graded using Lee criteria (Lee, 2014); Rate of IL-6 antagonist, corticosteroid, and rimiducid use; OS, PFS, TTR, minimal residual disease (MRD) negative rate

Exploratory Objectives: Exploratory objective of this study are to:

Phase 1—Evaluate the relationship between MM plasma cell BCMA expression, circulating soluble BCMA and clinical response

Phase 1 and 2—Characterize the expansion and functional persistence of the P-BCMA-101 cells; Evaluate the relationship between putative CRS markers and efficacy or safety; Evaluate the effect of rimiducid on P-BCMA-101-related adverse events, if indicated.

Exploratory Endpoints: The following exploratory endpoints will be assessed during the course of the study:

Phase 1—BCMA and/or other biomarkers in bone marrow; Soluble BCMA and/or other biomarker levels in blood

Phase 1 and 2—P-BCMA-101 cells (e.g. number of vector copies/mL in blood and bone marrow of P-BCMA-101 cells); P-BCMA-101 cell subset composition and clonality; CRS markers: C-reactive protein (CRP), ferritin, IL-6, IL-2, TNF-α, and interferon gamma (IFN-γ)

3. Investigational Plan

3.1. Overall Study Design

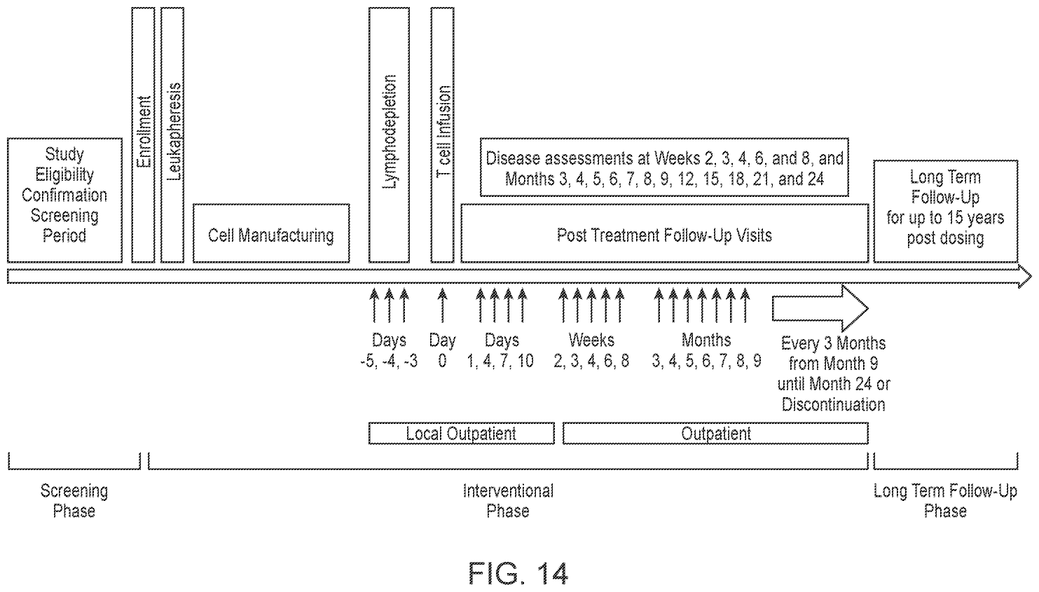

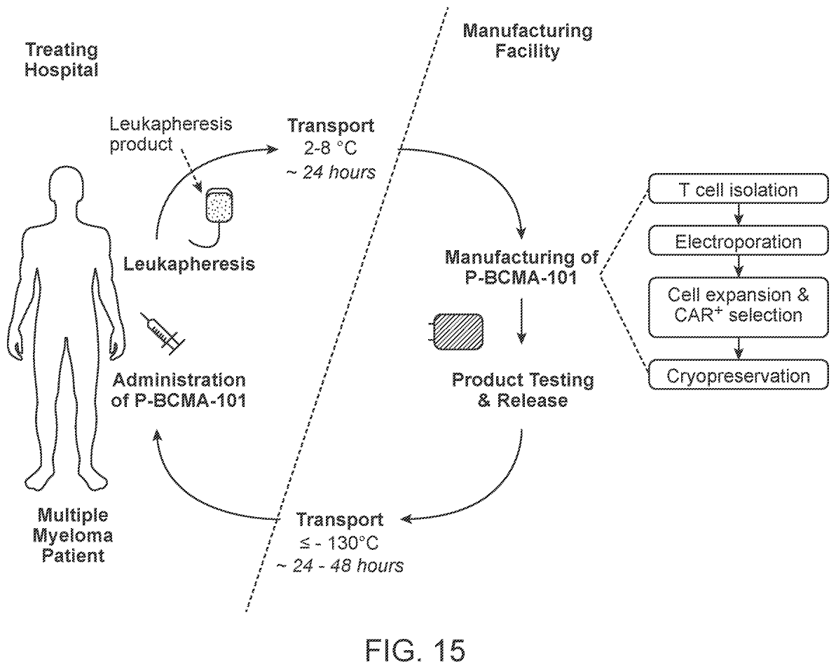

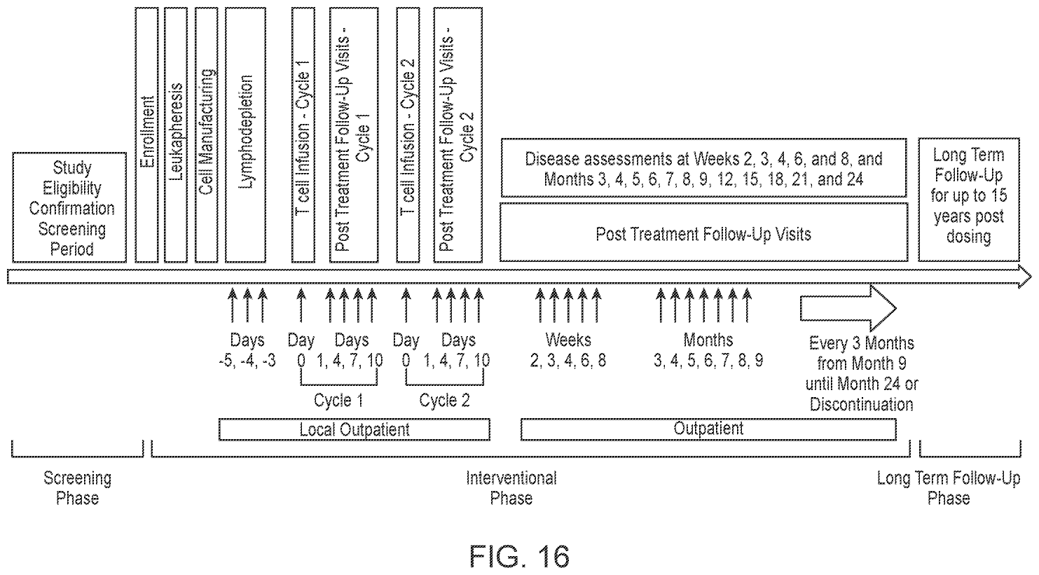

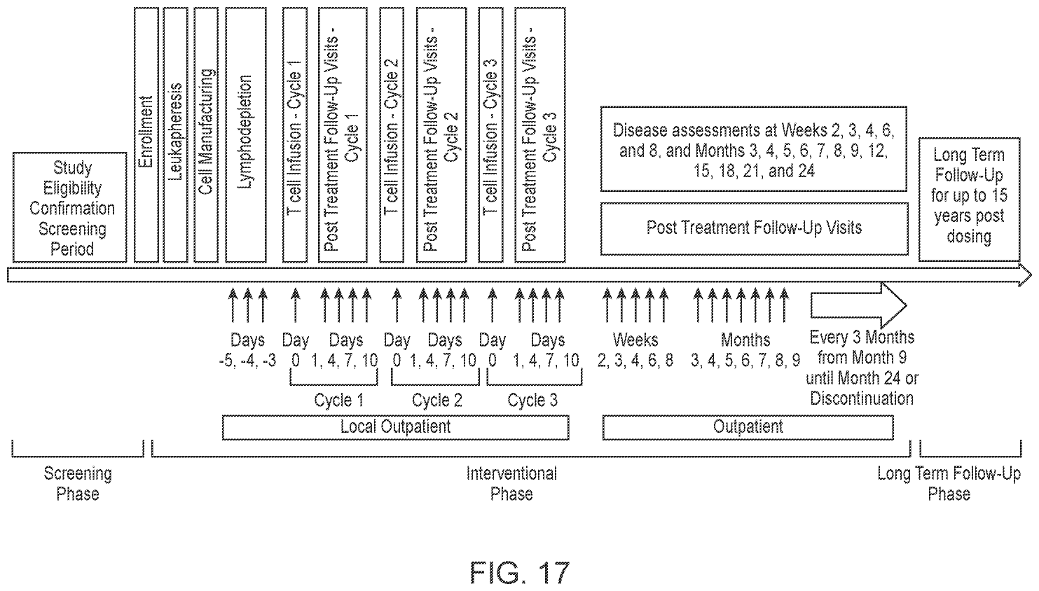

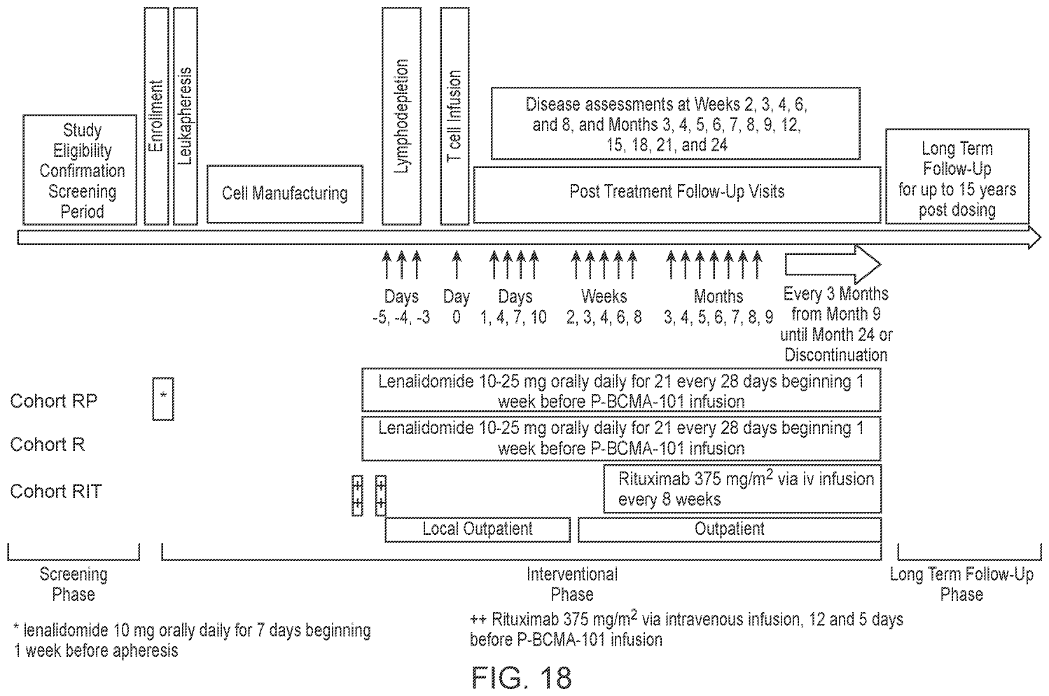

The study will be conducted in multiple parts: a Phase 1, open-label, SAD phase; a Phase 1, multiple dose cycle administration phase; a Phase 1, combination administration phase; and a Phase 2 open-label efficacy and safety phase, in adult subjects with relapsed/refractory MM. A schematic of the study design for single dose administration is shown in FIG. 14. Schematics for the Phase 1 cycle administration cohorts are shown in FIG. 16 and FIG. 17 in Section 15.5. A schematic of the study design for combination administration is shown in FIG. 18 in Section 15.6.

Only sites that are experienced in managing oncology subjects and stem-cell/bone marrow transplant with the resources to manage the types of acute emergent events expected with CAR-T cell administration will be selected to participate in this study. A Safety Committee will meet regularly to review data throughout the study.

Subjects meeting the protocol entry criteria will be eligible to enroll in the study and will follow the procedures outlined in Table 2 for single administration cohorts. Procedures for cycle administration cohorts are detailed in Section 15.5. Procedures for the Phase 1 combination administration are detailed in Section 15.6. After a subject enrolls, leukapheresis will be performed to obtain PBMCs which will be sent to a manufacturing site to produce P-BCMA-101 CARTyrin-T cells. Allowing approximately 4 weeks for P-BCMA-101 manufacturing, subjects are intended to return for conditioning chemotherapy and P-BCMA-101 administration approximately 4 weeks from the Leukapheresis Visit (this period may be extended as deemed necessary by the Investigator).

Before dosing with the P-BCMA-101 cell infusion, subjects will receive a lymphodepletion chemotherapy regimen of 300 mg/m² of cyclophosphamide and 30 mg/m² of fludarabine, with each agent given daily for 3 consecutive days starting at Day −5. Subjects may be maintained as an inpatient during lymphodepleting chemotherapy as the Investigator deems appropriate.

After 2 rest days following the lymphodepletion chemotherapy regimen, subjects will be dosed with P-BCMA-101 cells administered IV over approximately 5 to 20 mins on Day 0. Prior studies conducted with other CAR-T therapies have observed peak toxicity to occur within 3-7 days of investigational product administration. Study subjects will be closely monitored during and after the infusion and for approximately 7 days afterwards. This observation period will include serial assessments of adverse events (AEs), including the emergence of P-BCMA-101 cell-related toxicities, including CRS for all subjects. CRS will be graded using the Lee criteria (Lee, 2014).

Subjects may be admitted to the hospital for the P-BCMA-101 administration based on the Investigator's assessment of the individual patient's risks. Admission is not required, but subjects should remain within 50 miles of the hospital through approximately 14 days after last dose of P-BCMA-101 and assessed for admission in case of symptoms of CRS or neurotoxicity such as fever. If admitted, subjects will not be discharged until they are assessed as stable by the Investigator. Subjects may be maintained as an inpatient during lymphodepleting chemotherapy or after the above criteria are met as the Investigator deems appropriate.

Subjects will return for regular follow up and will undergo serial assessment of safety, tolerability and anti-myeloma response, as specified in the Schedule of Events (Table 3). Posttreatment follow-up visits in both Phase 1 and Phase 2 will occur at Day 10, Weeks 2, 3, 4, 6, 8, Months 3, 4, 5, 6, 7, 8, 9, and then every 3 months thereafter for up to 24 months after P-BCMA-101 administration. Follow-up procedures for cycle administration cohorts are detailed in Section 15.5. Follow-up procedures for combination administration cohorts are detailed in Section 15.6.

All consenting subjects who receive P-BCMA-101 and who complete or withdraw from the study will be encouraged to enter a long-term follow-up (LTFU) protocol (P-BCMA-101-002). Between this study and the LTFU study, a subject will be followed for 15 years from the time of the last P-BCMA-101 infusion. This LTFU is for observation of delayed AEs in accordance with FDA (FDA, 2006) requirements for gene therapy clinical trials. Subjects will continue to be followed for overall survival during the LTFU phase.

3.2 Study Dosing

3.2.1. Dose Escalation Guidelines for Phase 1

Phase 1 of the study is an open-label, multi-center, SAD multiple cohort study, a multiple dose cycle administration cohort study, and a combination administration study in approximately 120 adult subjects. Initially, up to 6 dose levels of P-BCMA-101 will be administered intravenously as a single dose.

Proposed Doses (P-BCMA-101 cells/kg/dose) include:
Cohort minus 1: 0.25×10⁶

### Cohort 1: 0.75×10⁶

### Cohort 2: 2×10⁶

### Cohort 3: 6×10⁶

### Cohort 4: 10×10⁶

### Cohort 5: 15×10⁶

Phase 1 of the study will follow a 3+3 design of dose-escalating cohorts, wherein 3 subjects are initially planned to be dosed with P-BCMA-101 T cells for each cohort. Additional subjects may be dosed in a cohort depending on the outcomes observed in those subjects.

For each of the first 2 cohorts, dosing of the first 3 subjects will be staggered rather than concurrently, with at least a 14-day interval between subjects to assess safety, as the peak of toxicity in CAR-T studies occurs in 3-7 days of administration, with the vast majority within 2 weeks and resolving by 2-3 weeks (Frey, 2016).

At the discretion of the Safety Committee, beginning with the 3rd cohort, dosing of the first and second subject in each cohort will be staggered. Dose escalation guidelines are outlined in Table 1.

Administration of P-BCMA-101 cells will be conducted in escalating dose cohorts in a 3+3 design. Beginning with Cohort 1, at least 3 subjects will be dosed in the cohort. If no P-BCMA-101 cell-related DLT through Day 28 is observed in the first 3 subjects, then escalation may proceed to the next cohort. If a P-BCMA-101 cell-related DLT is observed in 1 of the first 3 subjects, then at least 3 additional subjects will be treated at this dose level. If DLTs are observed in 2 or more subjects, the MTD will be considered to be at or below the next lower dose level and further enrollment may take place at a lower dose level, or an intermediate dose level may be tested at the discretion of the Safety Committee, otherwise escalation may proceed (i.e. the MTD is the highest dose cohort assessed in which this has not occurred). In the event that 2 or more subjects experience DLTs in Cohort 1, the Safety Committee, after review of all available data, may elect to dose 3 subjects in Cohort −1 with the same 3+3 expansion rules. In the event that 2 or more subjects experience DLTs in Cohort −1, the Safety Committee, based on consideration of safety and efficacy data to assess risk vs. benefit, may elect to dose 3 subjects at a lower dose with the same 3+3 expansion rules or discontinue the study.

Additional subjects may be added to a cohort by the Safety Committee based on the safety and efficacy data from that cohort to further evaluate the safety and anti-myeloma effects of P-BCMA-101, provided the dose does not exceed the MTD. If Cohort 5 is completed without concluding an overall MTD, the Safety Committee may elect to assess further escalation cohorts in 5-10×10⁶ P-BCMA-101 cells/kg increments.

3.2.2. Dosing in Cycle Administration

In Phase 1—Cycle Administration, multiple doses of P-BCMA-101 will be administered intravenously in 2 cycles (Cohort A and Cohort C) or 3 cycles (Cohort B) of 2 weeks each. The total dose administered will follow the 3+3 design starting at <the MTD as determined during single administration escalation. In the first cycle ⅓ the total dose will be administered. In Cohort A up to ⅔ the total dose will be administered in the 2nd cycle. In Cohort B up to ⅓ the total dose will be administered in each of the 2nd and 3rd cycles. In Cohort C up to ⅔ the total dose will be administered in the 1st cycle and up to ⅓ the total dose will be administered in the 2nd cycle. Details of the procedures are provided in Section 15.5. Prior to each infusion Serum creatinine should be ≤2.0 mg/dL, serum glutamic oxaloacetic transaminase (SGOT)≤3×the upper limit of normal and total bilirubin≤2.0 mg/dL or have medical monitor approval to proceed with P-BCMA-101 infusion.

3.2.3. Dosing in Combination Administration

In Phase 1—Combination Administration, P-BCMA-101 will be administered in combination with approved therapies:

Lenalidomide

Cohort R: lenalidomide 10 mg orally daily for 21 of every 28 days beginning 1 week before P-BCMA-101 infusion; and

Cohort RP: lenalidomide 10 mg orally daily for 7 days beginning 1 week before apheresis and for 21 of every 28 days beginning 1 week before P-BCMA-101 infusion.

Dosing with lenalidomide will continue for Cohort R and Cohort RP unless disease progresses. Refer to the lenalidomide package insert for prescribing information (Lenalidomide, 2019) (note particularly that lenalidomide is a presumed teratogen, pregnancy avoidance and monitoring is necessary). The following are additional recommendations specific to this protocol. If no DLTs are reported and platelets are ≥50,000/μL and neutrophils>1000/μL 28 days after P-BCMA-101 administration the dose may be increased to 25 mg orally daily for 21 of every 28 days. If <2 DLTs are reported in the first 6 patients treated at this dose, the starting dose in all patients may be increased to 25 mg orally daily for 21 of every 28 days at the determination of the Safety Committee. During treatment if neutrophils decrease to <1000/μL hold lenalidomide until they are ≥1000/μL, then restart at a 5 mg lower dose. During treatment if platelets decrease to <30,000/μL hold lenalidomide until they are ≥30,000/μL, then restart at a 5 mg lower dose. If creatinine clearance is 30-60 mL/min the maximum lenalidomide dose should be 10 mg daily. If creatinine clearance is <30 mL/min hold lenalidomide. Lenalidomide should be discontinued in case of DLT. The lowest dose allowed on this study is 5 mg daily. The investigator and Safety Committee may decide to discontinue lenalidomide at time based on other safety findings. Patients should receive concomitant anticoagulation as indicated (eg. aspirin 325 mg orally daily). Do not administer glucocorticoids with lenalidomide.

Rituximab

Cohort RIT: 375 mg/m² via intravenous infusion, 12 and 5 days before P-BCMA-101 infusion, then every 8 weeks unless disease progresses. Refer to the rituximab package insert for prescribing information (Rituximab, 2019). The following are additional recommendations specific to this protocol. Rituximab should only be administered by a healthcare professional with appropriate medical support to manage severe infusion-related reactions that can be fatal if they occur. First Infusion: Initiate infusion at a rate of 50 mg/hr. In the absence of infusion toxicity, increase infusion rate by 50 mg/hr increments every 30 minutes, to a maximum of 400 mg/hr. Subsequent Infusions: Standard Infusion: Initiate infusion at a rate of 100 mg/hr. In the absence of infusion toxicity, increase rate by 100 mg/hr increments at 30-minute intervals, to a maximum of 400 mg/hr. Administer only as an intravenous infusion. Do not administer as an intravenous push or bolus. Premedicate before each infusion with acetaminophen, an antihistamine, and 100 mg intravenous methylprednisolone to be completed 30 minutes prior to each infusion. Rituximab should be discontinued in case of infusion reaction or DLT. The investigator and Safety Committee may decide to discontinue rituximab at any time based on other safety findings. Prophylaxis and observation for infectious diseases such as Pneumocystis pneumonia (PCP) should be considered for patients during and following treatment per rituximab prescribing information.

The dose of P-BCMA-101 administered will escalate or de-escalate following the 3+3 design starting at ≤the MTD as determined during dose escalation. Details of the procedures are provided in Section 15.6.

3.2.4. Dosing in Phase 2

Phase 2 of the study is an open-label, multi-center study in approximately 100 adult subjects with relapsed/refractory MM. Subjects in Phase 2 will receive a total dose of 6-15×10⁶ cells/kg (per the schedule determined in Phase 1).

3.2.5. Repeat Dosing

If sufficient P-BCMA-101 cells remain from manufacturing when a subject's disease progresses, with Safety Committee approval additional cells may be administered up to the highest dose level that has successfully completed dose-limiting toxicity assessment. In order to receive an additional P-BCMA-101 T-cell infusion, subjects will be assigned a new subject identification number, they will have to meet all eligibility criteria as described for the initial dosing, and will undergo the same screening, enrollment, conditioning chemotherapy, and follow-up procedures except for leukapheresis. Details regarding retreatment procedures are provided in Section 15.4.

3.3. Evaluation of Dose-Limiting Toxicity

DLT is defined as any National Cancer Institute Common Terminology Criteria for Adverse Events (NCI CTCAE) Grade≥3 event at least possibly related to P-BCMA-101 cell therapy, including uncontrollable expansion of P-BCMA-101 cells, and not attributable to the underlying disease or lymphodepleting chemotherapy regimen with onset within the first 28 days following the last P-BCMA-101 cell infusion with the following exceptions:

Grade 3 or 4 neutropenia with or without neutropenic fever resolving within 28 days following the last P-BCMA-101 cell infusion;
Grade 3 fever;
Grade 3 or 4 thrombocytopenia, with or without bleeding due to thrombocytopenia, resolving within 28 days following the last P-BCMA-101 cell infusion;
Grade 3 or 4 anemia and lymphopenia;
Grade 3 or 4 hypogammaglobulinemia;

### Alopecia;

Grade 3 or 4 nausea, vomiting, or diarrhea which responds to medical treatment within 24 hours
Immediate hypersensitivity reactions (fever, rash, bronchospasm) occurring within 2 hours of cell infusion (related to cell infusion) that are reversible to a Grade 2 or less within 6 hours of cell administration with standard antihistamine based therapy;
Grade 3 encephalopathy that recovers to less than Grade 2 within 28 days;
Grade 3 CRS per Lee criteria (Lee, 2014) that resolves within 14 days;
Grade 3 non-hematological laboratory abnormalities that recover to ≤Grade 2 in 14 days;
Grade 4 non-hematological laboratory abnormalities that recover to ≤Grade 2 in 7 days.

3.4. Number of Subjects and Duration of Study

In Phase 1, up to 120 subjects at up to 20 sites are planned to be enrolled. In Phase 2, approximately 100 subjects at up to 20 sites are planned to be enrolled. Enrolled subjects will undergo serial measurements of safety, tolerability and response (myeloma staging). These measures will be obtained during a period between Screening and up to 24 months after P-BCMA-101 administration according to the schedule of events described in Table 2 and Table 3 for single administration, and in Section 15.5 for cycle administration cohorts and Section 15.6 for combination administration cohorts. Subjects who experience disease progression may receive an additional infusion of P-BCMA-101 according to the schedule of events described in Table 8 and Table 9.

After completing or withdrawing from this protocol, consenting subjects who have received P-BCMA-101 will be encouraged to enroll in a separate protocol (P-BCMA-101-002) that allows for continued follow-up for a total of 15 years after the last dosing to evaluate long-term safety.

4. Selection of Study Population, Withdrawal, Completion, and Stopping Criteria

4.1. Inclusion Criteria

A subject must meet the following inclusion criteria to be eligible for participation in this study:

1. Must have signed written, informed consent.
2. Males or females, ≥18 years of age.
3. Must have a confirmed diagnosis of active MM as defined by the IMWG criteria at initial diagnosis (Rajkumar, 2014).
4. Must have measurable MM as defined by at least 1 of the following criteria:

### Phase 1:

Serum M-protein greater or equal to 0.5 g/dL (5 g/L);

Urine M-protein greater or equal to 200 mg/24 h;

Serum free light chain (FLC) assay: involved FLC level greater or equal to 10 mg/dL (100 mg/L) provided serum FLC ratio is abnormal;

Bone marrow plasma cells>30% of total bone marrow cells, or other measurable bone disease (e.g., plasmacytomas measurable by PET or CT) (with medical monitor approval) Phase 2:

Serum M-protein greater or equal to 1.0 g/dL (10 g/L);

Urine M-protein greater or equal to 200 mg/24 h;

Serum FLC assay: involved FLC level greater or equal to 10 mg/dL (100 mg/L) provided serum FLC ratio is abnormal;

5. Must have relapsed/refractory MM as defined by the following:

### Phase 1:

Received at least 3 prior lines of therapy, which must have contained a proteasome inhibitor and immunomodulatory agent (IMiD); OR

Received at least 2 prior lines of therapy if “double-refractory” to a proteasome inhibitor and IMiD, defined as progression on or within 60 days of treatment with these agents.

### Phase 2:

Received at least 3 prior lines of therapy which must have contained a proteasome inhibitor, an IMiD, and CD38 targeted therapy with at least 2 of the lines in the form of triplet combinations, and undergone >2 cycles of each line unless PD was the best response;

Refractory to the most recent line of therapy; AND

Undergone ASCT or not be a candidate for ASCT.

Note: induction therapy, autologous stem cell transplant, and maintenance therapy, if given sequentially without intervening progression, should be considered as single line.

6. Must be willing to practice birth control from the time of Screening and for the duration of the study (both males and females of childbearing potential).

Females on cohorts R, RP or RIT must commit either to abstain continuously from sexual intercourse or to use two methods of reliable birth control, beginning 4 weeks prior to initiating treatment, during therapy, during dose interruptions and continuing for 4 weeks following discontinuation of lenalidomide and 12 months after last dose of rituximab.

Males in cohort R or RP must always use a latex or synthetic condom during any sexual contact with females of reproductive potential while taking lenalidomide and for up to 4 weeks after discontinuing lenalidomide, even if they have undergone a successful vasectomy. Male patients taking lenalidomide must not donate sperm.

7. Must have a negative serum pregnancy test at Screening and a negative urine pregnancy test within 3 days prior to initiating the lymphodepletion chemotherapy regimen (females of childbearing potential).

Female subjects in cohort R and RP must have two negative pregnancy tests prior to initiating lenalidomide. The first test should be performed within 10-14 days and the second test within 24 hours prior to subject starting lenalidomide therapy and then weekly during the first month, then monthly thereafter in females with regular menstrual cycles or every 2 weeks in females with irregular menstrual cycles

8. Must be at least 90 days since autologous stem cell transplant, if performed.
9. Must have adequate vital organ function, defined as follows (or medical monitor approval):

Serum creatinine≤2.0 mg/dL and estimated creatinine clearance≥30 mL/min as calculated using the Cockcroft-Gault formula and not dialysis-dependent.

Absolute neutrophil count≥1000/μL and platelet count≥50,000/μL (≥30,000/μL if bone marrow plasma cells are ≥50% of cellularity).

Adequate absolute CD3 count estimated for obtaining target cell dose based on dosage cohort. (Phase 2: absolute lymphocyte count≥300/μL)

Hemoglobin>8 g/dL (transfusion and/or growth factor support is allowable).

Serum glutamic oxaloacetic transaminase (SGOT)≤3×the upper limit of normal and total bilirubin≤2.0 mg/dL (unless there is a molecularly documented history of Gilbert's syndrome).

Left ventricular ejection fraction (LVEF)≥45%. LVEF assessment must have been performed within 4 weeks of enrollment.

10. Must have recovered from toxicities due to prior therapies, with the exception of peripheral neuropathy, to Grade≤2 according to the NCI CTCAE version 4.03 criteria or to the subject's prior baseline.
11. Subjects must have an Eastern Cooperative Oncology Group (ECOG) performance status of 0-1.

4.2 Exclusion Criteria

1. Is pregnant or lactating
2. Has inadequate venous access and/or contraindications to leukapheresis.
3. Has active hemolytic anemia, plasma cell leukemia, Waldenstrom's macroglobulinemia, POEMS syndrome (polyneuropathy, organomegaly, endocrinopathy, monoclonal protein, and skin changes), disseminated intravascular coagulation, leukostasis, or amyloidosis.
4. Has an active second malignancy (not disease-free for at least 5 years) in addition to MM, excluding low-risk neoplasms such as non-metastatic basal cell or squamous cell skin carcinoma.
5. Has active autoimmune disease, such as psoriasis, multiple sclerosis, lupus, rheumatoid arthritis, etc. (the medical monitor will determine if a disease is active and autoimmune).
6. Has a history of significant central nervous system (CNS) disease, such as stroke, epilepsy, etc. (the medical monitor will determine if significant).
7. Has an active systemic infection (e.g. causing fevers or requiring antimicrobial treatment).
8. Has hepatitis B or C virus, human immunodeficiency virus (HIV) or human T-lymphotropic virus (HTLV) infection, or any immunodeficiency syndrome.
9. Has New York Heart Association (NYHA) Class III or IV heart failure, unstable angina, or a history of myocardial infarction or significant arrhythmia (e.g. atrial fibrillation, sustained [>30 seconds] ventricular tachyarrhythmias, etc.)
10. Has any psychiatric or medical disorder (e.g. cardiovascular, endocrine, renal, gastrointestinal, genitourinary, immunodeficiency or pulmonary disorder not otherwise specified) that would, in the opinion of the Investigator or medical monitor, preclude safe participation in and/or adherence to the protocol (including medical conditions or laboratory findings that indicate a significant probability of not qualifying for or being unable to undergo adequate leukapheresis, conditioning chemotherapy and/or CAR-T cell administration).
11. Has received prior gene therapy or gene-modified cellular immunotherapy (or have approval of the medical monitor). Subject may have received non-gene-modified autologous T-cells or stem cells in association with an anti-myeloma treatment.
12. Has received anti-cancer medications within 2 weeks or 5 half-lives (whichever is longer or have medical monitor approval) of the time of initiating conditioning chemotherapy.
13. Has received immunosuppressive medications within 2 weeks of the time of initiating leukapheresis, and/or expected to require them while on study (the medical monitor will determine if a medication is considered immunosuppressive). Generally, all non-essential medications (including supplements, herbal medications, etc.) should be discontinued from 2 weeks before leukapheresis until 2 months after P-BCMA-101 administration due to the potential for unappreciated immunosuppressive effects.
14. Has received systemic corticosteroid therapy≥5 mg/day of prednisone or equivalent dose of another corticosteroid within 2 weeks of either the required leukapheresis or 1 week or 5 half-lives (whichever is shorter) of the administration of P-BCMA-101 or is expected to require it during the course of the study. (Topical and inhaled steroids are permitted. Systemic corticosteroids are contraindicated after receiving P-BCMA-101 cells outside of study specific guidance).
15. Has CNS metastases or symptomatic CNS involvement (including leptomeningeal carcinomatosis, cranial neuropathies or mass lesions and spinal cord compression) of their myeloma.
16. Has a history of severe immediate hypersensitivity reaction to any of the agents used in this study.
17. Has a history of having undergone allogeneic stem cell transplantation, or any other allogenic or xenogeneic transplant, or has undergone autologous transplantation within 90 days.
18. Unable to take acetylsalicylic acid (ASA) (325 mg) daily as prophylactic anticoagulation. Patients intolerant to ASA may use warfarin or low molecular weight heparin) (Cohorts R and RP only).
19. History of thromboembolic disease within the past 6 months, regardless of anticoagulation (Cohorts R and RP only).

4.3 Subject Withdrawal

Subjects who are enrolled and do not complete the study protocol will be considered to have prematurely discontinued the study. The reasons for premature discontinuation (for example, voluntary withdrawal, toxicity, death) must be recorded on the case report form. Final study evaluations will be completed at the time of discontinuation. If a subject discontinues from this study, the events for the next visit scheduled should be performed (including all safety and efficacy evaluations) prior to initiating alternative medical, radiation or surgical intervention and recorded as an end-of-study visit for this study. Subjects who withdraw from the protocol after having received P-BCMA-101 will be encouraged to enroll in the companion long-term follow-up protocol P-BCMA-101-002. Potential reasons for premature discontinuation include:

1. The subject is lost to follow-up.
2. The judgment of the Investigator is that the subject is too ill to continue.
3. Subject noncompliance with study therapy and/or clinic appointments.

### 4. Pregnancy

5. Voluntary withdrawal; a subject may remove himself/herself from the study at any time without prejudice. A subject may withdraw from the study at any time they wish to withdraw consent.
6. Significant progression of malignancy, requiring alternative medical, radiation or surgical intervention. If disease markers decrease from P-BCMA-101 cell activity after reaching progressive disease instead of confirming a prior increase, they should subsequently increase from that level for consideration of progressive disease to discontinue the subject from the protocol unless another systemic myeloma therapy is indicated.
7. Technical difficulties are encountered in the T cell genetic modification and expansion procedure that precludes the generation of clinical cell doses that meet all Quality Control criteria.
8. Termination of the study by the Principal Investigator, the sponsor, the study funder, the Institutional Review Board (IRB)/Independent Ethics Committee (IEC), or the Food and Drug Administration (FDA).

4.4. Study Termination

The Sponsor may suspend or terminate the study at any time for any reason. If the study is suspended or terminated, the Sponsor will ensure applicable sites, regulatory agencies and IRBs/IECs are notified as appropriate.

If the Investigator stops/terminates the study at their site the Sponsor must be notified. The Sponsor will ensure Regulatory Agencies and IRBs/IECs are notified as appropriate.

The Sponsor will ensure appropriate end of study declarations are made to the relevant Regulatory Agencies/IECs in accordance with local regulations.

5. Study Treatments

5.1. Leukapheresis

Subjects who complete all screening procedures and meet all eligibility criteria will undergo leukapheresis to harvest peripheral blood mononuclear cells (PBMCs) for the manufacture of P-BCMA-101. This visit should occur within ˜28 days of the Screening Visit. Subjects enrolled in the study will undergo standard leukapheresis procedure using Spectra Optia system (Terumo BCT), or equivalent leukapheresis machine, at the enrolling hospital. Immediately following the procedure, leukapheresed cells will be shipped in validated, temperature-controlled conditions to the manufacturing and analytic sites. The intent is to perform a 10-15 liter (minimum and maximum defined by site policies) apheresis and harvest a target of 1.5-3×10¹⁰ (1.5×10¹⁰ minimum and 5×10¹⁰ maximum) white blood cells (WBC) (count is also acceptable as total nucleated cells [TNC]). For additional detail, please refer to the Study Reference Manual (Apheresis Center Manual). As it is recognized that volume, cell number and other output parameters reached at the end of apheresis will vary between patients, machines, methods and operators, this is provided as guidance as opposed to absolute requirements.

The manufacturing of P-BCMA-101 is diagramed in FIG. 15. The collected apheresis products will be transported by courier to the manufacturer for immediate manufacturing. Manufacturing of P-BCMA-101, which includes T cell isolation, electroporation with piggyBac DNA plasmid (P-BCMA-101 plasmid encoding anti-BCMA CARTyrin) and Super piggyBac transposase mRNA (SPB mRNA), CARTyrin+ T cell selection, and cell expansion, will be completed in approximately 4 weeks. Final product will be cryopreserved in bags. The final formulation will have up to 10% (v/v) dimethyl sulfoxide (DMSO).

Following product release for infusion, frozen P-BCMA-101 product will be shipped by courier to the pharmacy or applicable cell therapy facility of the enrolling study center. P-BCMA-101 will be stored there at ≤−130° C. until time of administration.

If P-BCMA-101 cells that meet release criteria cannot be manufactured from the leukapheresis sample, a second leukapheresis and manufacturing may be attempted. If the second attempt also fails, the subject will be withdrawn from the study and considered not to have undergone study treatment. If sufficient P-BCMA-101 cells remain from manufacturing when a subject's disease progresses, with Safety Committee approval additional cells may be administered up to the highest dose level that has successfully completed dose-limiting toxicity assessment.

Once the product is manufactured, subjects will return to the clinic for the Conditioning Chemotherapy and P-BCMA-101 Administration Periods approximately 4 weeks (this period may be extended at the discretion of the Investigator and medical monitor) after the Leukapheresis Visit.

Subjects who experience rapid disease progression following the Leukapheresis Visit and prior to admission for the Conditioning Chemotherapy and P-BCMA-101 Administration Period may be administered salvage therapy at the discretion of the Investigator. Salvage therapy should not be used unless necessary and will be determined by the subject's clinical history (previously used agents are preferred, and medical monitor approval needed) at the discretion of the Investigator. If a subject receives salvage therapy, the Conditioning Chemotherapy and P-BCMA-101 Administration Period should be scheduled at least 2 weeks or 5 half-lives after the date of the last treatment of salvage therapy and the subject should meet the criteria described in Section 4 and Section 6 regarding entry criteria and concomitant medications. The subject's response to the salvage therapy will be evaluated by the Investigator and medical monitor in order to determine whether the subject will remain eligible to receive the Investigational Product.

Subjects will be permitted to receive radiation therapy or plasmapheresis and exchange for palliative purposes throughout the study period.

5.2. Conditioning Chemotherapy

Before dosing with the P-BCMA-101 cell infusion, subjects will receive a conditioning lymphodepletion chemotherapy regimen of 300 mg/m² of cyclophosphamide and 30 mg/m² of fludarabine, with each chemotherapy agent given sequentially IV over 30 minutes daily for 3 consecutive days (Day −5 through Day −3). Subjects should continue to meet entry criteria at the time of initiation of conditioning chemotherapy or have medical monitor approval. For subjects in Cohort R, Cohort RP and Cohort RIT, the combination therapy should be administered prior to conditioning chemotherapy on applicable days. The following assessments should be repeated within 72 hours prior to Day −5: Mini Mental Status Exam (MMSE), physical exam, vital signs, chemistry panel including electrolytes and magnesium, hematology including B and T cell counts, coagulation, assessment of circulating myeloma/plasma cells, and pregnancy test (if applicable). A baseline myeloma response assessment must be conducted within 7 days prior to initiating conditioning chemotherapy. Subjects may be admitted and treated as inpatients at this point at the discretion of the Investigator.

5.3. P-BCMA-101 Administration

5.3.1. Description

P-BCMA-101 is comprised of activated T cells genetically modified through an electroporation-based, non-viral (DNA transposon) gene delivery system called the piggyBac (PB) DNA modification system. The PB DNA modification system efficiently moves DNA from a plasmid to a chromosome via a “cut and paste” mechanism. P-BCMA-101 has been designed to include three major components: an anti-BCMA Centyrin CAR (CARTyrin) gene, a DHFR selection gene (Rushworth, 2016), and an iCasp9-based safety switch gene (Straathof, 2005). These components, along with a 5′ promoter and 3′ poly-A signal, are flanked by two non-translated cis-regulatory insulator elements that can stabilize transgenes by blocking improper gene activation or silencing (Mossine, 2013).

The CARTyrin expression cassette encodes for an extracellular BCMA-binding Centyrin protein fused to a CD8a signal/leader peptide, CD8a hinge/spacer, a CD8a transmembrane domain, an intracellular 4-1BB signaling domain and a TCR (chain signaling domain. As compared with binding domains consisting of an antibody-based single chain variable region (scFv), the CARTyrin binding domain is a fully human protein that is smaller, more stable and potentially less immunogenic, but with similar antigen binding characteristics that enable recognition and killing of BCMA-expressing MM cells.

The DHFR selection gene is used during manufacturing of the T cells for ex vivo selection of CARTyrin expressing T cells to enhance potency and efficacy of the final product.

The iC9 safety switch gene is an extra feature not found in most CAR-T cells designed to allow for rapid ablation of P-BCMA-101 cells by intravenous administration of rimiducid, a synthetic dimerizer drug.

5.3.2. Product Labeling

Each product bag will be labeled with product name (P-BCMA-101), date of product manufacturing, product lot number, storage conditions, part number, and at least two non-personal subject identifiers (such as the subject's initials, birth date, and/or study subject identification number). In addition, the product bags will be labeled with the following for caution: “FOR AUTOLOGOUS USE ONLY”, “NOT EVALUATED FOR INFECTIOUS SUBSTANCES”, and “Caution: New Drug-Limited by Federal law to Investigational Use”.

5.3.3. Storage

Upon receipt of the investigational product and any related study treatment supplies, an inventory must be performed, and a drug receipt log filled out and signed by the person accepting the shipment. It is important that the designated study staff inventories, counts and verifies that the shipment contains all the items noted in the shipment inventory. Any damaged or unusable investigational product study drug in a given shipment will be documented in the study files. The Investigator must notify study sponsor or designee of any damaged or unusable study treatments that were supplied to the Investigator's site. P-BCMA-101 cells may require return to the sponsor or designee for various reasons, including but not limited to: 1) mislabeled product; 2) condition of subject prohibits infusion/injection, and 3) subject refuses infusion/injection. Overall, any unused product will be returned to the sponsor or designee.

Bags containing P-BCMA-101 cells will be stored at the site in blood bank conditions in a monitored ≤−130° C. freezer until the subject is ready for infusion.

5.3.4. Preparation

Recommended pre-medications prior to T cell infusion include 650 mg acetaminophen and 25-50 mg diphenhydramine hydrochloride. These medications may be given every 6 hours, as needed. Non-steroidal anti-inflammatory agents may be given for fever not controlled by acetaminophen, however, use should be carefully considered with regards to factors potentially impacting the subject's tolerance, such as bleeding risk and renal function. Evaluation for sepsis should be considered for fever of unexpected severity or duration, or other suggestive symptomology. Systemic corticosteroids should not be administered unless necessitated as described in supportive care guidance (Section 6.3), due to the potential for adverse impact on the efficacy of the T cell-based investigational product.

Subjects should continue to meet liver and renal laboratory entry requirements prior to dosing (i.e. prior to initiating conditioning chemotherapy). Additionally, there should continue to be no evidence of active infection, or significant cardiac (e.g. hypotension requiring pressor or uncontrollable arrhythmia) or pulmonary compromise (e.g. supplemental oxygen requirement or significant progressive infiltrates on chest x-ray).

5.3.5. Dosing and Administration

In Phase 1, dose levels of P-BCMA-101 will be administered intravenously as a single dose or multiple doses. Dose levels will be tested by cohort in the 3+3 escalation design described in the Dose Escalation Guidelines (Section 3.2). In Phase 2, subjects will receive a total dose of 6 to 15×10⁶ cells/kg (per the schedule determined in Phase 1).

Cryopreserved P-BCMA-101 is transported to the enrolling study center and stored at <−130° C. until infusion. If the label on a cryobag received for a subject indicates the contents are more than the assigned dose of P-BCMA-101 cells for the cohort (+/−5%), only a volume of the product corresponding to the assigned dose should be administered. If the label on a cryobag received for a subject indicates the contents are less than the assigned dose of P-BCMA-101 cells for the cohort (+/−5%), and multiple cryobags have been provided for the subject, a volume of the product corresponding to the assigned dose should be administered from multiple cryobags (this may include cryobags from different leukaphereses/manufacturings). If the label on a cryobag received for a subject indicates the contents are less than the prescribed dose of P-BCMA-101 cells for the cohort (+/−5%), and a sufficient number of cryobags have not been provided for a volume of the product corresponding to the assigned dose for the subject, the product may be administered, however the dose for that subject will be recorded as the dose received, not the cohort level dose, and all data from that subject accorded thus. Immediately prior to infusion, P-BCMA-101 is thawed and infused as detailed below. No further processing of P-BCMA-101 will be needed prior to infusion. Cells should be logged in the investigational pharmacy or applicable cell therapy facility.

After 2 rest days following the lymphodepletion chemotherapy regimen, subjects will be dosed with P-BCMA-101 administered IV. Subjects may be admitted to the hospital for the P-BCMA-101 administration based on the investigator's assessment. Admission is not required, but subjects should remain within 50 miles of the hospital through approximately 14 days after last dose of P-BCMA-101 and assessed for admission in case of symptoms of CRS or neurotoxicity such as fever. Immediately prior to infusion, P-BCMA-101 cells are transferred at ≤−130° C. (in vapor phase liquid nitrogen) to the subject bedside. The cells will be thawed at the bedside just prior to infusing using a water bath maintained at 36° C. to 38° C. (thawing at a different location and transporting to bedside at 2-8° C. must be pre-approved by the sponsor). The bag will be gently massaged until the cells have just thawed. There should be no frozen clumps left in the container. If the bag appears to be damaged or leaking, or otherwise compromised, it should not be infused, and should be returned to the sponsor or sponsor's designee. The infusion bag label will have at least 2 unique identifiers including the subject's study identification number and initials or birth date. Prior to the infusion, 2 individuals will independently verify all this information in the presence of the subject and so confirm that the information is correctly matched to the participant. Emergency medical equipment (i.e., emergency trolley) will be available during the infusion in case the subject has an allergic response, or severe hypotensive crisis, or any other reaction to the infusion. An intensive care unit should be within a reasonable distance of the study administration site.

The P-BCMA-101 cells will generally be provided in 250 mL infusion bags at a concentration ranging approximately from 3×10⁵/mL to 2.4×10⁷/mL, dependent on the dose. They should be administered by intravenous infusion at a flow rate of approximately 1 mL to 20 mL per minute through an 18-gauge latex-free, Y-type blood set with a 3-way stopcock or butterfly needle as appropriate for the dose and volume. The duration of the infusion should be approximately 5-20 minutes, as appropriate for the volume. It is intended P-BCMA-101 cells be infused within 2 hours of thawing, and the maximum stability assessed is 4 hours. For additional detail, see also Study Reference Manual (Pharmacy and Administration Manual).

Vital signs (temperature, respiration rate, pulse, and blood pressure) will be taken before and after infusion, then every 15 minutes for at least one hour and until these signs are satisfactory and stable.

Empty bags and remaining cells should be disposed of per institutional biosafety guidelines. In case of unused or damage product/packaging, the sponsor should be contacted to determine disposition.

Prior studies conducted with other CAR-T therapies have observed peak toxicity to occur within 3-7 days of investigational product administration. Study subjects will be closely monitored during and after the infusion and for approximately 7 days afterwards. This observation period will include serial assessments of AEs, including the emergence of P-BCMA-101-related toxicities, such as CRS for all subjects.

If admitted, subjects will not be discharged until they are assessed as stable by the Investigator. Subjects may be maintained as an inpatient during lymphodepleting chemotherapy or after the above criteria are met as the Investigator deems appropriate. Subjects will return for regular follow up and will undergo serial assessment of safety, tolerability and anti-myeloma response as specified in the Schedule of Events tables.

6. Concomitant Medication and Treatment

6.1. Prohibited Concomitant Medications and Treatments

May not receive on study or have received anticancer/anti-myeloma medications after P-BCMA-101 administration or within 2 weeks or 5 half-lives (whichever is longer or have medical monitor approval) of the time of initiating leukapheresis or conditioning chemotherapy. Salvage treatment should not be used unless necessary but may be administered between leukapheresis and conditioning chemotherapy if deemed necessary by the Investigator and approved by the medical monitor. The intervals described in the exclusion criteria need to be met for the relevant agents.

May not have received immunosuppressive medications within 2 weeks or 5 half-lives (whichever is longer) at the time of initiating leukapheresis, and/or is expected to require them while enrolled in the study, or receive them after P-BCMA-101 administration (the medical monitor will determine when and if a potentially immunosuppressive medication is allowed).

May not have received systemic corticosteroid therapy≥5 mg/day of prednisone or equivalent dose of another corticosteroid within 2 weeks of either the required leukapheresis or 1 week or 5 half-lives (whichever is shorter) of the administration of P-BCMA-101 or is expected to require it during the course of the study (topical, and inhaled steroids are permitted). Systemic corticosteroids are generally contraindicated after receiving P-BCMA-101 outside of study specific guidance regarding the management of AEs, directed use with combination therapies, or with medical monitor approval.

May not receive G-CSF or GM-CSF within 2 weeks of the time of initiating leukapheresis, within 5 half-lives before planned administration of P-BCMA-101, or within 2 months after administration of P-BCMA-101 without medical monitor approval.

Generally, all non-essential medications (including supplements, herbal medications, etc.) should be discontinued from 2 weeks before leukapheresis until 2 months after P-BCMA-101 administration due to the potential for unappreciated immunosuppressive effects.

6.2. Permitted Concomitant Medications and Treatments

Subjects will be permitted to receive radiation therapy or plasmapheresis and exchange for palliative purposes throughout the study period. Other diagnostics or treatments that the Investigator considers necessary for a subject's welfare may be conducted at the discretion of the Investigator in keeping with standards of medical care and in adherence to the protocol.

All prescription and nonprescription medication, vitamins, herbal and nutritional supplements, taken by the subject during the 30 days prior to Screening will be recorded at the Screening Visit. At every visit, concomitant medications will be recorded in the medical record and on the appropriate electronic Case Report Form (eCRF). Any additions, deletions, or changes of these medications will be documented.

6.3. Supportive Care Guidance

Recommended pre-medications prior to P-BCMA-101 infusion include 650 mg acetaminophen and 25 to 50 mg diphenhydramine hydrochloride. These medications may be given every 6 hours as needed. Non-steroidal anti-inflammatory agents may be given for fever not controlled by acetaminophen, however, use should be carefully considered with regards to factors potentially impacting the subjects' tolerance, such as bleeding risk and renal function. Work-up for sepsis should be considered for fever of unexpected severity or duration, or other suggestive symptomology. Systemic corticosteroids should not be administered unless necessitated by a severe or life-threatening AE as described below, due to the known adverse impact on the survival of the T cells and consequent efficacy of the T cell-based investigational product.

The Investigator must use appropriate medical judgment in the management of AEs, including expected events such as those of conditioning chemotherapy, as well as those of CAR-T cell therapies including CRS. CRS is perhaps the most common AE associated with CAR-T cell administration and is characterized by cytokines being released into circulation by active T-cells and the downstream effects on multiple organ systems. CRS has also been seen following the infusion of therapeutic monoclonal antibodies (mAbs), systemic interleukin-2 (IL-2), and the bispecific CD19-CD3 T-cell engaging antibody, blinatumomab. The incidence and severity of CRS has been reported to correlate with disease burden in patients with acute lymphoblastic leukemia. Clinical and laboratory measures range from mild CRS (e.g. constitutional symptoms, high fever) to severe CRS and/or potentially life threatening (e.g. high fever, malaise, fatigue, myalgia, nausea, anorexia, tachycardia/hypotension, hypoxia, capillary leak, cytopenias, cardiac dysfunction, renal impairment, hepatic failure, disseminated intravascular coagulation. Neurologic changes and/or cerebral edema have been associated with the potential for severe or fatal outcomes). The goal of CRS management in CAR-T cell therapy is to prevent life-threatening conditions while preserving the benefits of antitumor effects, thus therapy is generally carefully tailored to symptoms and/or markers of CRS. For example, corticosteroids and other aggressive immunosuppressive agents are effective treatments for CRS, but also frequently toxic the CAR-T cells. Suggested management of CRS is generally per Lee et al. (Lee, 2014) and Brudno et al. (Brudno, 2016). For detailed toxicity management recommendations see the Study Reference Manual (Toxicity Reference Manual). In summary: Symptomatic treatment of grade 1 CRS is suggested. CRS is defined as Grade 2 when the subject develops hypotension responsive to fluids or 1 low-dose vasopressor or mild respiratory symptoms responsive to low flow oxygen (40% FiO2) or Grade 2 organ toxicity. Because hypotension is a major driver of severity grading, it is imperative that a clear baseline blood pressure be established prior to initiation of therapy. The decision to intervene with immunosuppressive agents (tocilizumab+/−corticosteroids) for subjects with Grade 2 CRS is influenced by the degree to which the subject is judged to be able to tolerate the altered hemodynamics and organ stresses associated with the syndrome. In older subjects and subjects with significant comorbidities, depending on clinical judgment, it may be appropriate to intervene with immunosuppression in subjects with Grade 2 CRS. Subjects in whom fluid therapy and 1 low-dose vasopressor are not sufficient to reverse hypotension are classified as severe or Grade 3 CRS. Similarly, subjects who require more than low flow oxygen or show evidence for Grade 3 organ toxicity, including but not limited to coagulopathy, renal, or cardiac dysfunction, should be considered Grade 3. Subjects with Grade 3 CRS need to be monitored very closely, likely in an intensive care unit with 1:1 nursing care. Importantly, in subjects with Grade 2 or higher CRS, careful attention should be paid to cardiac function, as cardiac decompensation may occur and may not be readily evident without careful monitoring. Frequent echocardiographic monitoring may be indicated in subjects in whom there is a concern of cardiac dysfunction. Subjects with Grade 3 CRS should be treated with immunosuppressive agents such as tocilizumab and corticosteroids (e.g. 10 mg dexamethasone IV every 6 hours) because of the risk for progression and the potential for irreversible organ dysfunction, with the goal of preventing progression to Grade 4. Grade 4 CRS occurs when subjects experience toxicity that is immediately life threatening, including a need for mechanical ventilation or Grade 4 organ toxicity. It is recommended that all subjects with Grade 4 CRS be treated with immunosuppressive and/or cytotoxic agents such as tocilizumab (typically 8 mg/kg daily for 1 to 2 days), corticosteroids (e.g. methylprednisolone 1 g daily for 3 days), rimiducid (typically 0.4 mg/kg) and/or an aggressive immunosuppressive/cytotoxic agent such as cyclophosphamide (e.g. 1.5 g/m²) (generally use of rimiducid would be prioritized over use of a systemically toxic cytotoxic agent) in an attempt to suppress the inflammatory cascade and to prevent irreversible organ dysfunction. These options may also be considered for Grade 3 toxicity unresponsive to other measures.

When indicated, tocilizumab is typically administered intravenously over 1 hour at a dose of 8 mg/kg, with an option to repeat the dose if clinical improvement does not occur within 24 to 48 hours. In subjects with CRS who respond to tocilizumab, fever and hypotension often resolve within a few hours, and pressors and other supportive care measures can potentially be weaned quickly thereafter. In some cases, however, symptoms may not completely resolve, and continued aggressive support may be necessary for several days.

If a subject develops uncontrollable P-BCMA-101 T cell expansion or other clinically significant Grade 3-4 toxicities possibly related to P-BCMA-101, it is recommended the Investigator review the clinical scenario and potential confounding factors, and consider treating with immunosuppressive and/or cytotoxic agents, such as corticosteroids (e.g. methylprednisolone 1 g daily for 3 days), rimiducid (typically 0.4 mg/kg) and/or an aggressive immunosuppressive/cytotoxic agent such as cyclophosphamide (e.g. 1.5 g/m²).

7. Schedule of Assessments and Procedures

7.1. Schedule of Procedures

The Schedule of Procedures for this study will be the same for subjects in single administration, and is provided in Table 2 for procedures from Screening through conditioning chemotherapy and Table 3 for procedures from P-BCMA-101 administration through follow-up. The Schedule of Procedures tables for cycle administration cohorts are provided in Section 15.5. The Schedule of Procedures tables for combination administration are provided in Section 15.6. Following obtaining consent, screening and confirmation of eligibility, all subjects will undergo leukapheresis, conditioning chemotherapy, and P-BCMA-101 administration as described in Section 5.3. Subjects will return for regular follow up and will undergo serial assessment of safety, tolerability and anti-myeloma response as specified in each respective Schedule of Events table. Post-treatment follow-up visits will occur at Day 10, Weeks 2, 3, 4, 6, 8, Months 3, 4, 5, 6, 7, 8, 9, 12, 15, 18, 21, and 24.

7.2. Clinical Assessments

Clinical assessments and procedures to be performed throughout the study are outlined in the Schedule of Events tables. Assessment timing windows not described in these tables may be found in the Study Reference Manual.

7.2.1. Medical History

General medical history and demographics will be recorded at Screening, as well as any baseline symptoms or medical conditions that would be considered AEs on study. MM disease history, including results of MM assessments obtained in the 6 months prior to Screening, as described in Inclusion Criterion #4, will also be obtained. Myeloma response will be collected as described in Section 7.2.10.

7.2.2. Physical and Neurological Examination

A complete physical examination, including a neurological examination, will be performed at Screening; within 72 hours prior to start of conditioning chemotherapy; on Day −5, on Day 0 before and approximately 1 hour after P-BCMA-101 administration; on Days 1, 4, 7, 10; Weeks 2, 3, 4, 6, and 8, and starting at Month 3 every 3 months for 24 months. After administration of P-BCMA-101, physical examination, including a neurological examination (or any other assessments deemed appropriate by the Investigator) should be repeated as often as clinically indicated by the AEs observed or by the institution's standards, but at minimum once per study visit.

7.2.3. Vital Signs

Vital signs, including blood pressure, heart rate, respiration rate, 02 saturation and temperature will be obtained at visits as outlined in the Schedule of Events tables. On Day 0, vital signs (temperature, respiration rate, heart rate, 02 saturation and blood pressure) will be taken before and approximately one hour after P-BCMA-101 administration, then every 15 minutes (+/−5 minutes) for at least one hour and until these signs are satisfactory and stable. Weight will be obtained at Screening, Enrollment and Leukapheresis. Height will be recorded only at Screening.

7.2.4. Performance Status

ECOG performance status will be assessed at Screening and Day −5 using the ECOG performance scale described in Appendix 15.1.

7.2.5. Clinical Safety Assessments

Subjects will be assessed for AEs throughout the study. AEs will be graded by the NCI CTCAE Version 4.03 criteria. Details on assessment and reporting of AEs and SAEs are provided in Section 8.

7.2.6. Cardiac Assessments

A 12-lead electrocardiogram (ECG) will be obtained at Screening, Days 0 (before and approximately 1 hour after P-BCMA-101 administration), 1, 4, 7, Week 4, and starting at Month 3 every 3 months for 24 months. Echocardiogram will be obtained at Screening only.

7.2.7. Laboratory Assessments

7.2.7.1. Clinical Chemistry and Hematology

Clinical chemistry and hematology laboratory evaluations will be performed as outlined in the Schedule of Events tables (Chemistry Panel, Hematology including B and T cells, Coagulation).

The chemistry panel will include sodium, potassium, chloride, magnesium, bicarbonate, blood urea nitrogen, creatinine, calcium, albumin, glucose, lactate dehydrogenase (LDH), total protein, alanine aminotransferase (ALT), aspartate aminotransferase (AST)/Serum glutamic oxaloacetic transaminase (SGOT), bilirubin (total and direct), and alkaline phosphatase. Coagulation evaluations will include PTT (partial thromboplastin time) and PT (prothrombin time) or international normalized ratio (INR). Assessments for tumor lysis (uric acid and phosphate) will be performed at baseline and Days 1, 4, 7, then as clinically indicated.

On Day 0, chemistry panel, hematology including B and T cells, and coagulation evaluations should be obtained before and approximately 1 hour after P-BCMA-101 administration. After administration of P-BCMA-101, these (or any other assessments deemed appropriate by the Investigator) should be repeated as often as clinically indicated by the AEs observed or by the institution's standards, but at minimum once daily on Days 1, 4, 7 and 10 and as outlined in the Schedule of Events tables.

Hematology laboratory evaluation will include complete blood count, platelets, B (CD19) and T (CD3) cell counts and CD4 and CD8 (at all timepoints except Days −3 and −4), and assessment of circulating myeloma/plasma cells (e.g., by flow cytometry or CBC with manual differential) is required at timepoints prior to P-BCMA-101 administration. Contact the sponsor and refer to exclusion criteria #3 and #10 if circulating myeloma/plasma cells are identified in the Enrollment & Baseline sample.

7.2.7.2. Pregnancy Testing

Female subjects of childbearing potential must have a negative serum pregnancy test at Screening and a negative urine pregnancy test within 72 hours prior to initiating the conditioning chemotherapy. Urine pregnancy tests will be performed prior to each dose of P-BCMA-101 and at subsequent post-treatment visits starting at Week 2.

Female subjects in cohort R and RP must have two negative pregnancy tests prior to initiating lenalidomide. The first test should be performed within 10-14 days and the second test within 24 hours prior to subject starting lenalidomide therapy and then weekly during the first month, then monthly thereafter in females with regular menstrual cycles or every 2 weeks in females with irregular menstrual cycles.

7.2.7.3. Infectious Disease Screening

Laboratory test will be performed at Screening for the presence of HIV 1 and 2 antibody, hepatitis B surface antigen, hepatitis C antibody, and HTLV.

7.2.8. Cytokine Release Syndrome

Blood samples for detection of CRS markers: CRP, ferritin, cytokines IL-6, IL-2, IL-15, TNF-α, and IFN-γ will be obtained as indicated in the Schedule of Events tables.

7.2.9. Mini Mental Status Exam

Mini Mental Status Exam (MMSE) will be performed as indicated in the Schedule of Events tables. The MMSE should include a sample of the subject's handwriting at all timepoints.

7.2.10. Disease Response Assessments

7.2.10.1. PET-CT Assessment

Assessment of disease by PET-CT scan will be performed at the Baseline visit within 7 days prior to initiating conditioning chemotherapy, then as clinically indicated (e.g. every 8 weeks if soft tissue plasmacytomas are found at baseline that require imaging as part of response assessments or to confirm response).

7.2.10.2. Response and Response Rates

Laboratory samples for evaluation of myeloma response will be drawn at Screening, Baseline visit within 7 days of initiating conditioning chemotherapy, Day 0 (before P-BCMA-101 administration), Weeks 2, 3, 4, 6, 8, Months 3, 4, 5, 6, 7, 8, 9, 12, 15, 18, 21, and 24.

Myeloma response will be assessed per the International Myeloma Working Group criteria and based on the standard assessments, as clinically indicated (e.g., ≤1 week after an assessment demonstrating a response for confirmation), for each subject, such as serum protein electrophoresis (SPEP), urine protein electrophoresis (UPEP), serum immunofixation (SIFE), urine immunofixation (UIFE), serum free light chains (FLC), PET/CT (Positron Emission Tomography/Computed Tomography, liver background) and/or bone marrow biopsy/aspirate. A baseline myeloma response assessment must be done within 7 days prior to initiating conditioning chemotherapy. Fresh bone marrow biopsy and aspirate must be collected at either screening or baseline (typically within 7 days of initiating conditioning chemotherapy) and at Week 4, Months 3, 6 and 12, then as clinically indicated, or have a medical monitor exemption. Response rate will be determined as the number of subjects in each best overall response category divided by the number of subjects in the study population.

7.2.10.3. Time to Response

For subjects with a response, the time to response will be assessed from time of P-BCMA-101 administration to time of first documented response (partial response [PR] or better).

7.2.10.4. Duration of Response

For subjects with a response, the duration of response will be assessed from time of first documented response (PR or better) to time of confirmed disease progression (deaths from other causes will be censored).

7.2.10.5 Progression Free Survival

Progression Free Survival (PFS) from time of P-BCMA-101 administration to time of confirmed disease progression or death will be assessed for all subjects.

7.2.10.6 Overall Survival

Overall survival (OS) from time of P-BCMA-101 administration to time of death will be assessed for all subjects.

7.2.11 Long-Term Follow-Up

All subjects treated with P-BCMA-101 in this study will be followed for up to 2 years in this study. After they discontinue this protocol, consenting subjects will roll over into a separate long-term safety follow-up protocol (P-BCMA-101-002) and be followed for a total of 15 years post-last dosing with P-BCMA-101.

7.3 Exploratory Assessments

7.3.1. Expansion and Persistence of P-BCMA-101 T Cells

P-BCMA-101 T cells (e.g. vector copies/mL of blood) will be quantified in the Blood Sample for P-BCMA-101 T cells described in the Schedule of Events tables.

7.3.2. P-BCMA-101 T Cell Composition

P-BCMA-101 T cells (e.g. phenotype, clonality etc.) will be assessed as indicated using the Blood Sample for P-BCMA-101 T cells described in the Schedule of Events tables.

7.3.3. Evaluation of Immune Response

Samples for assessing immunogenicity to P-BCMA-101 will be collected as indicated in the Schedule of Events tables (Blood Sample for Immunogenicity Assays).

7.3.4. Soluble BCMA Levels

Soluble BCMA will be measured in blood samples as indicated in the Schedule of Events tables (Blood Sample for BCMA and Other Biomarkers).

7.3.5. BCMA Expression on MM Cells

Cells from an archival or fresh tumor specimen (e.g. bone marrow) will be assessed for BCMA at baseline and as indicated in the Schedule of Events tables (Archival Tumor to Central Lab, Fresh Sample of Bone Marrow and Tumor and/or Blood Sample for BCMA and Other Biomarkers).

7.3.6. Relationship Between BCMA Antigen Density/Expression, Circulating Soluble BCMA and Clinical Response

Soluble BCMA and BCMA expression results will be correlated with clinical response.

7.3.7. Levels of Recipient B and T Cells

Levels of recipient B and T (CD4+ and CD8+) cells will be quantified in hematology samples as indicated in the Schedule of Events tables (Chemistry Panel, Hematology including B and T cell counts, coagulation).

8. Recording Adverse Events

The Principal Investigators are responsible for detecting, documenting, and reporting events that meet the definition of an AE or a serious adverse event (SAE). Individual AEs should be evaluated by the Investigator and reported to the Sponsor via the eCRF. This includes the evaluation of the intensity, the causality between the investigational product and/or concomitant therapy and the AE, seriousness, etc.

8.1 Time Period for Collecting AE and SAE Information

All AEs and SAEs will be collected from enrollment until the Month 24 visit, or withdrawal from the protocol, whichever is shorter.

8.2 Definition of Adverse Event

In accordance with the International Conference of Harmonization (ICH), an AE is any untoward medical occurrence in a subject or clinical investigation subject who receives a pharmaceutical product. The event does not necessarily have a causal relationship with study treatment to be an AE. An AE can therefore be any unfavorable and unintended sign (including an abnormal laboratory finding), symptom, or disease temporally associated with the use of an investigational product, whether or not considered related to the investigational product. Preexisting conditions should only be reported as AEs if they worsen during the study. Progression of the cancer under study and symptoms thereof are not considered an AE unless it is considered to be drug related by the Investigator. New abnormal laboratory findings should only be considered AEs if they necessitate treatment or are considered clinically significant by the Investigator.

AEs should be recorded in the eCRF using a diagnosis or possible diagnosis, and rated for intensity, causality, and seriousness. In the absence of a diagnosis, individual symptoms or findings may be recorded and the eCRF updated to reflect a final diagnosis once additional information becomes available.

All AEs should be followed until:

Resolved or improved to baseline.

Investigator confirms no further improvement can be expected.

On completion or discontinuation from the study, AEs will be followed for 30 days or until one of the above criteria is met. Ongoing AEs will continue to be recorded and monitored into the long-term follow up study.

8.2.1. Assessment of Intensity

Adverse events will be graded according to the NCI CTCAE version 4.03. The Investigator will assess intensity of all AEs using this five-point scale (Grade 1-5) and record on the eCRF. AEs not specifically listed on the NCI CTCAE should be graded according to Table 4:

TABLE 4
Grading of AEs Not Specified in NCI CTCAE version 4.03

| NCI CTCAE | | |
|---|---|---|
| Grade | Equivalent to | Definition |
| Grade 1 | Mild | Discomfort noticed but no disruption |
| | | of normal daily activity. |
| Grade 2 | Moderate | Discomfort sufficient to reduce or |
| | | affect daily activity; minimal medical |
| | | intervention is indicated. |
| Grade 3 | Severe | Incapacitating with inability to work |
| | | or perform normal daily activity; |
| | | treatment or medical intervention is |
| | | indicated in order to improve the |
| | | overall well-being or symptoms; |
| | | delaying the onset of treatment is |
| | | not putting the survival of the |
| | | subject at direct risk. |
| Grade 4 | Life-threatening/ | An immediate threat to life that |
| | disabling | requires urgent medical intervention. |
| Grade 5 | Death | AE resulting in death. |

8.2.2. Assessment of Causality

The Investigator will assess the causal relationship between the AE and investigational product (P-BCMA-101 and/or rimiducid, if administered) according to his/her best clinical judgement. An assessment of possibly/probably/definitely related is meant to convey there is evidence of a causal relationship, not that a relationship cannot be ruled out. The Investigator should consider alternative causes such as natural history of the underlying disease, lymphodepleting chemotherapy, concomitant medications, and other risk factors when making an assessment. The following scale will be used as guidance:

Not related—The subject did not receive the investigational product; the temporal sequence of the AE onset relative to administration of the investigational product is not reasonable; or there is another highly likely cause of the AE.

Unlikely related—The AE is not reasonably, temporally correlated with T-cell infusion; the AE is more likely explained by another cause or causes, but a theoretical impact of T cell infusion on the onset or severity of the event is remotely possible in a multi-factorial context

Possibly related—There is evidence of exposure to the investigational product; the temporal sequence of the AE onset relative to T cell infusion is plausible; there is a reasonable explanation for the investigational product to have elicited the AE; and the investigational product is equally likely to have caused the AE as other explanations.

Probably related—There is evidence of exposure to the investigational product; the temporal sequence of the AE onset relative to T cell infusion is plausible; the AE shows a pattern consistent with previous knowledge of the investigational product; or the AE is more likely explained by the investigational product than any other cause.

Definitely related—There is evidence of exposure to the investigational product; the temporal sequence of the AE onset relative to T cell infusion is plausible; the AE shows a pattern consistent with previous knowledge of the investigational product, or the AE is most likely explained by the investigational product and any other cause is improbable.

The Investigator may change his/her opinion of causality if additional information is received and amend the AE eCRF accordingly. The Investigator causality assessment is one of the criteria the sponsor will use to determine regulatory reporting requirements for an SAE.

8.3 Reporting Serious Adverse Events (SAEs)

An SAE is any AE that:

Results in death (NOTE: death should be recorded as the outcome, and initiating event leading to death as the event).

Is life-threatening (NOTE: the term “life-threatening” refers to an event in which the subject was at immediate risk of death at the time of the event; it does not refer to an event which could hypothetically have caused a death had it been more severe).

Requires hospitalization or prolongation of existing hospitalization, except for elective or diagnostic procedures associated with pre-existing conditions that have not worsened, including the disease under study (e.g. study specified procedures and hospitalization).

Results in a persistent or significant incapacity.

Is a congenital anomaly/birth defect.

Is medically significant or requires intervention to prevent one or the outcomes listed above.

Medical and scientific judgment should be exercised in deciding if an AE is of significant enough medical importance to be classified as serious outside the above definitions. Important medical events that may not result in death, be life-threatening, or require hospitalization may be considered serious when, based upon appropriate medical judgment, they may jeopardize the subject and may require medical or surgical intervention to prevent one of the outcomes listed above. For example, drug overdose or abuse, a seizure that did not result in inpatient hospitalization, or intensive treatment of bronchospasm in an emergency department would typically be considered serious.

The study will comply with all local regulatory requirements and adhere to the full requirements of the ICH Guideline for Clinical Safety Data Management, Definitions and Standards for Expedited Reporting, Topic E2 and FDA Safety Reporting Requirements for INDs, 21 CFR 312.32.

8.4. Adverse Events of Special Interest (AESI)

AESIs are events that are of special interest with regards to CAR-T cell products and therefore have unique reporting requirements as described in Section 8.5. AESI for P-BCMA-101 currently include:

1. Any death, regardless of attribution, that occurs within 30 days of the infusion of P-BCMA-101.
2. Grade 4 or greater product infusion reactions.
3. Grade 4 or greater cytokine release syndrome.
4. Grade 4 or greater neurologic toxicity.
5. New malignancies.
6. New incidence or exacerbation of a pre-existing neurological disorder.
7. New incidence or exacerbation of a prior rheumatologic or other autoimmune disorder.
8. New incidence of hematologic disorder.

8.5. Regulatory Reporting Requirements for SAEs and AESIs

An SAE/AESI must be reported to the Sponsor or designee by emailing or faxing a completed SAE/AESI Report form within 24 hours of the study personnel's discovery of the event. The SAE/AESI Report form will be the primary source of data for safety reporting. Reporting procedures are described in the Study Reference Manual. Shortly following the submission of the SAE/AESI Report form, an eCRF entry within Medidata will be required for each event term. Data fields must match those reported on the SAE/AESI Report form.

8.6. Pregnancy

As described in the entry criteria, subjects must practice birth control from the time of Screening and throughout the study (both males and females of childbearing potential).

Females on cohorts R, RP or RIT must commit either to abstain continuously from sexual intercourse or to use two methods of reliable birth control, beginning 4 weeks prior to initiating treatment, during therapy, during dose interruptions and continuing for 4 weeks following discontinuation of lenalidomide and 12 months after last dose of rituximab.

Lenalidomide can cause embryo-fetal harm when administered to a pregnant female and is contraindicated during pregnancy based on the mechanism of action and findings. Lenalidomide is present in the semen of patients receiving the drug. Therefore, males in cohort R or RP must always use a latex or synthetic condom during any sexual contact with females of reproductive potential while taking lenalidomide and for up to 4 weeks after discontinuing lenalidomide, even if they have undergone a successful vasectomy. Male patients taking lenalidomide must not donate sperm.

There is no preclinical or clinical trial data of P-BCMA-101 or rimiducid in pregnant women; however, this overall treatment regimen could theoretically be embryotoxic. The effects on breast milk are unknown, therefore, breastfeeding should be discontinued for the duration of the study starting at Screening and for at least 12 months after receiving the last dose of investigational product (P-BCMA-101 or rimiducid, lenalidomide or rituximab), or four months after there is no evidence of persistence/gene modified cells in the subject's blood, whichever is longer.

Pregnancy (or pregnancy of a male subject's partner) is not considered an AE/SAE unless there is reason to believe the pregnancy may be the result of failure of a contraceptive due to interaction with the investigational product, or there is an adverse fetal outcome in the pregnancy. However, the Investigator shall report all pregnancies immediately to the Sponsor. A woman who becomes and remains pregnant during the study will be discontinued from the study and would enter into the LTFU study. The outcome of the pregnancy must also be reported to the Sponsor.

9. Safety Monitoring

9.1. Safety Committee

A Safety Committee comprised of the Investigators and a clinical representative of the sponsor will be established and will review data regularly for all subjects and following each cohort to determine dose escalation. The Safety Committee may recommend expansion of a cohort to further evaluate safety and, if DLT is observed in the first cohort, exploration of a lower dose during Phase 1 and continue to manage safety, stopping and pausing criteria during Phase 2.

9.2. Criteria for Pausing Dosing or Stopping the Study

If a study-defined DLT or any treatment-related death occurs, dosing of new subjects will be paused until the Safety Committee meets, reviews the event(s) and determines forward plans, which might include stopping the study, reducing subsequent dose levels, instituting additional safety procedures or a study amendment, continuing the study as planned or other measures as appropriate to the event. As previously described, if 2 or more of 6 subjects have DLTs in a cohort during Phase 1, and a >10% incidence of >Grade 4 or >30% incidence of >Grade 3 CRS or neurotoxicity at or below a corresponding dose level in Phase 2 with >10 patients treated, that dose level will have exceeded the MTD and any further dosing would take place at a lower dose level.

10. Statistical and Data Analysis

Phase 1 of this study is a standard 3+3 design of dose cohorts intended to determine a dose below which a 33% incidence of DLTs occurs.

Phase 2 of this study is an open-label, single dose, efficacy and safety evaluation. Details of the analyses for all endpoints will be provided in the Statistical Analysis Plan (SAP).

10.1. Study Populations

The Intent-to-Treat (ITT) population will include all subjects enrolled into the study.

The Safety population will include all subjects who receive P-BCMA-101 administration.

The Per Protocol population will include all subjects who receive the protocol directed dose of P-BCMA-101 (e.g., patients who receive less than the protocol directed dose would be analyzed as a separate subgroup).

10.2. Sample Size Calculation

The Phase 1 part of the study is a standard 3+3 design of dose cohorts intended to determine a dose below which a 33% incidence of DLTs occurs. Thus, up to 120 subjects may be enrolled to include the possibility of 18 cohorts of 6 subjects during dose escalation, cycle administration and combination administration as well as subjects who might be enrolled to replace those who discontinue prior to completion of the DLT evaluation period or further assess findings in a cohort.

For the Phase 2 part of the study, response rate endpoints will be tested to exclude a response rate of ≤30% as obtained with the recently approved standard of care agent daratumumab at p<0.05. With a 100-subject sample, the Phase 2 part of the study will have 90% power to detect a 15-percentage point improvement over a 30% response rate. This power calculation is based on an exact test for a binomial proportion with a 1-sided 0.05 significance level.

10.3. Statistical Methods

The demographic and baseline characteristics, safety, and efficacy data will be summarized using appropriate descriptive statistics. Data analyses will be provided by dose cohort, as well as for all subjects combined where appropriate. Descriptive statistics, including means, medians, standard deviations and ranges will be calculated for continuous variables, and categorical data will be summarized using counts and percentages. For response rate endpoints, point estimates and two-sided exact binomial 95% confidence intervals will be computed. Time-to-event variables will be summarized using the Kaplan-Meier method.

Treatment-emergent AEs (TEAEs) will be summarized using counts and percentages of subjects by cohort and for all subjects combined. TEAEs will also be summarized by severity and relationship. Concomitant medications will be summarized using counts and percentages of subjects by dose cohort.

Vital signs, ECG measurements and laboratory results will be summarized using descriptive statistics for observed values and change from baseline values by cohort. Laboratory results will also be summarized relative to the normal range (below, within, or above) by cohort.

Response rates will be determined by comparing each subject's best response after P-BCMA-101 administration to the corresponding baseline values per the International Myeloma Working Group Uniform Response Criteria (Appendix 15.2) (Rajkumar, 2011; Kumar, 2016; Cavo, 2017). The overall response rate (ORR) will be determined from subjects having received P-BCMA-101 and attaining a PR, very good partial response (VGPR), complete response (CR), or stringent complete response (sCR), over all subjects having received P-BCMA-101. The response rate for each individual response category will also be determined. Likewise, rates of stable disease (SD) at 8 weeks, and minimal response (MR) will also be determined. Time to response and duration of response will be determined for subjects who have response. OS, and PFS will also be determined for all subjects per the International Myeloma Working Group criteria.

A futility analysis will be conducted once 35 subjects are enrolled, received P-BCMA-101, and followed up for 4 months or progressed prior to 4-month follow-up. This analysis set is called Futility Analysis Set (FAS). The futility analysis will use Futility Index (FI) which is equal to 1 minus the Conditional Power (CP) based on the observed proportion of BOR in FAS. The study may be stopped if FI is above 0.80 (that is, if CP falls below 0.20).

Subjects who receive additional infusions of P-BCMA-101 T cells will be analyzed as separate subgroups for all outcomes.

11. Data Handling and Record Keeping

11.1. Confidentiality

Information about study subjects will be kept confidential and managed according to the requirements of the Health Insurance Portability and Accountability Act of 1996 (HIPAA) and any other applicable laws, regulation, and guidelines. Those regulations require a signed subject authorization informing the subject of the following:

What protected health information (PHI) will be collected from subjects in this study

Who will have access to that information and why

Who will use or disclose that information

The rights of a research subject to revoke their authorization for use of their PHI

In the event that a subject revokes authorization to collect or use PHI, the Investigator, by regulation, retains the ability to use all information collected prior to the revocation of subject authorization. For subjects that have revoked authorization to collect or use PHI, attempts should be made to obtain permission to collect at least vital status (i.e. that the subject is alive) at the end of their scheduled study period.

11.2. Data Management

An EDC system will be used to collect data pertaining to this trial. Trial data will be captured through an eCRF. Within the EDC system the eCRF data will be entered by the site staff and all source document verification and data cleaning will be performed by the Sponsor or designee (e.g. Contract Research Organization [CRO]).

The specifications for the EDC system will be documented and approved before the EDC system is released for live use. The validation of the eCRF data will be defined in a Data Management Plan. As data are entered into the eCRF, the validation checks will be performed, and where necessary, queries will be raised. All queries raised will be held in the EDC database.

The EDC system is a validated software program that has been designed to comply with CFR21 Part 11 requirements. All users will access the system via unique username and password. A full audit history of all actions performed within the system is maintained. User accounts ensure that each user can only perform the tasks applicable to their role and only have access to the data applicable to their role.

Standard coding dictionaries, World Health Organization (WHO) Drug and Medical Dictionary for Regulatory Activities (MedDRA) will be used to code medications, AEs and medical history.

When all data have been entered and all data cleaning is complete the data will be locked and made available for analysis and reporting.

On completion of the study, all eCRF data, including all associated queries and audit history, will be made available on a CD or USB drive to both the study Sponsor and the sites.

11.3. Source Documents

Source data is all information, original records of clinical findings, observations or other activities in a clinical trial necessary for the reconstruction and evaluation of the trial. Source data are contained in source documents. Examples of these original documents and data records include: hospital records, clinical and office charts, laboratory notes, memoranda, subjects' diaries or evaluation checklists, pharmacy dispensing records, recorded data from automated instruments, copies or transcriptions certified after verification as being accurate and complete, microfiches, photographic negatives, microfilm or magnetic media, x-rays, subject files, records kept at the pharmacy, at the laboratories and at medico-technical departments involved in the clinical trial, etc.

The Investigator must ensure the availability of source documents from which the information on the eCRF was derived.

The Investigator must permit authorized representatives of the Sponsor, the respective national, local or foreign regulatory authorities, the IRB/IEC and auditors to inspect facilities and to have direct access to the Investigator Site File and all source documents relevant to this study regardless of the type of media.

11.4. Case Report Forms

For each subject enrolled, the completed eCRF must be reviewed and signed by the Principal Investigator or authorized delegate. If a subject withdraws from the study, the reason must be noted on the eCRF.

The Investigator should ensure the accuracy, completeness, legibility and timeliness of the data reported to the Sponsor in the eCRFs and in all required reports.

11.5. Records Retention

It is the Investigator's responsibility to retain study essential documents for at least 2 years after the last approval of a marketing application in their country and until there are no pending or contemplated marketing applications in their country or at least 2 years have elapsed since the formal discontinuation of clinical development of the investigational product. These documents should be retained for a longer period if required by an agreement with the sponsor. In such an instance, it is the responsibility of the sponsor to inform the Investigator/institution as to when these documents no longer need to be retained.

12. Study Monitoring, Auditing, and Inspecting

12.1. Study Monitoring Plan

This study will be monitored according to a written monitoring plan. The Investigator will allocate adequate time for such monitoring activities. The Investigator will also ensure that the monitor or other compliance or quality assurance reviewer is given access to all the above noted study-related documents and study related facilities (e.g. pharmacy, diagnostic laboratory, etc.) and has adequate space to conduct the monitoring visit.

12.2. Audits and Inspections

The Investigator will permit study-related monitoring, audits, and inspections by the IRB/IEC, the sponsor, government regulatory bodies, and university compliance and quality assurance groups of all study related documents (e.g. source documents, regulatory documents, data collection instruments, study data etc.). The Investigator will ensure the capability for inspections of applicable study-related facilities (e.g. pharmacy, diagnostic laboratory, etc.). Participation as an Investigator in this study implies acceptance of potential inspection by government regulatory authorities and applicable university compliance and quality assurance offices.

15. Appendices

15.1. ECOG Performance status

TABLE 16
ECOG Performance status

| Grade | ECOG |
|---|---|
| 0 | Fully active, able to carry on all pre-disease |
| | performance without restriction |
| 1 | Restricted in physically strenuous activity but |
| | ambulatory and able to carry out work of a light or |
| | sedentary nature, e.g. light house work, office work |
| 2 | Ambulatory and capable of all self-care but unable to |
| | carry out any work activities. Up and about more than |
| | 50% of waking hours |
| 3 | Capable of only limited self-care, confined to bed |
| | or chair more than 50% of waking hours |
| 4 | Completely disabled. Cannot carry on any self-care. |
| | Totally confined to bed or chair |
| 5 | Dead |

15.2. IMWG Uniform Response Criteria

TABLE 17
IMWG uniform response criteria by response subcategory for multiple myeloma (Kumar, 2016)*

| | Stringent | | | | | |
|---|---|---|---|---|---|---|
| | complete | | | Minimal | | |
| | response | | | Response | | |
| CR | (sCR) | VGPR | PR | (MR) | SD | PD¶¶, ∥∥ |
| Negative | CR as defined, | Serum and urine | ≥50% reduction | ≥25% but ≤49% | Not meeting | Increase of 25% |
| immunofixation | plus | M-component | of serum M- | reduction of | criteria for CR, | from lowest |
| of serum and | | detectable by | protein and | serum M protein | VGPR, PR, MR | confirmed |
| urine, and | | immunofixation | reduction in 24- | or urine M- | orPD | response value |
| | | but not on | hour urinary M- | protein by | | in any of the |
| | | electrophoresis, | protein by ≥90% | 50%-89% | | following: |
| | | or | or to <200 | | | |
| | | | mg/24 hours | | | |
| Disappearance | Normal FLC | ≥90% reduction | If the serum and | In addition to | | Serum M- |
| of any soft | ratio** and | in serum M- | urine M-protein | the above | | protein (absolute |
| tissue | | component plus | are | criteria, if | | increase must be |
| plasmacytomas, | | urine M- | unmeasurable, a | present at | | ≥0.5 g/dL), |
| and | | component | decrease ≥50% | baseline, >50% | | and/or Serum |
| | | <100 mg/24 h | in the difference | reduction in the | | M-protein |
| | | | between | size (SPD)§§ of | | increase ≥1 |
| | | | involved and | soft tissue | | g/dL, if the |
| | | | uninvolved FLC | plasmacytomas | | lowest M |
| | | | levels is | is also required | | component was |
| | | | required in place | | | ≥5 g/dL |
| | | | of the M-protein | | | |
| | | | criteria | | | |
| <5% PCs in | Absence of | | If serum and | | | Urine M-protein |
| bone marrow | clonal PCs in | | urine M-protein | | | (absolute |
| aspirates | bone marrow | | are | | | increase must be |
| | biopsy by | | unmeasurable, | | | ≥200 mg/24 h), |
| | immunohisto- | | and serum free | | | and/or |
| | chemistry (κ/λ | | light assay is | | | |
| | ratio ≥4:1 or | | also | | | |
| | ≥1:2 for κ and λ | | unmeasurable, | | | |
| | patients, | | ≥50% reduction | | | |
| | respectively, | | in bone marrow | | | |
| | after counting | | PCs is required | | | |
| | ≥100 plasma | | in place of M- | | | |
| | cells)†† | | protein, | | | |
| | | | provided | | | |
| | | | baseline | | | |
| | | | percentage was | | | |
| | | | ≥30% | | | |
| | | | In addition to | | | Only in patients |
| | | | the above | | | without |
| | | | criteria, if | | | measurable |
| | | | present at | | | serum and urine |
| | | | baseline, >50% | | | M protein |
| | | | reduction in the | | | levels: the |
| | | | size (SPD)§§ of | | | difference |
| | | | soft tissue | | | between |
| | | | plasmacytomas | | | involved and |
| | | | is also required | | | uninvolved FLC |
| | | | | | | levels (absolute |
| | | | | | | increase must be |
| | | | | | | >10 mg/dL) |
| | | | | | | Only in patients |
| | | | | | | without |
| | | | | | | measurable |
| | | | | | | serum and urine |
| | | | | | | M protein levels |
| | | | | | | and without |
| | | | | | | measurable |
| | | | | | | disease by FLC |
| | | | | | | levels, bone |
| | | | | | | marrow PC |
| | | | | | | percentage |
| | | | | | | (absolute |
| | | | | | | percentage must |
| | | | | | | be ≥10%) |
| | | | | | | Appearance of a |
| | | | | | | new lesion(s), |
| | | | | | | ≥50% increase |
| | | | | | | from nadir in |
| | | | | | | SPD§§ of >1 |
| | | | | | | lesion, or ≥50% |
| | | | | | | increase in the |
| | | | | | | longest diameter |
| | | | | | | of a previous |
| | | | | | | lesion >1 cm in |
| | | | | | | short axis; ≥50% |
| | | | | | | increase in |
| | | | | | | circulating |
| | | | | | | plasma cells |
| | | | | | | (minimum of |
| | | | | | | 200 cells per |
| | | | | | | μL) if this is the |
| | | | | | | only measure of |
| | | | | | | disease |

IMWG = International Myeloma Working Group.
MRD = minimal residual disease.
FLC = free light chain.
M-protein = myeloma protein.
SPD = sum of the products of the maximal perpendicular diameters of measured lesions.
SUVₘₐₓ = maximum standardised uptake value.
′8F-FDG PET = ′8F-fluorodeoxyglucose PET.
*All response categories require two consecutive assessments made any time before starting any new therapy.
∥Derived from international uniform response criteria for multiple myeloma (see Durie et al., Leukemia 2006; 20: 1467-73).
When the only method to measure disease is by serum FLC levels: complete response canbe defined as a normal FLC ratio of 0.26 to 1.65 in addition to the complete response criteria listed previously.
Very good partial response in such patients requires a ≥90% decrease in the difference between involved and uninvolved FLC levels.
All response categories require two consecutive assessments made at any time before the institution of any new therapy; all categories also require no known evidence of progressive or new bone lesions or extramedullary plasmacytomas if radiographic studies were performed.
Radiographic studies are not required to satisfy these response requirements.
Bone marrow assessments do not need to be confirmed.
Each category, except for stable disease, will be considered unconfirmed until the confirmatory test is performed.
The date of the initial test is considered as the date of response for evaluation of time dependent outcomes such as duration of response.
**All recommendations regarding clinical uses relating to serum FLC levels or FLC ratio are based on results obtained with the validated Freelite test (Binding Site, Birmingham, UK).
††Presence/absence of clonal cells on immunohistochemistry is based upon the κ/λ/L ratio.
An abnormal κ/λ ratio by immunohistochemistry requires a minimum of 100 plasma cells for analysis.
An abnormal ratio reflecting presence of an abnormal clone is κ/λ of >4:1 or <1:2.
§§Plasmacytoma measurements should be taken from the CT portion of the PET/CT, or MRI scans, or dedicated CT scans where applicable.
For patients with only skin involvement, skin lesions should be measured with a ruler.
Measurement of tumour size will be determined by the SPD.
¶¶Positive immunofixation alone in a patient previously classified as achieving a complete response will not be considered progression.
For purposes of calculating time to progression and progression-free survival, patients who have achieved a complete response and are MRD-negative should be evaluated using criteria listed for progressive disease.
Criteria for relapse from a complete response or relapse from MRD should be used only when calculating disease-free survival.
In the case where a value is felt to be a spurious result per physician discretion (eg, a possible laboratory error), that value will not be considered when determining the lowest value.

### Example 5—Rimiducid and P-BCMA-101 Combination Treatment

15.3. Rimiducid

15.3.1. Introduction

Rimiducid (a.k.a. AP1903) (See also Rimiducid Investigator's Brochure) is an investigational small molecule drug that has been previously evaluated in Phase 1 human studies as the activation agent for cell therapies transduced with an inducible caspase 9 (iC9) safety switch gene. Rimiducid is a member of a class of compounds termed dimerizer drugs that act by inducing clustering of engineered proteins inside cells. Rimiducid-inducible cell death is achieved by expressing a chimeric protein comprising a human FK506-binding protein 12 (FKBP12) domain linked via a flexible linker to human delta caspase-9. Rimiducid is a cell-permeable synthetic ligand that binds to an engineered high-affinity version of FKBP12 that interacts minimally with endogenous FKBP. This chimeric protein is quiescent inside cells until administration of rimiducid, which cross-links the FKBP12 domains, initiating dimerization of the modified caspase-9 molecule, and results in cell apoptosis (Zhou, 2015a). A single intravenous infusion of rimiducid triggers apoptosis and eventual cell death in cells expressing the iC9 gene but does not affect non-transduced cells and has no therapeutic benefit on its own. The proposed use of rimiducid is in conjunction with P-BCMA-101 as a ‘rescue therapy’, if such selective cell killing is clinically indicated in case certain severe or life-threatening adverse events occur in a subject. P-BCMA-101 is a proprietary CAR-T product that consists of autologous T-cells that have been genetically altered to target and eliminate myeloma cancer cells expressing BCMA, and these T cells also carry an iC9-based safety switch gene. Rimiducid is administered as a single intravenous dose of 0.4 mg/kg. Rimiducid drug product is supplied as a sterile solution for injection that contains 40 mg of rimiducid in 8 mL of solution (5 mg/mL) and is then further diluted in 0.9% normal sterile saline for injection prior to infusion administration.

Nonclinical studies with rimiducid demonstrated that primary human T lymphocytes transduced with the rimiducid-based safety switch; (1) retain their function, and (2) can be eliminated by exposure to rimiducid with high efficiency, potency, and specificity (Thomis, 2001). In vitro and in vivo studies have been conducted by Poseida with rimiducid and the iC9-based safety switch-containing CAR-T cell product P-BCMA-101 with similar results. A GLP repeat dose intravenous toxicity study in rats has recently been conducted by Poseida. The results showed a NOAEL of >15 mg/kg, after repeated (three times) intravenous administration. No noticeable toxic effect of rimiducid was observed. (see Rimiducid Investigator's Brochure for further detail).

Rimiducid has previously been evaluated in phase 1 clinical studies, including healthy volunteers, and for Graft versus Host Disease (GVHD) in transplant patients treated with other genetically modified T cell products carrying an iC9-based safety switch gene, which support the potential for clinical use of rimiducid in conjunction with P-BCMA-101. In clinical studies published to date with rimiducid, there were no significant adverse events and no clinically significant changes in vital signs, ECGs, serum biochemistry, hematology, coagulation parameters, or urinalysis results. In subjects who developed GVHD and received 0.4 mg/kg of rimiducid as a 2-hour infusion, 90% of the modified T cells were eliminated within 30 minutes following rimiducid infusion, with a further log depletion during the next 24 hours. GVHD completely resolved in these patients without recurrence (Zhou, 2015b; Iuliucci, 2001; Di Stasi, 2011; Kapoor, 2016; Zhou, 2016).

The risk benefit of rimiducid for its intended use appears to be highly favorable. Rimiducid appears to have consistent and linear pharmacokinetics with effectively no toxicity, within and above the effective dose range (0.01-1.0 mg/kg). This balances extraordinarily well with clinical data demonstrating elimination of a serious disease (GVHD) elicited by iC9-based safety switch-transduced allogeneic T-cells, and the expectation of similar elimination of autologous CAR-T cells carrying the iC9-based safety switch gene, if clinically required.

15.3.2. Investigational Use

Rimiducid may be used for subjects that experience significant adverse reactions to P-BCMA-101 as described in Section 6.3 and the Study Reference Manual (Toxicity Reference Manual). Generally, subjects with Grade 4 CRS may be treated with rimiducid in addition to other standard measures (e.g. tocilizumab, steroids and/or cytotoxic/immunosuppressive agents) (generally, use of rimiducid would be prioritized over use of a systemically toxic cytotoxic agent). This option may also be considered for Grade 3 toxicity unresponsive to other measures. Rimiducid use would also be considered in addition to other standard measures if subject develops uncontrollable P-BCMA-101 T cell expansion or other clinically significant Grade 3-4 toxicities possibly related to P-BCMA-101. It is recommended the Investigator review the clinical scenario and potential confounding factors before administration. The study medical monitor should be consulted if time permits and a rimiducid use form completed. There are no absolute predefined inclusion or exclusion criteria.

15.3.3. Study Treatments

15.3.3.1. Rimiducid Administration

15.3.3.1.1. Description

The dosage form for the rimiducid drug product is a sterile solution for injection. The drug product contains 40 mg of rimiducid and is intended to be further diluted in 0.9% normal sterile saline for injection prior to administration by intravenous infusion.

The sterile rimiducid solution, 5 mg/mL, is provided as an 8 ml fill in a Type 1 glass 10 mL vial, with a gray butyl stopper and aluminum overseal. The quantitative composition of rimiducid injection, 5 mg/mL is provided in Table 5.

TABLE 5
Qualitative and Quantitative Composition
of Rimiducid Drug Product

| | Quality | | Quantity/Unit |
|---|---|---|---|
| Component | Reference | Function | Dose (mg/vial) |
| Rimiducid | In-house | Active Ingredient | 40.0a |
| Polyoxyl 15 | USP | Surfactant | 2000 |
| hydroxystearateb | | | |
| Water for Injection | USP | Diluent | QS to 8 mL |

aThe actual quantity will be adjusted based on the purity of the drug substance.
bAlso referred to as Kollilphor^{R} HS 15 or solutol HS 15.

15.3.3.1.2. Supply and Storage

The drug product should be stored refrigerated at 2-8° C. Prior to dilution and administration, the drug product should be brought to room temperature and mixed several times by inversion to ensure a clear and homogenous solution, in accordance with protocol directions below.

15.3.3.1.3. Preparation

1. Calculate the dose volume of Rimiducid Injection to be used based on subject weight using a dose of 0.4 mg/kg and a concentration of rimiducid injection of 5 mg/mL—Volume (mL)=0.4 mg/kg×subject weight in kg/5 mg/mL (examples are shown in Table 6). Each vial contains 8 mL of rimiducid, so more than 1 vial may be required.
2. Bring vial(s) to ambient room temperature. Each vial should be mixed by inversion to ensure a clear and homogeneous solution prior to use. Check visually for any visible particulates or cloudiness. Aseptically transfer the calculated amount of Rimiducid Injection from the vial using a sterile needle and a sterile 10 mL B.D. Luer-Lok syringe into a commercially available 100 mL EVA DEHP-free infusion bag containing 0.9% sodium chloride injection, USP with tubing. Mix by inversion.
3. Administer at an infusion rate of ˜50 mL/hour.

TABLE 6
Rimiducid Dosing Calculation Examples

| | |
|---|---|
| | Dose Volume - Amount of |
| Subject Weight | Rimiducid (5 mg/mL) Required |

| | | | |
|---|---|---|---|
| 60 | kg | 5 | mL |
| 75 | kg | 6 | mL |
| 100 | kg | 8 | mL |
| 120 | kg | 10 | mL |

15.3.3.1.4. Dosing and Administration

If indicated, rimiducid should be administered at a dose of 0.4 mg/kg as a ˜2-hour intravenous infusion.

15.3.4. Concomitant Medications and Treatment

There are no limitations or requirements for concomitant medications and treatments with rimiducid.

15.3.5. Schedule of Assessments and Procedures

The schedule of events and procedures to be followed in case of the use of rimiducid is shown in Table 7.

In the event of the occurrence of a specific AE as described in Section 6.3, the use of rimiducid should be considered. The rimiducid use form is to be completed and, if time permits, reviewed and approved by the Medical Monitor. The consent for rimiducid will be included in the main study ICF.

Blood samples for P-BCMA-101 T cells will be collected prior to rimiducid infusion, then at 1, 2, 4, 8, 24 hours; Days 2, 4, 7; Weeks, 2, 3, 4, and for rimiducid pharmacokinetics at 1, 2, 4, 8 and 24 hours. Clinical chemistry, hematology and coagulation laboratory evaluations will be performed at Days 1, 2, 4, 7; and Weeks 2, 3, 4. Visits may be adjusted within the proscribed windows to coincide with P-BCMA-101 required study visits and laboratories assessments conducted for the P-BCMA-101 Schedule of Assessments do not need to be duplicated for the rimiducid Schedule of Assessments.

Follow-up will continue consistent with the P-BCMA-101 Schedule of Events (Table 7), with visits to occur at Weeks 6, 8, Month 3, and every 3 months thereafter.

15.3.6. Recording Adverse Events

See Section 8 for procedures for recording adverse events. Adverse events are to be recorded on study CRFs and marked with attribution assigned to rimiducid if appropriate.

15.4. Retreatment with P-BCMA-101 If sufficient P-BCMA-101 cells remain from manufacturing when a subject's disease progresses, with Safety Committee approval additional cells may be administered up to the highest dose level that has successfully completed dose-limiting toxicity assessment. In order to receive an additional P-BCMA-101 cell infusion, subjects will be assigned a new subject identification number, they will have to meet all eligibility criteria as outlined in Section 4, and will undergo the same screening, enrollment, conditioning chemotherapy, and follow-up procedures except for leukapheresis, as outlined in Table 8 and Table 9.

15.5. Cycle Administration

During Phase 1—Cycle Administration, multiple doses of P-BCMA-101 will be administered intravenously in 2 cycles (Cohort A and Cohort C) or 3 cycles (Cohort B) of 2 weeks. The total dose administered may start at ≤the MTD as determined during Phase 1 single dose escalation.

In the first cycle for both cohorts A and B, ⅓ the total dose will be administered. In Cohort A up to ⅔ the total dose will be administered in the 2nd cycle. In Cohort B up to ⅓ the total dose will be administered in each of the 2nd and 3rd cycles. In Cohort C up to ⅔ the total dose will be administered in the 1st cycle and up to ⅓ the total dose will be administered in the 2nd cycle. Schematics of the study design are shown in FIG. 16 for Cohort A and Cohort C, and FIG. 17 for Cohort B.

The same 3+3 dose escalation and/or de-escalation rules described for single administration will be utilized. For example, if the current maximum dose for single administration is 15×10⁶ P-BCMA-101 cells/kg, Cohort A 15×10⁶ P-BCMA-101 cells/kg will consist of an infusion of 5×10⁶ P-BCMA-101 cells/kg followed in 2 weeks by an infusion of up to 10×10⁶ P-BCMA-101 cells/kg, and when the results of this cohort or a single administration cohort at 20×10⁶ P-BCMA-101 cells/kg meet the criteria for escalation, Cohort A 20×10⁶ P-BCMA-101 cells/kg may be initiated.

The Schedule of Events for Screening through conditioning chemotherapy for Cohorts A, B, and C is shown in Table 10. The Schedule of Events for P-BCMA-101 administration for cycle dosing is shown in Table 11 for Cohort A and Cohort C, and Table 12 for Cohort B. The Schedule of Events for post-treatment follow-up for Cohorts A, B, and C is shown in Table 13.

TABLE 7
Schedule of Events (Rimiducid)
(USED ONLY IN CASE OF SPECIFIC ADVERSE EVENTS AS DESCRIBED IN SECTION 6.3 AND STUDY REFERENCE MANUAL)

| | Pre- | Day 0 | | | | | | | | |
|---|---|---|---|---|---|---|---|---|---|---|
| | Rimiducid | Rimiducid | | | | | Week 2 | Week 3 | Week 4 | Follow- |
| Procedures | Infusion | Infusion | Day 1 | Day 2 | Day 4 | Day 7 | (±2 days) | (±2 days) | (±7 days) | Up⁵ |
| AE confirmed¹ | X | | | | | | | | | |
| Rimiducid use form completed | X | | | | | | | | | |
| Rimiducid use form reviewed by Medical | X | | | | | | | | | |
| Monitor | | | | | | | | | | |
| Informed Consent for rimiducid² | | X | | | | | | | | |
| Rimiducid Infusion | | X | | | | | | | | |
| Blood sample for P-BCMA-101 T cells^{3,5} | X | X | X | X | X | X | X | X | X | X |
| Blood sample for rimiducid3,5 | X | X | X | | | | | | | |
| Chemistry Panel, Hematology including B | | | X | X | X | X | X | X | X | X |
| and T cell counts, Coagulation^{3,4,5} | | | | | | | | | | |
| Rimiducid AEs^{5,6} | | X | X | X | X | X | X | X | X | X |

¹Confirmation of an AE consistent with Section 6.3 and the Study Reference Manual (Toxicity Management Manual), and review with Medical Monitor before first rimiducid administration if time permits.
²Consent for rimiducid to be included in main study ICF and should be completed by the subject as soon as possible following IRB approval.
³Blood sample for P-BCMA-101 T cells to be collected pre-rimiducid infusion, at 1, 2, 4, 8, 24 hours post-rimiducid infusion, then at Days 2, 4, 7, Weeks, 2, 3, 4. Blood sample for rimiducid pharmacokinetics to be collected pre-rimiducid infusion and at 1, 2, 4, 8 and 24 hours post-rimiducid infusion.
⁴Blood chemistries will include sodium, potassium, chloride, magnesium, bicarbonate, blood urea nitrogen, creatinine, calcium, albumin, glucose, LDH, total protein, ALT, AST, bilirubin (total and direct), alkaline phosphatase, PTT and PT or INR.
Hematological studies include:
Complete blood count
Platelets
B (CD19) and T cell counts (CD3) and CD4 and CD8
⁵Continued follow-up consistent with P-BCMA-101 Schedule of Events; visits to occur at Weeks 6, 8, Month 3, and every 3 months thereafter. If visit coincides with visits on the P-BCMA-101 Schedule of Events, they need not be repeated if within the assigned visit windows.
⁶AE(s) to be recorded in study eCRF and marked with attribution assigned to rimiducid if appropriate.

TABLE 8
Schedule of Events-Additional Infusion: Screening/Enrollment through Conditioning Chemotherapy

| | Screening/ | | | | | | | |
|---|---|---|---|---|---|---|---|---|
| | Enrollment | Baseline | Baseline | | | | | |
| | (within 28 days | (within 7 days | (within 72 hrs | Conditioning | | | | |
| | prior to | prior to | prior to | Chemotherapy¹⁰ | | | | |
| | P-BCMA-101 | Conditioning | Conditioning | Days | | | | |
| Procedures | Administration) | Chemotherapy) | Chemotherapy)¹⁰ | −5 | −4 | −3 | −2 | −1 |
| Informed Consent | X | | | | | | | |
| Inclusion/Exclusion Criteria | X | | | X | | | | |
| Medical History and Disease Assessment¹ | X | | | | | | | |
| ECOG Performance Status | X | | | X | | | | |
| Mini Mental Status Exam (MMSE) | X | | X | X | | | | |
| Height and Weight | X | | | | | | | |
| 12-Lead ECG² | X | | | | | | | |
| Echocardiogram | X | | | | | | | |
| Archival Tumor to Central Lab | X | | | | | | | |
| Samples for Myeloma Response³,¹¹ | X | X³ | | | | | | |
| Fresh Sample of Bone Marrow and Tumor³,¹¹ | X³ | X³ | | | | | | |
| Blood Sample for BCMA and Other Biomarkers¹¹ | X | | | | | | | |
| Physical Exam (Including Neurological Exam) | X | | X | X | | | | |
| Vital Signs (BP, HR, RR, O2 sat, temp) | X | | X | X | X | X | | |
| Pregnancy Test (serum or urine)⁴ | X | | X | | | | | |
| Blood Sample for HIV, Hepatitis B and C, | X | | | | | | | |
| and HTLV Screening¹¹ | | | | | | | | |
| Chemistry Panel, Hematology including B and | X | | X | | X | X | | |
| T cell counts, Coagulation⁵ | | | | | | | | |
| Assessment of circulating myeloma/plasma cells⁵ | X | | X | | | | | |
| Fludarabine/Cyclophosphamide | | | | X | X | X | | |
| Blood Sample for P-BCMA-101 T cells⁶,¹¹ | X | | | | | | | |
| Blood Sample for Serum IgG¹¹ | X | | | | | | | |
| Blood Sample for Immunogenicity Assays¹¹ | X | | | | | | | |
| Blood Sample for Cytokine Release Syndrome | X | | | | | | | |
| markers (CRS)⁷: e.g. CRP, Ferritin, Cytokines | | | | | | | | |
| IL-6, IL-2, IL-15, TNF-α, andlFN-γ⁷,¹¹ | | | | | | | | |
| PET/CT⁸ | | X⁸ | | | | | | |
| Adverse Events | X⁹ | X | X | X | X | X | | |
| Concomitant Medication⁹ | X | X | X | X | X | X | | |

¹Including MM measurement results for the 6 months prior to Enrollment required (including serum protein electrophoresis [SPEP], urine protein electrophoresis [UPEP], serum immunofixation [SIFE], urine immunofixation [UIFE], serum free light chains [FLC], MRD and as clinically indicated, PET/CT and/or bone marrow/tumor biopsy/aspirate [Bone marrow biopsy], if available).
²12-Lead ECG and echocardiogram obtained at Screening/Enrollment.
³Myeloma Response will be assessed for Screening/Enrollment (may be obtained from any result within the last month), baseline within 7 days of initiating conditioning chemotherapy (note: 2 or more assessments must be performed between completion of the last myeloma therapy, including salvage therapy, and initiation of lymphodepleting chemotherapy), Days 0 (before P-BCMA-101 administration-thus the 24-hr urine collection is to be started on Day −1 so as to be completed before P-BCMA-101 administration), and as clinically indicated per the International Myeloma Working Group criteria and based on the standard assessments, including SPEP, UPEP, SIFE, UIFE, and serum FLC. Bone marrow biopsy, aspirate and minimal residual disease (MRD) (analyzed by central lab) must be done within 7 days of initiating conditioning chemotherapy, then as clinically indicated, or have a medical monitor exemption. A sample of bone marrow aspirate and a sample of bone marrow biopsy are to be submitted (as described in the Study Reference Manual) for assessment of MRD, plasma cell frequency, P-BCMA-101 cells, BCMA, and other biomarkers. A portion of any extramedullary biopsy should also be submitted for assessment of P-BCMA-101 cells, BCMA, and other biomarkers. If a new bone marrow biopsy/aspirate was performed and provided during screening, this does not need to be repeated during the Baseline visit. PET/CT is to be conducted as clinically indicated
⁴Females of childbearing potential will have a serum pregnancy test at Enrollment and a urine pregnancy test within 72 hours (3 days) prior to initiating conditioning chemotherapy.
⁵Blood chemistries will include sodium, potassium, chloride, magnesium, bicarbonate, blood urea nitrogen, creatinine, calcium, albumin, glucose, LDH, total protein, ALT, AST, bilirubin (total and direct), alkaline phosphatase, PTT and PT or INR.
Hematological studies include:
Complete blood count
Platelets
B (CD19) and T cell counts (CD3) and CD4 and CD8 at all timepoints except Days −3, −4
Assessment of circulating myeloma/plasma cells (e.g. by flow cytometry or CBC with manual differential) is required at timepoints prior to P-BCMA-101 administration. Contact the sponsor and refer to exclusion criteria #3 and #10 if circulating myeloma/plasma cells are identified in the Enrollment & Baseline sample.
⁶P-BCMA-101 cells will be assessed; e.g. number of vector copies/mL of blood of P-BCMA-101 cells, surface marker phenotype, clonality, etc. at Enrollment. The cellular characterization and phenotyping assessments conducted on the sample at each time point will vary depending on the temporal relevance of each assay.
⁷CRS will be graded using Lee criteria (Lee, 2014).
⁸PET/CT to be obtained at Baseline within 7 days of initiating conditioning chemotherapy.
⁹Allergies and all prescription and nonprescription medication, vitamins, herbal and nutritional supplements, taken by the subject during the 30 days prior to Enrollment will be recorded at the Enrollment Visit. All medications, supplements and allergies (including severity of any current symptoms) should be recorded at each visit. Unnecessary medications/supplements should be discontinued prior to enrollment, if possible, at the investigator’s judgement.
¹⁰Subjects should continue to meet entry criteria at the time of initiation of conditioning chemotherapy or have medical monitor approval. The following assessments should be repeated within 72 hours of Day −5 to re-assess entry criteria: MMSE (should include sample of handwriting by the subject); physical exam; vital signs; pregnancy; and chemistry panel, hematology including B and T cell counts, coagulation.
¹¹Laboratory studies labeled as “sample for/of” or “blood sample for” are sent to a central laboratory, whereas those not so labeled are generally conducted at the investigational site. Subjects enrolled prior to the approval of P-BCMA-101-001 A3 (amendment 3) will continue to have assessments for myeloma response; HIV, Hepatitis B and C, andHTLV Screening; Serum IgG; and Cytokine Release Syndrome markers (CRS) conducted at the investigational site.

TABLE 9
Schedule of Events-Additional Infusion: P-BCMA-101 Administration and Follow-Up

| | | | | | | Post-Treatment Follow-up | | | | |
|---|---|---|---|---|---|---|---|---|---|---|
| | P-BCMA-101 | | | | | Visits (visit windows) relative to Day 0⁸ | | | | |
| | Administration ⁸ | | | | | Week | | Week | | q3 months |
| | Days | | | | | 2 & 3 | Week 4 | 6 & 8 | Month 3 | (± 2 weeks) |
| Procedures | 0⁷ | 1 | 4 | 7 | 10⁹ | (±2 days) | (±7 days) | (±7 days) | (±2 weeks) | to Month 24 |
| Mini Mental Status Exam (MMSE) | X | X | X | X | X | X | X | X | | X |
| 12-Lead ECG¹ | X | X | X | X | | | X | | X | X |
| Samples for Myeloma Response^{2,11} | X² | | | | | X | X | X | Mo 3, 4, 5 | +Mo 6, 7, 8, 9 |
| Fresh Sample of Bone Marrow and Tumor^{2,11} | | | | | | | X | | X | Mo 6, 12 |
| Blood Sample for BCMA and Other Biomarkers¹¹ | X | | | | | X | X | X | X | X |
| Physical Exam (Including Neurological Exam) | X | X | X | X | X | X | X | X | X | X |
| Vital Signs (BP, HR, RR, O2 sat, temp) | X | X | X | X | X | X | X | X | X | X |
| Pregnancy Test (urine)³ | | | | | | X | X | X | X | X |
| Chemistry Panel, Hematology including B and | X | X | X | X | X | X | X | X | X | X |
| T cell counts, Coagulation⁴ | | | | | | | | | | |
| P-BCMA-101 Cell Infusion (IV) | X | | | | | | | | | |
| Blood Sample for P-BCMA-101 T cells^{5,11} | X⁵ | | X | X | X | X | X | X | Mo 3, 4, 5 | +Mo 6, 7, 8, 9 |
| Blood Sample for Serum IgG¹¹ | X | | | | | | X | X | X | X |
| Blood Sample for Immunogenicity Assays¹¹ | | | | | | X | X | | X | X |
| Blood Sample for Cytokine Release Syndrome | X | X | X | X | X | X | X | X | Mo 3, 4, 5 | X |
| markers (CRS)⁶ e.g. CRP, Ferritin, Cytokines | | | | | | | | | | |
| IL-6, IL-2, IL-15, TNF-α, and IFN-γ^{6,11} | | | | | | | | | | |
| PET/CT¹² | | | | | | | | (X¹²) | (X¹²) | (X¹²) |
| Adverse Events | X | X | X | X | X | X | X | X | X | X |
| Concomitant Medication¹⁰ | X | X | X | X | X | X | X | X | X | X |

¹12-Lead ECG conducted at Days 0, 1, 4, 7, Week 4, Month 3 and every 3 months thereafter.
²Myeloma Response will be assessed at Days 0 (before P-BCMA-101 administration-thus the 24-hr urine collection is to be started on Day −1 so as to be completed before P-BCMA-101 administration), Weeks 2, 3, 4, 6, 8 and Months 3, 4, 5, 6, 7, 8, 9, 12,15, 18, 21 and 24 and as clinically indicated (e.g. ≤1 week after an assessment demonstrating a response for confirmation) per the International Myeloma Working Group criteria and based on the standard assessments, including serum protein electrophoresis (SPEP), urine protein electrophoresis (UPEP), serum immunofixation (SIFE), urine immunofixation (UIFE), and serum free hght chains (FLC). Bone marrow biopsy, aspirate and minimal residual disease (MRD) (analyzed by central labs) must be done at Week 4, Months 3, 6 and 12 then as clinically indicated, or have a medical monitor exemption. A sample of bone marrow aspirate and a sample of bone marrow biopsy are to be submitted (as described in the Study Reference Manual) for assessment of MRD, plasma cell frequency, P-BCMA-101 cells, BCMA, and other biomarkers. A portion of any extramedullary biopsy should also be submitted for assessment of P-BCMA-101 cells, BCMA, and other biomarkers. PET/CT is to be conducted as clinically indicated.
³Females of childbearing potential will have a urine pregnancy test at all indicated visits after Screening.
⁴Blood chemistries will include sodium, potassium, chloride, magnesium, bicarbonate, blood urea nitrogen, creatinine, calcium, albumin, glucose, LDH, total protein, ALT, AST, bilirubin (total and direct), alkaline phosphatase, PTT and PT or INR. Assessments for tumor lysis (uric acid and phosphate) will be performed at baseline and Days 1,4,7, then as clinically indicated.
Hematological studies include:
Complete blood count
Platelets
B (CD 19) and T cell counts (CD3) and CD4 and CD8
⁵P-BCMA-101 cells will be assessed; e.g. number of vector copies/mL of blood of P-BCMA-101 cells, surface marker phenotype, clonality, etc. At Baseline, Days 0 (1 hour after P-BCMA-101 administration), 4, 7, 10, Weeks, 2, 3, 4, 6, 8, Month 3, 4, 5, 6, 7, 8, 9 and every 3 months thereafter. The cellular characterization and phenotyping assessments conducted on the sample at each time point will vary depending on the temporal relevance of each assay.
⁶CRS will be graded using Lee criteria (Lee, 2014).
⁷On Day 0 MMSE (should include sample of handwriting by the subject), 12-Lead ECG, physical exam, chemistry panel, hematology and coagulation should be obtained before and approximately 1 hour after P-BCMA-101 administration (+/−15 minutes). Vital signs (temperature, respiration rate, pulse, O2 saturation and blood pressure) will be taken before and after P-BCMA-101 administration, then every 15 minutes (+/−5 minutes) for at least one hour and until these signs are satisfactory and stable. Other Day 0 assessments and samples should be obtained before P-BCMA-101 administration unless otherwise indicated. After administration of P-BCMA-101 these assessments (or any other assessments deemed clinically indicated by the Investigator) will be conducted as described in Table 9 and as often as clinically indicated by the adverse events observed or by the institution’s inpatient standards.
⁸If a subject discontinues from this study after P-BCMA-101 administration, the events for the next visit scheduled should be performed prior to initiating alternative medical, radiation or surgical intervention and recorded as an end-of-study visit for this study. If the subject is enrolling into Protocol P-BCMA-101-002, the subject should be consented and enrolled into that protocol following the subject discontinuing from Protocol P-BCMA-101-001 and the events for the first visit of Protocol P-BCMA-101-002 performed and recorded per the schedule prescribed by that protocol relative to the administration of P-BCMA-101 on this protocol.
⁹Day 10 assessments may be performed up to 2 days after Day 10 if needed, but not on the same day as week 2 assessments.
¹⁰All medications, supplements and allergies (including severity of any current symptoms) should be recorded at each visit.
¹¹Laboratory studies labeled as “sample for/of” or “blood sample for” are sent to a central laboratory, whereas those not so labeled are generally conducted at the investigational site. Subjects enrolled prior to the approval of P-BCMA-101-001 A3 (amendment 3) will continue to have assessments for myeloma response; HIV, Hepatitis B and C, and HTLV Screening; Serum IgG; and Cytokine Release Syndrome markers (CRS) conducted at the investigational site.
¹²PET/CT to be obtained at Baseline within 7 days of initiating conditioning chemotherapy, then as clinically indicated (e.g. every 8 weeks if soft tissue plasmacytomas are found at baseline that require imaging as part of response assessments or to confirm response).

TABLE 10
Schedule of Events-Screening through Conditioning Chemotherapy: Cycle Administration, Cohorts A, B, and C

| | | | | | Baseline | | | | | |
|---|---|---|---|---|---|---|---|---|---|---|
| | Screening | Enrollments | | Baseline (within | (within 72 | Conditioning | | | | |
| | (within 28 days | (2 weeks ± 3 | | 7 days prior to | hours prior to | Chemotherapy11 | | | | |
| | prior to | days prior to | Leuka- | Conditioning | Conditioning | Days | | | | |
| Procedures | leukapheresis) | leukapheresis) | pheresis¹³ | Chemotherapy) | Chemotherapy) | −5 | −4 | −3 | −2 | −1 |
| Informed Consent | X | | | | | | | | | |
| Inclusion/Exclusion Criteria | X | | | | | X | | | | |
| Medical History and Disease Assessment¹ | X | | | | | | | | | |
| ECOG Performance Status | X | | | | | X | | | | |
| Mini Mental Status Exam (MMSE) | X | | | | X | X | | | | |
| Height and Weight² | X | X¹² | X | | | | | | | |
| 12-Lead ECG³ | X | | | | | | | | | |
| Echocardiogram | X | | | | | | | | | |
| Archival Tumor to Central Lab | X | | | | | | | | | |
| Samples for Myeloma Response^{4,14} | X | | | X⁴ | | | | | | |
| Fresh Sample of Bone Marrow and Tumor^{4,14} | X⁴ | | | X⁴ | | | | | | |
| Blood Sample for BCMA and Other | | X¹² | | | | | | | | |
| Biomarkers¹⁴ | | | | | | | | | | |
| Physical Exam (Including Neurological | X | | | | X | X | | | | |
| Exam) | | | | | | | | | | |
| Vital Signs (BP, HR, RR, O2 sat, temp) | X | | | | X | X | X | X | | |
| Pregnancy Test (serum or urine)⁵ | X | | | | X | | | | | |
| Blood Sample for HIV, Hepatitis B and C, | X | | | | | | | | | |
| and HTLV Screening¹⁴ | | | | | | | | | | |
| Chemistry Panel, Hematology including B | X | X | | | X | | X | X | | |
| and T cell counts, Coagulation⁶ | | | | | | | | | | |
| Assessment of circulating myeloma/plasma | X | X | | | X | | | | | |
| cells⁶ | | | | | | | | | | |
| Leukapheresis | | | X¹³ | | | | | | | |
| Fludarabine/Cyclophosphamide | | | | | | X | X | X | | |
| Blood Sample for P-BCMA-101 T cells^{7,14} | | X¹² | | | | | | | | |
| Blood Sample for Serum IgG¹⁴ | | X¹² | | | | | | | | |
| Blood Sample for Immunogenicity Assays¹⁴ | | X¹² | | | | | | | | |
| Blood Sample for Cytokine Release | | X¹² | | | | | | | | |
| Syndrome markers (CRS)8: e.g. CRP, | | | | | | | | | | |
| Ferritin, Cytokines IL-6, IL-2, IL-15, | | | | | | | | | | |
| TNF-α, and IFN-γ^{8,14} | | | | | | | | | | |
| PET/CT^{4,9} | | | | X^{4,9} | | | | | | |
| Adverse Events | | X | X | X | X | X | X | X | | |
| Concomitant Medication¹⁰ | X¹⁰ | X¹² | X | X | X | X | X | X | | |

¹Including MM measurement results for the 6 months prior to Screening required (including serum protein electrophoresis SPEP], urine protein electrophoresis [UPEP], serum immunofixation [SIFE], urine immunofixation [UIFE], serum free light chains [FLC], minimal residual disease [MRD] and as clinically indicated, PET/CT and/or bone marrow/tumor biopsy/aspirate [Bone marrow biopsy], if available).
²Height obtained at Screening Visit only
³12-Lead ECG and echocardiogram obtained at Screening.
⁴Myeloma Response will be assessed for Screening (may be obtained from any result within the last month), baseline within 7 days of initiating conditioning chemotherapy) note: 2 or more assessments must be performed between completion of the last myeloma therapy, including salvage therapy, and initiation of lymphodepleting chemotherapy, Days 0 (before P-BCMA-101 administration-thus the 24-hr urine collection is to be started on Day −1 so as to be completed before P-BCMA-101 administration), and as clinically indicated per the International Myeloma Working Group criteria and based on the standard assessments, including SPEP, UPEP, SIFE, UIFE, and serum FLC. Bone marrow biopsy, aspirate and MRD (analyzed by central lab) must be done within 7 days of initiating conditioning chemotherapy, then as clinically indicated, or have a medical monitor exemption. A sample of bone marrow aspirate and a sample of bone marrow biopsy are to be submitted (as described in the Study Reference Manual) for assessment of MRD, plasma cell frequency, P-BCMA-101 cells, BCMA, and other biomarkers. A portion of any extramedullary biopsy should also be submitted for assessment of P-BCMA-101 cells, BCMA, and other biomarkers. If a new bone marrow biopsy/aspirate was performed and provided during screening, this does not need to be repeated during the Baseline visit. PET/CT is to be conducted as clinically indicated.
⁵Females of childbearing potential will have a serum pregnancy test at Screening, and a urine pregnancy test within 72 hours (3 days) priot to initating conditioning chemotherapy.
⁶Blood chemistries will include sodium, potassium, chloride, magnesium, bicarbonate, blood urea nitrogen, creatinine, calcium, albumin, glucose, LDH, total protein, ALT, AST, bilirubin (total and direct), alkaline phosphatase, PTT and PT or INR.
Hematological studies include:
Complete blood count
Platelets
B (CD19) and T cell counts (CD3) and CD4 and CD8 at all timepoints except Days −3, −4
Assessment of circulating myeloma/plasma cells (e.g. by flow cytometry or CBC with manual differential) is required at timepoints priot to P-BCMA-101 administration. Contact the sponsor and refer to exclusion criteria #3 and #10 if circulating myeloma/plasma cells are identified in the Enrollment & Baseline sample.
⁷P-BCMA-101 cells will be assessed; e.g. number of vector copies/mL of blood of P-BCMA-101 cells, surface marker phenotype, clonality, etc. at Enrollment. The cellular characterization and phenotyping assessments conducted on the sample at each time point will vary depending on the temporal relevance of each assay.
⁸CRS will be graded using Lee criteria (Lee, 2014).
⁹PET/CT to be obtained at Baseline within 7 days of initiating conditioning chemotherapy.
¹⁰Allergies and all prescription and nonprescription medication, vitamins, herbal and nutritional supplements, taken by the subject during the 30 days prior to Screening will be recorded at the Screening Visit. All medications, supplements and allergies (including severity of any current symptoms) should be recorded at each visit. Unneccessary medications/supplements should be discontinued prior to Screening, if possible, at the investigator’s judgement.
¹¹Subjects should continue to meet entry criteria at the time of initiation of conditioning chemotherapy or have medical monitor approval. The following assessments should be repeated within 72 hours of Day −5 to re-assess entry criteria: MMSE (should include sample of handwriting by the subject); physical exam; vital signs; pregnancy; and chemistry panel, hematology including B and T cell counts, coagulation.
¹²Enrollment assessments are to be conducted 14 days ± 3 days prior to leukapheresis or have medical monitor approval.
¹³Leukapheresis should take place within~28 days following screening or have medical monitor approval. Guidance for conducting leukapheresis may be found in Section 5.1 and the Study Reference Manual (in particular characterization of the product and process including midpoint and end counts with CBC with manual diff, platelets, B and T celss (CD4 and 8), myeloma cells, flow cytometry, machine performance are performed at the investigative site apheresis center during apheresis and reported prior to shipment of the apheresis product from the apheresis center to other locations for analysis, characterization and manufacturing of P-BCMA-101 cells).
¹⁴Laboratory studies labeled as “sample for/of” or “blood sample for” are sent to a central laboratory, whereas those not so labeled are generally conducted at the investigational site. A subject enrolled prior to the approval of P-BCMA-101-001 A3 (amendment 3) will continue to have assessments for myeloma response; HIV, Hepatitis B and C, and HTLV Screening; Serum IgG; and Cytokine Release Syndrome markers (CRS) conducted at the investigational site.

TABLE 11
Schedule of Events-P-BCMA-101 Dosing: Cycle Administration, Cohort A and Cohort C

| | P-BCMA-101 | | | | | P-BCMA-101 | | | | |
|---|---|---|---|---|---|---|---|---|---|---|
| | Administration | | | | | Administration | | | | |
| | Cycle 1 ^{8,12} | | | | | Cycle 2 ^{8,12} | | | | |
| | Days | | | | | Days | | | | |
| Procedures | 0⁷ | 1 | 4 | 7 | 10⁹ | 0⁷ | 1 | 4 | 7 | 10⁹ |
| Mini Mental Status Exam (MMSE) | X | X | X | X | X | X | X | X | X | X |
| 12-Lead ECG1 | X | X | X | X | | X | X | X | X | |
| Samples for Myeloma Response2, 11 | X2 | | | | | X2 | | | X2 | |
| Blood Sample for BCMA and Other | X | | | | | X | | | | |
| Biomarkers11 | | | | | | | | | | |
| Physical Exam (Including Neurological | X | X | X | X | X | X | X | X | X | X |
| Exam) | | | | | | | | | | |
| Vital Signs (BP, HR, RR, O2 sat, temp) | X | X | X | X | X | X | X | X | X | X |
| Pregnancy Test3 | X | | | | | X | | | | |
| Chemistry Panel, Hematology including B | X | X | X | X | X | X | X | X | X | X |
| and T cell counts, Coagulation4 | | | | | | | | | | |
| P-BCMA-101 Cell Infusion (IV) | X | | | | | X | | | | |
| Blood Sample for P-BCMA-101 T cells5, 11 | X5 | | X | X | X | X5 | | X | X | X |
| Blood Sample for Serum IgG11 | X | | | | | X | | | | |
| Blood Sample for Cytokine Release | X | X | X | X | X | X | X | X | X | X |
| Syndrome markers (CRS)6 e.g. CRP, Ferritin, | | | | | | | | | | |
| Cytokines IL-6, IL-2, IL-15, TNF-α, and | | | | | | | | | | |
| IFN-γ6, 11 | | | | | | | | | | |
| Adverse Events | X | X | X | X | X | X | X | X | X | X |
| Concomitant Medication10 | X | X | X | X | X | X | X | X | X | X |

112-Lead ECG conducted at Days 0, 1, 4, 7, Week 4, Month 3 and every 3 months thereafter.
2Myeloma Response will be assessed at Days 0 (before P-BCMA-101 administration-thus the 24-hr urine collection is to be started on Day −1 so as to be completed before P-BCMA-101 administration), Day 7 of Cycle 2, Weeks 2, 3, 4, 6, 8 and Months 3, 4, 5, 6, 7, 8, 9,12, 15, 18, 21 and 24 and as clinically indicated (e.g. ≤1 week after an assessment demonstrating a response for confirmation) per the International Myeloma Working Group criteria and based on the standard assessments, including serum protein electrophoresis (SPEP), urine protein electrophoresis (UPEP), serum immunofixation (SIFE), urine immunofixation (UIFE), and serum free light chains (FLC).
3Females of childbearing potential will have a urine pregnancy test prior to each dosing cycle.
4Blood chemistries will include sodium, potassium, chloride, magnesium, bicarbonate, blood urea nitrogen, creatinine, calcium, albumin, glucose, LDH, total protein, ALT, AST, bilimbin (total and direct), alkaline phosphatase, PTT and PT or INR. Assessments for tumor lysis (uric acid and phosphate) will be performed at baseline and Days 1, 4, 7, then as clinically indicated.
Hematological studies include:
• Complete blood count
• Platelets
• B (CD19) and T cell counts (CD3) and CD4 and CD8
5P-BCMA-101 cells will be assessed; e.g. number of vector copies/mL of blood of P-BCMA-101 cells, surface marker phenotype, clonality, etc. At Baseline, Days 0 (1 hour after P-BCMA-101 administration), 4, 7, 10, Weeks, 2, 3, 4, 6, 8, Month 3, 4, 5, 6, 7, 8, 9 and every 3 months thereafter. The cellular characterization and phenotyping assessments conducted on the sample at each time point will vary depending on the temporal relevance of each assay.
6CRS will be graded using Lee criteria (Lee, 2014).
⁷On Day 0 MMSE (should include sample of handwriting by the subject), 12-Lead ECG, physical exam, chemistry panel, hematology and coagulation should be obtained before and approximately 1 hour after P-BCMA-101 administration (+/−15 minutes). Serum creatinine should be <2.0 mg/dL, serum glutamic oxaloacetic transaminase (SGOT) <3 × the upper limit of normal and total bilirubin <2.0 mg/dL or have medical monitor approval to proceed with P-BCMA-101 infusion. Vital signs (temperature, respiration rate, pulse, O2 saturation and blood pressure) will be taken before and after P-BCMA-101 administration, then every 15 minutes (+/−5 minutes) for at least one hour and until these signs are satisfactory and stable. Other Day 0 assessments and samples should be obtained before P-BCMA-101 administration unless otherwise indicated. After administration of P-BCMA-101 these assessments (or any other assessments deemed clinically indicated by the Investigator) will be conducted as described in Table 11 and Table 13 and as often as clinically indicated by the adverse events observed or by the institution’s standards.
⁸ If a subject discontinues from this study after Cycle 2 Day 10, the events for the next visit scheduled should be performed prior to initiating alternative medical, radiation or surgical intervention and recorded as an end-of-study visit for this study. If a subject discontinues from this study after initial dose but prior to Cycle 2 Day 10, the assessments for Week 2 visit should be performed prior to initiating alternative medical, radiation or surgical intervention and recorded as an end-of-study visit for this study. If the subject is enrolling into Protocol P-BCMA-101-002, the subject should be consented and enrolled into that protocol following the subject discontinuing from Protocol P-BCMA-101-001 and the events for the first visit of Protocol P-BCMA-101-002 performed and recorded per the schedule prescribed by that protocol relative to the administration of P-BCMA-101 on this protocol.
⁹Day 10 assessments may be performed up to 2 days after Day 10 if needed, but not on the same day as next Day 0 or week 2 assessments.
10All medications, supplements and allergies (including severity of any current symptoms) should be recorded at each visit.
11Laboratory studies labeled as “sample for/of” or “blood sample for” are sent to a central laboratory, whereas those not so labeled are generally conducted at the investigational site. A subject enrolled prior to the approval of P-BCMA-101-001 A3 (amendment 3) will continue to have assessments for myeloma response; HIV, Hepatitis B and C, and HTLV Screening; Serum IgG; and Cytokine Release Syndrome markers (CRS) conducted at the investigational site.
¹² P-BCMA-101 Cycle administration will be repeated based on the Cohort the subject is in. Cohort A and Cohort C subjects will complete Cycle 1 (Days 0-10) for the 1st dose and Cycle 2 (Days 0-10) for the 2nd dose. Once Cycle 2 Day 10 has been completed, the subject moves on to Week 2.

TABLE 12
Schedule of Events-P-BCMA-101 Dosing: Cycle Administration, Cohort B

| | P-BCMA-101 | | | | | P-BCMA-101 | | | | | P-BCMA-101 | | | | |
|---|---|---|---|---|---|---|---|---|---|---|---|---|---|---|---|
| | Administration | | | | | Administration | | | | | Administration | | | | |
| | Cycle 1 ^{8,12} | | | | | Cycle 2 ^{8,12} | | | | | Cycle 3 ^{8,12} | | | | |
| | Days | | | | | Days | | | | | Days | | | | |
| Procedures | 0⁷ | 1 | 4 | 7 | 10⁹ | 0⁷ | 1 | 4 | 7 | 10⁹ | 0⁷ | 1 | 4 | 7 | 10⁹ |
| Mini Mental Status Exam | X | X | X | X | X | X | X | X | X | X | X | X | X | X | X |
| (MMSE) | | | | | | | | | | | | | | | |
| 12-Lead ECG1 | X | X | X | X | | X | X | X | X | | X | X | X | X | |
| Samples for Myeloma | X2 | | | | | X2 | | | X2 | | X2 | | | | |
| Response2, 11 | | | | | | | | | | | | | | | |
| Blood Sample for BCMA | X | | | | | X | | | | | X | | | | |
| and Other Biomarkers11 | | | | | | | | | | | | | | | |
| Physical Exam (Including | X | X | X | X | X | X | X | X | X | X | X | X | X | X | X |
| Neurological Exam) | | | | | | | | | | | | | | | |
| Vital Signs (BP, HR, RR, | X | X | X | X | X | X | X | X | X | X | X | X | X | X | X |
| O2 sat, temp) | | | | | | | | | | | | | | | |
| Pregnancy Test3 | X | | | | | X | | | | | X | | | | |
| Chemistry Panel, | X | X | X | X | X | X | X | X | X | X | X | X | X | X | X |
| Hematology including B | | | | | | | | | | | | | | | |
| and T cell counts, | | | | | | | | | | | | | | | |
| Coagulation4 | | | | | | | | | | | | | | | |
| P-BCMA-101 Cell | X | | | | | X | | | | | X | | | | |
| Infusion (IV) | | | | | | | | | | | | | | | |
| Blood Sample for P- | X5 | | X | X | X | X5 | | X | X | X | X5 | | X | X | X |
| BCMA-101 T cells5, 11 | | | | | | | | | | | | | | | |
| Blood Sample for Serum | X | | | | | X | | | | | X | | | | |
| IgG11 | | | | | | | | | | | | | | | |
| Blood Sample for | X | X | X | X | X | X | X | X | X | X | X | X | X | X | X |
| Cytokine Release | | | | | | | | | | | | | | | |
| Syndrome markers | | | | | | | | | | | | | | | |
| (CRS)6 e.g. CRP, | | | | | | | | | | | | | | | |
| Ferritin, Cytokines IL-6, | | | | | | | | | | | | | | | |
| IL-2, IL-15, TNF-α, and | | | | | | | | | | | | | | | |
| IFN-γ6, 11 | | | | | | | | | | | | | | | |
| Adverse Events | X | X | X | X | X | X | X | X | X | X | X | X | X | X | X |
| Concomitant | X | X | X | X | X | X | X | X | X | X | X | X | X | X | X |
| Medication10 | | | | | | | | | | | | | | | |

112-Lead ECG conducted at Days 0, 1, 4, 7, Week 4, Month 3 and every 3 months thereafter.
2Myeloma Response will be assessed at Days 0 (before P-BCMA-101 administration-thus the 24-hr urine collection is to be started on Day −1 so as to be completed before P-BCMA-101 administration), Day 7 of Cycle 2, Weeks 2, 3, 4, 6, 8 and Months 3, 4, 5, 6, 7, 8, 9, 12, 15, 18, 21 and 24 and as clinically indicated (e.g. ≤1 week after an assessment demonstrating a response for confirmation) per the International Myeloma Working Group criteria and based on the standard assessments, including serum protein electrophoresis (SPEP), urine protein electrophoresis (UPEP), serum immunofixation (SIFE), urine immunofixation (UIFE), and serum free light chains (FLC).
3Females of childbearing potential will have a mine pregnancy test prior to each dosing cycle.
4Blood chemistries will include sodium, potassium, chloride, magnesium, bicarbonate, blood urea nitrogen, creatinine, calcium, albumin, glucose, LDH, total protein, ALT, AST, bilirubin (total and direct), alkaline phosphatase, PTT and PT or INR. Assessments for tumor lysis (uric acid and phosphate) will be performed at baseline and Days 1, 4, 7, then as clinically indicated.
Hematological studies include:
Complete blood count
Platelets
B (CD19) and T cell counts (CD3) and CD4 and CD8
5P-BCMA-101 cells will be assessed; e.g. number of vector copies/mL of blood of P-BCMA-101 cells, surface marker phenotype, clonality, etc. At Baseline, Days 0 (1 hour after P-BCMA-101 administration), 4, 7, 10, Weeks, 2, 3, 4, 6, 8, Month 3, 4, 5, 6, 7, 8, 9 and every 3 months thereafter. The cellular characterization and phenotyping assessments conducted on the sample at each time point will vary depending on the temporal relevance of each assay.
6CRS will be graded using Lee criteria (Lee, 2014).
⁷On Day 0 MMSE (should include sample of handwriting by the subject), 12-Lead ECG, physical exam, chemistry panel, hematology and coagulation should be obtained before and approximately 1 hour after P-BCMA-101 administration (+/−15 minutes). Serum creatinine should be <2.0 mg/dL, serum glutamic oxaloacetic transaminase (SGOT) <3 × the upper limit of normal and total bilirubin <2.0 mg/dL or have medical monitor approval to proceed with P-BCMA-101 infusion. Vital signs (temperature, respiration rate, pulse, O2 saturation and blood pressure) will be taken before and after P-BCMA-101 administration, then every 15 minutes (+/−5 minutes) for at least one hour and until these signs are satisfactory and stable. Other Day 0 assessments and samples should be obtained before P-BCMA-101 administration unless otherwise indicated. After administration of P-BCMA-101 these assessments (or any other assessments deemed clinically indicated by the Investigator) will be conducted as described in Table 12 and Table 13, and as often as clinically indicated by the adverse events observed or by the institution’s standards.
⁸ If a subject discontinues from this study after Cycle 3 Day 10, the events for the next visit scheduled should be performed prior to initiating alternative medical, radiation or surgical intervention and recorded as an end-of-study visit for this study. If a subject discontinues from this study after initial dose but prior to Cycle 3 Day 10, the assessments for Week 2 visit should be performed prior to initiating alternative medical, radiation or surgical intervention and recorded as an end-of-study visit for this study If the subject is emolling into Protocol P-BCMA-101-002, the subject should be consented and enrolled into that protocol following the subject discontinuing from Protocol P-BCMA-101-001 and the events for the first visit of Protocol P-BCMA-101-002 performed and recorded per the schedule prescribed by that protocol relative to the administration of P-BCMA-101 on this protocol.
⁹Day 10 assessments may be performed up to 2 days after Day 10 if needed, but not on the same day as week 2 assessments.
10All medications, supplements and allergies (including severity of any current symptoms) should be recorded at each visit.
11Laboratory studies labeled as “sample for/of” or “blood sample for” are sent to a central laboratory, whereas those not so labeled are generally conducted at the investigational site. A subject enrolled prior to the approval of P-BCMA-101-001 A3 (amendment 3) will continue to have assessments for myeloma response; HIV, Hepatitis B and C, and HTLV Screening; Serum IgG; and Cytokine Release Syndrome markers (CRS) conducted at the investigational site.
12 P-BCMA-101 Cycle administration will be repeated based on the Cohort the subject is in. Cohort B subjects will complete Cycle 1 (Days 0-10) for the 1st dose, Cycle 2 (Days 0-10) for the 2nd dose and Cycle 3 (Days 0-10) for the 3rd dose. Once Cycle 3 Day 10 has been completed, the subject moves on to Week 2.

TABLE 13
Schedule of Events - Post Treatment Follow-up: Cycle Administration Cohorts A, B, and C

| | Post-Treatment Follow-up Visits (visit windows) | | | | |
|---|---|---|---|---|---|
| | relative to Day 0 of last Dosing Cycle ^{7, 11} | | | | |
| | Week | | Week | | q3 months |
| | 2 & 3 | Week 4 | 6 & 8 | Month 3 | (±2 weeks) |
| Procedures | (±2 days) | (±7 days) | (±7 days) | (±2 weeks) | to Month 24 |
| Mini Mental Status Exam (MMSE) | X | X | X | | X |
| 12-Lead ECG¹ | | X | | X | X |
| Samples for Myeloma Response^{2, 9} | X | X | X | Mo 3, | +Mo 6, |
| | | | | 4, 5 | 7, 8, 9 |
| Fresh Sample of Bone Marrow and | | X | | X | Mo 6, 12 |
| Tumor^{2, 9} | | | | | |
| Blood Sample for BCMA and | X | X | X | X | X |
| Other Biomarkers⁹ | | | | | |
| Physical Exam (Including | X | X | X | X | X |
| Neurological Exam) | | | | | |
| Vital Signs (BP, HR, RR, | X | X | X | X | X |
| O2 sat, temp) | | | | | |
| Pregnancy Test (urine)³ | X | X | X | X | X |
| Chemistry Panel, Hematology | X | X | X | X | X |
| including B and T cell counts, | | | | | |
| Coagulation⁴ | | | | | |
| Blood Sample for P-BCMA-101 | X | X | X | Mo 3, | +Mo 6, |
| T cells^{5, 9} | | | | 4, 5 | 7, 8, 9 |
| Blood Sample for Serum IgG⁹ | | X | X | X | X |
| Blood Sample for Immunogenicity | X | X | | X | X |
| Assays⁹ | | | | | |
| Blood Sample for Cytokine Release | X | X | X | Mo 3, | X |
| Syndrome markers (CRS)6 e.g. | | | | 4, 5 | |
| CRP, Ferritin, Cytokines IL-6, | | | | | |
| IL-2, IL-15, TNF-α, and IFN-γ^{6, 9} | | | | | |
| PET/CT¹⁰ | | | (X¹⁰) | (X¹⁰) | (X¹⁰) |
| Adverse Events | X | X | X | X | X |
| Concomitant Medication⁸ | X | X | X | X | X |

¹12-Lead ECG conducted at Days 0, 1, 4, 7, Week 4, Month 3 and every 3 months thereafter.
²Myeloma Response will be assessed at Days 0 (before P-BCMA-101 administration- thus the 24-hr urine collection is to be started on Day −1 so as to be completed before P-BCMA-101 administration), Day 7 of Cycle 2, Weeks 2, 3, 4, 6, 8 and Months 3, 4, 5, 6, 7, 8, 9, 12, 15, 18, 21 and 24 and as clinically indicated (e.g. ≤1 week after an assessment demonstrating a response for confirmation) per the International Myeloma Working Group criteria and based on the standard assessments, including serum protein electrophoresis (SPEP), urine protein electrophoresis (UPEP), serum immunofixation (SIFE), urine immunofixation (UIFE), and serum free light chains (FLC). Bone marrow biopsy, aspirate and minimal residual disease (MRD) (analyzed by central labs) must be done at Week 4, Months 3, 6 and 12 then as clinically indicated, or have a medical monitor exemption. A sample of marrow aspirate and a sample of bone marrow biopsy are to be submitted (as described in the Study Reference Manual) for assessment of MRD, plasma cell frequency, P-BCMA-101 cells, BCMA, and other biomarkers. A portion of any extramedullary biopsy should also be submitted for assessment of P-BCMA-101 cells, BCMA, and other biomarkers. PET/CT is to be conducted as clinically indicated.
³Females of childbearing potential will have a urine pregnancy test at all indicated visits after Screening.
⁴Blood chemistries will include sodium, potassium, chloride, magnesium, bicarbonate, blood urea nitrogen, creatinine, calcium, albumin, glucose, LDH, total protein, ALT, AST, bilirubin (total and direct), alkaline phosphatase, PTT and PT or INR. Assessments for tumor lysis (uric acid and phosphate) will be performed at baseline and Days 1, 4, 7, then as clinically indicated. Hematological studies include:
Complete blood count
Platelets
B (CD19) and T cell counts (CD3) and CD4 and CD8
⁵P-BCMA-101 cells will be assessed; e.g. number of vector copies/mL of blood of P-BCMA-101 cells, surface marker phenotype, clonality, etc. At Baseline, Days 0 (1 hour after P-BCMA-101 administration), 4, 7, 10, Weeks, 2, 3, 4, 6, 8, Month 3, 4, 5, 6, 7, 8, 9 and every 3 months thereafter. The cellular characterization and phenotyping assessments conducted on the sample at each time point will vary depending on the temporal relevance of each assay.
⁶CRS will be graded using Lee criteria (Lee, 2014).
⁷ If a subject discontinues from this study after P-BCMA-101 administration, the events for the next visit scheduled should be performed prior to initiating alternative medical, radiation or surgical intervention and recorded as an end-of-study visit for this study. If the subject is enrolling into Protocol P-BCMA-101-002, the subject should be consented and enrolled into that protocol following the subject discontinuing from Protocol P-BCMA-101-001 and the events for the first visit of Protocol P-BCMA-101-002 performed and recorded per the schedule prescribed by that protocol relative to the administration of P-BCMA-101 on this protocol.
⁸All medications, supplements and allergies (including severity of any current symptoms) should be recorded at each visit.
⁹Laboratory studies labeled as “sample for/of” or “blood sample for” are sent to a central laboratory, whereas those not so labeled are generally conducted at the investigational site. A subject enrolled prior to the approval of P-BCMA-101-001 A3 (amendment 3) will continue to have assessments for myeloma response; HIV, Hepatitis B and C, and HTLV Screening; Serum IgG; and Cytokine Release Syndrome markers (CRS) conducted at the investigational site.
¹⁰PET/CT to be obtained at Baseline within 7 days of initiating conditioning chemotherapy, then as clinically indicated (e.g. every 8 weeks if soft tissue plasmacytomas are found at baseline that require imaging as part of response assessments or to confirm response).
¹¹ Once subjects complete all Cycles of Dosing (2 for Cohort A and Cohort C and 3 for Cohort B), they will continue on with the Week 2 visit (14 days from most recent Day 0).

### Example 6—P-BCMA-101 Combination Administration

15.6. Phase 1—Combination Administration

In Phase 1—Combination Administration, P-BCMA-101 will be administered in combination with approved therapies:

Lenalidomide

Cohort R: lenalidomide 10 mg orally daily for 21 of every 28 days beginning 1 week before P-BCMA-101 infusion; and

Cohort RP: lenalidomide 10 mg orally daily for 7 days beginning 1 week before apheresis and for 21 of every 28 days beginning 1 week before P-BCMA-101 infusion.

Dosing with lenalidomide will continue for Cohort R and Cohort RP unless disease progresses. Refer to the lenalidomide package insert for prescribing information (Lenalidomide, 2019) (note particularly that lenalidomide is a presumed teratogen, pregnancy avoidance and monitoring is necessary). The following are additional recommendations specific to this protocol. If no DLTs are reported and platelets are >50,000/μL and neutrophils>1000/μL 28 days after P-BCMA-101 administration the dose may be increased to 25 mg orally daily for 21 of every 28 days. If <2 DLTs are reported in the first 6 patients treated at this dose, the starting dose in all patients may be increased to 25 mg orally daily for 21 of every 28 days at the determination of the Safety Committee. During treatment if neutrophils decrease to <1000/μL hold lenalidomide until they are >1000/μL, then restart at a 5 mg lower dose. During treatment if platelets decrease to <30,000/μL hold lenalidomide until they are >30,000/μL, then restart at a 5 mg lower dose. If creatinine clearance is 30-60 mL/min the maximum lenalidomide dose should be 10 mg daily. If creatinine clearance is <30 mL/min hold lenalidomide. Lenalidomide should be discontinued in case of DLT. The lowest dose allowed on this study is 5 mg daily. The investigator and Safety Committee may decide to discontinue lenalidomide at time based on other safety findings. Patients should receive concomitant anticoagulation as indicated (eg. aspirin 325 mg orally daily). Do not administer glucocorticoids with lenalidomide.

Rituximab

Cohort RIT: 375 mg/m² via intravenous infusion, 12 and 5 days before P-BCMA-101 infusion, then every 8 weeks unless disease progresses. Refer to the rituximab package insert for prescribing information (Rituximab, 2019). The following are additional recommendations specific to this protocol. Rituximab should only be administered by a healthcare professional with appropriate medical support to manage severe infusion-related reactions that can be fatal if they occur. First Infusion: Initiate infusion at a rate of 50 mg/hr. In the absence of infusion toxicity, increase infusion rate by 50 mg/hr increments every 30 minutes, to a maximum of 400 mg/hr. Subsequent Infusions: Standard Infusion: Initiate infusion at a rate of 100 mg/hr. In the absence of infusion toxicity, increase rate by 100 mg/hr increments at 30-minute intervals, to a maximum of 400 mg/hr. Administer only as an intravenous infusion. Do not administer as an intravenous push or bolus. Premedicate before each infusion with acetaminophen, an antihistamine, and 100 mg intravenous methylprednisolone to be completed 30 minutes prior to each infusion. Rituximab should be discontinued in case of infusion reaction or DLT. The investigator and Safety Committee may decide to discontinue rituximab at any time based on other safety findings. Pneumocystis pneumonia (PCP) prophylaxis should be considered for patients during and following treatment.

The dose of P-BCMA-101 administered will escalate or de-escalate following the 3+3 design starting at ≤the MTD as determined during dose escalation.

The Schedule of Events for Screening through conditioning chemotherapy for combination administration is shown in Table 14. The Schedule of Events for P-BCMA-101 administration and follow-up for combination administration is shown in Table 15.

TABLE 14
Schedule of Events-Screening through Conditioning Chemotherapy
(Combination Administration)

| | | | | Baseline | Baseline | | | | | |
|---|---|---|---|---|---|---|---|---|---|---|
| | Screening | Enrollment ¹² | | (within 7 | (within 72 | Conditioning | | | | |
| | (within 28 | (2 weeks ± 3 | | days prior to | hours prior to | Chemotherapy¹¹ | | | | |
| | days prior to | days prior to | Leuka- | Conditioning | Conditioning | Days | | | | |
| Procedures | leukapheresis) | leukapheresis) | pheresis¹³ | Chemotherapy) | Chemotherapy) | −5 | −4 | −3 | −2 | −1 |
| Informed Consent | X | | | | | | | | | |
| Inclusion/Exclusion | X | | | | | X | | | | |
| Criteria | | | | | | | | | | |
| Medical History and | X | | | | | | | | | |
| Disease Assessment¹ | | | | | | | | | | |
| ECOG Performance | X | | | | | X | | | | |
| Status | | | | | | | | | | |
| Mini Mental Status | X | | | | X | X | | | | |
| Exam (MMSE) | | | | | | | | | | |
| Height and Weight² | X | X¹² | X | | | | | | | |
| 12-Lead ECG³ | X | | | | | | | | | |
| Echocardiogram | X | | | | | | | | | |
| Archival Tumor to | X | | | | | | | | | |
| Central Lab | | | | | | | | | | |
| Samples for Myeloma | X⁴ | | | X⁴ | | | | | | |
| Response^{4,14} | | | | | | | | | | |
| Fresh Sample of Bone | X⁴ | | | X⁴ | | | | | | |
| Marrow and Tumor^{4,14} | | | | | | | | | | |
| Blood Sample for | | X¹² | | | | | | | | |
| BCMA and Other | | | | | | | | | | |
| Biomarkers¹⁴ | | | | | | | | | | |
| Physical Exam | X | | | | X | X | | | | |
| (Including Neurological | | | | | | | | | | |
| Exam) | | | | | | | | | | |
| Vital Signs (BP, HR, RR, | X | | | | X | X | X | X | | |
| O2 sat, temp) | | | | | | | | | | |
| Pregnancy Test (serum | X | RP | RP | RP/R | X | | | | | |
| or urine)⁵ | | | | | | | | | | |
| Blood Sample for HIV, | X | | | | | | | | | |
| Hepatitis B and C, and | | | | | | | | | | |
| HTLV Screening¹⁴ | | | | | | | | | | |
| Chemistry Panel, | X | X | | | X | | X | X | | |
| Hematology including B | | | | | | | | | | |
| and T cell counts, | | | | | | | | | | |
| Coagulation⁶ | | | | | | | | | | |
| Assessment of | X | X | | | X | | | | | |
| circulating | | | | | | | | | | |
| myeloma/plasma cells⁶ | | | | | | | | | | |
| Leukapheresis | | | X¹³ | | | | | | | |
| Lenalidomide | 10-25 mg orally daily for 7 days beginning 1 week before apheresis and 21 of every 28 days | | | | | | | | | |
| (Cohort RP)^{15,18} | beginning 1 week before P-BCMA-101 infusion | | | | | | | | | |
| Lenalidomide | | | | | 10-25 mg orally daily for 21 of | | | | | |
| (Cohort R)^{16,18} | | | | | every 28 days beginning 1 week | | | | | |
| | | | | | before P-BCMA-101 infusion | | | | | |
| Rituximab | | | | 375 mg/m² via infusion 12 and 5 | | | | | | |
| (Cohort RIT)^{17,18} | | | | days before P-BCMA-101 | | | | | | |
| | | | | infusion | | | | | | |
| Fludarabine/ | | | | | | X | X | X | | |
| Cyclophosphamide | | | | | | | | | | |
| Blood Sample for P- | | X¹² | | | | | | | | |
| BCMA-101 T cells^{7,14} | | | | | | | | | | |
| Blood Sample for Serum | | X¹² | | | | | | | | |
| IgG¹⁴ | | | | | | | | | | |
| Blood Sample for | | X¹² | | | | | | | | |
| Immunogenicity | | | | | | | | | | |
| Assays¹⁴ | | | | | | | | | | |
| Blood Sample for | | X¹² | | | | | | | | |
| Cytokine Release | | | | | | | | | | |
| Syndrome markers | | | | | | | | | | |
| (CRS)8: e.g. CRP, | | | | | | | | | | |
| Ferritin, Cytokines IL-6, | | | | | | | | | | |
| IL-2, IL-15, TNF-α, and | | | | | | | | | | |
| IFN-γ^{8,14} | | | | | | | | | | |
| PET/CT^{4,9} | | | | X4, 9 | | | | | | |
| Adverse Events | | X | X | X | X | X | X | X | | |
| Concomitant | X¹⁰ | X¹² | X | X | X | X | X | X | | |
| Medication¹⁰ | | | | | | | | | | |

¹Including MM measurement results for the 6 months prior to Screening required (including serum protein electrophoresis [SPEP], urine protein electrophoresis [UPEP], serum immunofixation [SIFE], urine immunofixation [UIFE], serum free hght chains [FLC], minimal residual disease [MRD] and as clinically indicated, PET/CT and/or bone marrow/tumor biopsy/aspirate [Bone marrow biopsy], if available).
²Height obtained at Screening Visit only
³12-Lead ECG and echocardiogram obtained at Screening.
⁴Myeloma Response will be assessed for Screening (may be obtained from any result within the last month), baseline within 7 days of initiating conditioning chemotherapy (note: 2 or more assessments must be performed between completion of the last myeloma therapy, including salvage therapy, and initiation of lymphodepleting chemotherapy), Days 0 (before P-BCMA-101 administration-thus the 24-hr urine collection is to be started on Day −1 so as to becompleted before P-BCMA-101 administration), and as clinically indicated per the International Myeloma Working Group criteria and based on the standard assessments, including SPEP, UPEP, SIFE, UIFE, and serum FLC. Bone marrow biopsy, aspirate and MRD (analyzed by central lab) must be done within 7 days of initiating conditioning chemotherapy, then as clinically indicated, or have a medical monitor exemption. A sample of bone marrow aspirate and a sample of bone marrow biopsy are to be submitted (as described in the Study Reference Manual) for assessment of MRD, plasma cell frequency, P-BCMA-101 cells, BCMA, and other biomarkers. A portion of any extramedullary biopsy should also be submitted for assessment of P-BCMA-101 cells, BCMA, and other biomarkers. If a new bone marrow biopsy/aspirate was performed and provided during screening, this does not need to be repeated during the Baseline visit. PET/CT is to be conducted as clinically indicated.
⁵All female subjects in R and RP cohort: Two negative pregnancy tests must be obtained prior to initiating therapy. The first test should be performed within 10-14 days and the second test within 24 hours prior to prescribing Lenalidomide therapy and then weekly during the first month, then monthly thereafter in females with regular menstrual cycles or every 2 weeks in females with irregular menstrual cycles
⁶Blood chemistries will include sodium, potassium, chloride, magnesium, bicarbonate, blood urea nitrogen, creatinine, calcium, albumin, glucose, LDH, total protein, ALT, AST, bilirubin (total and direct), alkaline phosphatase, PTT and PT or INR.
Hematological studies include:
Complete blood count (CBC)
Platelets
B (CD19) and T cell counts (CD3) and CD4 and CD8 at all timepoints except Days −3, −4
Assessment of circulating myeloma/plasma cells (e.g. by flow cytometry or CBC with manual differential) is required at timepoints prior to P-BCMA-101 administration. Contact the sponsor and refer to exclusion criteria #3 and #10 if circulating myeloma/plasma cells are identified in the Enrollment & Baseline sample.
⁷P-BCMA-101 cells will be assessed; e.g. number of vector copies/mL of blood of P-BCMA-101 cells, surface marker phenotype, clonality, etc. at Enrollment. The cellular characterization and phenotyping assessments conducted on the sample at each time point will vary depending on the temporal relevance of each assay.
⁸CRS will be graded using Lee criteria (Lee, 2014).
⁹PET/CT to be obtained at Baseline within 7 days of initiating conditioning chemotherapy.
¹⁰Allergies and all prescription and nonprescription medication, vitamins, herbal and nutritional supplements, taken by the subject during the 30 days prior to Screening will be recorded at the Screening Visit. All medications, supplements and allergies (including severity of any current symptoms) should be recorded at each visit. Unnecessary medications/supplements should be discontinued prior to Screening, if possible, at the investigator’s judgement.
¹¹Subjects should continue to meet entry criteria at the time of initiation of conditioning chemotherapy or have medical monitor approval. The following assessments should be repeated within 72 hours of Day −5 to re-assess entry criteria: MMSE (should include sample of handwriting by the subject); physical exam; vital signs; pregnancy; and chemistry panel, hematology including B and T cell counts, coagulation.
¹²Enrollment assessments are to be conducted 14 days ± 3 days prior to leukapheresis or have medical monitor approval.
¹³Leukapheresis should take place within~28 days following screening or have medical monitor approval. Guidance for conducting leukapheresis may be found in Section 5.1 and the Study Reference Manual (in particular characterization of the product and process including midpoint and end counts with CBC with manual diff, platelets, B and T cells (CD4 and 8), myeloma cells, flow cytometry, machine performance are performed at the investigative site apheresis centerduring apheresis and reported prior to shipment of the apheresis product from the apheresis center to other locations for analysis, characterization and manufacturing of P-BCMA-101 cells).
¹⁴Laboratory studies labeled as “sample for/of” or “blood sample for” are sent to a central laboratory, whereas those not so labeled are generally conducted at the investigational site. A subject enrolled prior to the approval of P-BCMA-101-001 A3 (amendment 3) will continue to have assessments for myeloma response; HIV, Hepatitis B and C, and HTLV Screening; Serum IgG; and Cytokine Release Syndrome markers (CRS) conducted at the investigational site.
¹⁵Lenalidomide in Cohort R: 10-25 mg orally daily for 21 of every 28 days beginning 1 week before P-BCMA-101 infusion.
¹⁶Lenalidomide in Cohort RP: 10 mg orally daily for 7 days before apheresis, then 10-25 mg orally daily for 21 of every 28 days beginning 1 week before P-BCMA-101 infusion
¹⁷Rituximab in Cohort RIT: 375 mg/m2 via infusion 12 and 5 days before P-BCMA-101 infusion, then every 8 weeks
¹⁸For subjects in Cohort R, Cohort RP and Cohort RIT, the combination therapy should be administered prior to conditioning chemotherapy on applicable days

TABLE 15
Schedule of Events-P-BCMA-101 Administration and Follow-Up (Combination Administration)

| | | | | | | Post-Treatment Follow-up | | | | |
|---|---|---|---|---|---|---|---|---|---|---|
| | P-BCMA-101 | | | | | Visits (visit windows) relative to Day 0 ⁸ | | | | |
| | Administration ⁸ | | | | | Week | | Week | | q3 months |
| | Days | | | | | 2 & 3 | Week 4 | 6 & 8 | Month 3 | (±2 weeks) |
| Procedures | 0⁷ | 1 | 4 | 7 | 10⁹ | (±2 days) | (±7 days) | (±7 days) | (±2 weeks) | to Month 24 |
| Mini Mental Status Exam (MMSE) | X | X | X | X | X | X | X | X | | X |
| 12-Lead ECG¹ | X | X | X | X | | | X | | X | X |
| Samples for Myeloma Response^{2,11} | X² | | | | | X | X | X | Mo 3, 4, 5 | +Mo 6, 7, 8, 9 |
| Fresh Sample of Bone Marrow and Tumor^{2,11} | | | | | | | X | | X | Mo 6, 12 |
| Blood Sample for BCMA and Other Biomarkers¹¹ | X | | | | | X | X | X | X | X |
| Physical Exam (Including Neurological Exam) | X | X | X | X | X | X | X | X | X | X |
| Vital Signs (BP, HR, RR, O2 sat, temp) | X | X | X | X | X | X | X | X | X | X |
| Pregnancy Test (urine)³ | R/RP | | | R/RP | | X | X | X | X | X |
| Chemistry Panel, Hematology including B and | X | X | X | X | X | X | X | X | X | X |
| T cell counts, Coagulation⁴ | | | | | | | | | | |
| P-BCMA-101 Cell Infusion (IV) | X | | | | | | | | | |
| Blood Sample for P-BCMA-101 T cells^{5,11} | X⁵ | | X | X | X | X | X | X | Mo 3, 4, 5 | +Mo 6, 7, 8, 9 |
| Blood Sample for Serum IgG¹¹ | X | | | | | | X | X | X | X |
| Blood Sample for Immunogenicity Assays¹¹ | | | | | | X | X | | X | X |
| Blood Sample for Cytokine Release Syndrome | X | X | X | X | X | X | X | X | Mo 3, 4, 5 | X |
| markers (CRS)6 e.g. CRP, Ferritin, Cytokines | | | | | | | | | | |
| IL-6, IL-2, IL-15, TNF-α, and IFN-γ^{6,11} | | | | | | | | | | |
| PET/CT¹² | | | | | | | | (X¹²) | (X¹²) | (X¹²) |
| Adverse Events | X | X | X | X | X | X | X | X | X | X |
| Concomitant Medication¹⁰ | X | X | X | X | X | X | X | X | X | X |

| | |
|---|---|
| Lenalidomide (Cohort RP)¹³ | 10-25 mg orally daily for 21 of every 28 days beginning 1 week |
| | before P-BCMA-101 infusion |
| Lenalidomide (Cohort R)¹⁴ | 10-25 mg orally daily for 21 of every 28 days beginning 1 week |
| | before P-BCMA-101 infusion |
| Rituximab (Cohort RIT)¹⁵ | 375 mg/m² via infusion 12 and 5 days before P-BCMA-101 infusion, |
| | then every 8 weeks unless disease progresses |

¹12-Lead ECG conducted at Days 0, 1, 4, 7, Week 4, Month 3 and every 3 months thereafter.
²Myeloma Response will be assessed at Days 0 (before P-BCMA-101 administration-thus the 24-hr urine collection is to be started on Day −1 so as to be completed before P-BCMA-101 administration), Weeks 2, 3, 4, 6, 8 and Months 3, 4, 5, 6, 7, 8, 9, 12, 15, 18, 21 and 24 and as clinically indicated (e.g. ≤1 week after an assessment demonstrating a response for confirmation) per the International Myeloma Working Group criteria and based on the standard assessments, including serum protein electrophoresis (SPEP), urine protein electrophoresis (UPEP), serum immunofixation (SIFE), urine immunofixation (UIFE), and serum free light chains (FLC). Bone marrow biopsy, aspirate and minimal residual disease (MRD) (analyzed by central labs) must be done at Week 4, Months 3, 6 and 12 then as clinically indicated, or have a medical monitor exemption. A sample of marrow aspirate and a sample of bone marrow biopsy are to be submitted (as described in the Study Reference Manual) for assessment of MRD, plasma cell frequency, P-BCMA-101 cells, BCMA, and other biomarkers. A portion of any extramedullary biopsy should also be submitted for assessment of P-BCMA-101 cells, BCMA, and other biomarkers. PET/CT is to be conducted as clinically indicated.
³Females of childbearing potential will have a urine pregnancy test at all indicated visits after Screening. All female subjects in R and RP cohort: Two negative pregnancy tests must be obtained prior to initiating therapy. The first test should be performed within 10-14 days and the second test within 24 hours prior to prescribing lenalidomide therapy and then weekly during the first month, then monthly thereafter in females with regular menstrual cycles or every 2 weeks in females with irregular menstrual cycles
⁴Blood chemistries will include sodium, potassium, chloride, magnesium, bicarbonate, blood urea nitrogen, creatinine, calcium, albumin, glucose, LDH, total protein, ALT, AST, bilirubin (total and direct), alkaline phosphatase, PTT and PT or INR. Assessments for tumor lysis (uric acid and phosphate) will be performed at baseline and Days 1, 4, 7, then as clinically indicated.
Hematological studies include:
Complete blood count
Platelets
B (CD 19) and T cell counts (CD3) and CD4 and CD8
⁵P-BCMA-101 cells will be assessed; e.g. number of vector copies/mL ofblood of P-BCMA-101 cells, surface marker phenotype, clonality, etc. At Baseline, Days 0 (1 hour after P-BCMA-101 administration), 4, 7, 10, Weeks, 2, 3, 4, 6, 8, Month 3, 4, 5, 6, 7, 8, 9 and every 3 months thereafter. The cellular characterization and phenotyping assessments conducted on the sample at each time point will vary depending on the temporal relevance of each assay.
⁶CRS will be graded using Lee criteria (Lee, 2014).
⁷On Day 0 MMSE (should include sample of handwriting by the subject), 12-Lead, ECG, physical exam, chemistry panel, hematology and coagulation should be obtained before and approximately 1 hour after P-BCMA-101 administration (+/−15 minutes). Vital signs (temperature, respiration rate, pulse, O2 saturation and blood pressure) will be taken before and afterP-BCMA-101 administration, then every 15 minutes (+/−5 minutes) for at least one hour and until these signs are satisfactory and stable. Other Day 0 assessments and samples should be obtained before P-BCMA-101 administration unless otherwise indicated. After administration of P-BCMA-101 these assessments (or any other assessments deemed clinically indicated by the Investigator) will be conducted as described in Table 3, and as often as clinically indicated by the adverse events observed or by the institution’s standards.
⁸If a subject discontinues from this study after P-BCMA-101 administration, the events for the next visit scheduled should be performed prior to initiating alternative medical, radiation or surgical intervention and recorded as an end-of-study visit for this study. If the subject is enrolling into Protocol P-BCMA-101-002, the subject should be consented and enrolled into that protocol following the subject discontinuing from Protocol P-BCMA-101-001 and the events for the first visit of Protocol P-BCMA-101-002 performed and recorded per the schedule prescribed by that protocol relative to the administration of P-BCMA-101 on this protocol.
⁹Day 10 assessments may be performed up to 2 days after Day 10 if needed, but not on the same day as week 2 assessments.
¹⁰All medications, supplements and allergies (including severity of any current symptoms) should be recorded at each visit.
¹¹Laboratory studies labeled as “sample for/of” or “blood sample for” are sent to a central laboratory, whereas those not so labeled are generally conducted at the investigational site. A subject enrolled prior to the approval of P-BCMA-101-001 A3 (amendment 3) will continue to have assessments for myeloma response; HIV, Hepatitis B and C, and HTLV Screening; Serum IgG; and Cytokine Release Syndrome markers (CRS) conducted at the investigational site.
¹²PET/CT to be obtained at Baseline within 7 days of initiating conditioning chemotherapy, then as clinically indicated (e.g. every 8 weeks if soft tissue plasmacytomas are found at baseline that require imaging as part of response assessments or to confirm response).
¹³Lenalidomide in Cohort R: 10-25 mg orally daily for 21 of every 28 days beginning 1 week before P-BCMA-101 infusion.
¹⁴Lenalidomide in Cohort RP: 10 mg orally daily for 7 days before apheresis, then 10-25 mg orally daily for 21 of every 28 days beginning 1 week before P-BCMA-101 infusion
¹⁵Rituximab in Cohort RIT: 375 mg/m2 via infusion 12 and 5 days before P-BCMA-101 infusion, then every 8 weeks

### Example 7—Anti-Drug Antibody Response and Pharmacokinetic Assessment of Multiple Myeloma Patients Treated with P-BCMA 101

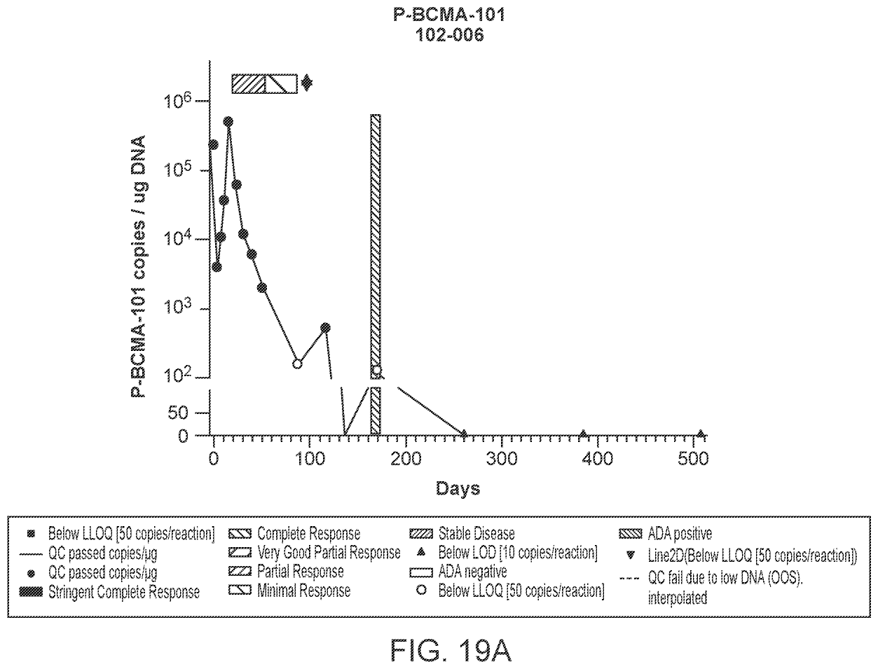

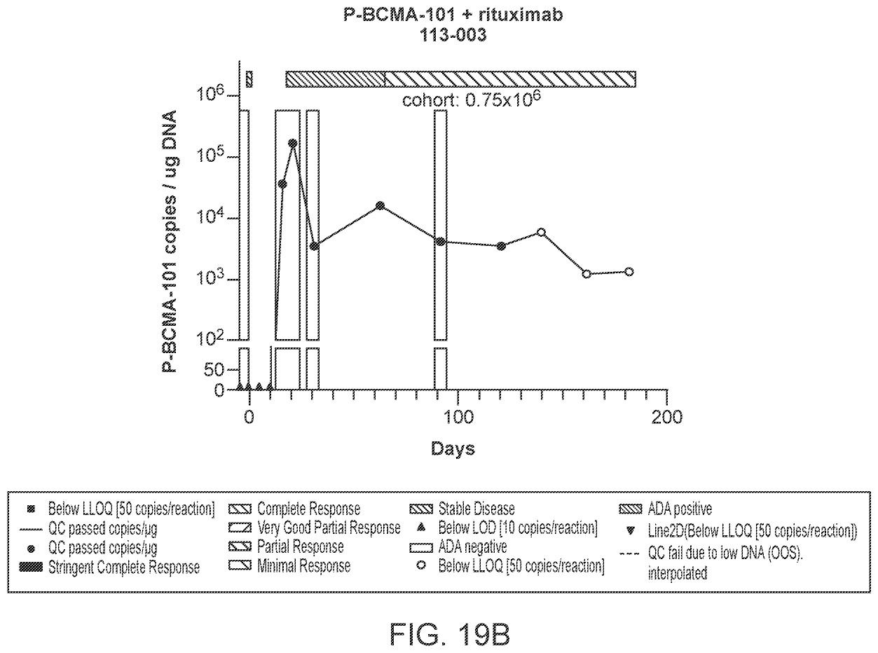

P-BCMA-101 was administered to patients with myeloma alone or in combination with rituximab according to the schedule shown in FIG. 18. P-BCMA-101 was administered on Day 0. Rituximab was administered 12 days and 5 days before P-BCMA-101, then every 8 weeks until myeloma progression. The pharmacokinetics of P-BCMA-101 were assessed by measuring the copies of P-BCMA-101/μg of DNA circulating in peripheral blood over time using qPCR. Anti-drug antibody (ADA) responses to P-BCMA-101 in peripheral blood were assessed over time using meso scale discovery (MSD) assay. In patient 102-006 without rituximab (FIG. 19A), an antibody response against P-BCMA-101 is seen, and P-BCMA-101 persists in peripheral blood for a short period of time accompanied by a short response of the myeloma. In patient 113-003 with rituximab (FIG. 19B), no antibody response against P-BCMA-101 is seen, and P-BCMA-101 persists in peripheral blood for a long period of time at a high level, accompanied by a long response of the myeloma (extending to the last time points assessed). While the peak P-BCMA-101 copies/ug DNA is at about 30 days for both patients (with or without rituximab), the P-BCMA-101 copies/ug DNA falls off to baseline at about 130 days in a patient treated without rituximab, whereas the P-BCMA-101 copies/ug DNA persists to the 190 days in a patient treated with rituximab. Furthermore, in the patient without rituximab, the clinical prognosis changes from stable disease to minimal response as the P-BCMA-101 copies/ug DNA decreases, and also has an ADA positive response. By contrast, in the patient with rituximab, the clinical prognosis changes from stable disease to partial response category as the P-BCMA-101 copies/ug DNA persists, and has an ADA negative response.

A total of nine patients were treated with a combination of P-BCMA-101 and rituximab and their response to treatment was annotated as the following: stringent complete response (sCR); complete response (CR); very good partial response (VGPR); partial response (PR); or minimal response (MR).

### INCORPORATION BY REFERENCE

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### OTHER EMBODIMENTS

While particular embodiments of the disclosure have been illustrated and described, various other changes and modifications can be made without departing from the spirit and scope of the disclosure. The scope of the appended claims includes all such changes and modifications that are within the scope of this disclosure.

What is claimed is:

1. A method of treating cancer comprising administering to the subject:
a first composition comprising a population of T-cells expressing a chimeric antigen receptor (CAR), wherein the CAR comprises an antigen recognition domain that specifically binds to B-cell maturation antigen (BCMA); and
a second composition comprising an anti-CD20 agent.

2. The method of claim 1, wherein there is an at least 50% decrease in anti-drug antibody (ADA) response against the first composition in the patient in comparison to a patient that is administered with the first composition but is not administered with the second composition.

3. The method of claim 1, wherein there is an at least 75% increase in persistence of the first composition in the patient in comparison to a patient that is administered with the first composition but is not administered with the second composition.

4. The method of claim 1, wherein there is an at least 90% increase in persistence of the first composition in the patient in comparison to a patient that is administered with the first composition but is not administered with the second composition.

5. The method of claim 3 or claim 4, wherein a measure of persistence is the area under the curve (AUC) of a plasma concentration curve.

6. The method of any one of the preceding claims, further comprising a third composition comprising at least one lymphodepletion agent.

7. The method of any one of the preceding claims, wherein the anti-CD20 agent is rituximab, ofatumumab, ocrelizumab, iodine i131 tositumomab, obinutuzumab or ibritumomab.

8. The method of claim 7, wherein the anti-CD20 agent is rituximab.

9. The method of any one of the preceding claims, wherein the antigen recognition domain comprises a Centyrin, an scFv, a single domain antibody, a VH or a VHH.

10. The method of claim 9, wherein the antigen binding domain comprises a Centyrin.

11. The method of claim 9, wherein the antigen binding domain comprises a VH.

12. The method of any one of the preceding claims, wherein the first composition is administered as multiple infusions, wherein the multiple infusion comprises a total dose that is split into a first infusion and a second infusion, and wherein
i) the first infusion comprises about one-third of the total dose; and
ii) the second infusion comprises about two-thirds of the total dose and is administered at least 10 days after the first infusion.

13. The method of any one of the preceding claims, wherein the first composition is administered as multiple infusions, wherein the multiple infusion comprises a total dose that is split into a first infusion, a second infusion and a third infusion, and wherein
i) the first infusion comprises about one-third of the total dose;
ii) the second infusion comprises about one-third of the total dose and is administered at least 10 days after the first infusion; and
iii) the third infusion comprises about one-third of the total dose and is administered at least 10 days after the second infusion.

14. The method of any one of claim 12 or 13, wherein the time in between the first infusion and the second infusion or the time in between the second infusion and the third infusion is at least 1 week, 2 weeks, 3 weeks, 4 weeks, 5 weeks or 6 weeks.

15. The method of any one of the preceding claims, wherein first composition, the second composition and/or the third composition are administered sequentially.

16. The method of any one of the preceding claims, wherein the first composition, the second composition and/or the second composition are administered concurrently.

17. The method of any one of claims 3-16, wherein the third composition is administered prior to the first composition.

18. The method of any one of claims 3-17, wherein the third composition is administered in more than one dose.

19. The method of claim 18, wherein the third composition is administered once a day and wherein the first dose of the third composition is administered at least 5 days prior to the first infusion of the first composition.

20. The method of claim 19, wherein the third composition is administered 3 days, 4 days and 5 days prior to the first infusion of the first composition.

21. The method of any one of the preceding claims, wherein the second composition is administered prior to the first composition.

22. The method of any one of the preceding claims, wherein the second composition is administered in more than one dose.

23. The method of claim 22, wherein a first dose of the second composition is administered at 12 days prior to the first infusion of the first composition, wherein a second dose of the second composition is administered at 5 days prior to the first infusion of the first composition, and wherein subsequent doses are administered once per week after the first infusion of the first composition for at least 8 weeks.

24. The method of any one of the preceding claims, wherein the subject has not previously been treated with an anti-cancer agent.

25. The method of any one of the preceding claims, wherein a first lymphodepletion agent of the third composition and a second lymphodepletion agent of the third composition is administered concurrently.

26. The method of any one of the preceding claims, wherein a first lymphodepletion agent of the third composition and a second lymphodepletion agent of the third composition are administered sequentially.

27. The method of claim 26, wherein the first lymphodepletion agent and the second lymphodepletion agent are administered on the same day, wherein the first lymphodepletion agent is administered intravenously over a 30 minute time period and wherein the second lymphodepletion agent is administered intravenously over a 30 minute time period.

28. The method of claim 26 or 27, wherein the first lymphodepletion agent or the second lymphodepletion agent is cyclophosphamide or fludarabine.

29. The method of claim 26, wherein a dose of the third composition comprises
i) 100 mg/m², 200 mg/m², 300 mg/m², 400 mg/m² or 500 mg/m² of cyclophosphamide;
ii) 10 mg/m², 20 mg/m², 30 mg/m², 40 mg/m² or 50 mg/m² of fludarabine; or a combination thereof.

30. The method of claim 29, wherein the dose of the third composition comprises 300 mg/m² of cyclophosphamide and 30 mg/m² of fludarabine.

31. The method of any one of the preceding claims, wherein the first composition is administered at a total dose of at least 0.1×10⁶, 0.2×10⁶, 0.25×10⁶, 0.5×10⁶, 0.6×10⁶, 0.7×10⁶, 0.75×10⁶, 0.8×10⁶, 0.9×10⁶, 1×10⁶, 2×10⁶, 3×10⁶, 4×10⁶, 5×10⁶, 6×10⁶, 7×10⁶, 8×10⁶, 9×10⁶, 10×10⁶, 11×10⁶, 12×10⁶, 13×10⁶, 14×10⁶, 15×10⁶, 16×10⁶, 17×10⁶, 18×10⁶, 19×10⁶ or 20×10⁶ cells/kg of the subject's body weight.

32. The method of any one of claims 12-31, wherein the first infusion, the second infusion and/or the third infusion of the first composition is administered using a infusion bag that comprises the first composition at a concentration of about 1×10⁵ cells/mL to about 5×10⁷ cells/mL.

33. The method of claim 32, wherein the infusion bag comprises the first composition at a concentration of about 3×10⁵ cells/mL to about 2.4×10⁷ cells/mL.

34. The method of any one of the preceding claims, wherein a dose of the second composition comprises 100 mg/m², 125 mg/m², 150 mg/m², 175 mg/m², 200 mg/m², 225 mg/m², 275 mg/m², 300 mg/m², 325 mg/m², 375 mg/m², 400 mg/m², 425 mg/m², 450 mg/m², 475 mg/m² or 500 mg/m² of rituximab.

35. The method of claim 34, wherein the dose of the second composition is 375 mg/m² of rituximab.

36. The method of claim 35, wherein the second composition is administered by intravenous infusion and wherein the flow rate of the intravenous infusion is about 25 mg/hr to about 500 mg/hr.

37. The method of claim 36, wherein the first dose of the second composition is administered by intravenous infusion at a flow rate of 50 mg/hr and wherein the flow rate is increased every 30 minutes to a maximum of 400 mg/hr.

38. The method of claim 36, wherein the second dose and the subsequent dose of the second composition is administered by intravenous infusion at a flow rate of about 100 mg/hr and wherein the flow rate is increased every 30 minutes to a maximum of about 400 mg/hr.

39. The method of any one of the preceding claims, wherein the cancer is a hematological cancer.

40. The method of claim 39, wherein the cancer is multiple myeloma.

41. The method of claim 40, wherein the multiple myeloma is relapsed multiple myeloma or refractory multiple myeloma.

42. A unit dose infusion bag comprising 250 mL of a composition comprising:
a population of T-cells expressing a CAR, wherein the CAR comprises an antigen recognition domain comprising a Centyrin that specifically binds to BCMA, wherein the concentration of the composition is about 3×10⁵ cells/mL to about 2.4×10⁷ cells/mL.

43. A unit dose infusion bag comprising 250 mL of a composition comprising:
a population of T-cells expressing a CAR, wherein the CAR comprises an antigen recognition domain comprising a VH that specifically binds to BCMA, wherein the concentration of the composition is about 3×10⁵ cells/mL to about 2.4×10⁷ cells/mL.
